(19) United States
(12) Patent Application Publication
(10) Pub. No.: US 2026/0199536 A1
(43) Pub. Date: Jul. 16, 2026

(54) MESOTHELIN-SPECIFIC BINDING CONSTRUCTS AND THEIR USE IN RADIOTHERAPY

(71) Applicants: MOLECULAR PARTNERS AG, Schlieren (CH); Orano Med Theranostics, Chatillon (FR)

(72) Inventors: Delphine BUFFET, Schlieren (CH); Clara DOMKE, Schlieren (CH); Stefanie RIESENBERG, Schlieren (CH); Amal SAIDI, Chatillon (FR); Julien TORGUE, Gaithersburg, MD (US); Aaron SCHATZMANN, Burleson, TX (US)

(21) Appl. No.: 19/445,918

(22) Filed: Jan. 12, 2026

(30) Foreign Application Priority Data
Jan. 10, 2025 (EP) 25305028.0
Jul. 30, 2025 (EP) 25306237.6

(51) Int. Cl. A61K 51/10; A61P 35/00

(52) U.S. Cl. CPC A61K 51/1093; A61K 51/1045; A61P 35/00; A61K 2121/00

(57) ABSTRACT

The present invention relates to MSLN-specific binding constructs comprising a designed ankyrin repeat domain with binding specificity for MSLN and a chelator capable of bonding to a radionuclide, as well as to such MSLN-specific binding constructs comprising a half-life extending moiety with binding specificity for serum albumin. The invention further relates to methods of producing such radio-labelled MSLN-specific binding constructs, pharmaceutical compositions comprising such constructs, and the use of such constructs or pharmaceutical compositions in methods for treating, imaging or diagnosing diseases, such as cancer.

### REFERENCE TO AN ELECTRONIC SEQUENCE LISTING

The instant application contains a Sequence Listing which has been submitted electronically in XML file format and is hereby incorporated by reference in its entirety. Said XML copy, created on Jan. 7, 2026, is named AJ4272_EPA_S3_sequencelisting.xml and is 66,310 bytes in size.

### CROSS-REFERENCE TO RELATED APPLICATIONS

This application claims priority from European Patent Application No. 25305028.0 filed on Jan. 10, 2025 and European Patent Application No. 25306237.6 filed Jul. 30, 2025. The priority of said European Patent Applications are claimed. Each of prior mentioned applications is hereby incorporated by reference herein in its entirety.

### FIELD

The present invention relates to mesothelin-specific binding constructs comprising a designed ankyrin repeat domain with binding specificity for mesothelin and a chelator capable of bonding to a radionuclide, such as Pb-212, as well as to such mesothelin-specific binding constructs comprising a half-life extending moiety with binding specificity for serum albumin. The invention further relates to methods of producing such radio-labelled mesothelin-specific binding constructs, pharmaceutical compositions comprising such constructs, and the use of such constructs or pharmaceutical compositions in methods for treating, imaging or diagnosing diseases, such as cancer.

### BACKGROUND

Mesothelin (MSLN) is a cell surface glycoprotein that is normally expressed in serosal tissues, such as pleura, pericardium and peritoneum, but not in the parenchyma of any vital organs. Mesothelin is overexpressed in a variety of cancers, and hence it is commonly expressed on the surface of tumor cells, e.g. in ovarian cancer, mesothelioma, pancreatic adenocarcinoma, gastric cancer, and other malignancies.

Due to its strong differential expression between normal and tumor tissues and its presence on the cell surface, mesothelin is considered a promising target for cancer therapy and diagnosis (Hagerty and Takabe, World J Oncol. 2023; 14(5):340-349).

The mesothelin gene encodes a precursor protein, known as pre-pro mesothelin, which is 622 amino acids long (69 kDa). Upon expression, this precursor protein is processed, among others by cleavage by the endoprotease furin, yielding a mature GPI-anchored, membrane-bound fragment of about 40 kDa (the membrane-bound mesothelin (MSLN)) and an about 31 kDa secreted fragment, also referred to as “megakaryocyte-potentiating factor” (MPF).

Similar to other GPI-anchored proteins, the membrane-bound mesothelin undergoes shedding in the extracellular environment, which is rich in proteases. Shedding can occur through proteolytic cleavage at one of several cleavage sites in the membrane-proximal region of mesothelin, resulting in truncated mesothelin protein that remains on the cell surface (tMSLN) as well as soluble mesothelin (sMSLN).

The specific biological function of mesothelin in normal tissue is still poorly understood. However, various studies have suggested that mesothelin plays a role in regulating tumor cell proliferation, dissemination, metastasis, and chemotherapy resistance (Chen et al., Discover Oncology 15:289 (2024); Hagerty and Takabe, World J Oncol. 2023; 14(5):340-349). Furthermore, the expression of mesothelin has also been linked to the prognosis of cancer patients. In many cases, higher levels of mesothelin expression correlate with more aggressive disease and poorer clinical outcomes.

Various different therapeutic approaches targeting mesothelin have been developed and explored in clinical trials for cancer treatment, including monoclonal antibodies, bispecific antibodies, immunotoxins, chimeric antigen receptor-T (CAR-T) cells, vaccines, and antibody-drug conjugates (ADCs) (Chen et al., Discover Oncology 15:289 (2024); Hagerty and Takabe, World J Oncol. 2023; 14(5):340-349; Hagerty et al., Biomolecules 2020, 10, 973; Chu, Curr Oncol Rep 2023 April; 25(4):309-323). However, in many cases, the therapeutic benefit has been modest and/or the administration has been limited by on-target, off-tumor toxicities. Some of these clinical programs were discontinued, mainly due to adverse effects or lack of efficacy. Taken together, there remains a need for new mesothelin-specific therapeutic and/or diagnostic agents and their use in treating and/or diagnosing diseases, such as cancer.

### SUMMARY OF THE INVENTION

Designing targeted radioisotope delivering platforms, including for alpha-particle emitting, beta-particle emitting or Auger electron emitting radioisotopes, and/or related drug candidates, requires simultaneous optimization of multiple aspects of such platforms or drug candidates. These aspects include, e.g., stability, target specificity, serum half-life, biodistribution, tissue penetration, pharmacodynamic properties, ease of manufacturing, acceptable therapeutic window and/or immunogenicity.

As an example, despite the excellent specificity of antibodies, such as IgGs, to their antigens, which makes antibodies an outstanding targeting platform for therapeutics, the typical serum half-life of an IgG of at least three weeks is disadvantageous for the delivery of radioisotopes, including alpha-emitting isotopes such as actinium-225 (225Ac or Ac-225) or lead-212 (212Pb or Pb-212) and beta-emitting isotopes such as lutetium-177 (177Lu or Lu-177) and yttrium-90 (90Y or Y-90), in particular due to prolonged exposure and chronic off-target toxicities. Smaller antibody formats (e.g. monomeric scFv's, heavy-chain only antibodies, or single-domain antibody fragments) with a molecular weight of, e.g., 15 to 30 kDa have been engineered, which provide similarly good specificity as a full-size antibody, such as an IgG (about 150 kDa), but have a much shorter serum half-life (e.g., 30 minutes to 2 hours). However, such short half-lives do not provide sufficient time for efficacious target binding due to poor retention and tumor uptake, and furthermore plasma clearance of such small antibody formats by the renal system can lead to isotope accumulation in renal tissues and problematic off target toxicities.

Thus, despite the general potential of targeted radioisotope delivering therapy, further elucidation of biochemical, immunological, pharmacological, and molecular aspects of targeted radioisotope delivering platforms must be pursued to better design and develop effective targeted radioisotope delivering drug candidates. In this pursuit, various aspects may play a role, including the choice of the target antigen, of the target-specific delivery system, of the radionuclide payload, of the chelator used to bind the radionuclide payload, of the chemistry used to connect the chelator to the delivery system, and/or of the molecular mechanism or entity used to modulate pharmacokinetic properties.

Applicant has found that designed ankyrin repeat proteins (DARPins) with binding specificity for MSLN can be formatted into targeted radioisotope delivering conjugates with beneficial properties. Such DARPin-based radioisotope delivering conjugates targeting MSLN, and methods of using such conjugates, are disclosed herein.

Based on the disclosure provided herein, those skilled in the art will recognize, or be able to ascertain using no more than routine experimentation, many equivalents to the specific embodiments of the invention described herein. Such equivalents are intended to be encompassed by the following embodiments (E).
E1. A conjugate or pharmaceutically acceptable salt thereof, the conjugate comprising (i) an ankyrin repeat domain with binding specificity for MSLN, (ii) a chelator, and (iii) a radionuclide.
E2. The conjugate or pharmaceutically acceptable salt thereof, of E1, wherein said chelator is covalently connected to said ankyrin repeat domain, wherein said radionuclide is bound to said chelator.
E3. The conjugate or pharmaceutically acceptable salt thereof, of E1 or E2, wherein said radionuclide is Ac-225, Lu-177, Pb-203, Pb-212, In-111, Tb-161, Cu-64, Cu-67, Zr-89, Y-86, or Ce-134.
E4. The conjugate or pharmaceutically acceptable salt thereof, of any one of E1 to E3, wherein said radionuclide is Ac-225, Lu-177, Pb-203, Pb-212, or In-111.
E5. A conjugate or pharmaceutically acceptable salt thereof, the conjugate comprising (i) an ankyrin repeat domain with binding specificity for MSLN, (ii) a chelator, and (iii) a radionuclide, wherein said chelator is covalently connected to said ankyrin repeat domain, wherein said radionuclide is bound to said chelator, and wherein said radionuclide is Ac-225.
E6. A conjugate or pharmaceutically acceptable salt thereof, the conjugate comprising (i) an ankyrin repeat domain with binding specificity for MSLN, (ii) a chelator, and (iii) a radionuclide, wherein said chelator is covalently connected to said ankyrin repeat domain, wherein said radionuclide is bound to said chelator, and wherein said radionuclide is Lu-177.
E7. A conjugate or pharmaceutically acceptable salt thereof, the conjugate comprising (i) an ankyrin repeat domain with binding specificity for MSLN, (ii) a chelator, and (iii) a radionuclide, wherein said chelator is covalently connected to said ankyrin repeat domain, wherein said radionuclide is bound to said chelator, and wherein said radionuclide is In-111.
E8. A conjugate or pharmaceutically acceptable salt thereof, the conjugate comprising (i) an ankyrin repeat domain with binding specificity for MSLN, (ii) a chelator, and (iii) a radionuclide, wherein said chelator is covalently connected to said ankyrin repeat domain, wherein said radionuclide is bound to said chelator, and wherein said radionuclide is Pb-212 or Pb-203.
E9. The conjugate or pharmaceutically acceptable salt of any one of E1 to E8, wherein said conjugate has the formula: M-Ch-R, wherein M is said ankyrin repeat domain with binding specificity for MSLN, Ch is said chelator, and R is said radionuclide.
E10 The conjugate or pharmaceutically acceptable salt of any one of E1 to E9, wherein said conjugate further comprises a connector, wherein said connector is covalently connected to said ankyrin repeat domain and to said chelator.
E11. The conjugate or pharmaceutically acceptable salt of E10, wherein said conjugate has the formula: M-Co-Ch-R, wherein M is said ankyrin repeat domain with binding specificity for MSLN, Co is said connector, Ch is said chelator, and R is said radionuclide.
E12. The conjugate or pharmaceutically acceptable salt of any one of E1 to E11, wherein said ankyrin repeat domain with binding specificity for MSLN binds human MSLN with a K_{D} value of or below 100 nM, of or below 10 nM, of or below 1 nM, of or below 350 μM, of or below 100 μM, of or below 35 μM, or of or below 10 μM.
E13. The conjugate or pharmaceutically acceptable salt of any one of E1 to E12, wherein said ankyrin repeat domain with binding specificity for MSLN comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 1 to 4, or 42 to 44.
E14. The conjugate or pharmaceutically acceptable salt of any one of E1 to E13, wherein said ankyrin repeat domain with binding specificity for MSLN comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 1.
E15. The conjugate or pharmaceutically acceptable salt of any one of E1 to E13, wherein said ankyrin repeat domain with binding specificity for MSLN comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 2.
E16. The conjugate or salt of any one of E1 to E13, wherein said ankyrin repeat domain with binding specificity for MSLN comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 3.
E17. The conjugate or pharmaceutically acceptable salt of any one of E1 to E13, wherein said ankyrin repeat domain with binding specificity for MSLN comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 4.
E18. The conjugate or pharmaceutically acceptable salt of any one of E1 to E13, wherein said ankyrin repeat domain with binding specificity for MSLN comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 42.
E19. The conjugate or pharmaceutically acceptable salt of any one of E1 to E13, wherein said ankyrin repeat domain with binding specificity for MSLN comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 43.
E20. The conjugate or pharmaceutically acceptable salt of any one of E1 to E13, wherein said ankyrin repeat domain with binding specificity for MSLN comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 44.
E21. The conjugate or pharmaceutically acceptable salt of any one E1 to E20, wherein said chelator is diethylenetriaminepentaacetic acid (DTPA), 1,4,7-triazacyclononane-1,4,7-triacetic acid (NOTA), 1,4,7,10-tetraazacyclododecane-1,4,7,10-tetraacetic acid (DOTA), 1,4,7,10-Tetraazacyclododecane-1,4,7,10-tetraacetamide (TCMC), or a derivative thereof.
E22. The conjugate or pharmaceutically acceptable salt of any one E1 to E21, wherein said chelator is DOTA (1,4,7,10-tetraazacyclododecane-1,4,7,10-tetraacetic acid) or TCMC (1,4,7,10-Tetraazacyclododecane-1,4,7,10-tetraacetamide), or a derivative thereof.
E23. The conjugate or pharmaceutically acceptable salt of any one of E1 to E22, wherein said chelator has a structure of Formula (I):

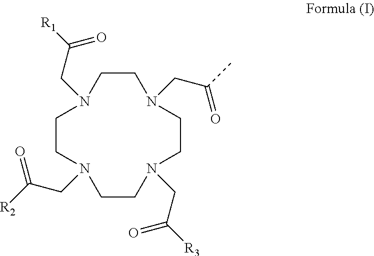

wherein R1, R2 and R3 are independently NH₂ or OH, and wherein the dotted line represents the covalent connection to said ankyrin repeat domain with binding specificity for MSLN or said connector.
E24. The conjugate or pharmaceutically acceptable salt of any one of E1 to E23, wherein said chelator has a structure of Formula (II):

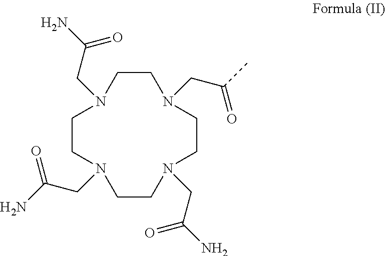

wherein the dotted line represents the covalent connection to said ankyrin repeat domain with binding specificity for MSLN or said connector.
E25. The conjugate or pharmaceutically acceptable salt of any one E1 to E22, wherein said chelator has a structure of Formula (III):

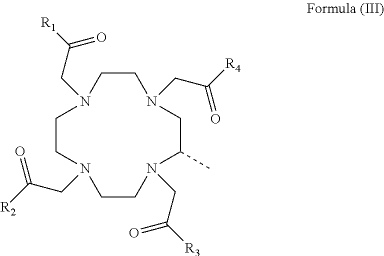

wherein R1, R2, R3 and R4 are independently NH₂ or OH, and wherein the dotted line represents the covalent connection to said ankyrin repeat domain with binding specificity for MSLN or said connector.
E26. The conjugate or pharmaceutically acceptable salt of any one of E1 to E22 and E25, wherein said chelator has a structure of Formula (IV):

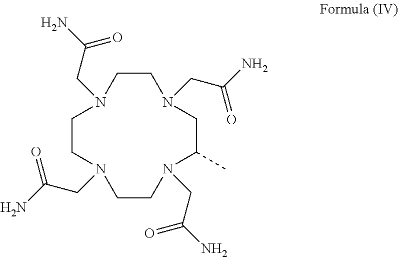 [SMILES: C[C@H]1CN(CC(N)=O)CCN(CC(N)=O)CCN(CC(N)=O)CCN1CC(N)=O]

wherein the dotted line represents the covalent connection to said ankyrin repeat domain with binding specificity for MSLN or said connector.
E27. The conjugate or pharmaceutically acceptable salt of any one of E1 to E26, further comprising a tag, wherein said tag comprises a Cysteine.
E28. The conjugate or pharmaceutically acceptable salt of E27, wherein said tag is located at the C-terminal side of said ankyrin repeat domain with binding specificity for MSLN.
E29. The conjugate or pharmaceutically acceptable salt of any one of E27 to E28, wherein said conjugate has the formula: M-T-Co-Ch-R, wherein M is said ankyrin repeat domain with binding specificity for MSLN, T is said tag, Co is said connector, Ch is said chelator, and R is said radionuclide.
E30. The conjugate or pharmaceutically acceptable salt of any one of E27 to E29, wherein said tag comprises the amino acid sequence of SEQ ID NO: 16 or a variant thereof.
E31. The conjugate or pharmaceutically acceptable salt of any one of E27 to E30, wherein said connector is covalently bound to said tag via a thioether bond.
E32. The conjugate or pharmaceutically acceptable salt of any one of E10 to E31, wherein said connector comprises a maleimide or a derivative thereof.
E33. The conjugate or pharmaceutically acceptable salt of any one of E10 to E31, wherein said connector has a structure of Formula (V):

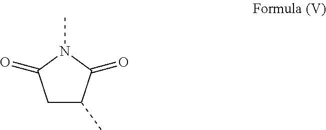

wherein the dotted line originating from N represents the covalent connection to said chelator, and wherein the dotted line originating from a carbon atom represents the covalent connection to said ankyrin repeat domain with binding specificity for MSLN or said tag.
E34. The conjugate or pharmaceutically acceptable salt of any one of E1 to E33, further comprising a half-life extending moiety.
E35. The conjugate or pharmaceutically acceptable salt of E34, wherein said conjugate has the formula: H-M-Ch-R, wherein H is said half-life extending moiety, M is said ankyrin repeat domain with binding specificity for MSLN, Ch is said chelator, and R is said radionuclide.
E36. The conjugate or pharmaceutically acceptable salt of E34, wherein said conjugate has the formula: M-H-Ch-R, wherein H is said half-life extending moiety, M is said ankyrin repeat domain with binding specificity for MSLN, Ch is said chelator, and R is said radionuclide.
E37. The conjugate or pharmaceutically acceptable salt of E34, wherein said conjugate has the formula: H-M-Co-Ch-R, wherein H is said half-life extending moiety, M is said ankyrin repeat domain with binding specificity for MSLN, Co is said connector, Ch is said chelator, and R is said radionuclide.
E38. The conjugate or pharmaceutically acceptable salt of E34, wherein said conjugate has the formula: H-M-T-Co-Ch-R, wherein H is said half-life extending moiety, M is said ankyrin repeat domain with binding specificity for MSLN, T is said tag, Co is said connector, Ch is said chelator, and R is said radionuclide.
E39. The conjugate or pharmaceutically acceptable salt of E34, wherein said conjugate has the formula: M-H-Co-Ch-R, wherein H is said half-life extending moiety, M is said ankyrin repeat domain with binding specificity for MSLN, Co is said connector, Ch is said chelator, and R is said radionuclide.
E40. The conjugate or pharmaceutically acceptable salt of E34, wherein said conjugate has the formula: M-H-T-Co-Ch-R, wherein H is said half-life extending moiety, M is said ankyrin repeat domain with binding specificity for MSLN, T is said tag, Co is said connector, Ch is said chelator, and R is said radionuclide.
E41. The conjugate or pharmaceutically acceptable salt of any one of E34 to E40, wherein said half-life extending moiety has binding specificity for human serum albumin.
E42. The conjugate or pharmaceutically acceptable salt of any one of E34 to E41, wherein said half-life extending moiety is an ankyrin repeat domain with binding specificity for human serum albumin.
E43. The conjugate or pharmaceutically acceptable salt of E42, wherein said ankyrin repeat domain with binding specificity for human serum albumin is covalently connected to said ankyrin repeat domain with binding specificity for MSLN at the C-terminal side of said ankyrin repeat domain with binding specificity for MSLN.
E44. The conjugate or pharmaceutically acceptable salt of any one of E42 to E43, wherein said ankyrin repeat domain with binding specificity for human serum albumin binds human serum albumin with a K_{D} value of or below 500 nM, of or below 250 nM, or of or below 100 nM.
E45. The conjugate or pharmaceutically acceptable salt of any one of E42 to E44, wherein said ankyrin repeat domain with binding specificity for human serum albumin comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 5 to 7 or 45.
E46. The conjugate or pharmaceutically acceptable salt of any one of E42 to E45, wherein said ankyrin repeat domain with binding specificity for human serum albumin comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 5.
E47. The conjugate or pharmaceutically acceptable salt of any one of E42 to E45, wherein said ankyrin repeat domain with binding specificity for human serum albumin comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 6.
E48. The conjugate or pharmaceutically acceptable salt of any one of E42 to E46, wherein said ankyrin repeat domain with binding specificity for human serum albumin comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 7.
E49. The conjugate or pharmaceutically acceptable salt of any one of E42 to E46, wherein said ankyrin repeat domain with binding specificity for human serum albumin comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 45.
E50. The conjugate or pharmaceutically acceptable salt of any one of E42 to E49, wherein said tag is located at the C-terminal side of said ankyrin repeat domain with binding specificity for human serum albumin.
E51. The conjugate or pharmaceutically acceptable salt of any one of E42 to E50, wherein said ankyrin repeat domain with binding specificity for human serum albumin is covalently connected to said ankyrin repeat domain with binding specificity for MSLN by a peptide linker.
E52. The conjugate or pharmaceutically acceptable salt of E51, wherein said peptide linker comprises the amino acid sequence of any one of SEQ ID NOs: 9 to 15 or a variant thereof.
E53. The conjugate or pharmaceutically acceptable salt of any one of E51 to E52, wherein said peptide linker comprises the amino acid sequence of SEQ ID NO: 10 or a variant thereof.
E54. The conjugate or pharmaceutically acceptable salt of any one of E51 to E52, wherein said peptide linker comprises the amino acid sequence of SEQ ID NO: 11 or a variant thereof.
E55. The conjugate or pharmaceutically acceptable salt of any one of E51 to E54, wherein said peptide linker has a length of 1 to 24 amino acids, of 1 to 22 amino acids, of 1 to 20 amino acids, of 1 to 19 amino acids, of 1 to 18 amino acids, of 1 to 17 amino acids, of 1 to 16 amino acids, of 1 to 15 amino acids, of 1 to 14 amino acids, of 1 to 13 amino acids, of 1 to 12 amino acids, of 1 to 11 amino acids, of 1 to 10 amino acids, of 1 to 9 amino acids, of 1 to 8 amino acids, of 1 to 7 amino acids, of 1 to 6 amino acids, of 1 to 5 amino acids, of 1 to 4 amino acids, of 1 to 3 amino acids, of 1 to 2 amino acids, or of 1 amino acid or about 2 to 20 amino acids, of about 2 to 15 amino acids, of about 2 to 11 amino acids, of about 2 to 10 amino acids, of about 2 to 9 amino acids, of about 2 to 8 amino acids, of about 3 to 11 amino acids, of about 3 to 10 amino acids, of about 3 to 9 amino acids, or of about 3 to 8 amino acids.
E56. The conjugate or pharmaceutically acceptable salt of any one of E51 to E55, wherein said conjugate has the formula: H-L-M-Co-Ch-R, wherein H is said half-life extending moiety, L is said peptide linker, M is said ankyrin repeat domain with binding specificity for MSLN, Co is said connector, Ch is said chelator, and R is said radionuclide.
E57. The conjugate or pharmaceutically acceptable salt of any of E51 to E55, wherein said conjugate has the formula: H-L-M-T-Co-Ch-R, wherein H is said half-life extending moiety, L is said peptide linker, M is said ankyrin repeat domain with binding specificity for MSLN, T is said tag, Co is said connector, Ch is said chelator, and R is said radionuclide.
E58. The conjugate or pharmaceutically acceptable salt of any one of E51 to E55, wherein said conjugate has the formula: M-L-H-Co-Ch-R, wherein H is said half-life extending moiety, L is said peptide linker, M is said ankyrin repeat domain with binding specificity for MSLN, Co is said connector, Ch is said chelator, and R is said radionuclide.
E59. The conjugate or pharmaceutically acceptable salt of any one of E51 to E55, wherein said conjugate has the formula: M-L-H-T-Co-Ch-R, wherein H is said half-life extending moiety, L is said peptide linker, M is said ankyrin repeat domain with binding specificity for MSLN, T is said tag, Co is said connector, Ch is said chelator, and R is said radionuclide.
E60. The conjugate or pharmaceutically acceptable salt of any one of E1 to E59, wherein said conjugate comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 17 to 26, or 46 to 50.
E61. The conjugate or pharmaceutically acceptable salt of any one of E1 to E60, wherein said conjugate or salt comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 17.
E62. The conjugate or pharmaceutically acceptable salt of any one of E1 to E60, wherein said conjugate or salt comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 18.
E63. The conjugate or pharmaceutically acceptable salt of any one of E1 to E60, wherein said conjugate or salt comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 19.
E64. The conjugate or pharmaceutically acceptable salt of any one of E1 to E60, wherein said conjugate or salt comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 20.
E65. The conjugate or pharmaceutically acceptable salt of any one of E1 to E60, wherein said conjugate or salt comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 21.
E66. The conjugate or pharmaceutically acceptable salt of any one of E1 to E60, wherein said conjugate or salt comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 22.
E67. The conjugate or pharmaceutically acceptable salt of any one of E1 to E60, wherein said conjugate or salt comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 23.
E68. The conjugate or pharmaceutically acceptable salt of any one of E1 to E60, wherein said conjugate or salt comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 24.
E69. The conjugate or pharmaceutically acceptable salt of any one of E1 to E60, wherein said conjugate or salt comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 25.
E70. The conjugate or pharmaceutically acceptable salt of any one of E1 to E60, wherein said conjugate or salt comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 26.
E71. The conjugate or pharmaceutically acceptable salt of any one of E1 to E60, wherein said conjugate or salt comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 46.
E72. The conjugate or pharmaceutically acceptable salt of any one of E1 to E60, wherein said conjugate or salt comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 47.
E73. The conjugate or pharmaceutically acceptable salt of any one of E1 to E60, wherein said conjugate or salt comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 48.
E74. The conjugate or pharmaceutically acceptable salt of any one of E1 to E60, wherein said conjugate or salt comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 49.
E75. The conjugate or pharmaceutically acceptable salt of any one of E1 to E60, wherein said conjugate or salt comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 50.
E76. The conjugate or pharmaceutically acceptable salt of any one of E60 to E75, wherein said amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 17 to 26, or 46 to 50 is covalently bound at its N-terminal end to Glycine-Serine (GS).
E77. The conjugate or pharmaceutically acceptable salt of any one of E60 to E76, wherein said amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 17 to 26, or 46 to 50 is covalently bound at its C-terminal end to the amino acid sequence of SEQ ID NO: 16.
E78. A conjugate or pharmaceutically acceptable salt thereof, the conjugate comprising (i) an ankyrin repeat protein comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 29 to 34, 38, or 51 to 56, (ii) a connector, (iii) a chelator, and (iv) a radionuclide, wherein said ankyrin repeat protein has binding specificity for MSLN and for human serum albumin, wherein said connector is covalently connected to said ankyrin repeat protein, wherein said chelator is covalently connected to said connector, wherein said radionuclide is bound to said chelator, and wherein said radionuclide is Ac-225, Lu-177, Pb-203, Pb-212, In-111, Tb-161, Cu-64, Cu-67, Zr-89, Y-86, or Ce-134, preferably Ac-225, Lu-177, Pb-203, Pb-212, or In-111.
E79. A conjugate or pharmaceutically acceptable salt thereof, the conjugate comprising (i) an ankyrin repeat protein comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 29 to 34, 38, or 51 to 56, (ii) a connector, (iii) a chelator, and (iv) a radionuclide, wherein said ankyrin repeat protein has binding specificity for MSLN and for human serum albumin, wherein said connector is covalently connected to said ankyrin repeat protein, wherein said chelator is covalently connected to said connector, wherein said radionuclide is bound to said chelator, and wherein said radionuclide is Pb-212 or Pb-203, or wherein said radionuclide is Lu-177 or Ac-225.
E80. The conjugate or pharmaceutically acceptable salt of E78 or E79, wherein said conjugate comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 29.
E81. The conjugate or pharmaceutically acceptable salt of E78 or E79, wherein said conjugate comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 30.
E82. The conjugate or pharmaceutically acceptable salt of E78 or E79, wherein said conjugate comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 31.
E83. The conjugate or pharmaceutically acceptable salt of E78 or E79, wherein said conjugate comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 32.
E84. The conjugate or pharmaceutically acceptable salt of E78 or E79, wherein said conjugate comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 33.
E85. The conjugate or pharmaceutically acceptable salt of E78 or E79, wherein said conjugate comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 34.
E86. The conjugate or pharmaceutically acceptable salt of E78 or E79, wherein said conjugate comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 38.
E87. The conjugate or pharmaceutically acceptable salt of E78 or E79, wherein said conjugate comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 51.
E88. The conjugate or pharmaceutically acceptable salt of E78 or E79, wherein said conjugate comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 52.
E89. The conjugate or pharmaceutically acceptable salt of E78 or E79, wherein said conjugate comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 53.
E90. The conjugate or pharmaceutically acceptable salt of E78 or E79, wherein said conjugate comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 54.
E91. The conjugate or pharmaceutically acceptable salt of E78 or E79, wherein said conjugate comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 55.
E92. The conjugate or pharmaceutically acceptable salt of E78 or E79, wherein said conjugate comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 56.
E93. The conjugate or pharmaceutically acceptable salt of any one of E78 to E92, wherein said ankyrin repeat protein comprises a Cysteine, wherein said connector comprises maleimide or a derivative thereof, and wherein said Cysteine is covalently bound to said connector via a thioether bond.
E94. The conjugate or pharmaceutically acceptable salt of E93, wherein said Cysteine is located at the C-terminal end of said amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 29 to 34, 38, or 51 to 56.
E95. The conjugate or pharmaceutically acceptable salt of any one of E78 to E94, wherein said chelator is diethylenetriaminepentaacetic acid (DTPA), 1,4,7-triazacyclononane-1,4,7-triacetic acid (NOTA), 1,4,7,10-tetraazacyclododecane-1,4,7,10-tetraacetic acid (DOTA), 1,4,7,10-Tetraazacyclododecane-1,4,7,10-tetraacetamide (TCMC), or a derivative thereof.
E96. The conjugate or pharmaceutically acceptable salt of any one of E78 to E95, wherein said chelator is DOTA (1,4,7,10-tetraazacyclododecane-1,4,7,10-tetraacetic acid) or TCMC (1,4,7,10-Tetraazacyclododecane-1,4,7,10-tetraacetamide), or a derivative thereof.
E97. The conjugate or pharmaceutically acceptable salt of any one of E78 to E96, wherein said chelator has a structure of Formula (I):

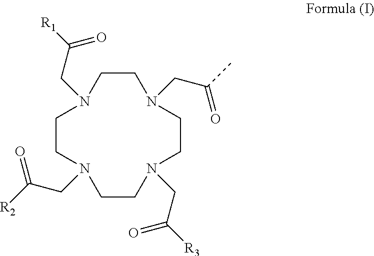

wherein R1, R2 and R3 are independently NH₂ or OH, and wherein the dotted line represents the covalent connection to said connector.
E98. The conjugate or pharmaceutically acceptable salt of any one of E78 to E97, wherein said chelator has a structure of Formula (II):

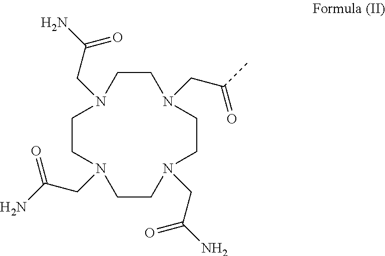

wherein the dotted line represents the covalent connection to said connector.
E99. The conjugate or pharmaceutically acceptable salt of any one of E78 to E96, wherein said chelator has a structure of Formula (III):

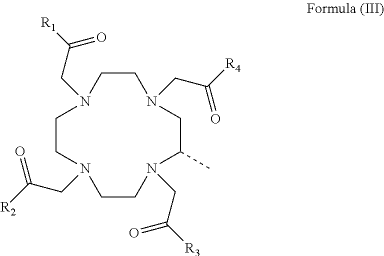

wherein R1, R2, R3 and R4 are independently NH₂ or OH, and wherein the dotted line represents the covalent connection to said connector.
E100. The conjugate or pharmaceutically acceptable salt of any one of E78 to E96 and E99, wherein said chelator has a structure of Formula (IV):

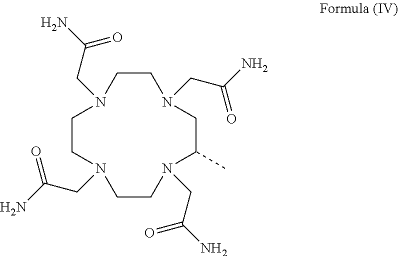 [SMILES: C[C@H]1CN(CC(N)=O)CCN(CC(N)=O)CCN(CC(N)=O)CCN1CC(N)=O]

wherein the dotted line represents the covalent connection to said connector.
E101. A conjugate or pharmaceutically acceptable salt thereof, wherein said conjugate has a structure of Formula (VI):

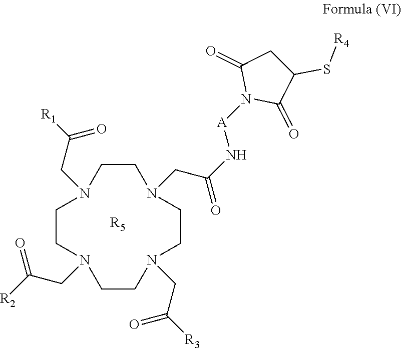

wherein R1, R2, and R3 are independently NH-2 or OH;
wherein A is CₐH_{b}N_{c}O_{d}, wherein a, b, c, and d are integers;
wherein R4 is an ankyrin repeat protein comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 29 to 34, 38, or 51 to 56, wherein said ankyrin repeat protein has binding specificity for MSLN and for human serum albumin, and wherein said ankyrin repeat protein comprises a Cysteine, and wherein R5 is a chelated radionuclide, wherein said radionuclide is Ac-225, Lu-177, Pb-203, Pb-212, In-111, Tb-161, Cu-64, Cu-67, Zr-89, Y-86, or Ce-134, preferably Ac-225, Lu-177, Pb-203, Pb-212, or In-111.
E102. A conjugate or pharmaceutically acceptable salt thereof, wherein said conjugate has a structure of Formula (VI):

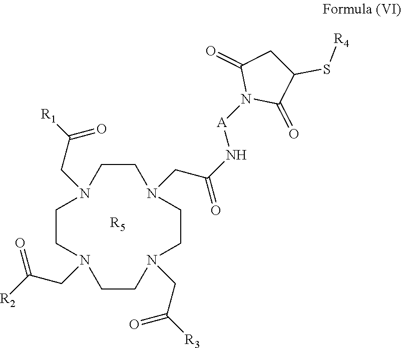

wherein R1, R2, and R3 are independently NH₂ or OH;
wherein A is CₐH_{b}N_{c}O_{d}, wherein a, b, c, and d are integers;
wherein R4 is an ankyrin repeat protein comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 29 to 34, 38, or 51 to 56, wherein said ankyrin repeat protein has binding specificity for MSLN and for human serum albumin, and wherein said ankyrin repeat protein comprises a Cysteine;
and wherein R5 is a chelated radionuclide, wherein said radionuclide is Pb-212 or Pb-203.
E103. A conjugate or pharmaceutically acceptable salt thereof, wherein said conjugate has a structure of Formula (VI):

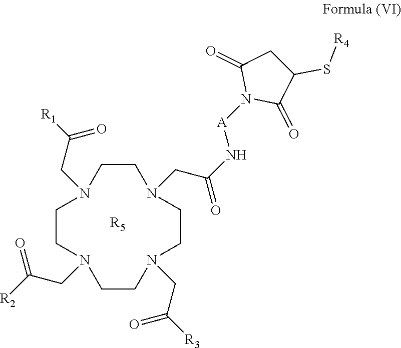

wherein R1, R2, and R3 are independently NH₂ or OH;
wherein A is CₐH_{b}N_{c}O_{d}, wherein a, b, c, and d are integers;
wherein R4 is an ankyrin repeat protein comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 29 to 34, 38, or 51 to 56, wherein said ankyrin repeat protein has binding specificity for MSLN and for human serum albumin, and wherein said ankyrin repeat protein comprises a Cysteine, and wherein R5 is a chelated radionuclide, wherein said radionuclide is Ac-225 or Lu-177.
E104. The conjugate or pharmaceutically acceptable salt of any one of E101 to E103, wherein said Cysteine is connected to a heterocyclic ring structure by a thioether bond, wherein said heterocyclic ring structure connects A and R4.
E105. A conjugate or pharmaceutically acceptable salt thereof, wherein said conjugate has a structure of Formula (VII):

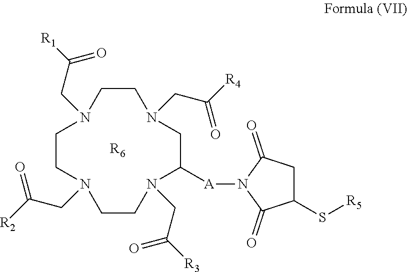

wherein R1, R2, R3 and R4 are independently NH₂ or OH;
wherein A is CₐH_{b}N_{c}O_{d}, wherein a, b, c, and d are integers;
wherein R5 is an ankyrin repeat protein comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 29 to 34, 38, or 51 to 56, wherein said ankyrin repeat protein has binding specificity for MSLN and for human serum albumin, and wherein said ankyrin repeat protein comprises a Cysteine, and wherein R6 is a chelated radionuclide, wherein said radionuclide is Ac-225, Lu-177, Pb-203, Pb-212, In-111, Tb-161, Cu-64, Cu-67, Zr-89, Y-86, or Ce-134, preferably Ac-225, Lu-177, Pb-203, Pb-212, or In-111.
E106. A conjugate or pharmaceutically acceptable salt thereof, wherein said conjugate has a structure of Formula (VII):

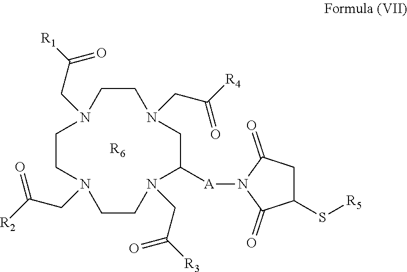

wherein R1, R2, R3 and R4 are independently NH₂ or OH;
wherein A is CₐH_{b}N_{c}O_{d}, wherein a, b, c, and d are integers;
wherein R5 is an ankyrin repeat protein comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 29 to 34, 38, 51 to 56 wherein said ankyrin repeat protein has binding specificity for MSLN and for human serum albumin, and wherein said ankyrin repeat protein comprises a Cysteine;
and wherein R6 is a chelated radionuclide, wherein said radionuclide is Pb-212 or Pb-203.
E107. A conjugate or pharmaceutically acceptable salt thereof, wherein said conjugate has a structure of Formula (VII):

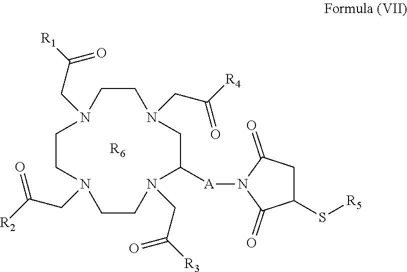

wherein R1, R2, R3 and R4 are independently NH₂ or OH;
wherein A is CₐH_{b}N_{c}O_{d}, wherein a, b, c, and d are integers;
wherein R5 is an ankyrin repeat protein comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 29 to 34, 38, or 51 to 56, wherein said ankyrin repeat protein has binding specificity for MSLN and for human serum albumin, and wherein said ankyrin repeat protein comprises a Cysteine, and wherein R6 is a chelated radionuclide, wherein said radionuclide is Ac-225 or Lu-177.
E108. The conjugate or pharmaceutically acceptable salt of any one of E105 to E107, wherein said Cysteine is connected to a heterocyclic ring structure by a thioether bond, wherein said heterocyclic ring structure connects A and R5.
E109. The conjugate or pharmaceutically acceptable salt of any one of E101 to E108, wherein said conjugate or salt comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 29.
E110. The conjugate or pharmaceutically acceptable salt of any one of E101 to E108, wherein said conjugate or salt comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 30.
E111. The conjugate or pharmaceutically acceptable salt of any one of E101 to E108, wherein said conjugate or salt comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 31.
E112. The conjugate or pharmaceutically acceptable salt of any one of E101 to E108, wherein said conjugate or salt comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 32.
E113. The conjugate or pharmaceutically acceptable salt of any one of E101 to E108, wherein said conjugate or salt comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 33.
E114. The conjugate or pharmaceutically acceptable salt of any one of E101 to E108, wherein said conjugate or salt comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 34.
E115. The conjugate or pharmaceutically acceptable salt of any one of E101 to E108, wherein said conjugate or salt comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 38.
E116. The conjugate or pharmaceutically acceptable salt of any one of E101 to E108, wherein said conjugate or salt comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 51.
E117. The conjugate or pharmaceutically acceptable salt of any one of E101 to E108, wherein said conjugate or salt comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 52.
E118. The conjugate or pharmaceutically acceptable salt of any one of E101 to E108, wherein said conjugate or salt comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 53.
E119. The conjugate or pharmaceutically acceptable salt of any one of E101 to E108, wherein said conjugate or salt comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 54.
E120. The conjugate or pharmaceutically acceptable salt of any one of E101 to E108, wherein said conjugate or salt comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 55.
E121. The conjugate or pharmaceutically acceptable salt of any one of E101 to E108, wherein said conjugate or salt comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 56.
E122. The conjugate or pharmaceutically acceptable salt of any one of E101 to E121, wherein said Cysteine is located at the C-terminal end of said amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 29 to 34, 38, or 51 to 56.
E123. The conjugate or pharmaceutically acceptable salt of any one of E78 to E122, wherein said ankyrin repeat protein binds human MSLN with a K_{D} value of or below 100 nM, of or below 10 nM, of or below 1 nM, of or below 350 μM, of or below 100 μM, of or below 35 μM, or of or below 10 μM.
E124. The conjugate or salt of any one of E78 to E123, wherein said ankyrin repeat protein binds human serum albumin with a K_{D} value of or below 500 nM, of or below 250 nM, or of or below 100 nM.
E125. The conjugate or pharmaceutically acceptable salt of any one of E1 to E124, wherein said conjugate binds to cells expressing human MSLN on their surface.
E126. The conjugate or pharmaceutically acceptable salt of any one of E1 to E125, wherein said conjugate does not specifically bind to human soluble MSLN (sMSLN).
E127. The conjugate or pharmaceutically acceptable salt of any one of E1 to E126, wherein said conjugate binds to cells expressing human MSLN on their surface, and wherein said cells are OVCAR-3 cells or OVCAR-8 cells.
E128. The conjugate or pharmaceutically acceptable salt of E127, wherein said conjugate binds said OVCAR-3 cells with an EC50 of about or below 5×10⁻⁷M, of about or below 1.5×10⁻⁷M, of about or below 5×10⁻⁸M, of about or below 1.5×10⁻⁸M, of about or below 5×10⁻⁹M, of about or below 2×10⁻⁹M, of about or below 10⁻⁹M, or of about or below 5×10⁻¹⁰M.
E129. The conjugate or pharmaceutically acceptable salt of any one of E1 to E128, wherein said conjugate binds to cells expressing human MSLN on their surface, and wherein said binding to cells expressing human MSLN on their surface is not inhibited by the presence of human soluble MSLN (sMSLN).
E130. The conjugate or pharmaceutically acceptable salt of any one of E1 to E129, wherein said conjugate has a terminal half-life in a mouse model of at least about 5 hours, at least about 7.5 hours, at least about 10 hours, at least about 12.5 hours, at least about 15 hours, at least about 17.5 hours, at least about 20 hours, at least about 22.5 hours, at least about 25 hours, at least about 27.5 hours, at least about 30 hours, or at least about 32.5 hours.
E131. The conjugate or pharmaceutically acceptable salt of any one of E1 to E130, wherein said conjugate has a terminal half-life in a mouse model of about 5 to 45 hours, about 5 to 40 hours, about 5 to 35 hours, about 10 to 30 hours, or about 13 to 27 hours.
E132. The conjugate or pharmaceutically acceptable salt of any one of E130 and E131, wherein said mouse model is a BALB/c mouse model.
E133. The conjugate or pharmaceutically acceptable salt of any one of E1 to E132, wherein said radionuclide is Pb-212 or Ac-225, and wherein said conjugate is capable of inhibiting tumor growth in a human MSLN-expressing mouse tumor model.
E134. The conjugate or pharmaceutically acceptable salt of E133, wherein said human MSLN-expressing mouse tumor model comprises tumors formed by human MSLN-expressing MC38 colon carcinoma cells, by Capan-2 pancreatic carcinoma cells, by OVCAR-3 ovarian carcinoma cells, or by OVCAR-8 ovarian carcinoma cells.
E135. A pharmaceutical composition comprising the conjugate or pharmaceutically acceptable salt of any one of E1 to E134, and optionally a pharmaceutically acceptable carrier, excipient, stabilizer and/or diluent.
E136. A kit comprising (i) a first container containing the conjugate or pharmaceutically acceptable salt of any one of E1 to E134 or the pharmaceutical composition of E135; and (ii) a second container containing a buffered solution.
E137. The conjugate or pharmaceutically acceptable salt of any of E1 to E134 or the pharmaceutical composition of E135 for use in a method of imaging, diagnosing and/or treating a medical condition, the method comprising the step of administering to a subject in need thereof an amount of said conjugate or salt or said pharmaceutical composition effective for imaging, diagnosing and/or treating said medical condition.
E138. The conjugate or pharmaceutically acceptable salt or the pharmaceutical composition for use in a method of imaging, diagnosing and/or treating a medical condition of E137, wherein said medical condition is cancer.
E139. The conjugate or pharmaceutically acceptable salt or the pharmaceutical composition for use in a method of imaging, diagnosing and/or treating a medical condition of E138, wherein said cancer comprises cells that express MSLN on their surface.
E140. The conjugate or pharmaceutically acceptable salt or the pharmaceutical composition for use in a method of imaging, diagnosing and/or treating a medical condition of any one of E138 to E139, wherein said cancer is ovarian cancer, mesothelioma, pancreatic cancer, gastric cancer, or breast cancer.
E141. The conjugate or pharmaceutically acceptable salt or the pharmaceutical composition for use in a method of imaging, diagnosing and/or treating a medical condition of any one of E138 to E140, wherein said cancer is epithelial ovarian cancer, epithelioid mesothelioma, pancreatic adenocarcinoma, gastric adenocarcinoma, or triple negative breast cancer.
E142. The conjugate or pharmaceutically acceptable salt or the pharmaceutical composition for use in a method of imaging, diagnosing and/or treating a medical condition of any one of E138 to E141, wherein said cancer is ovarian cancer.
E143. The conjugate or pharmaceutically acceptable salt or the pharmaceutical composition for use in a method of imaging, diagnosing and/or treating a medical condition of any one of E138 to E141, wherein said cancer is pancreatic cancer.
E144. The conjugate or pharmaceutically acceptable salt or the pharmaceutical composition for use in a method of imaging, diagnosing and/or treating a medical condition of any one of E137 to E143, wherein said subject is a mammal, preferably a human.
E145. The conjugate or pharmaceutically acceptable salt or the pharmaceutical composition for use in a method of imaging, diagnosing and/or treating a medical condition of any one of E137 to E144, wherein said radionuclide is Ac-225, Lu-177, Pb-203, Pb-212, In-111, Tb-161, Cu-64, Cu-67, Zr-89, Y-86, or Ce-134, preferably Ac-225, Lu-177, Pb-203, Pb-212, or In-111.
E146. The conjugate or pharmaceutically acceptable salt or the pharmaceutical composition for use in a method of imaging, diagnosing and/or treating a medical condition of any one of E137 to E145, wherein said radionuclide is Pb-212.
E147. The conjugate or pharmaceutically acceptable salt or the pharmaceutical composition for use in a method of imaging, diagnosing and/or treating a medical condition of any one of E137 to E145, wherein said radionuclide is Pb-203.
E148. The conjugate or pharmaceutically acceptable salt or the pharmaceutical composition for use in a method of imaging, diagnosing and/or treating a medical condition of any one of E137 to E145, wherein said radionuclide is Ac-225.
E149. The conjugate or pharmaceutically acceptable salt or the pharmaceutical composition for use in a method of imaging, diagnosing and/or treating a medical condition of any one of E137 to E145, wherein said radionuclide is Lu-177.
E150. The conjugate or pharmaceutically acceptable salt or the pharmaceutical composition for use in a method of imaging, diagnosing and/or treating a medical condition of any one of E137 to E145, wherein said radionuclide is In-111.
E151. The conjugate or pharmaceutically acceptable salt or the pharmaceutical composition for use in a method of imaging, diagnosing and/or treating a medical condition of any one of E137 to E145, wherein said radionuclide is Tb-161.
E152. The conjugate or pharmaceutically acceptable salt or the pharmaceutical composition for use in a method of imaging, diagnosing and/or treating a medical condition of any one of E137 to E145, wherein said radionuclide is Cu-64.
E153. The conjugate or pharmaceutically acceptable salt or the pharmaceutical composition for use in a method of imaging, diagnosing and/or treating a medical condition of any one of E137 to E145, wherein said radionuclide is Cu-67.
E154. The conjugate or pharmaceutically acceptable salt or the pharmaceutical composition for use in a method of imaging, diagnosing and/or treating a medical condition of any one of E137 to E145, wherein said radionuclide is Zr-89.
E155. The conjugate or pharmaceutically acceptable salt or the pharmaceutical composition for use in a method of imaging, diagnosing and/or treating a medical condition of any one of E137 to E145, wherein said radionuclide is Y-86.
E156. The conjugate or pharmaceutically acceptable salt or the pharmaceutical composition for use in a method of imaging, diagnosing and/or treating a medical condition of any one of E137 to E145, wherein said radionuclide is Ce-134.
E157. A method of treating a medical condition, the method comprising the step of administering to a subject in need thereof a therapeutically effective amount of the conjugate or pharmaceutically acceptable salt of any one of E1 to E134 or the pharmaceutical composition of E135.
E158. The method of E157, wherein said radionuclide is Ac-225, Lu-177, Tb-161, Pb-212 or Cu-67.
E159. The method of E157, wherein said radionuclide is Pb-212.
E160. The method of E157, wherein said radionuclide is Ac-225.
E161. The method of E157, wherein said radionuclide is Lu-177.
E162. The method of E157, wherein said radionuclide is Tb-161.
E163. The method of E157, wherein said radionuclide is Cu-67.
E164. A method of imaging and/or diagnosing a medical condition, the method comprising the steps of: (i) administering to a subject an amount of the conjugate or pharmaceutically acceptable salt of any one of E1 to E134, or of the pharmaceutical composition of E135, effective for binding of the conjugate or salt to cells expressing MSLN on their surface, and (ii) detecting cells bound by the conjugate or salt thereof or tissues comprising cells bound by the conjugate or salt thereof.
E165. The method of E164, wherein said detecting in step (ii) is performed by in vivo imaging.
E166. The method of E165, wherein said in vivo imaging uses single photon emission computed tomography (SPECT).
E167. The method of any one of E164 to E166, wherein said radionuclide is In-111, Cu-64, Zr-89, Y-86, Lu-177, Tb-161, Pb-203 or Ce-134.
E168. The method of any one of E164 to E167, wherein said radionuclide is Pb-203.
E169. The method of any one of E164 to E167, wherein said radionuclide is Lu-177.E170. The method of any one of E157 to E169, wherein said medical condition is cancer.
E171. The method of E170, wherein said cancer comprises cells that express MSLN on their surface.
E172. The method of any one of E170 to E171, wherein said cancer is ovarian cancer, mesothelioma, pancreatic cancer, gastric cancer, or breast cancer.
E173. The method of any one of E170 to E172, wherein said cancer is epithelial ovarian cancer, epithelioid mesothelioma, pancreatic adenocarcinoma, gastric adenocarcinoma, or triple negative breast cancer.
E174. The method of any one of E170 to E173, wherein said cancer is ovarian cancer.
E175. The method of any one of E170 to E173, wherein said cancer is pancreatic cancer.
E176. The method of any one of E157 to E175, wherein said subject is a mammal, preferably a human.
E177. Use of the conjugate or pharmaceutically acceptable salt thereof, of any of E1 to E134 or the pharmaceutical composition of E135 in the manufacture of a medicament.
E178. Use of the conjugate or pharmaceutically acceptable salt thereof or the pharmaceutical composition of E177, wherein said medicament is for treatment of cancer, optionally for treatment of ovarian cancer, mesothelioma, pancreatic cancer, gastric cancer, or breast cancer.
E179. A method of manufacturing a medicament for the treatment of a medical condition, wherein the conjugate or pharmaceutically acceptable salt of any one of E1 to E134 or the pharmaceutical composition of E135 is an active ingredient of said medicament.
E180. The method of manufacturing a medicament for the treatment of a medical condition of E179, wherein said medical condition is cancer, optionally ovarian cancer, mesothelioma, pancreatic cancer, gastric cancer, or breast cancer.

### BRIEF DESCRIPTION OF THE DRAWINGS

FIGS. 1A and 1B: Illustration of the structures of exemplary embodiments of the MSLN-specific conjugates disclosed herein. FIG. 1A: R1, R2, and R3 are independently NH₂ or OH; A is CₐH_{b}N_{c}O_{d}, wherein a, b, c, and d are integers; R4 is an ankyrin repeat protein comprising the amino acid sequence of any one of SEQ ID NOs: 29 to 34, 38, or 51 to 56 or a variant thereof; R5 is a chelated radionuclide, either Pb-212 or Pb-203. FIG. 1B: R1, R2, R3 and R4 are independently NH₂ or OH; A is CₐH_{b}N_{c}O_{d}, wherein a, b, c, and d are integers; R5 is an ankyrin repeat protein comprising the amino acid sequence of any one of SEQ ID NOs: 29 to 34, 38, or 51 to 56 or a variant thereof; R6 is a chelated radionuclide, either Pb-212 or Pb-203.

FIGS. 2A to 2H: Binding of different concentrations of selected single domain (1D) and two domain (2D) DARPins to Ovcar-3 cells and MC38 cells expressing human MSLN (hMSLN-MC38), in absence and presence of 10 μM human serum albumin. Anetumab was used as a positive control, and a non-binding DARPin (SEQ ID NO: 41) as a negative control. DARPin #1, DARPin #11 and DARPin #13 on Ovcar-3 cells, with HSA (FIG. 2A) and without HSA (FIG. 2C), and on hMSLN-MC38 cells, with HSA (FIG. 2E) and without HSA (FIG. 2G). DARPin #4, DARPin #16 and DARPin #18 on Ovcar-3 cells, with HSA (FIG. 2B) and without HSA (FIG. 2D), and on hMSLN-MC38 cells, with HSA (FIG. 2F) and without HSA (FIG. 2H).

FIG. 3: Binding of different concentrations of selected two domain (2D) DARPins (DARPin #25 and DARPin #26), which have a different positioning of the two ankyrin repeat domains within the proteins, to Ovcar-3 cells, in absence and presence of 10 μM human serum albumin.

FIGS. 4A to 4D: Binding of different concentrations of selected two domain (2D) DARPins, which comprise different linkers connecting the two ankyrin repeat domains, to Ovcar-3 cells and hMSLN-MC38 cells, in absence of human serum albumin. DARPin #10, DARPin #11, DARPin #12, and DARPin #13 on hMSLN-MC38 cells (FIG. 4A) and Ovcar-3 cells (FIG. 4B); DARPin #14, DARPin #16, DARPin #17, and DARPin #18 on hMSLN-MC38 cells (FIG. 4C) and Ovcar-3 cells (FIG. 4D).

FIGS. 5A and 5B: In vivo biodistribution of Pb-212 labelled DARPin conjugates. Radio-labelled DARPin (DARPin #21, DARPin #24) conjugate was injected iv. at 0.01 mg/kg (10 μCi) into athymic nude mice xenografted subcutaneously with hMSLN-MC38 cells. Blood, small intestine, colon, kidneys, liver, lung, heart, tail and tumor were collected and weighed 4 h and 24 h post injection and radioactivity of each sample was measured using a y-counter instrument (Wizard²2470, Perkin Elmer). FIG. 5A: DARPin #21 conjugate; FIG. 5B: DARPin #24 conjugate.

FIG. 6: Pharmacokinetic analysis of natural lead-labelled DARPin conjugates in WT mice. Natural lead-labeled DARPin conjugates (DARPin #21, DARPin #24, DARPin #27) were injected i.v. at 0.01 mg/kg or 1 mg/kg into the tail vein of WT Athymic nude mice. Serum was collected 5 min, 4 h, 24 h, 48 h, 72 h, 96 h and 168 h post injection. DARPin was detected and measured by ELISA. LLOQ: Lower Limit of Quantification.

FIG. 7: Pharmacokinetic analysis of natural lead-labelled DARPin conjugates in tumor-bearing mice. Athymic nude mice were subcutaneously engrafted with hMSLN-MC38 cells, and once tumor volumes reached the predetermined volume (~300-500 mm³), animals were randomized and injected with DARPins. Natural lead-labeled DARPin conjugates (DARPin #21, DARPin #27) were injected i.v. at 0.01 mg/kg or 1 mg/kg into the tail vein of the tumor-bearing mice. Serum was collected 4 h, 24 h, 72 h and 96 h post injection. DARPin was detected and measured by ELISA. LLOQ: Lower Limit of Quantification.

FIGS. 8A to 8C: Binding of different concentrations of selected two domain (2D) DARPins to MC38 cells expressing human MSLN (hMSLN-MC38) (FIG. 8A) and OVCAR-3 cells (FIG. 8B, 8C) in absence and presence of 10 μM human serum albumin. DARPin #33, DARPin #34, DARPin #35, and DARPin #11 with and without HSA binding to hMSLN-MC38 cells (FIG. 8A) and to OVCAR-3 cells (FIG. 8B). His-tagged or DOTAM-conjugated DARPin #33 (His-tagged), DARPin #38 (DOTAM-conjugated), DARPin #35 (His-tagged), DARPin #40 (DOTAM-conjugated), and DARPin #19 (His-tagged) binding to OVCAR-3 cells in the presence of 10 μM human serum albumin (HSA). Anetumab and 15B6 were used as a positive control. MFI: mean fluorescence intensity.

### DETAILED DESCRIPTION OF THE INVENTION

Designed ankyrin repeat domains are structural units of designed ankyrin repeat proteins. Designed repeat protein libraries, including designed ankyrin repeat protein libraries (WO2002020565; Binz et al., Nat. Biotechnol. 22, 575-582, 2004; Stumpp et al., Drug Discov. Today 13, 695-701, 2008), can be used for the selection of target-specific designed repeat domains that bind to their target with high affinity. Such target-specific designed repeat domains in turn can be used as valuable components of recombinant binding proteins for the treatment and/or diagnosis of diseases.

Designed ankyrin repeat proteins are a class of binding molecules which have the potential to overcome limitations of monoclonal antibodies, hence allowing novel therapeutic and/or diagnostic approaches. Such ankyrin repeat proteins may comprise a single designed ankyrin repeat domain, or may comprise a combination of two, three, four, five or more designed ankyrin repeat domains with the same or different target specificities (Stumpp et al., Drug Discov. Today 13, 695-701, 2008; U.S. Pat. No. 9,458,211). Ankyrin repeat proteins comprising only a single designed ankyrin repeat domain are small proteins (14 kDa) which can be selected to bind a given target protein with high affinity and specificity. These characteristics, and the possibility of combining two, three, four, five or more designed ankyrin repeat domains in one protein, make designed ankyrin repeat proteins ideal agonistic, antagonistic and/or inhibitory drug candidates. Furthermore, such ankyrin repeat proteins can be engineered to carry various effector functions, e.g. cytotoxic agents or half-life extending agents, enabling completely new drug formats. Taken together, designed ankyrin repeat proteins are an example of the next generation of protein therapeutics with the potential to surpass existing antibody drugs.

Various approaches have been explored to harness the potential of mesothelin as a therapeutic target, including antibody-drug conjugates (ADCs), immunotherapies including chimeric antigen receptor (CAR) T-cell therapy, and targeted drug delivery systems. However, soluble mesothelin (sMSLN) can act as a significant antigen sink and thereby reduce the efficacy of therapeutic agents targeting mesothelin. At the same time, targeting membrane-proximal epitopes of mesothelin, which are part of the truncated mesothelin protein (tMSLN) that remains on the cell surface after shedding and/or which are not part of soluble mesothelin (sMSLN), is not amenable to all therapeutic approaches.

The inventors of the present invention have found that designed ankyrin repeat domains with binding specificity for MSLN can be covalently combined with other moieties to form MSLN-specific binding constructs that can be loaded with various radionuclides, including actinium-225 (²²⁵Ac, Ac-225, t_{1/2}/2=9.92 days), lutetium-177 (¹⁷⁷Lu or Lu-177, t_{1/2}=6.647 days), and the theranostic pair of lead-203 (²⁰³Pb or Pb-203, t_{1/2}=51.9 h) and lead-212 (²¹²Pb or Pb-212, t_{1/2}=10.6 h). These radio-labelled MSLN-specific DARPin conjugates have beneficial properties that make them useful for applications in imaging, diagnosing and/or treating medical conditions characterized by MSLN expression on the surface of cells, such as certain cancers. Actinium-225 is an alpha emitter with a short range (a few cell diameters) of alpha particles in tissue and high energy, making it effective in targeting and killing cancer cells. The approximately 10-day half-life of 225Ac is long enough to facilitate treatment, but short enough that little remains in the body months after treatment. Lutetium-177 (Lu177) is a medium-energy β-emitter (Emax of 498.3 keV) and low-energy gamma emitter (Ey max of 208 keV) with maximal tissue penetration of <3 mm of diameter, enabling localized cytotoxic radiation in relatively small tumors as well as metastatic lesions. Additionally, Lu177's relatively long half-life, allows delivery to sites distant from the reactor production facility with minimal decay loss. Lead-212 serves as an in vivo generator of alpha-particle emitters through its daughter nuclides (²¹²Bi and ²¹²Po), which undergo a-decay The higher linear-energy transfer of alpha-particles (compared to beta-particles) may result in increased incidence of double-strand DNA breaks and improved localized cancer cell damage, while Pb-203 provides imaging capabilities for the theranostic pair. Moreover, the inventors were able to design MSLN-specific DARPin conjugates with binding specificity to membrane-proximal epitopes of mesothelin, which are part of the truncated mesothelin protein (tMSLN) that remains on the cell surface after shedding and/or which are not part of soluble mesothelin (sMSLN). These MSLN-specific DARPin conjugates of the invention bind to membrane-bound mesothelin (full length or truncated forms) on the cell surface, but do not specifically bind to soluble mesothelin (sMSLN) and hence are not affected by a sMSLN antigen sink.

Conjugates or Pharmaceutically Acceptable Salts Thereof In one aspect, provided is a conjugate or pharmaceutically acceptable salt thereof, the conjugate comprising (i) an ankyrin repeat domain with binding specificity for MSLN, (ii) a chelator, and (iii) a radionuclide. In one embodiment, said chelator is covalently connected to said ankyrin repeat domain, wherein said radionuclide is bound to said chelator. Said radionuclide is Ac-225, Lu-177, Pb-203, Pb-212, In-111, Tb-161, Cu-64, Cu-67, Zr-89, Y-86, or Ce-134, preferably Ac-225, Lu-177, Pb-203, Pb-212, or In-111.

Accordingly, provided is a conjugate or pharmaceutically acceptable salt thereof, the conjugate comprising (i) an ankyrin repeat domain with binding specificity for MSLN, (ii) a chelator, and (iii) a radionuclide, wherein said chelator is covalently connected to said ankyrin repeat domain, wherein said radionuclide is bound to said chelator, and wherein said radionuclide is Ac-225.

Also provided is a conjugate or pharmaceutically acceptable salt thereof, the conjugate comprising (i) an ankyrin repeat domain with binding specificity for MSLN, (ii) a chelator, and (iii) a radionuclide, wherein said chelator is covalently connected to said ankyrin repeat domain, wherein said radionuclide is bound to said chelator, and wherein said radionuclide is Lu-177.

Also provided is a conjugate or pharmaceutically acceptable salt thereof, the conjugate comprising (i) an ankyrin repeat domain with binding specificity for MSLN, (ii) a chelator, and (iii) a radionuclide, wherein said chelator is covalently connected to said ankyrin repeat domain, wherein said radionuclide is bound to said chelator, and wherein said radionuclide is In-111.

In one main aspect, the invention relates to a conjugate or pharmaceutically acceptable salt thereof, the conjugate comprising (i) an ankyrin repeat domain with binding specificity for MSLN, (ii) a chelator, and (iii) a radionuclide, wherein said chelator is covalently connected to said ankyrin repeat domain, wherein said radionuclide is bound to said chelator, and wherein said radionuclide is Pb-212 or Pb-203. In one embodiment, said radionuclide is Pb-212. In another embodiment, said radionuclide is Pb-203.

In another aspect, the invention relates to such a conjugate or pharmaceutically acceptable salt thereof, wherein said conjugate has the formula: M-Ch-R, wherein M is said ankyrin repeat domain with binding specificity for MSLN, Ch is said chelator, and R is said radionuclide. Different methods of covalently connecting a polypeptide to a chelator have been described (see, e.g., in Morais and Ma, Drug Discovery Today: Technologies, Antibody—Drug Conjugates (ADC), Vol. 30, pp. 91-104, 2018; Tsuchikama and An, Protein Cell 2018, 9(1):33-46; Kang et al., Chem. Sci., 2021, 12, 13613).

In another aspect, the invention relates to a conjugate or pharmaceutically acceptable salt thereof, the conjugate comprising (i) an ankyrin repeat domain with binding specificity for MSLN, (ii) a connector, (iii) a chelator, and (iv) a radionuclide, wherein said connector is covalently connected to said ankyrin repeat domain, wherein said chelator is covalently connected to said connector, wherein said radionuclide is bound to said chelator, and wherein said radionuclide is Pb-212 or Pb-203. In one embodiment, said radionuclide is Pb-212. In another embodiment, said radionuclide is Pb-203.

In another aspect, the invention relates to a conjugate or pharmaceutically acceptable salt thereof, the conjugate comprising (i) an ankyrin repeat domain with binding specificity for MSLN, (ii) a connector, (iii) a chelator, and (iv) a radionuclide, wherein said connector is covalently connected to said ankyrin repeat domain, wherein said chelator is covalently connected to said connector, wherein said radionuclide is bound to said chelator, and wherein said radionuclide is Ac-225, Lu-177, In-111, Tb-161, Cu-64, Cu-67, Zr-89, Y-86, or Ce-134. In one embodiment, said radionuclide is Ac-225. In another embodiment, said radionuclide is Lu-177. In another embodiment, said radionuclide is In-111.

In another aspect, the invention relates to such a conjugate or pharmaceutically acceptable salt thereof, wherein said conjugate has the formula: M-Co-Ch-R, wherein M is said ankyrin repeat domain with binding specificity for MSLN, Co is said connector, Ch is said chelator, and R is said radionuclide.

In another aspect, the invention relates to such a conjugate or pharmaceutically acceptable salt thereof, wherein said ankyrin repeat domain with binding specificity for MSLN binds human MSLN (hMSLN) with a K_{D} value of or below 100 nM, of or below 30 nM, of or below 10 nM, of or below 3 nM, or of or below 1 nM, or of or below 350 μM, of or below 300 μM, of or below 100 μM, of or below 35 μM, of or below 30 μM, or of or below 10 μM. Thus, in one embodiment, said ankyrin repeat domain binds hMSLN with a K_{D} value of or below 100 nM. In one embodiment, said ankyrin repeat domain binds hMSLN with a K_{D} value of or below 30 nM. In one embodiment, said ankyrin repeat domain binds hMSLN with a K_{D} value of or below 10 nM. In one embodiment, said ankyrin repeat domain binds hMSLN with a K_{D} value of or below 3 nM. In one embodiment, said ankyrin repeat domain binds hMSLN with a K_{D} value or of or below 1 nM. In one embodiment, said ankyrin repeat domain binds hMSLN with a K_{D} value of or below 350 μM. In one embodiment, said ankyrin repeat domain binds hMSLN with a K_{D} value of or below 300 μM. In one embodiment, said ankyrin repeat domain binds hMSLN with a K_{D} value of or below 100 μM. In one embodiment, said ankyrin repeat domain binds hMSLN with a K_{D} value of or below 35 μM. In one embodiment, said ankyrin repeat domain binds hMSLN with a K_{D} value of or below 30 μM. In one embodiment, said ankyrin repeat domain binds hMSLN with a K_{D} value of or below 10 μM. Furthermore, in one embodiment, said ankyrin repeat domain binds to the extracellular domain of hMSLN. In one embodiment, said ankyrin repeat domain binds to a membrane-proximal epitope of mesothelin, which is part of truncated mesothelin protein (tMSLN) that remains on the cell surface after shedding and/or is not part of soluble mesothelin (sMSLN).

In another aspect, the invention relates to such a conjugate or pharmaceutically acceptable salt thereof, wherein said ankyrin repeat domain with binding specificity for MSLN comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 1 to 4, or 42 to 44. Thus, in one embodiment, said ankyrin repeat domain comprises an amino acid sequence that is at least 80% identical to any one of SEQ ID NOs: 1 to 4, or 42 to 44. In one embodiment, said ankyrin repeat domain comprises an amino acid sequence that is at least 85% identical to any one of SEQ ID NOs: 1 to 4, or 42 to 44. In one embodiment, said ankyrin repeat domain comprises an amino acid sequence that is at least 88% identical to any one of SEQ ID NOs: 1 to 4, or 42 to 44. In one embodiment, said ankyrin repeat domain comprises an amino acid sequence that is at least 90% identical to any one of SEQ ID NOs: 1 to 4, or 42 to 44. In one embodiment, said ankyrin repeat domain comprises an amino acid sequence that is at least 91% identical to any one of SEQ ID NOs: 1 to 4, or 42 to 44. In one embodiment, said ankyrin repeat domain comprises an amino acid sequence that is at least 92% identical to any one of SEQ ID NOs: 1 to 4, or 42 to 44. In one embodiment, said ankyrin repeat domain comprises an amino acid sequence that is at least 93% identical to any one of SEQ ID NOs: 1 to 4, or 42 to 44. In one embodiment, said ankyrin repeat domain comprises an amino acid sequence that is at least 94% identical to any one of SEQ ID NOs: 1 to 4, or 42 to 44. In one embodiment, said ankyrin repeat domain comprises an amino acid sequence that is at least 95% identical to any one of SEQ ID NOs: 1 to 4, or 42 to 44. In one embodiment, said ankyrin repeat domain comprises an amino acid sequence that is at least 96% identical to any one of SEQ ID NOs: 1 to 4, or 42 to 44. In one embodiment, said ankyrin repeat domain comprises an amino acid sequence that is at least 97% identical to any one of SEQ ID NOs: 1 to 4, or 42 to 44. In one embodiment, said ankyrin repeat domain comprises an amino acid sequence that is at least 98% identical to any one of SEQ ID NOs: 1 to 4, or 42 to 44. In one embodiment, said ankyrin repeat domain comprises an amino acid sequence that is at least 99% identical to any one of SEQ ID NOs: 1 to 4, or 42 to 44. In one embodiment, any amino acid sequence differences between said ankyrin repeat domain and said any one of SEQ ID NOs: 1 to 4, or 42 to 44 represent amino acid substitutions in framework positions. In one embodiment, said ankyrin repeat domain comprises an amino acid sequence that is 100% identical to any one of SEQ ID NOs: 1 to 4, or 42 to 44.

In another aspect, the invention relates to such a conjugate or pharmaceutically acceptable salt thereof, wherein said ankyrin repeat domain with binding specificity for MSLN comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 1. Thus, in one embodiment, said ankyrin repeat domain comprises an amino acid sequence that is at least 80% identical to SEQ ID NO: 1. In one embodiment, said ankyrin repeat domain comprises an amino acid sequence that is at least 81% identical to SEQ ID NO: 1. In one embodiment, said ankyrin repeat domain comprises an amino acid sequence that is at least 82% identical to SEQ ID NO: 1. In one embodiment, said ankyrin repeat domain comprises an amino acid sequence that is at least 83% identical to SEQ ID NO: 1. In one embodiment, said ankyrin repeat domain comprises an amino acid sequence that is at least 84% identical to SEQ ID NO: 1. In one embodiment, said ankyrin repeat domain comprises an amino acid sequence that is at least 85% identical to SEQ ID NO: 1. In one embodiment, said ankyrin repeat domain comprises an amino acid sequence that is at least 86% identical to SEQ ID NO: 1. In one embodiment, said ankyrin repeat domain comprises an amino acid sequence that is at least 87% identical to SEQ ID NO: 1. In one embodiment, said ankyrin repeat domain comprises an amino acid sequence that is at least 88% identical to SEQ ID NO: 1. In one embodiment, said ankyrin repeat domain comprises an amino acid sequence that is at least 89% identical to SEQ ID NO: 1. In one embodiment, said ankyrin repeat domain comprises an amino acid sequence that is at least 90% identical to SEQ ID NO: 1. In one embodiment, said ankyrin repeat domain comprises an amino acid sequence that is at least 91% identical to SEQ ID NO: 1. In one embodiment, said ankyrin repeat domain comprises an amino acid sequence that is at least 92% identical to SEQ ID NO: 1. In one embodiment, said ankyrin repeat domain comprises an amino acid sequence that is at least 93% identical SEQ ID NO: 1. In one embodiment, said ankyrin repeat domain comprises an amino acid sequence that is at least 94% identical to SEQ ID NO: 1. In one embodiment, said ankyrin repeat domain comprises an amino acid sequence that is at least 95% identical to SEQ ID NO: 1. In one embodiment, said ankyrin repeat domain comprises an amino acid sequence that is at least 96% identical to SEQ ID NO: 1. In one embodiment, said ankyrin repeat domain comprises an amino acid sequence that is at least 97% identical to SEQ ID NO: 1. In one embodiment, said ankyrin repeat domain comprises an amino acid sequence that is at least 98% identical to SEQ ID NO: 1. In one embodiment, said ankyrin repeat domain comprises an amino acid sequence that is at least 99% identical to SEQ ID NO: 1. In one embodiment, any amino acid sequence differences between said ankyrin repeat domain and SEQ ID NO: 1 represent amino acid substitutions in framework positions. In one embodiment, said ankyrin repeat domain comprises an amino acid sequence that is 100% identical to SEQ ID NO: 1.

In another aspect, the invention relates to such a conjugate or pharmaceutically acceptable salt thereof, wherein said ankyrin repeat domain with binding specificity for MSLN comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 2. Thus, in one embodiment, said ankyrin repeat domain comprises an amino acid sequence that is at least 80% identical to SEQ ID NO: 2. In one embodiment, said ankyrin repeat domain comprises an amino acid sequence that is at least 85% identical to SEQ ID NO: 2. In one embodiment, said ankyrin repeat domain comprises an amino acid sequence that is at least 88% identical to SEQ ID NO: 2. In one embodiment, said ankyrin repeat domain comprises an amino acid sequence that is at least 90% identical to SEQ ID NO: 2. In one embodiment, said ankyrin repeat domain comprises an amino acid sequence that is at least 91% identical to SEQ ID NO: 2. In one embodiment, said ankyrin repeat domain comprises an amino acid sequence that is at least 92% identical to SEQ ID NO: 2. In one embodiment, said ankyrin repeat domain comprises an amino acid sequence that is at least 93% identical SEQ ID NO: 2. In one embodiment, said ankyrin repeat domain comprises an amino acid sequence that is at least 94% identical to SEQ ID NO: 2. In one embodiment, said ankyrin repeat domain comprises an amino acid sequence that is at least 95% identical to SEQ ID NO: 2. In one embodiment, said ankyrin repeat domain comprises an amino acid sequence that is at least 96% identical to SEQ ID NO: 2. In one embodiment, said ankyrin repeat domain comprises an amino acid sequence that is at least 97% identical to SEQ ID NO: 2. In one embodiment, said ankyrin repeat domain comprises an amino acid sequence that is at least 98% identical to SEQ ID NO: 2. In one embodiment, said ankyrin repeat domain comprises an amino acid sequence that is at least 99% identical to SEQ ID NO: 2. In one embodiment, any amino acid sequence differences between said ankyrin repeat domain and SEQ ID NO: 2 represent amino acid substitutions in framework positions. In one embodiment, said ankyrin repeat domain comprises an amino acid sequence that is 100% identical to SEQ ID NO: 2.

In another aspect, the invention relates to such a conjugate or pharmaceutically acceptable salt thereof, wherein said ankyrin repeat domain with binding specificity for MSLN comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 3. Thus, in one embodiment, said ankyrin repeat domain comprises an amino acid sequence that is at least 80% identical to SEQ ID NO: 3. In one embodiment, said ankyrin repeat domain comprises an amino acid sequence that is at least 85% identical to SEQ ID NO: 3. In one embodiment, said ankyrin repeat domain comprises an amino acid sequence that is at least 88% identical to SEQ ID NO: 3. In one embodiment, said ankyrin repeat domain comprises an amino acid sequence that is at least 90% identical to SEQ ID NO: 3. In one embodiment, said ankyrin repeat domain comprises an amino acid sequence that is at least 91% identical to SEQ ID NO: 3. In one embodiment, said ankyrin repeat domain comprises an amino acid sequence that is at least 92% identical to SEQ ID NO: 3. In one embodiment, said ankyrin repeat domain comprises an amino acid sequence that is at least 93% identical SEQ ID NO: 3. In one embodiment, said ankyrin repeat domain comprises an amino acid sequence that is at least 94% identical to SEQ ID NO: 3. In one embodiment, said ankyrin repeat domain comprises an amino acid sequence that is at least 95% identical to SEQ ID NO: 3. In one embodiment, said ankyrin repeat domain comprises an amino acid sequence that is at least 96% identical to SEQ ID NO: 3. In one embodiment, said ankyrin repeat domain comprises an amino acid sequence that is at least 97% identical to SEQ ID NO: 3. In one embodiment, said ankyrin repeat domain comprises an amino acid sequence that is at least 98% identical to SEQ ID NO: 3. In one embodiment, said ankyrin repeat domain comprises an amino acid sequence that is at least 99% identical to SEQ ID NO: 3. In one embodiment, any amino acid sequence differences between said ankyrin repeat domain and SEQ ID NO: 3 represent amino acid substitutions in framework positions. In one embodiment, said ankyrin repeat domain comprises an amino acid sequence that is 100% identical to SEQ ID NO: 3.

In another aspect, the invention relates to such a conjugate or pharmaceutically acceptable salt thereof, wherein said ankyrin repeat domain with binding specificity for MSLN comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 4. Thus, in one embodiment, said ankyrin repeat domain comprises an amino acid sequence that is at least 80% identical to SEQ ID NO: 4. In one embodiment, said ankyrin repeat domain comprises an amino acid sequence that is at least 85% identical to SEQ ID NO: 4. In one embodiment, said ankyrin repeat domain comprises an amino acid sequence that is at least 88% identical to SEQ ID NO: 4. In one embodiment, said ankyrin repeat domain comprises an amino acid sequence that is at least 90% identical to SEQ ID NO: 4. In one embodiment, said ankyrin repeat domain comprises an amino acid sequence that is at least 91% identical to SEQ ID NO: 4. In one embodiment, said ankyrin repeat domain comprises an amino acid sequence that is at least 92% identical to SEQ ID NO: 4. In one embodiment, said ankyrin repeat domain comprises an amino acid sequence that is at least 93% identical SEQ ID NO: 4. In one embodiment, said ankyrin repeat domain comprises an amino acid sequence that is at least 94% identical to SEQ ID NO: 4. In one embodiment, said ankyrin repeat domain comprises an amino acid sequence that is at least 95% identical to SEQ ID NO: 4. In one embodiment, said ankyrin repeat domain comprises an amino acid sequence that is at least 96% identical to SEQ ID NO: 4. In one embodiment, said ankyrin repeat domain comprises an amino acid sequence that is at least 97% identical to SEQ ID NO: 4. In one embodiment, said ankyrin repeat domain comprises an amino acid sequence that is at least 98% identical to SEQ ID NO: 4. In one embodiment, said ankyrin repeat domain comprises an amino acid sequence that is at least 99% identical to SEQ ID NO: 4. In one embodiment, any amino acid sequence differences between said ankyrin repeat domain and SEQ ID NO: 4 represent amino acid substitutions in framework positions. In one embodiment, said ankyrin repeat domain comprises an amino acid sequence that is 100% identical to SEQ ID NO: 4.

In one embodiment, said ankyrin repeat domain with binding specificity for MSLN comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 42. Thus, in one embodiment, said ankyrin repeat domain comprises an amino acid sequence that is at least 80% identical to SEQ ID NO: 42. In one embodiment, said ankyrin repeat domain comprises an amino acid sequence that is at least 85% identical to SEQ ID NO: 42. In one embodiment, said ankyrin repeat domain comprises an amino acid sequence that is at least 88% identical to SEQ ID NO: 42. In one embodiment, said ankyrin repeat domain comprises an amino acid sequence that is at least 90% identical to SEQ ID NO: 42. In one embodiment, said ankyrin repeat domain comprises an amino acid sequence that is at least 91% identical to SEQ ID NO: 42. In one embodiment, said ankyrin repeat domain comprises an amino acid sequence that is at least 92% identical to SEQ ID NO: 42. In one embodiment, said ankyrin repeat domain comprises an amino acid sequence that is at least 93% identical SEQ ID NO: 42. In one embodiment, said ankyrin repeat domain comprises an amino acid sequence that is at least 94% identical to SEQ ID NO: 42. In one embodiment, said ankyrin repeat domain comprises an amino acid sequence that is at least 95% identical to SEQ ID NO: 42. In one embodiment, said ankyrin repeat domain comprises an amino acid sequence that is at least 96% identical to SEQ ID NO: 42. In one embodiment, said ankyrin repeat domain comprises an amino acid sequence that is at least 97% identical to SEQ ID NO: 42. In one embodiment, said ankyrin repeat domain comprises an amino acid sequence that is at least 98% identical to SEQ ID NO: 42. In one embodiment, said ankyrin repeat domain comprises an amino acid sequence that is at least 99% identical to SEQ ID NO: 42. In one embodiment, any amino acid sequence differences between said ankyrin repeat domain and SEQ ID NO: 42 represent amino acid substitutions in framework positions. In one embodiment, said ankyrin repeat domain comprises an amino acid sequence that is 100% identical to SEQ ID NO: 42.

In one embodiment, said ankyrin repeat domain with binding specificity for MSLN comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 43. Thus, in one embodiment, said ankyrin repeat domain comprises an amino acid sequence that is at least 80% identical to SEQ ID NO: 43. In one embodiment, said ankyrin repeat domain comprises an amino acid sequence that is at least 85% identical to SEQ ID NO: 43. In one embodiment, said ankyrin repeat domain comprises an amino acid sequence that is at least 88% identical to SEQ ID NO: 43. In one embodiment, said ankyrin repeat domain comprises an amino acid sequence that is at least 90% identical to SEQ ID NO: 43. In one embodiment, said ankyrin repeat domain comprises an amino acid sequence that is at least 91% identical to SEQ ID NO: 43. In one embodiment, said ankyrin repeat domain comprises an amino acid sequence that is at least 92% identical to SEQ ID NO: 43. In one embodiment, said ankyrin repeat domain comprises an amino acid sequence that is at least 93% identical SEQ ID NO: 43. In one embodiment, said ankyrin repeat domain comprises an amino acid sequence that is at least 94% identical to SEQ ID NO: 43. In one embodiment, said ankyrin repeat domain comprises an amino acid sequence that is at least 95% identical to SEQ ID NO: 43. In one embodiment, said ankyrin repeat domain comprises an amino acid sequence that is at least 96% identical to SEQ ID NO: 43. In one embodiment, said ankyrin repeat domain comprises an amino acid sequence that is at least 97% identical to SEQ ID NO: 43. In one embodiment, said ankyrin repeat domain comprises an amino acid sequence that is at least 98% identical to SEQ ID NO: 43. In one embodiment, said ankyrin repeat domain comprises an amino acid sequence that is at least 99% identical to SEQ ID NO: 43. In one embodiment, any amino acid sequence differences between said ankyrin repeat domain and SEQ ID NO: 43 represent amino acid substitutions in framework positions. In one embodiment, said ankyrin repeat domain comprises an amino acid sequence that is 100% identical to SEQ ID NO: 43.

In one embodiment, said ankyrin repeat domain with binding specificity for MSLN comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 44. Thus, in one embodiment, said ankyrin repeat domain comprises an amino acid sequence that is at least 80% identical to SEQ ID NO: 44. In one embodiment, said ankyrin repeat domain comprises an amino acid sequence that is at least 85% identical to SEQ ID NO: 44. In one embodiment, said ankyrin repeat domain comprises an amino acid sequence that is at least 88% identical to SEQ ID NO: 44. In one embodiment, said ankyrin repeat domain comprises an amino acid sequence that is at least 90% identical to SEQ ID NO: 44. In one embodiment, said ankyrin repeat domain comprises an amino acid sequence that is at least 91% identical to SEQ ID NO: 44. In one embodiment, said ankyrin repeat domain comprises an amino acid sequence that is at least 92% identical to SEQ ID NO: 44. In one embodiment, said ankyrin repeat domain comprises an amino acid sequence that is at least 93% identical SEQ ID NO: 44. In one embodiment, said ankyrin repeat domain comprises an amino acid sequence that is at least 94% identical to SEQ ID NO: 44. In one embodiment, said ankyrin repeat domain comprises an amino acid sequence that is at least 95% identical to SEQ ID NO: 44. In one embodiment, said ankyrin repeat domain comprises an amino acid sequence that is at least 96% identical to SEQ ID NO: 44. In one embodiment, said ankyrin repeat domain comprises an amino acid sequence that is at least 97% identical to SEQ ID NO: 44. In one embodiment, said ankyrin repeat domain comprises an amino acid sequence that is at least 98% identical to SEQ ID NO: 44. In one embodiment, said ankyrin repeat domain comprises an amino acid sequence that is at least 99% identical to SEQ ID NO: 44. In one embodiment, any amino acid sequence differences between said ankyrin repeat domain and SEQ ID NO: 44 represent amino acid substitutions in framework positions. In one embodiment, said ankyrin repeat domain comprises an amino acid sequence that is 100% identical to SEQ ID NO: 44.

In another aspect, the invention relates to such a conjugate or pharmaceutically acceptable salt thereof, wherein said ankyrin repeat domain with binding specificity for MSLN comprises an amino acid sequence selected from the group consisting of (1) any one of SEQ ID NOs: 1 to 4, or 42 to 44 and (2) sequences in which up to 25, or up to 24, or up to 23, or up to 22, or up to 21, or up to 20, or up to 19, or up to 18, or up to 17, or up to 16, or up to 15, or up to 14, or up to 13, or up to 12, or up to 11, or up to 10, or up to 9, or up to 8, or up to 7, or up to 6, or up to 5, or up to 4, or up to 3, or up to 2, or up to 1 amino acids in any one of SEQ ID NOs: 1 to 4, or 42 to 44 are substituted by other amino acids. Thus, in one embodiment, said ankyrin repeat domain comprises an amino acid sequence selected from the group consisting of (1) any one of SEQ ID NOs: 1 to 4, or 42 to 44 and (2) sequences in which up to 25 amino acids in any one of SEQ ID NOs: 1 to 4, or 42 to 44 are substituted by other amino acids. In one embodiment, said ankyrin repeat domain comprises an amino acid sequence selected from the group consisting of (1) any one of SEQ ID NOs: 1 to 4, or 42 to 44 and (2) sequences in which up to 20 amino acids in any one of SEQ ID NOs: 1 to 4, or 42 to 44 are substituted by other amino acids. In one embodiment, said ankyrin repeat domain comprises an amino acid sequence selected from the group consisting of (1) any one of SEQ ID NOs: 1 to 4, or 42 to 44 and (2) sequences in which up to 15 amino acids in any one of SEQ ID NOs: 1 to 4, or 42 to 44 are substituted by other amino acids. In one embodiment, said ankyrin repeat domain comprises an amino acid sequence selected from the group consisting of (1) any one of SEQ ID NOs: 1 to 4, or 42 to 44 and (2) sequences in which up to 10 amino acids in any one of SEQ ID NOs: 1 to 4, or 42 to 44 are substituted by other amino acids. In one embodiment, said ankyrin repeat domain comprises an amino acid sequence selected from the group consisting of (1) any one of SEQ ID NOs: 1 to 4, or 42 to 44 and (2) sequences in which up to 5 amino acids in any one of SEQ ID NOs: 1 to 4, or 42 to 44 are substituted by other amino acids. In one embodiment, said ankyrin repeat domain comprises an amino acid sequence selected from the group consisting of (1) any one of SEQ ID NOs: 1 to 4, or 42 to 44 and (2) sequences in which up to 4 amino acids in any one of SEQ ID NOs: 1 to 4, or 42 to 44 are substituted by other amino acids. In one embodiment, said ankyrin repeat domain comprises an amino acid sequence selected from the group consisting of (1) any one of SEQ ID NOs: 1 to 4, or 42 to 44 and (2) sequences in which up to 3 amino acids in any one of SEQ ID NOs: 1 to 4, or 42 to 44 are substituted by other amino acids. In one embodiment, said ankyrin repeat domain comprises an amino acid sequence selected from the group consisting of (1) any one of SEQ ID NOs: 1 to 4, or 42 to 44 and (2) sequences in which up to 2 amino acids in any one of SEQ ID NOs: 1 to 4, or 42 to 44 are substituted by other amino acids. In one embodiment, said ankyrin repeat domain comprises an amino acid sequence selected from the group consisting of (1) any one of SEQ ID NOs: 1 to 4, or 42 to 44 and (2) sequences in which up to 1 amino acid in any one of SEQ ID NOs: 1 to 4, or 42 to 44 is substituted by another amino acid. In one embodiment, all of said 25, 24, 23, 22, 21, 20, 19, 18, 17, 16, 15, 14, 13, 12, 11, 10, 9, 8, 7, 6, 5, 4, 3, 2, or 1 amino acid substitutions occur in framework positions. In one embodiment, said ankyrin repeat domain comprises an amino acid sequence selected from the group consisting of SEQ ID NOs: 1 to 4, or 42 to 44.

In any of the conjugates, or pharmaceutically acceptable salts thereof, of the invention described herein, said ankyrin repeat domain with binding specificity for MSLN may optionally further comprise a “G,” an “S,” or a “GS” sequence at its N-terminus. Accordingly, in some embodiments, said ankyrin repeat domain with binding specificity for MSLN (i) comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 1 to 4, or 42 to 44 and (ii) further comprises at its N-terminus, a G, an S, or a GS. In some embodiments, said ankyrin repeat domain with binding specificity for MSLN (i) comprises an amino acid sequence selected from the group consisting of (1) any one of SEQ ID NOs: 1 to 4, or 42 to 44 and (2) sequences in which up to 25, or up to 24, or up to 23, or up to 22, or up to 21, or up to 20, or up to 19, or up to 18, or up to 17, or up to 16, or up to 15, or up to 14, or up to 13, or up to 12, or up to 11, or up to 10, or up to 9, or up to 8, or up to 7, or up to 6, or up to 5, or up to 4, or up to 3, or up to 2, or up to 1 amino acids in any of SEQ ID NOs: 1 to 4, or 42 to 44 are substituted by other amino acids, and (ii) further comprises at its N-terminus, a G, an S, or a GS. Thus, in an exemplary embodiment, said ankyrin repeat domain with binding specificity for MSLN (i) comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 1 and (ii) further comprises a GS at its N-terminus. In another exemplary embodiment, said ankyrin repeat domain with binding specificity for MSLN (i) comprises an amino acid sequence selected from the group consisting of (1) SEQ ID NO: 1 and (2) sequences in which up to 25, or up to 24, or up to 23, or up to 22, or up to 21, or up to 20, or up to 19, or up to 18, or up to 17, or up to 16, or up to 15, or up to 14, or up to 13, or up to 12, or up to 11, or up to 10, or up to 9, or up to 8, or up to 7, or up to 6, or up to 5, or up to 4, or up to 3, or up to 2, or up to 1 amino acids in SEQ ID NO: 1 are substituted by other amino acids, and (ii) further comprises a GS at its N-terminus.

In another aspect, the invention relates to such a conjugate or pharmaceutically acceptable salt thereof, wherein said chelator comprises a 1,4,7,10-tetraazacyclododecane ring (PubChem CID 64963), or a derivative thereof. In one embodiment, said 1,4,7,10-tetraazacyclododecane ring comprises one or more side chains. In one embodiment, said one or more side chains are connected to one or more of the nitrogen atoms of said 1,4,7,10-tetraazacyclododecane ring. In one embodiment, said 1,4,7,10-tetraazacyclododecane ring comprises one, two, three or four side chains, wherein each of said side chains is connected to a nitrogen atom of said 1,4,7,10-tetraazacyclododecane ring. In one embodiment, at least one of said one or more side chains comprises a carboxyl group (—COOH) or an amide group (—CONH₂). In one embodiment, at least one of said one or more side chains comprises a —CH₂—COOH group or a —CH₂—CONH₂ group. In one embodiment, said 1,4,7,10-tetraazacyclododecane ring comprises four side chains, wherein each of said side chains is connected to a nitrogen atom of said 1,4,7,10-tetraazacyclododecane ring, and wherein each of said side chains comprises a carboxyl group (—COOH) or an amide group (—CONH₂). In one embodiment, said 1,4,7,10-tetraazacyclododecane ring comprises four side chains, wherein each of said side chains is connected to a nitrogen atom of said 1,4,7,10-tetraazacyclododecane ring, and wherein each of said side chains comprises a —CH₂—COOH group or a —CH₂—CONH₂ group. In one embodiment, said chelator is DOTA (1,4,7,10-tetraazacyclododecane-1,4,7,10-tetraacetic acid) or TCMC (1,4,7,10-Tetraazacyclododecane-1,4,7,10-tetraacetamide), or a derivative thereof. In one embodiment, said chelator is DOTA (1,4,7,10-tetraazacyclododecane-1,4,7,10-tetraacetic acid), or a derivative thereof. In one embodiment, said chelator is TCMC (1,4,7,10-Tetraazacyclododecane-1,4,7,10-tetraacetamide), or a derivative thereof. TCMC is also called DOTAM or DOTA-amide. Derivatives of TCMC include, for example, monoacid forms of TCMC. Thus, in one exemplary embodiment, said chelator comprises a 1,4,7,10-tetraazacyclododecane ring, wherein said 1,4,7,10-tetraazacyclododecane ring comprises four side chains, wherein each of said side chains is connected to a nitrogen atom of said 1,4,7,10-tetraazacyclododecane ring, and wherein one of said side chains comprises a —CH₂—COOH group and at least one of said side chains comprises a —CH₂—CONH₂ group.

In one embodiment, the chelator is diethylenetriaminepentaacetic acid (DTPA), 1,4,7-triazacyclononane-1,4,7-triacetic acid (NOTA), 1,4,7,10-tetraazacyclododecane-1,4,7,10-tetraacetic acid (DOTA), 1,4,7,10-Tetraazacyclododecane-1,4,7,10-tetraacetamide (TCMC), or a derivative thereof. In one embodiment, the chelator is DOTA (1,4,7,10-tetraazacyclododecane-1,4,7,10-tetraacetic acid), or a derivative thereof. In another embodiment, the chelator is TCMC (1,4,7,10-Tetraazacyclododecane-1,4,7,10-tetraacetamide), or a derivative thereof.

In another aspect, the invention relates to such a conjugate or pharmaceutically acceptable salt thereof, wherein said chelator comprises a 1,4,7,10-tetraazacyclododecane ring, wherein said 1,4,7,10-tetraazacyclododecane ring comprises one or more side chains, wherein said one or more side chains are connected to one or more of the nitrogen atoms of said 1,4,7,10-tetraazacyclododecane ring, and wherein said chelator is covalently connected to said connector via one of said side chains. In one embodiment, said chelator has a structure of Formula (I):

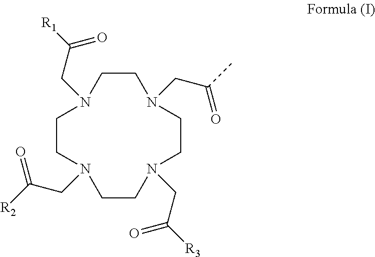

wherein R1, R2 and R3 are independently NH₂ or OH, and wherein the dotted line represents the covalent connection to said connector or to said ankyrin repeat domain with binding specificity for MSLN. In one embodiment, said chelator has a structure of Formula (II):

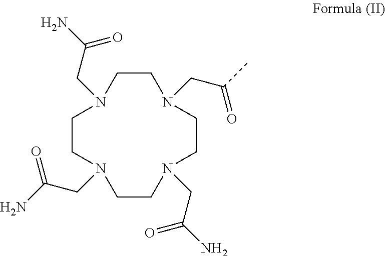

wherein the dotted line represents the covalent connection to said connector or to said ankyrin repeat domain with binding specificity for MSLN.

In another aspect, the invention relates to such a conjugate or pharmaceutically acceptable salt thereof, wherein said chelator comprises a 1,4,7,10-tetraazacyclododecane ring, and wherein said chelator is covalently connected to said connector via one of the carbon atoms of said 1,4,7,10-tetraazacyclododecane ring. In one embodiment, said chelator has a structure of Formula (III):

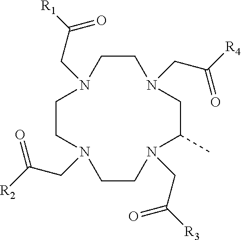

wherein R1, R2, R3 and R4 are independently NH₂ or OH, and wherein the dotted line represents the covalent connection to said connector or to said ankyrin repeat domain with binding specificity for MSLN.

In one embodiment, said chelator has a structure of Formula (IV):

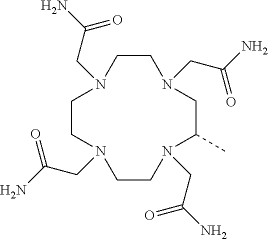 [SMILES: C[C@H]1CN(CC(N)=O)CCN(CC(N)=O)CCN(CC(N)=O)CCN1CC(N)=O]

wherein the dotted line represents the covalent connection to said connector or to said ankyrin repeat domain with binding specificity for MSLN.

In another aspect, the invention relates to such a conjugate or pharmaceutically acceptable salt thereof, wherein said conjugate further comprises a tag, wherein said tag comprises a Cysteine. In one embodiment, said tag is a peptide tag. In one embodiment, said tag is on one side covalently connected to said ankyrin repeat domain with binding specificity for MSLN and is on another side covalently connected to said chelator. In one embodiment, said tag is on one side covalently connected to said ankyrin repeat domain with binding specificity for MSLN and is on another side covalently connected to said connector. In one embodiment, said tag is located at the C-terminal side of said ankyrin repeat domain with binding specificity for MSLN. In one embodiment, said tag is covalently connected by a peptide bond to the C-terminal end of said ankyrin repeat domain with binding specificity for MSLN. In one embodiment, said conjugate has the formula: M-T-Ch-R, wherein M is said ankyrin repeat domain with binding specificity for MSLN, T is said tag, Ch is said chelator, and R is said radionuclide. In one embodiment, said conjugate has the formula: M-T-Co-Ch-R, wherein M is said ankyrin repeat domain with binding specificity for MSLN, T is said tag, Co is said connector, Ch is said chelator, and R is said radionuclide. In one embodiment, said tag comprises the amino acid sequence of SEQ ID NO: 16 or a variant thereof.

In another aspect, the invention relates to such a conjugate or pharmaceutically acceptable salt thereof, wherein said connector is covalently connected to said tag via a thioether bond. In one embodiment, said connector comprises a maleimide or a derivative thereof. In one embodiment, said thioether bond covalently connecting said tag and said connector is formed between said Cysteine comprised in said tag and said maleimide comprised in said connector. In one embodiment, said connector has a structure of Formula (V):

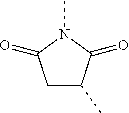

wherein the dotted line originating from N represents the covalent connection to said chelator, and wherein the dotted line originating from a carbon atom represents the covalent connection to said tag or to said ankyrin repeat domain with binding specificity for MSLN.

In any of the aspects or embodiments described herein, the pharmaceutically acceptable salt may be a trifluoroacetate salt. In another aspect, the invention relates to such a conjugate or pharmaceutically acceptable salt thereof, wherein said conjugate further comprises a half-life extending moiety. In one embodiment, said conjugate has the formula: H-M-Ch-R, wherein H is said half-life extending moiety, M is said ankyrin repeat domain with binding specificity for MSLN, Ch is said chelator, and R is said radionuclide. In one embodiment, said conjugate has the formula: M-H-Ch-R, wherein H is said half-life extending moiety, M is said ankyrin repeat domain with binding specificity for MSLN, Ch is said chelator, and R is said radionuclide. In one embodiment, said conjugate has the formula: H-M-T-Ch-R, wherein H is said half-life extending moiety, M is said ankyrin repeat domain with binding specificity for MSLN, T is said tag, Ch is said chelator, and R is said radionuclide. In one embodiment, said conjugate has the formula: M-H-T-Ch-R, wherein H is said half-life extending moiety, M is said ankyrin repeat domain with binding specificity for MSLN, T is said tag, Ch is said chelator, and R is said radionuclide. In one embodiment, said conjugate has the formula: H-M-Co-Ch-R, wherein H is said half-life extending moiety, M is said ankyrin repeat domain with binding specificity for MSLN, Co is said connector, Ch is said chelator, and R is said radionuclide. In one embodiment, said conjugate has the formula: H-M-T-Co-Ch-R, wherein H is said half-life extending moiety, M is said ankyrin repeat domain with binding specificity for MSLN, T is said tag, Co is said connector, Ch is said chelator, and R is said radionuclide. In one embodiment, said conjugate has the formula: M-H-Co-Ch-R, wherein H is said half-life extending moiety, M is said ankyrin repeat domain with binding specificity for MSLN, Co is said connector, Ch is said chelator, and R is said radionuclide. In one embodiment, said conjugate has the formula: M-H-T-Co-Ch-R, wherein H is said half-life extending moiety, M is said ankyrin repeat domain with binding specificity for MSLN, T is said tag, Co is said connector, Ch is said chelator, and R is said radionuclide. In one embodiment, said half-life extending moiety comprises an immunoglobulin domain. In one embodiment, the immunoglobulin domain comprises an Fc domain, or a variant thereof. In one embodiment, the Fc domain is derived from any one of the known heavy chain isotypes: IgG (y), IgM (p), IgD (6), IgE (E), or IgA (a). In another embodiment, the Fc domain is derived from any one of the known heavy chain isotypes or subtypes: IgG₁ (γ1), IgG₂ (γ²), IgG₃ (γ3), IgG₄ (γ4), IgA₁ (α1), or IgA₂ (α2). In one embodiment, the Fc domain is the Fc domain of human IgG₁, or a variant thereof.

In another aspect, the invention relates to such a conjugate or pharmaceutically acceptable salt thereof, wherein said conjugate further comprises a half-life extending moiety, and wherein said half-life extending moiety has binding specificity for human serum albumin. In one embodiment, said half-life extending moiety is an ankyrin repeat domain with binding specificity for human serum albumin. In one embodiment, said ankyrin repeat domain with binding specificity for human serum albumin binds human serum albumin with a K_{D} value of or below 500 nM, of or below 250 nM, or of or below 100 nM. In one embodiment, said ankyrin repeat domain with binding specificity for human serum albumin binds human serum albumin with a K_{D} value of or below 500 nM. In one embodiment, said ankyrin repeat domain with binding specificity for human serum albumin binds human serum albumin with a K_{D} value of or below 250 nM. In one embodiment, said ankyrin repeat domain with binding specificity for human serum albumin binds human serum albumin with a K_{D} value of or below 100 nM.

In another aspect, the invention relates to such a conjugate or pharmaceutically acceptable salt thereof, wherein said ankyrin repeat domain with binding specificity for human serum albumin comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 5 to 7, or 45. Thus, in one embodiment, said ankyrin repeat domain with binding specificity for human serum albumin comprises an amino acid sequence that is at least 80% identical to any one of SEQ ID NOs: 5 to 7, or 45. In one embodiment, said ankyrin repeat domain with binding specificity for human serum albumin comprises an amino acid sequence that is at least 85% identical to any one of SEQ ID NOs: 5 to 7, or 45. In one embodiment, said ankyrin repeat domain with binding specificity for human serum albumin comprises an amino acid sequence that is at least 88% identical to any one of SEQ ID NOs: 5 to 7, or 45. In one embodiment, said ankyrin repeat domain with binding specificity for human serum albumin comprises an amino acid sequence that is at least 90% identical to any one of SEQ ID NOs: 5 to 7, or 45. In one embodiment, said ankyrin repeat domain with binding specificity for human serum albumin comprises an amino acid sequence that is at least 91% identical to any one of SEQ ID NOs: 5 to 7, or 45. In one embodiment, said ankyrin repeat domain with binding specificity for human serum albumin comprises an amino acid sequence that is at least 92% identical to any one of SEQ ID NOs: 5 to 7, or 45. In one embodiment, said ankyrin repeat domain with binding specificity for human serum albumin comprises an amino acid sequence that is at least 93% identical to any one of SEQ ID NOs: 5 to 7, or 45. In one embodiment, said ankyrin repeat domain with binding specificity for human serum albumin comprises an amino acid sequence that is at least 94% identical to any one of SEQ ID NOs: 5 to 7, or 45. In one embodiment, said ankyrin repeat domain with binding specificity for human serum albumin comprises an amino acid sequence that is at least 95% identical to any one of SEQ ID NOs: 5 to 7, or 45. In one embodiment, said ankyrin repeat domain with binding specificity for human serum albumin comprises an amino acid sequence that is at least 96% identical to any one of SEQ ID NOs: 5 to 7, or 45. In one embodiment, said ankyrin repeat domain with binding specificity for human serum albumin comprises an amino acid sequence that is at least 97% identical to any one of SEQ ID NOs: 5 to 7, or 45. In one embodiment, said ankyrin repeat domain with binding specificity for human serum albumin comprises an amino acid sequence that is at least 98% identical to any one of SEQ ID NOs: 5 to 7, or 45. In one embodiment, said ankyrin repeat domain with binding specificity for human serum albumin comprises an amino acid sequence that is at least 99% identical to any one of SEQ ID NOs: 5 to 7, or 45. In one embodiment, any amino acid sequence differences between said ankyrin repeat domain with binding specificity for human serum albumin and any one of SEQ ID NOs: 5 to 7, or 45 represent amino acid substitutions in framework positions. In one embodiment, said ankyrin repeat domain with binding specificity for human serum albumin comprises an amino acid sequence that is 100% identical to any one of SEQ ID NOs: 5 to 7, or 45.

In another aspect, the invention relates to such a conjugate or pharmaceutically acceptable salt thereof, wherein said ankyrin repeat domain with binding specificity for human serum albumin comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 5. In one embodiment, any amino acid sequence differences between said ankyrin repeat domain with binding specificity for human serum albumin and SEQ ID NO: 5 represent amino acid substitutions in framework positions. In one embodiment, said ankyrin repeat domain with binding specificity for human serum albumin comprises an amino acid sequence that is 100% identical to SEQ ID NO: 5.

In another aspect, the invention relates to such a conjugate or pharmaceutically acceptable salt thereof, wherein said ankyrin repeat domain with binding specificity for human serum albumin comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 6. In one embodiment, any amino acid sequence differences between said ankyrin repeat domain with binding specificity for human serum albumin and SEQ ID NO: 6 represent amino acid substitutions in framework positions. In one embodiment, said ankyrin repeat domain with binding specificity for human serum albumin comprises an amino acid sequence that is 100% identical to SEQ ID NO: 6.

In another aspect, the invention relates to such a conjugate or pharmaceutically acceptable salt thereof, wherein said ankyrin repeat domain with binding specificity for human serum albumin comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 7. Thus, in one embodiment, said ankyrin repeat domain with binding specificity for human serum albumin comprises an amino acid sequence that is at least 80% identical to SEQ ID NO: 7. In one embodiment, said ankyrin repeat domain with binding specificity for human serum albumin comprises an amino acid sequence that is at least 85% identical to SEQ ID NO: 7. In one embodiment, said ankyrin repeat domain with binding specificity for human serum albumin comprises an amino acid sequence that is at least 88% identical to SEQ ID NO: 7. In one embodiment, said ankyrin repeat domain with binding specificity for human serum albumin comprises an amino acid sequence that is at least 90% identical to SEQ ID NO: 7. In one embodiment, said ankyrin repeat domain with binding specificity for human serum albumin comprises an amino acid sequence that is at least 91% identical to SEQ ID NO: 7. In one embodiment, said ankyrin repeat domain with binding specificity for human serum albumin comprises an amino acid sequence that is at least 92% identical to SEQ ID NO: 7. In one embodiment, said ankyrin repeat domain with binding specificity for human serum albumin comprises an amino acid sequence that is at least 93% identical to SEQ ID NO: 7. In one embodiment, said ankyrin repeat domain with binding specificity for human serum albumin comprises an amino acid sequence that is at least 94% identical to SEQ ID NO: 7. In one embodiment, said ankyrin repeat domain with binding specificity for human serum albumin comprises an amino acid sequence that is at least 95% identical to SEQ ID NO: 7. In one embodiment, said ankyrin repeat domain with binding specificity for human serum albumin comprises an amino acid sequence that is at least 96% identical to SEQ ID NO: 7. In one embodiment, said ankyrin repeat domain with binding specificity for human serum albumin comprises an amino acid sequence that is at least 97% identical to SEQ ID NO: 7. In one embodiment, said ankyrin repeat domain with binding specificity for human serum albumin comprises an amino acid sequence that is at least 98% identical to SEQ ID NO: 7. In one embodiment, said ankyrin repeat domain with binding specificity for human serum albumin comprises an amino acid sequence that is at least 99% identical to SEQ ID NO: 7. In one embodiment, said ankyrin repeat domain with binding specificity for human serum albumin comprises an amino acid sequence that is 100% identical to SEQ ID NO: 7. In one embodiment, any amino acid sequence differences between said ankyrin repeat domain with binding specificity for human serum albumin and SEQ ID NO: 7 represent amino acid substitutions in framework positions.

In one embodiment, said ankyrin repeat domain with binding specificity for human serum albumin comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 45. Thus, in one embodiment, said ankyrin repeat domain with binding specificity for human serum albumin comprises an amino acid sequence that is at least 80% identical to SEQ ID NO: 45. In one embodiment, said ankyrin repeat domain with binding specificity for human serum albumin comprises an amino acid sequence that is at least 85% identical to SEQ ID NO: 45. In one embodiment, said ankyrin repeat domain with binding specificity for human serum albumin comprises an amino acid sequence that is at least 88% identical to SEQ ID NO: 45. In one embodiment, said ankyrin repeat domain with binding specificity for human serum albumin comprises an amino acid sequence that is at least 90% identical to SEQ ID NO: 45. In one embodiment, said ankyrin repeat domain with binding specificity for human serum albumin comprises an amino acid sequence that is at least 91% identical to SEQ ID NO: 45. In one embodiment, said ankyrin repeat domain with binding specificity for human serum albumin comprises an amino acid sequence that is at least 92% identical to SEQ ID NO: 45. In one embodiment, said ankyrin repeat domain with binding specificity for human serum albumin comprises an amino acid sequence that is at least 93% identical to SEQ ID NO: 45. In one embodiment, said ankyrin repeat domain with binding specificity for human serum albumin comprises an amino acid sequence that is at least 94% identical to SEQ ID NO: 45. In one embodiment, said ankyrin repeat domain with binding specificity for human serum albumin comprises an amino acid sequence that is at least 95% identical to SEQ ID NO: 45. In one embodiment, said ankyrin repeat domain with binding specificity for human serum albumin comprises an amino acid sequence that is at least 96% identical to SEQ ID NO: 45. In one embodiment, said ankyrin repeat domain with binding specificity for human serum albumin comprises an amino acid sequence that is at least 97% identical to SEQ ID NO: 45. In one embodiment, said ankyrin repeat domain with binding specificity for human serum albumin comprises an amino acid sequence that is at least 98% identical to SEQ ID NO: 45. In one embodiment, said ankyrin repeat domain with binding specificity for human serum albumin comprises an amino acid sequence that is at least 99% identical to SEQ ID NO: 45. In one embodiment, said ankyrin repeat domain with binding specificity for human serum albumin comprises an amino acid sequence that is 100% identical to SEQ ID NO: 45. In one embodiment, any amino acid sequence differences between said ankyrin repeat domain with binding specificity for human serum albumin and SEQ ID NO: 45 represent amino acid substitutions in framework positions.

In another aspect, the invention relates to such a conjugate or pharmaceutically acceptable salt thereof, wherein said conjugate further comprises a peptide linker, and wherein said ankyrin repeat domain with binding specificity for human serum albumin is covalently connected to said ankyrin repeat domain with binding specificity for MSLN by said peptide linker. Different peptide linkers are known in the art. Examples of peptide linkers include PT-rich linkers, PAS-rich linkers and GS-rich linkers. Peptide linkers can have different lengths. Thus, in one embodiment, said peptide linker comprises the amino acid sequence of any one of SEQ ID NOs: 9 to 15, or a variant thereof. In one embodiment, said peptide linker comprises the amino acid sequence of SEQ ID NO: 9 or a variant thereof. In one embodiment, said peptide linker comprises the amino acid sequence of SEQ ID NO: 10 or a variant thereof. In one embodiment, said peptide linker comprises the amino acid sequence of SEQ ID NO: 11 or a variant thereof. In one embodiment, said peptide linker comprises the amino acid sequence of SEQ ID NO: 12 or a variant thereof. In one embodiment, said peptide linker comprises the amino acid sequence of SEQ ID NO: 13 or a variant thereof.

In one embodiment, said peptide linker comprises the amino acid sequence of SEQ ID NO: 14 or a variant thereof. In one embodiment, said peptide linker comprises the amino acid sequence of SEQ ID NO: 15 or a variant thereof. Said peptide linker may have a length of 1 to 24 amino acids, of 1 to 22 amino acids, of 1 to 20 amino acids, of 1 to 19 amino acids, of 1 to 18 amino acids, of 1 to 17 amino acids, of 1 to 16 amino acids, of 1 to 15 amino acids, of 1 to 14 amino acids, of 1 to 13 amino acids, of 1 to 12 amino acids, of 1 to 11 amino acids, of 1 to 10 amino acids, of 1 to 9 amino acids, of 1 to 8 amino acids, of 1 to 7 amino acids, of 1 to 6 amino acids, of 1 to 5 amino acids, of 1 to 4 amino acids, of 1 to 3 amino acids, of 1 to 2 amino acids, or of 1 amino acid or about 2 to 20 amino acids, of about 2 to 15 amino acids, of about 2 to 11 amino acids, of about 2 to 10 amino acids, of about 2 to 9 amino acids, of about 2 to 8 amino acids, of about 3 to 11 amino acids, of about 3 to 10 amino acids, of about 3 to 9 amino acids, or of about 3 to 8 amino acids In one embodiment, said conjugate has the formula: H-L-M-Ch-R, wherein H is said half-life extending moiety, L is said peptide linker, M is said ankyrin repeat domain with binding specificity for MSLN, Ch is said chelator, and R is said radionuclide. In another embodiment, said conjugate has the formula: M-L-H-Ch-R, wherein H is said half-life extending moiety, L is said peptide linker, M is said ankyrin repeat domain with binding specificity for MSLN, Ch is said chelator, and R is said radionuclide. In one embodiment, said conjugate has the formula: H-L-M-T-Ch-R, wherein H is said half-life extending moiety, L is said peptide linker, M is said ankyrin repeat domain with binding specificity for MSLN, T is said tag, Ch is said chelator, and R is said radionuclide. In another embodiment, said conjugate has the formula: M-L-H-T-Ch-R, wherein H is said half-life extending moiety, L is said peptide linker, M is said ankyrin repeat domain with binding specificity for MSLN, T is said tag, Ch is said chelator, and R is said radionuclide. In another embodiment, said conjugate has the formula: H-L-M-Co-Ch-R, wherein H is said half-life extending moiety, L is said peptide linker, M is said ankyrin repeat domain with binding specificity for MSLN, Co is said connector, Ch is said chelator, and R is said radionuclide. In another embodiment, said conjugate has the formula: H-L-M-T-Co-Ch-R, wherein H is said half-life extending moiety, L is said peptide linker, M is said ankyrin repeat domain with binding specificity for MSLN, T is said tag, Co is said connector, Ch is said chelator, and R is said radionuclide. In another embodiment, said conjugate has the formula: M-L-H-Co-Ch-R, wherein H is said half-life extending moiety, L is said peptide linker, M is said ankyrin repeat domain with binding specificity for MSLN, Co is said connector, Ch is said chelator, and R is said radionuclide. In another embodiment, said conjugate has the formula: M-L-H-T-Co-Ch-R, wherein H is said half-life extending moiety, L is said peptide linker, M is said ankyrin repeat domain with binding specificity for MSLN, T is said tag, Co is said connector, Ch is said chelator, and R is said radionuclide.

In another aspect, the invention relates to such a conjugate or pharmaceutically acceptable salt thereof, wherein said conjugate comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 17 to 26, or 46 to 50. Said amino acid sequence comprised in said conjugate comprises an ankyrin repeat domain with binding specificity for human MSLN at the N-terminal side and an ankyrin repeat domain with binding specificity for human serum albumin at the C-terminal side, and a peptide linker that connects said two ankyrin repeat domains. Thus, in one embodiment, said conjugate comprises an amino acid sequence that is at least 80% identical to any one of SEQ ID NOs: 17 to 26, or 46 to 50. In one embodiment, said conjugate comprises an amino acid sequence that is at least 85% identical to any one of SEQ ID NOs: 17 to 26, or 46 to 50. In one embodiment, said conjugate comprises an amino acid sequence that is at least 88% identical to any one of SEQ ID NOs: 17 to 26, or 46 to 50. In one embodiment, said conjugate comprises an amino acid sequence that is at least 90% identical to any one of SEQ ID NOs: 17 to 26, or 46 to 50. In one embodiment, said conjugate comprises an amino acid sequence that is at least 91% identical to any one of SEQ ID NOs: 17 to 26, or 46 to 50. In one embodiment, said conjugate comprises an amino acid sequence that is at least 92% identical to any one of SEQ ID NOs: 17 to 26, or 46 to 50. In one embodiment, said conjugate comprises an amino acid sequence that is at least 93% identical to any one of SEQ ID NOs: 17 to 26, or 46 to 50. In one embodiment, said conjugate comprises an amino acid sequence that is at least 94% identical to any one of SEQ ID NOs: 17 to 26, or 46 to 50. In one embodiment, said conjugate comprises an amino acid sequence that is at least 95% identical to any one of SEQ ID NOs: 17 to 26, or 46 to 50. In one embodiment, said conjugate comprises an amino acid sequence that is at least 96% identical to any one of SEQ ID NOs: 17 to 26, or 46 to 50. In one embodiment, said conjugate comprises an amino acid sequence that is at least 97% identical to any one of SEQ ID NOs: 17 to 26, or 46 to 50. In one embodiment, said conjugate comprises an amino acid sequence that is at least 98% identical to any one of SEQ ID NOs: 17 to 26, or 46 to 50. In one embodiment, said conjugate comprises an amino acid sequence that is at least 99% identical to any one of SEQ ID NOs: 17 to 26, or 46 to 50. In one embodiment, any amino acid sequence differences between said amino acid sequence comprised in said conjugate and said any one of SEQ ID NOs: 17 to 26, or 46 to 50 represent amino acid substitutions in framework positions of said two ankyrin repeat domains and/or in said peptide linker. In one embodiment, said conjugate comprises an amino acid sequence that is 100% identical to any one of SEQ ID NOs: 17 to 26, or 46 to 50. In one embodiment, said conjugate comprises said amino acid sequence described in any of the above embodiments, wherein said amino acid sequence is covalently bound at its N-terminal end to Glycine-Serine (GS). In one embodiment, said conjugate comprises said amino acid sequence described in any of the above embodiments, wherein said amino acid sequence is covalently bound at its C-terminal end to the amino acid sequence of SEQ ID NO: 16. In one embodiment, said amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 17 to 26, or 46 to 50 is covalently bound at its N-terminal end to Glycine-Serine (GS). In another embodiment, said amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 17 to 26, or 46 to 50 is covalently bound at its C-terminal end to the amino acid sequence of SEQ ID NO: 16.

In another aspect, the invention relates to such a conjugate or pharmaceutically acceptable salt thereof, wherein said conjugate comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 18. Thus, in one embodiment, said conjugate comprises an amino acid sequence that is at least 80% identical to SEQ ID NO: 18. In one embodiment, said conjugate comprises an amino acid sequence that is at least 81% identical to SEQ ID NO: 18. In one embodiment, said conjugate comprises an amino acid sequence that is at least 82% identical to SEQ ID NO: 18. In one embodiment, said conjugate comprises an amino acid sequence that is at least 83% identical to SEQ ID NO: 18. In one embodiment, said conjugate comprises an amino acid sequence that is at least 84% identical to SEQ ID NO: 18. In one embodiment, said conjugate comprises an amino acid sequence that is at least 85% identical to SEQ ID NO: 18. In one embodiment, said conjugate comprises an amino acid sequence that is at least 86% identical to SEQ ID NO: 18. In one embodiment, said conjugate comprises an amino acid sequence that is at least 87% identical to SEQ ID NO: 18. In one embodiment, said conjugate comprises an amino acid sequence that is at least 88% identical to SEQ ID NO: 18. In one embodiment, said conjugate comprises an amino acid sequence that is at least 89% identical to SEQ ID NO: 18. In one embodiment, said conjugate comprises an amino acid sequence that is at least 90% identical to SEQ ID NO: 18. In one embodiment, said conjugate comprises an amino acid sequence that is at least 91% identical to SEQ ID NO: 18. In one embodiment, said conjugate comprises an amino acid sequence that is at least 92% identical to SEQ ID NO: 18. In one embodiment, said conjugate comprises an amino acid sequence that is at least 93% identical SEQ ID NO: 18. In one embodiment, said conjugate comprises an amino acid sequence that is at least 94% identical to SEQ ID NO: 18. In one embodiment, said conjugate comprises an amino acid sequence that is at least 95% identical to SEQ ID NO: 18. In one embodiment, said conjugate comprises an amino acid sequence that is at least 96% identical to SEQ ID NO: 18. In one embodiment, said conjugate comprises an amino acid sequence that is at least 97% identical to SEQ ID NO: 18. In one embodiment, said conjugate comprises an amino acid sequence that is at least 98% identical to SEQ ID NO: 18. In one embodiment, said conjugate comprises an amino acid sequence that is at least 99% identical to SEQ ID NO: 18. In one embodiment, any amino acid sequence differences between said amino acid sequence comprised in said conjugate and SEQ ID NO: 18 represent amino acid substitutions in framework positions of said two ankyrin repeat domains and/or in said peptide linker. In one embodiment, said conjugate comprises an amino acid sequence that is 100% identical to SEQ ID NO: 18. In one embodiment, said conjugate comprises said amino acid sequence described in any of the above embodiments, wherein said amino acid sequence is covalently bound at its N-terminal end to Glycine-Serine (GS). In one embodiment, said conjugate comprises said amino acid sequence described in any of the above embodiments, wherein said amino acid sequence is covalently bound at its C-terminal end to the amino acid sequence of SEQ ID NO: 16.

In another aspect, the invention relates to such a conjugate or pharmaceutically acceptable salt thereof, wherein said conjugate comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 23. Thus, in one embodiment, said conjugate comprises an amino acid sequence that is at least 80% identical to SEQ ID NO: 23. In one embodiment, said conjugate comprises an amino acid sequence that is at least 85% identical to SEQ ID NO: 23. In one embodiment, said conjugate comprises an amino acid sequence that is at least 88% identical to SEQ ID NO: 23. In one embodiment, said conjugate comprises an amino acid sequence that is at least 90% identical to SEQ ID NO: 23. In one embodiment, said conjugate comprises an amino acid sequence that is at least 91% identical to SEQ ID NO: 23. In one embodiment, said conjugate comprises an amino acid sequence that is at least 92% identical to SEQ ID NO: 23. In one embodiment, said conjugate comprises an amino acid sequence that is at least 93% identical SEQ ID NO: 23. In one embodiment, said conjugate comprises an amino acid sequence that is at least 94% identical to SEQ ID NO: 23. In one embodiment, said conjugate comprises an amino acid sequence that is at least 95% identical to SEQ ID NO: 23. In one embodiment, said conjugate comprises an amino acid sequence that is at least 96% identical to SEQ ID NO: 23. In one embodiment, said conjugate comprises an amino acid sequence that is at least 97% identical to SEQ ID NO: 23. In one embodiment, said conjugate comprises an amino acid sequence that is at least 98% identical to SEQ ID NO: 23. In one embodiment, said conjugate comprises an amino acid sequence that is at least 99% identical to SEQ ID NO: 23. In one embodiment, any amino acid sequence differences between said amino acid sequence comprised in said conjugate and SEQ ID NO: 23 represent amino acid substitutions in framework positions of said two ankyrin repeat domains and/or in said peptide linker. In one embodiment, said conjugate comprises an amino acid sequence that is 100% identical to SEQ ID NO: 23. In one embodiment, said conjugate comprises said amino acid sequence described in any of the above embodiments, wherein said amino acid sequence is covalently bound at its N-terminal end to Glycine-Serine (GS). In one embodiment, said conjugate comprises said amino acid sequence described in any of the above embodiments, wherein said amino acid sequence is covalently bound at its C-terminal end to the amino acid sequence of SEQ ID NO: 16.

In one embodiment, said conjugate comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 47. Thus, in one embodiment, said conjugate comprises an amino acid sequence that is at least 80% identical to SEQ ID NO: 47. In one embodiment, said conjugate comprises an amino acid sequence that is at least 85% identical to SEQ ID NO: 47. In one embodiment, said conjugate comprises an amino acid sequence that is at least 88% identical to SEQ ID NO: 47. In one embodiment, said conjugate comprises an amino acid sequence that is at least 90% identical to SEQ ID NO: 47. In one embodiment, said conjugate comprises an amino acid sequence that is at least 91% identical to SEQ ID NO: 47. In one embodiment, said conjugate comprises an amino acid sequence that is at least 92% identical to SEQ ID NO: 47. In one embodiment, said conjugate comprises an amino acid sequence that is at least 93% identical SEQ ID NO: 47. In one embodiment, said conjugate comprises an amino acid sequence that is at least 94% identical to SEQ ID NO: 47. In one embodiment, said conjugate comprises an amino acid sequence that is at least 95% identical to SEQ ID NO: 47. In one embodiment, said conjugate comprises an amino acid sequence that is at least 96% identical to SEQ ID NO: 47. In one embodiment, said conjugate comprises an amino acid sequence that is at least 97% identical to SEQ ID NO: 47. In one embodiment, said conjugate comprises an amino acid sequence that is at least 98% identical to SEQ ID NO: 47. In one embodiment, said conjugate comprises an amino acid sequence that is at least 99% identical to SEQ ID NO: 47. In one embodiment, any amino acid sequence differences between said amino acid sequence comprised in said conjugate and SEQ ID NO: 47 represent amino acid substitutions in framework positions of said two ankyrin repeat domains and/or in said peptide linker. In one embodiment, said conjugate comprises an amino acid sequence that is 100% identical to SEQ ID NO: 47. In one embodiment, said conjugate comprises said amino acid sequence described in any of the above embodiments, wherein said amino acid sequence is covalently bound at its N-terminal end to Glycine-Serine (GS). In one embodiment, said conjugate comprises said amino acid sequence described in any of the above embodiments, wherein said amino acid sequence is covalently bound at its C-terminal end to the amino acid sequence of SEQ ID NO: 16.

In one embodiment, said conjugate comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 48. Thus, in one embodiment, said conjugate comprises an amino acid sequence that is at least 80% identical to SEQ ID NO: 48. In one embodiment, said conjugate comprises an amino acid sequence that is at least 85% identical to SEQ ID NO: 48. In one embodiment, said conjugate comprises an amino acid sequence that is at least 88% identical to SEQ ID NO: 48. In one embodiment, said conjugate comprises an amino acid sequence that is at least 90% identical to SEQ ID NO: 48. In one embodiment, said conjugate comprises an amino acid sequence that is at least 91% identical to SEQ ID NO: 48. In one embodiment, said conjugate comprises an amino acid sequence that is at least 92% identical to SEQ ID NO: 48. In one embodiment, said conjugate comprises an amino acid sequence that is at least 93% identical SEQ ID NO: 48. In one embodiment, said conjugate comprises an amino acid sequence that is at least 94% identical to SEQ ID NO: 48. In one embodiment, said conjugate comprises an amino acid sequence that is at least 95% identical to SEQ ID NO: 48. In one embodiment, said conjugate comprises an amino acid sequence that is at least 96% identical to SEQ ID NO: 48. In one embodiment, said conjugate comprises an amino acid sequence that is at least 97% identical to SEQ ID NO: 48. In one embodiment, said conjugate comprises an amino acid sequence that is at least 98% identical to SEQ ID NO: 48. In one embodiment, said conjugate comprises an amino acid sequence that is at least 99% identical to SEQ ID NO: 48. In one embodiment, any amino acid sequence differences between said amino acid sequence comprised in said conjugate and SEQ ID NO: 48 represent amino acid substitutions in framework positions of said two ankyrin repeat domains and/or in said peptide linker. In one embodiment, said conjugate comprises an amino acid sequence that is 100% identical to SEQ ID NO: 48. In one embodiment, said conjugate comprises said amino acid sequence described in any of the above embodiments, wherein said amino acid sequence is covalently bound at its N-terminal end to Glycine-Serine (GS). In one embodiment, said conjugate comprises said amino acid sequence described in any of the above embodiments, wherein said amino acid sequence is covalently bound at its C-terminal end to the amino acid sequence of SEQ ID NO: 16.

In one embodiment, said conjugate comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 49. Thus, in one embodiment, said conjugate comprises an amino acid sequence that is at least 80% identical to SEQ ID NO: 49. In one embodiment, said conjugate comprises an amino acid sequence that is at least 85% identical to SEQ ID NO: 49. In one embodiment, said conjugate comprises an amino acid sequence that is at least 88% identical to SEQ ID NO: 49. In one embodiment, said conjugate comprises an amino acid sequence that is at least 90% identical to SEQ ID NO: 49. In one embodiment, said conjugate comprises an amino acid sequence that is at least 91% identical to SEQ ID NO: 49. In one embodiment, said conjugate comprises an amino acid sequence that is at least 92% identical to SEQ ID NO: 49. In one embodiment, said conjugate comprises an amino acid sequence that is at least 93% identical SEQ ID NO: 49. In one embodiment, said conjugate comprises an amino acid sequence that is at least 94% identical to SEQ ID NO: 49. In one embodiment, said conjugate comprises an amino acid sequence that is at least 95% identical to SEQ ID NO: 49. In one embodiment, said conjugate comprises an amino acid sequence that is at least 96% identical to SEQ ID NO: 49. In one embodiment, said conjugate comprises an amino acid sequence that is at least 97% identical to SEQ ID NO: 49. In one embodiment, said conjugate comprises an amino acid sequence that is at least 98% identical to SEQ ID NO: 49. In one embodiment, said conjugate comprises an amino acid sequence that is at least 99% identical to SEQ ID NO: 49. In one embodiment, any amino acid sequence differences between said amino acid sequence comprised in said conjugate and SEQ ID NO: 49 represent amino acid substitutions in framework positions of said two ankyrin repeat domains and/or in said peptide linker. In one embodiment, said conjugate comprises an amino acid sequence that is 100% identical to SEQ ID NO: 49. In one embodiment, said conjugate comprises said amino acid sequence described in any of the above embodiments, wherein said amino acid sequence is covalently bound at its N-terminal end to Glycine-Serine (GS). In one embodiment, said conjugate comprises said amino acid sequence described in any of the above embodiments, wherein said amino acid sequence is covalently bound at its C-terminal end to the amino acid sequence of SEQ ID NO: 16.

In one embodiment, said conjugate comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 50. Thus, in one embodiment, said conjugate comprises an amino acid sequence that is at least 80% identical to SEQ ID NO: 50. In one embodiment, said conjugate comprises an amino acid sequence that is at least 85% identical to SEQ ID NO: 50. In one embodiment, said conjugate comprises an amino acid sequence that is at least 88% identical to SEQ ID NO: 50. In one embodiment, said conjugate comprises an amino acid sequence that is at least 90% identical to SEQ ID NO: 50. In one embodiment, said conjugate comprises an amino acid sequence that is at least 91% identical to SEQ ID NO: 50. In one embodiment, said conjugate comprises an amino acid sequence that is at least 92% identical to SEQ ID NO: 50. In one embodiment, said conjugate comprises an amino acid sequence that is at least 93% identical SEQ ID NO: 50. In one embodiment, said conjugate comprises an amino acid sequence that is at least 94% identical to SEQ ID NO: 50. In one embodiment, said conjugate comprises an amino acid sequence that is at least 95% identical to SEQ ID NO: 50. In one embodiment, said conjugate comprises an amino acid sequence that is at least 96% identical to SEQ ID NO: 50. In one embodiment, said conjugate comprises an amino acid sequence that is at least 97% identical to SEQ ID NO: 50. In one embodiment, said conjugate comprises an amino acid sequence that is at least 98% identical to SEQ ID NO: 50. In one embodiment, said conjugate comprises an amino acid sequence that is at least 99% identical to SEQ ID NO: 50. In one embodiment, any amino acid sequence differences between said amino acid sequence comprised in said conjugate and SEQ ID NO: 50 represent amino acid substitutions in framework positions of said two ankyrin repeat domains and/or in said peptide linker. In one embodiment, said conjugate comprises an amino acid sequence that is 100% identical to SEQ ID NO: 50. In one embodiment, said conjugate comprises said amino acid sequence described in any of the above embodiments, wherein said amino acid sequence is covalently bound at its N-terminal end to Glycine-Serine (GS). In one embodiment, said conjugate comprises said amino acid sequence described in any of the above embodiments, wherein said amino acid sequence is covalently bound at its C-terminal end to the amino acid sequence of SEQ ID NO: 16.

In one aspect, the invention relates to a conjugate or pharmaceutically acceptable salt thereof, the conjugate comprising (i) an ankyrin repeat protein comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 29 to 34, 38, or 51 to 56, (ii) a connector, (iii) a chelator, and (iv) a radionuclide, wherein said ankyrin repeat protein has binding specificity for human MSLN and for human serum albumin, wherein said connector is covalently connected to said ankyrin repeat protein, wherein said chelator is covalently connected to said connector, wherein said radionuclide is bound to said chelator, and wherein said radionuclide is Ac-225, Lu-177, Pb-203, Pb-212, In-111, Tb-161, Cu-64, Cu-67, Zr-89, Y-86, or Ce-134, preferably Ac-225, Lu-177, Pb-203, Pb-212, or In-111.

In another main aspect, the invention relates to a conjugate or pharmaceutically acceptable salt thereof, the conjugate comprising (i) an ankyrin repeat protein comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 29 to 34, 38, or 51 to 56, (ii) a connector, (iii) a chelator, and (iv) a radionuclide, wherein said ankyrin repeat protein has binding specificity for human MSLN and for human serum albumin, wherein said connector is covalently connected to said ankyrin repeat protein, wherein said chelator is covalently connected to said connector, wherein said radionuclide is bound to said chelator, and wherein said radionuclide is Pb-212, Pb-203, Lu-177, Ac-225, or In-111. An amino acid sequence of any one of SEQ ID NOs: 29 to 34, 38, or 51 to 56 comprises an ankyrin repeat domain with binding specificity for human serum albumin and an ankyrin repeat domain with binding specificity for human MSLN, wherein said ankyrin repeat domains are connected by a peptide linker, wherein said amino acid sequence comprises a Glycine-Serine (GS) at its N-terminus, and wherein said amino acid sequence comprises a Cysteine-containing tag at its C-terminus.

In one embodiment, said radionuclide is Pb-212. In one embodiment, said radionuclide is Pb-203. In one embodiment, said radionuclide is Lu-177. In one embodiment, said radionuclide is Ac-225. In one embodiment, said radionuclide is In-111. In one embodiment, said ankyrin repeat protein comprises an amino acid sequence that is at least at least 80% identical to any one of SEQ ID NOs: 29 to 34, 38, or 51 to 56. In one embodiment, said ankyrin repeat protein comprises an amino acid sequence that is at least 85% identical to any one of SEQ ID NOs: 29 to 34, 38, or 51 to 56. In one embodiment, said ankyrin repeat protein comprises an amino acid sequence that is at least 88% identical to any one of SEQ ID NOs: 29 to 34, 38, or 51 to 56. In one embodiment, said ankyrin repeat protein comprises an amino acid sequence that is at least 90% identical to any one of SEQ ID NOs: 29 to 34, 38, or 51 to 56. In one embodiment, said ankyrin repeat protein comprises an amino acid sequence that is at least 91% identical to any one of SEQ ID NOs: 29 to 34, 38, or 51 to 56. In one embodiment, said ankyrin repeat protein comprises an amino acid sequence that is at least 92% identical to any one of SEQ ID NOs: 29 to 34, 38, or 51 to 56. In one embodiment, said ankyrin repeat protein comprises an amino acid sequence that is at least 93% identical to any one of SEQ ID NOs: 29 to 34, 38, or 51 to 56. In one embodiment, said ankyrin repeat protein comprises an amino acid sequence that is at least 94% identical to any one of SEQ ID NOs: 29 to 34, 38, or 51 to 56. In one embodiment, said ankyrin repeat protein comprises an amino acid sequence that is at least 95% identical to any one of SEQ ID NOs: 29 to 34, 38, or 51 to 56. In one embodiment, said ankyrin repeat protein comprises an amino acid sequence that is at least 96% identical to any one of SEQ ID NOs: 29 to 34, 38, or 51 to 56. In one embodiment, said ankyrin repeat protein comprises an amino acid sequence that is at least 97% identical to any one of SEQ ID NOs: 29 to 34, 38, or 51 to 56. In one embodiment, said ankyrin repeat protein comprises an amino acid sequence that is at least 98% identical to any one of SEQ ID NOs: 29 to 34, 38, or 51 to 56. In one embodiment, said ankyrin repeat protein comprises an amino acid sequence that is at least 99% identical to any one of SEQ ID NOs: 29 to 34, 38, or 51 to 56. In one embodiment, any amino acid sequence differences between said amino acid sequence comprised in said ankyrin repeat protein and said any one of SEQ ID NOs: 29 to 34, 38, or 51 to 56 represent amino acid substitutions in positions other than positions of potential target interaction residues of ankyrin repeat domains. In one embodiment, any amino acid sequence differences between said amino acid sequence comprised in said ankyrin repeat protein and said any one of SEQ ID NOs: 29 to 34, 38, or 51 to 56 represent amino acid substitutions in (i) framework positions of ankyrin repeat domain(s), (ii) a peptide linker, (iii) a Glycine-Serine (GS), and/or (iv) a Cysteine-containing tag. In one embodiment, the C-terminal Cysteine of said any one of SEQ ID NOs: 29 to 34, 38, or 51 to 56 is not substituted with another amino acid. In one embodiment, said ankyrin repeat protein comprises an amino acid sequence that is 100% identical to any one of SEQ ID NOs: 29 to 34, 38, or 51 to 56.

In another aspect, the invention relates to such a conjugate or pharmaceutically acceptable salt thereof, wherein said ankyrin repeat protein with binding specificity for MSLN and for human serum albumin comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 29. Thus, in one embodiment, said ankyrin repeat protein comprises an amino acid sequence that is at least 80% identical to SEQ ID NO: 29. In one embodiment, said ankyrin repeat protein comprises an amino acid sequence that is at least 81% identical to SEQ ID NO: 29. In one embodiment, said ankyrin repeat protein comprises an amino acid sequence that is at least 82% identical to SEQ ID NO: 29. In one embodiment, said ankyrin repeat protein comprises an amino acid sequence that is at least 83% identical to SEQ ID NO: 29. In one embodiment, said ankyrin repeat protein comprises an amino acid sequence that is at least 84% identical to SEQ ID NO: 29. In one embodiment, said ankyrin repeat protein comprises an amino acid sequence that is at least 85% identical to SEQ ID NO: 29. In one embodiment, said ankyrin repeat protein comprises an amino acid sequence that is at least 86% identical to SEQ ID NO: 29. In one embodiment, said ankyrin repeat protein comprises an amino acid sequence that is at least 87% identical to SEQ ID NO: 29. In one embodiment, said ankyrin repeat protein comprises an amino acid sequence that is at least 88% identical to SEQ ID NO: 29. In one embodiment, said ankyrin repeat protein comprises an amino acid sequence that is at least 89% identical to SEQ ID NO: 29. In one embodiment, said ankyrin repeat protein comprises an amino acid sequence that is at least 90% identical to SEQ ID NO: 29. In one embodiment, said ankyrin repeat protein comprises an amino acid sequence that is at least 91% identical to SEQ ID NO: 29. In one embodiment, said ankyrin repeat protein comprises an amino acid sequence that is at least 92% identical to SEQ ID NO: 29. In one embodiment, said ankyrin repeat protein comprises an amino acid sequence that is at least 93% identical SEQ ID NO: 29. In one embodiment, said ankyrin repeat protein comprises an amino acid sequence that is at least 94% identical to SEQ ID NO: 29. In one embodiment, said ankyrin repeat protein comprises an amino acid sequence that is at least 95% identical to SEQ ID NO: 29. In one embodiment, said ankyrin repeat protein comprises an amino acid sequence that is at least 96% identical to SEQ ID NO: 29. In one embodiment, said ankyrin repeat protein comprises an amino acid sequence that is at least 97% identical to SEQ ID NO: 29. In one embodiment, said ankyrin repeat protein comprises an amino acid sequence that is at least 98% identical to SEQ ID NO: 29. In one embodiment, said ankyrin repeat protein comprises an amino acid sequence that is at least 99% identical to SEQ ID NO: 29. In one embodiment, any amino acid sequence differences between said amino acid sequence comprised in said ankyrin repeat protein and SEQ ID NO: 29 represent amino acid substitutions in positions other than positions of potential target interaction residues of ankyrin repeat domains. In one embodiment, any amino acid sequence differences between said amino acid sequence comprised in said ankyrin repeat protein and SEQ ID NO: 29 represent amino acid substitutions in (i) framework positions of ankyrin repeat domain(s), (ii) a peptide linker, (iii) a Glycine-Serine (GS), and/or (iv) a Cysteine-containing tag. In one embodiment, the C-terminal Cysteine of SEQ ID NO: 29 is not substituted with another amino acid. In one embodiment, said ankyrin repeat protein comprises an amino acid sequence that is 100% identical to SEQ ID NO: 29.

In another aspect, the invention relates to such a conjugate or pharmaceutically acceptable salt thereof, wherein said ankyrin repeat protein with binding specificity for MSLN and for human serum albumin comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 30. Thus, in one embodiment, said ankyrin repeat protein comprises an amino acid sequence that is at least 80% identical to SEQ ID NO: 30. In one embodiment, said ankyrin repeat protein comprises an amino acid sequence that is at least 81% identical to SEQ ID NO: 30. In one embodiment, said ankyrin repeat protein comprises an amino acid sequence that is at least 82% identical to SEQ ID NO: 30. In one embodiment, said ankyrin repeat protein comprises an amino acid sequence that is at least 83% identical to SEQ ID NO: 30. In one embodiment, said ankyrin repeat protein comprises an amino acid sequence that is at least 84% identical to SEQ ID NO: 30. In one embodiment, said ankyrin repeat protein comprises an amino acid sequence that is at least 85% identical to SEQ ID NO: 30. In one embodiment, said ankyrin repeat protein comprises an amino acid sequence that is at least 86% identical to SEQ ID NO: 30. In one embodiment, said ankyrin repeat protein comprises an amino acid sequence that is at least 87% identical to SEQ ID NO: 30. In one embodiment, said ankyrin repeat protein comprises an amino acid sequence that is at least 88% identical to SEQ ID NO: 30. In one embodiment, said ankyrin repeat protein comprises an amino acid sequence that is at least 89% identical to SEQ ID NO: 30. In one embodiment, said ankyrin repeat protein comprises an amino acid sequence that is at least 90% identical to SEQ ID NO: 30. In one embodiment, said ankyrin repeat protein comprises an amino acid sequence that is at least 91% identical to SEQ ID NO: 30. In one embodiment, said ankyrin repeat protein comprises an amino acid sequence that is at least 92% identical to SEQ ID NO: 30. In one embodiment, said ankyrin repeat protein comprises an amino acid sequence that is at least 93% identical SEQ ID NO: 30. In one embodiment, said ankyrin repeat protein comprises an amino acid sequence that is at least 94% identical to SEQ ID NO: 30. In one embodiment, said ankyrin repeat protein comprises an amino acid sequence that is at least 95% identical to SEQ ID NO: 30. In one embodiment, said ankyrin repeat protein comprises an amino acid sequence that is at least 96% identical to SEQ ID NO: 30. In one embodiment, said ankyrin repeat protein comprises an amino acid sequence that is at least 97% identical to SEQ ID NO: 30. In one embodiment, said ankyrin repeat protein comprises an amino acid sequence that is at least 98% identical to SEQ ID NO: 30. In one embodiment, said ankyrin repeat protein comprises an amino acid sequence that is at least 99% identical to SEQ ID NO: 30. In one embodiment, any amino acid sequence differences between said amino acid sequence comprised in said ankyrin repeat protein and SEQ ID NO: 30 represent amino acid substitutions in positions other than positions of potential target interaction residues of ankyrin repeat domains. In one embodiment, any amino acid sequence differences between said amino acid sequence comprised in said ankyrin repeat protein and SEQ ID NO: 30 represent amino acid substitutions in (i) framework positions of ankyrin repeat domain(s), (ii) a peptide linker, (iii) a Glycine-Serine (GS), and/or (iv) a Cysteine-containing tag. In one embodiment, the C-terminal Cysteine of SEQ ID NO: 30 is not substituted with another amino acid. In one embodiment, said ankyrin repeat protein comprises an amino acid sequence that is 100% identical to SEQ ID NO: 30.

In another aspect, the invention relates to such a conjugate or pharmaceutically acceptable salt thereof, wherein said ankyrin repeat protein with binding specificity for MSLN and for human serum albumin comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 31. Thus, in one embodiment, said ankyrin repeat protein comprises an amino acid sequence that is at least 80% identical to SEQ ID NO: 31. In one embodiment, said ankyrin repeat protein comprises an amino acid sequence that is at least 81% identical to SEQ ID NO: 31. In one embodiment, said ankyrin repeat protein comprises an amino acid sequence that is at least 82% identical to SEQ ID NO: 31. In one embodiment, said ankyrin repeat protein comprises an amino acid sequence that is at least 83% identical to SEQ ID NO: 31. In one embodiment, said ankyrin repeat protein comprises an amino acid sequence that is at least 84% identical to SEQ ID NO: 31. In one embodiment, said ankyrin repeat protein comprises an amino acid sequence that is at least 85% identical to SEQ ID NO: 31. In one embodiment, said ankyrin repeat protein comprises an amino acid sequence that is at least 86% identical to SEQ ID NO: 31. In one embodiment, said ankyrin repeat protein comprises an amino acid sequence that is at least 87% identical to SEQ ID NO: 31. In one embodiment, said ankyrin repeat protein comprises an amino acid sequence that is at least 88% identical to SEQ ID NO: 31. In one embodiment, said ankyrin repeat protein comprises an amino acid sequence that is at least 89% identical to SEQ ID NO: 31. In one embodiment, said ankyrin repeat protein comprises an amino acid sequence that is at least 90% identical to SEQ ID NO: 31. In one embodiment, said ankyrin repeat protein comprises an amino acid sequence that is at least 91% identical to SEQ ID NO: 31. In one embodiment, said ankyrin repeat protein comprises an amino acid sequence that is at least 92% identical to SEQ ID NO: 31. In one embodiment, said ankyrin repeat protein comprises an amino acid sequence that is at least 93% identical SEQ ID NO: 31. In one embodiment, said ankyrin repeat protein comprises an amino acid sequence that is at least 94% identical to SEQ ID NO: 31. In one embodiment, said ankyrin repeat protein comprises an amino acid sequence that is at least 95% identical to SEQ ID NO: 31. In one embodiment, said ankyrin repeat protein comprises an amino acid sequence that is at least 96% identical to SEQ ID NO: 31. In one embodiment, said ankyrin repeat protein comprises an amino acid sequence that is at least 97% identical to SEQ ID NO: 31. In one embodiment, said ankyrin repeat protein comprises an amino acid sequence that is at least 98% identical to SEQ ID NO: 31. In one embodiment, said ankyrin repeat protein comprises an amino acid sequence that is at least 99% identical to SEQ ID NO: 31. In one embodiment, any amino acid sequence differences between said amino acid sequence comprised in said ankyrin repeat protein and SEQ ID NO: 31 represent amino acid substitutions in positions other than positions of potential target interaction residues of ankyrin repeat domains. In one embodiment, any amino acid sequence differences between said amino acid sequence comprised in said ankyrin repeat protein and SEQ ID NO: 31 represent amino acid substitutions in (i) framework positions of ankyrin repeat domain(s), (ii) a peptide linker, (iii) a Glycine-Serine (GS), and/or (iv) a Cysteine-containing tag. In one embodiment, the C-terminal Cysteine of SEQ ID NO: 31 is not substituted with another amino acid. In one embodiment, said ankyrin repeat protein comprises an amino acid sequence that is 100% identical to SEQ ID NO: 31.

In another aspect, the invention relates to such a conjugate or pharmaceutically acceptable salt thereof, wherein said ankyrin repeat protein with binding specificity for MSLN and for human serum albumin comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 32. Thus, in one embodiment, said ankyrin repeat protein comprises an amino acid sequence that is at least 80% identical to SEQ ID NO: 32. In one embodiment, said ankyrin repeat protein comprises an amino acid sequence that is at least 81% identical to SEQ ID NO: 32. In one embodiment, said ankyrin repeat protein comprises an amino acid sequence that is at least 82% identical to SEQ ID NO: 32. In one embodiment, said ankyrin repeat protein comprises an amino acid sequence that is at least 83% identical to SEQ ID NO: 32. In one embodiment, said ankyrin repeat protein comprises an amino acid sequence that is at least 84% identical to SEQ ID NO: 32. In one embodiment, said ankyrin repeat protein comprises an amino acid sequence that is at least 85% identical to SEQ ID NO: 32. In one embodiment, said ankyrin repeat protein comprises an amino acid sequence that is at least 86% identical to SEQ ID NO: 32. In one embodiment, said ankyrin repeat protein comprises an amino acid sequence that is at least 87% identical to SEQ ID NO: 32. In one embodiment, said ankyrin repeat protein comprises an amino acid sequence that is at least 88% identical to SEQ ID NO: 32. In one embodiment, said ankyrin repeat protein comprises an amino acid sequence that is at least 89% identical to SEQ ID NO: 32. In one embodiment, said ankyrin repeat protein comprises an amino acid sequence that is at least 90% identical to SEQ ID NO: 32. In one embodiment, said ankyrin repeat protein comprises an amino acid sequence that is at least 91% identical to SEQ ID NO: 32. In one embodiment, said ankyrin repeat protein comprises an amino acid sequence that is at least 92% identical to SEQ ID NO: 32. In one embodiment, said ankyrin repeat protein comprises an amino acid sequence that is at least 93% identical SEQ ID NO: 32. In one embodiment, said ankyrin repeat protein comprises an amino acid sequence that is at least 94% identical to SEQ ID NO: 32. In one embodiment, said ankyrin repeat protein comprises an amino acid sequence that is at least 95% identical to SEQ ID NO: 32. In one embodiment, said ankyrin repeat protein comprises an amino acid sequence that is at least 96% identical to SEQ ID NO: 32. In one embodiment, said ankyrin repeat protein comprises an amino acid sequence that is at least 97% identical to SEQ ID NO: 32. In one embodiment, said ankyrin repeat protein comprises an amino acid sequence that is at least 98% identical to SEQ ID NO: 32. In one embodiment, said ankyrin repeat protein comprises an amino acid sequence that is at least 99% identical to SEQ ID NO: 32. In one embodiment, any amino acid sequence differences between said amino acid sequence comprised in said ankyrin repeat protein and SEQ ID NO: 32 represent amino acid substitutions in positions other than positions of potential target interaction residues of ankyrin repeat domains. In one embodiment, any amino acid sequence differences between said amino acid sequence comprised in said ankyrin repeat protein and SEQ ID NO: 32 represent amino acid substitutions in (i) framework positions of ankyrin repeat domain(s), (ii) a peptide linker, (iii) a Glycine-Serine (GS), and/or (iv) a Cysteine-containing tag. In one embodiment, the C-terminal Cysteine of SEQ ID NO: 32 is not substituted with another amino acid. In one embodiment, said ankyrin repeat protein comprises an amino acid sequence that is 100% identical to SEQ ID NO: 32.

In another aspect, the invention relates to such a conjugate or pharmaceutically acceptable salt thereof, wherein said ankyrin repeat protein with binding specificity for MSLN and for human serum albumin comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 33. Thus, in one embodiment, said ankyrin repeat protein comprises an amino acid sequence that is at least 80% identical to SEQ ID NO: 33. In one embodiment, said ankyrin repeat protein comprises an amino acid sequence that is at least 85% identical to SEQ ID NO: 33. In one embodiment, said ankyrin repeat protein comprises an amino acid sequence that is at least 88% identical to SEQ ID NO: 33. In one embodiment, said ankyrin repeat protein comprises an amino acid sequence that is at least 90% identical to SEQ ID NO: 33. In one embodiment, said ankyrin repeat protein comprises an amino acid sequence that is at least 91% identical to SEQ ID NO: 33. In one embodiment, said ankyrin repeat protein comprises an amino acid sequence that is at least 92% identical to SEQ ID NO: 33. In one embodiment, said ankyrin repeat protein comprises an amino acid sequence that is at least 93% identical SEQ ID NO: 33. In one embodiment, said ankyrin repeat protein comprises an amino acid sequence that is at least 94% identical to SEQ ID NO: 33. In one embodiment, said ankyrin repeat protein comprises an amino acid sequence that is at least 95% identical to SEQ ID NO: 33. In one embodiment, said ankyrin repeat protein comprises an amino acid sequence that is at least 96% identical to SEQ ID NO: 33. In one embodiment, said ankyrin repeat protein comprises an amino acid sequence that is at least 97% identical to SEQ ID NO: 33. In one embodiment, said ankyrin repeat protein comprises an amino acid sequence that is at least 98% identical to SEQ ID NO: 33. In one embodiment, said ankyrin repeat protein comprises an amino acid sequence that is at least 99% identical to SEQ ID NO: 33. In one embodiment, any amino acid sequence differences between said amino acid sequence comprised in said ankyrin repeat protein and SEQ ID NO: 33 represent amino acid substitutions in positions other than positions of potential target interaction residues of ankyrin repeat domains. In one embodiment, any amino acid sequence differences between said amino acid sequence comprised in said ankyrin repeat protein and SEQ ID NO: 33 represent amino acid substitutions in (i) framework positions of ankyrin repeat domain(s), (ii) a peptide linker, (iii) a Glycine-Serine (GS), and/or (iv) a Cysteine-containing tag. In one embodiment, the C-terminal Cysteine of SEQ ID NO: 33 is not substituted with another amino acid. In one embodiment, said ankyrin repeat protein comprises an amino acid sequence that is 100% identical to SEQ ID NO: 33.

In another aspect, the invention relates to such a conjugate or pharmaceutically acceptable salt thereof, wherein said ankyrin repeat protein with binding specificity for MSLN and for human serum albumin comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 34. Thus, in one embodiment, said ankyrin repeat protein comprises an amino acid sequence that is at least 80% identical to SEQ ID NO: 34. In one embodiment, said ankyrin repeat protein comprises an amino acid sequence that is at least 81% identical to SEQ ID NO: 34. In one embodiment, said ankyrin repeat protein comprises an amino acid sequence that is at least 82% identical to SEQ ID NO: 34. In one embodiment, said ankyrin repeat protein comprises an amino acid sequence that is at least 83% identical to SEQ ID NO: 34. In one embodiment, said ankyrin repeat protein comprises an amino acid sequence that is at least 84% identical to SEQ ID NO: 34. In one embodiment, said ankyrin repeat protein comprises an amino acid sequence that is at least 85% identical to SEQ ID NO: 34. In one embodiment, said ankyrin repeat protein comprises an amino acid sequence that is at least 86% identical to SEQ ID NO: 34. In one embodiment, said ankyrin repeat protein comprises an amino acid sequence that is at least 87% identical to SEQ ID NO: 34. In one embodiment, said ankyrin repeat protein comprises an amino acid sequence that is at least 88% identical to SEQ ID NO: 34. In one embodiment, said ankyrin repeat protein comprises an amino acid sequence that is at least 89% identical to SEQ ID NO: 34. In one embodiment, said ankyrin repeat protein comprises an amino acid sequence that is at least 90% identical to SEQ ID NO: 34. In one embodiment, said ankyrin repeat protein comprises an amino acid sequence that is at least 91% identical to SEQ ID NO: 34. In one embodiment, said ankyrin repeat protein comprises an amino acid sequence that is at least 92% identical to SEQ ID NO: 34. In one embodiment, said ankyrin repeat protein comprises an amino acid sequence that is at least 93% identical SEQ ID NO: 34. In one embodiment, said ankyrin repeat protein comprises an amino acid sequence that is at least 94% identical to SEQ ID NO: 34. In one embodiment, said ankyrin repeat protein comprises an amino acid sequence that is at least 95% identical to SEQ ID NO: 34. In one embodiment, said ankyrin repeat protein comprises an amino acid sequence that is at least 96% identical to SEQ ID NO: 34. In one embodiment, said ankyrin repeat protein comprises an amino acid sequence that is at least 97% identical to SEQ ID NO: 34. In one embodiment, said ankyrin repeat protein comprises an amino acid sequence that is at least 98% identical to SEQ ID NO: 34. In one embodiment, said ankyrin repeat protein comprises an amino acid sequence that is at least 99% identical to SEQ ID NO: 34. In one embodiment, any amino acid sequence differences between said amino acid sequence comprised in said ankyrin repeat protein and SEQ ID NO: 34 represent amino acid substitutions in positions other than positions of potential target interaction residues of ankyrin repeat domains. In one embodiment, any amino acid sequence differences between said amino acid sequence comprised in said ankyrin repeat protein and SEQ ID NO: 34 represent amino acid substitutions in (i) framework positions of ankyrin repeat domain(s), (ii) a peptide linker, (iii) a Glycine-Serine (GS), and/or (iv) a Cysteine-containing tag. In one embodiment, the C-terminal Cysteine of SEQ ID NO: 34 is not substituted with another amino acid. In one embodiment, said ankyrin repeat protein comprises an amino acid sequence that is 100% identical to SEQ ID NO: 34.

In another aspect, the invention relates to such a conjugate or pharmaceutically acceptable salt thereof, wherein said ankyrin repeat protein with binding specificity for MSLN and for human serum albumin comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 38. Thus, in one embodiment, said ankyrin repeat protein comprises an amino acid sequence that is at least 80% identical to SEQ ID NO: 38. In one embodiment, said ankyrin repeat protein comprises an amino acid sequence that is at least 81% identical to SEQ ID NO: 38. In one embodiment, said ankyrin repeat protein comprises an amino acid sequence that is at least 82% identical to SEQ ID NO: 38. In one embodiment, said ankyrin repeat protein comprises an amino acid sequence that is at least 83% identical to SEQ ID NO: 38. In one embodiment, said ankyrin repeat protein comprises an amino acid sequence that is at least 84% identical to SEQ ID NO: 38. In one embodiment, said ankyrin repeat protein comprises an amino acid sequence that is at least 85% identical to SEQ ID NO: 38. In one embodiment, said ankyrin repeat protein comprises an amino acid sequence that is at least 86% identical to SEQ ID NO: 38. In one embodiment, said ankyrin repeat protein comprises an amino acid sequence that is at least 87% identical to SEQ ID NO: 38. In one embodiment, said ankyrin repeat protein comprises an amino acid sequence that is at least 88% identical to SEQ ID NO: 38. In one embodiment, said ankyrin repeat protein comprises an amino acid sequence that is at least 89% identical to SEQ ID NO: 38. In one embodiment, said ankyrin repeat protein comprises an amino acid sequence that is at least 90% identical to SEQ ID NO: 38. In one embodiment, said ankyrin repeat protein comprises an amino acid sequence that is at least 91% identical to SEQ ID NO: 38. In one embodiment, said ankyrin repeat protein comprises an amino acid sequence that is at least 92% identical to SEQ ID NO: 38. In one embodiment, said ankyrin repeat protein comprises an amino acid sequence that is at least 93% identical SEQ ID NO: 38. In one embodiment, said ankyrin repeat protein comprises an amino acid sequence that is at least 94% identical to SEQ ID NO: 38. In one embodiment, said ankyrin repeat protein comprises an amino acid sequence that is at least 95% identical to SEQ ID NO: 38. In one embodiment, said ankyrin repeat protein comprises an amino acid sequence that is at least 96% identical to SEQ ID NO: 38. In one embodiment, said ankyrin repeat protein comprises an amino acid sequence that is at least 97% identical to SEQ ID NO: 38. In one embodiment, said ankyrin repeat protein comprises an amino acid sequence that is at least 98% identical to SEQ ID NO: 38. In one embodiment, said ankyrin repeat protein comprises an amino acid sequence that is at least 99% identical to SEQ ID NO: 38. In one embodiment, any amino acid sequence differences between said amino acid sequence comprised in said ankyrin repeat protein and SEQ ID NO: 38 represent amino acid substitutions in positions other than positions of potential target interaction residues of ankyrin repeat domains. In one embodiment, any amino acid sequence differences between said amino acid sequence comprised in said ankyrin repeat protein and SEQ ID NO: 38 represent amino acid substitutions in (i) framework positions of ankyrin repeat domain(s), (ii) a peptide linker, (iii) a Glycine-Serine (GS), and/or (iv) a Cysteine-containing tag. In one embodiment, the C-terminal Cysteine of SEQ ID NO: 38 is not substituted with another amino acid. In one embodiment, said ankyrin repeat protein comprises an amino acid sequence that is 100% identical to SEQ ID NO: 38.

In one embodiment, said ankyrin repeat protein with binding specificity for MSLN and for human serum albumin comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 51. Thus, in one embodiment, said ankyrin repeat protein comprises an amino acid sequence that is at least 80% identical to SEQ ID NO: 51. In one embodiment, said ankyrin repeat protein comprises an amino acid sequence that is at least 85% identical to SEQ ID NO: 51. In one embodiment, said ankyrin repeat protein comprises an amino acid sequence that is at least 88% identical to SEQ ID NO: 51. In one embodiment, said ankyrin repeat protein comprises an amino acid sequence that is at least 90% identical to SEQ ID NO: 51. In one embodiment, said ankyrin repeat protein comprises an amino acid sequence that is at least 91% identical to SEQ ID NO: 51. In one embodiment, said ankyrin repeat protein comprises an amino acid sequence that is at least 92% identical to SEQ ID NO: 51. In one embodiment, said ankyrin repeat protein comprises an amino acid sequence that is at least 93% identical SEQ ID NO: 51. In one embodiment, said ankyrin repeat protein comprises an amino acid sequence that is at least 94% identical to SEQ ID NO: 51. In one embodiment, said ankyrin repeat protein comprises an amino acid sequence that is at least 95% identical to SEQ ID NO: 51. In one embodiment, said ankyrin repeat protein comprises an amino acid sequence that is at least 96% identical to SEQ ID NO: 51. In one embodiment, said ankyrin repeat protein comprises an amino acid sequence that is at least 97% identical to SEQ ID NO: 51. In one embodiment, said ankyrin repeat protein comprises an amino acid sequence that is at least 98% identical to SEQ ID NO: 51. In one embodiment, said ankyrin repeat protein comprises an amino acid sequence that is at least 99% identical to SEQ ID NO: 51. In one embodiment, any amino acid sequence differences between said amino acid sequence comprised in said ankyrin repeat protein and SEQ ID NO: 51 represent amino acid substitutions in positions other than positions of potential target interaction residues of ankyrin repeat domains. In one embodiment, any amino acid sequence differences between said amino acid sequence comprised in said ankyrin repeat protein and SEQ ID NO: 51 represent amino acid substitutions in (i) framework positions of ankyrin repeat domain(s), (ii) a peptide linker, (iii) a Glycine-Serine (GS), and/or (iv) a Cysteine-containing tag. In one embodiment, the C-terminal Cysteine of SEQ ID NO: 51 is not substituted with another amino acid. In one embodiment, said ankyrin repeat protein comprises an amino acid sequence that is 100% identical to SEQ ID NO: 51.

In one embodiment, said ankyrin repeat protein with binding specificity for MSLN and for human serum albumin comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 52. Thus, in one embodiment, said ankyrin repeat protein comprises an amino acid sequence that is at least 80% identical to SEQ ID NO: 52. In one embodiment, said ankyrin repeat protein comprises an amino acid sequence that is at least 85% identical to SEQ ID NO: 52. In one embodiment, said ankyrin repeat protein comprises an amino acid sequence that is at least 88% identical to SEQ ID NO: 52. In one embodiment, said ankyrin repeat protein comprises an amino acid sequence that is at least 90% identical to SEQ ID NO: 52. In one embodiment, said ankyrin repeat protein comprises an amino acid sequence that is at least 91% identical to SEQ ID NO: 52. In one embodiment, said ankyrin repeat protein comprises an amino acid sequence that is at least 92% identical to SEQ ID NO: 52. In one embodiment, said ankyrin repeat protein comprises an amino acid sequence that is at least 93% identical SEQ ID NO: 52. In one embodiment, said ankyrin repeat protein comprises an amino acid sequence that is at least 94% identical to SEQ ID NO: 52. In one embodiment, said ankyrin repeat protein comprises an amino acid sequence that is at least 95% identical to SEQ ID NO: 52. In one embodiment, said ankyrin repeat protein comprises an amino acid sequence that is at least 96% identical to SEQ ID NO: 52. In one embodiment, said ankyrin repeat protein comprises an amino acid sequence that is at least 97% identical to SEQ ID NO: 52. In one embodiment, said ankyrin repeat protein comprises an amino acid sequence that is at least 98% identical to SEQ ID NO: 52. In one embodiment, said ankyrin repeat protein comprises an amino acid sequence that is at least 99% identical to SEQ ID NO: 52. In one embodiment, any amino acid sequence differences between said amino acid sequence comprised in said ankyrin repeat protein and SEQ ID NO: 52 represent amino acid substitutions in positions other than positions of potential target interaction residues of ankyrin repeat domains. In one embodiment, any amino acid sequence differences between said amino acid sequence comprised in said ankyrin repeat protein and SEQ ID NO: 52 represent amino acid substitutions in (i) framework positions of ankyrin repeat domain(s), (ii) a peptide linker, (iii) a Glycine-Serine (GS), and/or (iv) a Cysteine-containing tag. In one embodiment, the C-terminal Cysteine of SEQ ID NO: 52 is not substituted with another amino acid. In one embodiment, said ankyrin repeat protein comprises an amino acid sequence that is 100% identical to SEQ ID NO: 52.

In one embodiment, said ankyrin repeat protein with binding specificity for MSLN and for human serum albumin comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 53. Thus, in one embodiment, said ankyrin repeat protein comprises an amino acid sequence that is at least 80% identical to SEQ ID NO: 53. In one embodiment, said ankyrin repeat protein comprises an amino acid sequence that is at least 85% identical to SEQ ID NO: 53. In one embodiment, said ankyrin repeat protein comprises an amino acid sequence that is at least 88% identical to SEQ ID NO: 53. In one embodiment, said ankyrin repeat protein comprises an amino acid sequence that is at least 90% identical to SEQ ID NO: 53. In one embodiment, said ankyrin repeat protein comprises an amino acid sequence that is at least 91% identical to SEQ ID NO: 53. In one embodiment, said ankyrin repeat protein comprises an amino acid sequence that is at least 92% identical to SEQ ID NO: 53. In one embodiment, said ankyrin repeat protein comprises an amino acid sequence that is at least 93% identical SEQ ID NO: 53. In one embodiment, said ankyrin repeat protein comprises an amino acid sequence that is at least 94% identical to SEQ ID NO: 53. In one embodiment, said ankyrin repeat protein comprises an amino acid sequence that is at least 95% identical to SEQ ID NO: 53. In one embodiment, said ankyrin repeat protein comprises an amino acid sequence that is at least 96% identical to SEQ ID NO: 53. In one embodiment, said ankyrin repeat protein comprises an amino acid sequence that is at least 97% identical to SEQ ID NO: 53. In one embodiment, said ankyrin repeat protein comprises an amino acid sequence that is at least 98% identical to SEQ ID NO: 53. In one embodiment, said ankyrin repeat protein comprises an amino acid sequence that is at least 99% identical to SEQ ID NO: 53. In one embodiment, any amino acid sequence differences between said amino acid sequence comprised in said ankyrin repeat protein and SEQ ID NO: 53 represent amino acid substitutions in positions other than positions of potential target interaction residues of ankyrin repeat domains. In one embodiment, any amino acid sequence differences between said amino acid sequence comprised in said ankyrin repeat protein and SEQ ID NO: 53 represent amino acid substitutions in (i) framework positions of ankyrin repeat domain(s), (ii) a peptide linker, (iii) a Glycine-Serine (GS), and/or (iv) a Cysteine-containing tag. In one embodiment, the C-terminal Cysteine of SEQ ID NO: 53 is not substituted with another amino acid. In one embodiment, said ankyrin repeat protein comprises an amino acid sequence that is 100% identical to SEQ ID NO: 53.

In one embodiment, said ankyrin repeat protein with binding specificity for MSLN and for human serum albumin comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 54. Thus, in one embodiment, said ankyrin repeat protein comprises an amino acid sequence that is at least 80% identical to SEQ ID NO: 54. In one embodiment, said ankyrin repeat protein comprises an amino acid sequence that is at least 85% identical to SEQ ID NO: 54. In one embodiment, said ankyrin repeat protein comprises an amino acid sequence that is at least 88% identical to SEQ ID NO: 54. In one embodiment, said ankyrin repeat protein comprises an amino acid sequence that is at least 90% identical to SEQ ID NO: 54. In one embodiment, said ankyrin repeat protein comprises an amino acid sequence that is at least 91% identical to SEQ ID NO: 54. In one embodiment, said ankyrin repeat protein comprises an amino acid sequence that is at least 92% identical to SEQ ID NO: 54. In one embodiment, said ankyrin repeat protein comprises an amino acid sequence that is at least 93% identical SEQ ID NO: 54. In one embodiment, said ankyrin repeat protein comprises an amino acid sequence that is at least 94% identical to SEQ ID NO: 54. In one embodiment, said ankyrin repeat protein comprises an amino acid sequence that is at least 95% identical to SEQ ID NO: 54. In one embodiment, said ankyrin repeat protein comprises an amino acid sequence that is at least 96% identical to SEQ ID NO: 54. In one embodiment, said ankyrin repeat protein comprises an amino acid sequence that is at least 97% identical to SEQ ID NO: 54. In one embodiment, said ankyrin repeat protein comprises an amino acid sequence that is at least 98% identical to SEQ ID NO: 54. In one embodiment, said ankyrin repeat protein comprises an amino acid sequence that is at least 99% identical to SEQ ID NO: 54. In one embodiment, any amino acid sequence differences between said amino acid sequence comprised in said ankyrin repeat protein and SEQ ID NO: 54 represent amino acid substitutions in positions other than positions of potential target interaction residues of ankyrin repeat domains. In one embodiment, any amino acid sequence differences between said amino acid sequence comprised in said ankyrin repeat protein and SEQ ID NO: 54 represent amino acid substitutions in (i) framework positions of ankyrin repeat domain(s), (ii) a peptide linker, (iii) a Glycine-Serine (GS), and/or (iv) a Cysteine-containing tag. In one embodiment, the C-terminal Cysteine of SEQ ID NO: 54 is not substituted with another amino acid. In one embodiment, said ankyrin repeat protein comprises an amino acid sequence that is 100% identical to SEQ ID NO: 54.

In one embodiment, said ankyrin repeat protein with binding specificity for MSLN and for human serum albumin comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 55. Thus, in one embodiment, said ankyrin repeat protein comprises an amino acid sequence that is at least 80% identical to SEQ ID NO: 55. In one embodiment, said ankyrin repeat protein comprises an amino acid sequence that is at least 85% identical to SEQ ID NO: 55. In one embodiment, said ankyrin repeat protein comprises an amino acid sequence that is at least 88% identical to SEQ ID NO: 55. In one embodiment, said ankyrin repeat protein comprises an amino acid sequence that is at least 90% identical to SEQ ID NO: 55. In one embodiment, said ankyrin repeat protein comprises an amino acid sequence that is at least 91% identical to SEQ ID NO: 55. In one embodiment, said ankyrin repeat protein comprises an amino acid sequence that is at least 92% identical to SEQ ID NO: 55. In one embodiment, said ankyrin repeat protein comprises an amino acid sequence that is at least 93% identical SEQ ID NO: 55. In one embodiment, said ankyrin repeat protein comprises an amino acid sequence that is at least 94% identical to SEQ ID NO: 55. In one embodiment, said ankyrin repeat protein comprises an amino acid sequence that is at least 95% identical to SEQ ID NO: 55. In one embodiment, said ankyrin repeat protein comprises an amino acid sequence that is at least 96% identical to SEQ ID NO: 55. In one embodiment, said ankyrin repeat protein comprises an amino acid sequence that is at least 97% identical to SEQ ID NO: 55. In one embodiment, said ankyrin repeat protein comprises an amino acid sequence that is at least 98% identical to SEQ ID NO: 55. In one embodiment, said ankyrin repeat protein comprises an amino acid sequence that is at least 99% identical to SEQ ID NO: 55. In one embodiment, any amino acid sequence differences between said amino acid sequence comprised in said ankyrin repeat protein and SEQ ID NO: 55 represent amino acid substitutions in positions other than positions of potential target interaction residues of ankyrin repeat domains. In one embodiment, any amino acid sequence differences between said amino acid sequence comprised in said ankyrin repeat protein and SEQ ID NO: 55 represent amino acid substitutions in (i) framework positions of ankyrin repeat domain(s), (ii) a peptide linker, (iii) a Glycine-Serine (GS), and/or (iv) a Cysteine-containing tag. In one embodiment, the C-terminal Cysteine of SEQ ID NO: 55 is not substituted with another amino acid. In one embodiment, said ankyrin repeat protein comprises an amino acid sequence that is 100% identical to SEQ ID NO: 55.

In one embodiment, said ankyrin repeat protein with binding specificity for MSLN and for human serum albumin comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 56. Thus, in one embodiment, said ankyrin repeat protein comprises an amino acid sequence that is at least 80% identical to SEQ ID NO: 56. In one embodiment, said ankyrin repeat protein comprises an amino acid sequence that is at least 85% identical to SEQ ID NO: 56. In one embodiment, said ankyrin repeat protein comprises an amino acid sequence that is at least 88% identical to SEQ ID NO: 56. In one embodiment, said ankyrin repeat protein comprises an amino acid sequence that is at least 90% identical to SEQ ID NO: 56. In one embodiment, said ankyrin repeat protein comprises an amino acid sequence that is at least 91% identical to SEQ ID NO: 56. In one embodiment, said ankyrin repeat protein comprises an amino acid sequence that is at least 92% identical to SEQ ID NO: 56. In one embodiment, said ankyrin repeat protein comprises an amino acid sequence that is at least 93% identical SEQ ID NO: 56. In one embodiment, said ankyrin repeat protein comprises an amino acid sequence that is at least 94% identical to SEQ ID NO: 56. In one embodiment, said ankyrin repeat protein comprises an amino acid sequence that is at least 95% identical to SEQ ID NO: 56. In one embodiment, said ankyrin repeat protein comprises an amino acid sequence that is at least 96% identical to SEQ ID NO: 56. In one embodiment, said ankyrin repeat protein comprises an amino acid sequence that is at least 97% identical to SEQ ID NO: 56. In one embodiment, said ankyrin repeat protein comprises an amino acid sequence that is at least 98% identical to SEQ ID NO: 56. In one embodiment, said ankyrin repeat protein comprises an amino acid sequence that is at least 99% identical to SEQ ID NO: 56. In one embodiment, any amino acid sequence differences between said amino acid sequence comprised in said ankyrin repeat protein and SEQ ID NO: 56 represent amino acid substitutions in positions other than positions of potential target interaction residues of ankyrin repeat domains. In one embodiment, any amino acid sequence differences between said amino acid sequence comprised in said ankyrin repeat protein and SEQ ID NO: 56 represent amino acid substitutions in (i) framework positions of ankyrin repeat domain(s), (ii) a peptide linker, (iii) a Glycine-Serine (GS), and/or (iv) a Cysteine-containing tag. In one embodiment, the C-terminal Cysteine of SEQ ID NO: 56 is not substituted with another amino acid. In one embodiment, said ankyrin repeat protein comprises an amino acid sequence that is 100% identical to SEQ ID NO: 56.

In another aspect, the invention relates to such a conjugate or pharmaceutically acceptable salt thereof, wherein said ankyrin repeat protein comprises a Cysteine, wherein said connector comprises maleimide or a derivative thereof, and wherein said Cysteine is covalently bound to said connector via a thioether bond.

In one embodiment, said Cysteine is located at the C-terminal end of said amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 29 to 34, 38, or 51 to 56.

In another aspect, the invention relates to such a conjugate or pharmaceutically acceptable salt thereof, wherein said chelator comprises a 1,4,7,10-tetraazacyclododecane ring (PubChem CID 64963), or a derivative thereof. In one embodiment, said 1,4,7,10-tetraazacyclododecane ring comprises one or more side chains. In one embodiment, said one or more side chains are connected to one or more of the nitrogen atoms of said 1,4,7,10-tetraazacyclododecane ring. In one embodiment, said 1,4,7,10-tetraazacyclododecane ring comprises one, two, three or four side chains, wherein each of said side chains is connected to a nitrogen atom of said 1,4,7,10-tetraazacyclododecane ring. In one embodiment, at least one of said one or more side chains comprises a carboxyl group (—COOH) or an amide group (—CONH₂). In one embodiment, at least one of said one or more side chains comprises a —CH₂—COOH group or a —CH₂—CONH₂ group. In one embodiment, said 1,4,7,10-tetraazacyclododecane ring comprises four side chains, wherein each of said side chains is connected to a nitrogen atom of said 1,4,7,10-tetraazacyclododecane ring, and wherein each of said side chains comprises a carboxyl group (—COOH) or an amide group (—CONH₂). In one embodiment, said 1,4,7,10-tetraazacyclododecane ring comprises four side chains, wherein each of said side chains is connected to a nitrogen atom of said 1,4,7,10-tetraazacyclododecane ring, and wherein each of said side chains comprises a —CH₂—COOH group or a —CH₂—CONH₂ group. In one embodiment, said chelator is DOTA (1,4,7,10-tetraazacyclododecane-1,4,7,10-tetraacetic acid) or TCMC (1,4,7,10-Tetraazacyclododecane-1,4,7,10-tetraacetamide), or a derivative thereof. In one embodiment, said chelator is DOTA (1,4,7,10-tetraazacyclododecane-1,4,7,10-tetraacetic acid), or a derivative thereof. In one embodiment, said chelator is TCMC (1,4,7,10-Tetraazacyclododecane-1,4,7,10-tetraacetamide), or a derivative thereof. TCMC is also called DOTAM or DOTA-amide. Derivatives of TCMC include, for example, monoacid forms of TCMC. Thus, in one embodiment, said chelator comprises a 1,4,7,10-tetraazacyclododecane ring, wherein said 1,4,7,10-tetraazacyclododecane ring comprises four side chains, wherein each of said side chains is connected to a nitrogen atom of said 1,4,7,10-tetraazacyclododecane ring, and wherein one of said side chains comprises a —CH₂—COOH group and at least one of said side chains comprises a —CH₂—CONH₂ group.

In one embodiment, the chelator is diethylenetriaminepentaacetic acid (DTPA), 1,4,7-triazacyclononane-1,4,7-triacetic acid (NOTA), 1,4,7,10-tetraazacyclododecane-1,4,7,10-tetraacetic acid (DOTA), 1,4,7,10-Tetraazacyclododecane-1,4,7,10-tetraacetamide (TCMC), or a derivative thereof. In one embodiment, the chelator is 1,4,7,10-tetraazacyclododecane-1,4,7,10-tetraacetic acid (DOTA), or a derivative thereof. In another embodiment, the chelator is 1,4,7,10-Tetraazacyclododecane-1,4,7,10-tetraacetamide (TCMC), or a derivative thereof.

In another aspect, the invention relates to such a conjugate or pharmaceutically acceptable salt thereof, wherein said chelator has a structure of Formula (I):

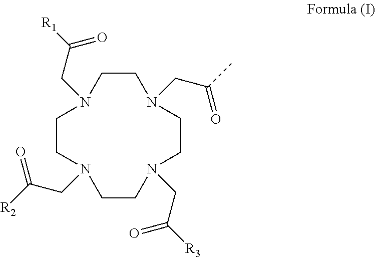

wherein R1, R2 and R3 are independently NH₂ or OH, and wherein the dotted line represents the covalent connection to said connector or to said ankyrin repeat domain with binding specificity for MSLN; or of Formula (III):

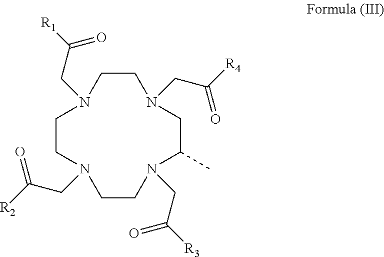

wherein R1, R2, R3 and R4 are independently NH₂ or OH, and wherein the dotted line represents the covalent connection to said connector or to said ankyrin repeat domain with binding specificity for MSLN.

In another aspect, the invention relates to such a conjugate or pharmaceutically acceptable salt thereof, wherein said chelator has a structure of Formula (I):

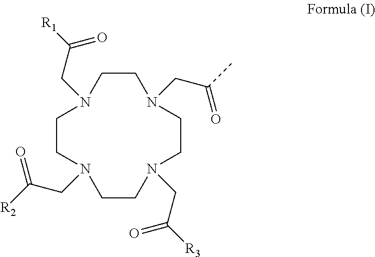

wherein R1, R2 and R3 are independently NH₂ or OH, and wherein the dotted line represents the covalent connection to said connector or to said ankyrin repeat domain with binding specificity for MSLN. In one embodiment, said chelator has a structure of Formula (II):

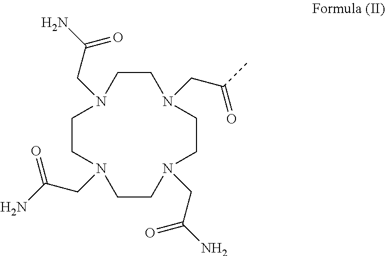

wherein the dotted line represents the covalent connection to said connector or to said ankyrin repeat domain with binding specificity for MSLN.

In another aspect, the invention relates to such a conjugate or pharmaceutically acceptable salt thereof, wherein said chelator has a structure of Formula (III):

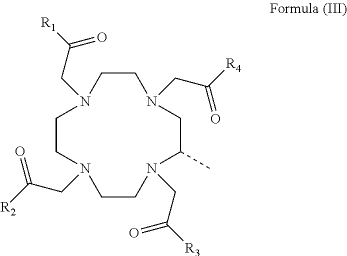

wherein R1, R2, R3 and R4 are independently NH₂ or OH, and wherein the dotted line represents the covalent connection to said connector or to said ankyrin repeat domain with binding specificity for MSLN.

In one embodiment, said chelator has a structure of Formula (IV):

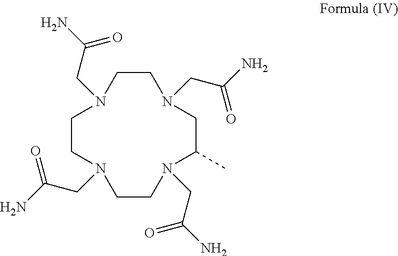 [SMILES: C[C@H]1CN(CC(N)=O)CCN(CC(N)=O)CCN(CC(N)=O)CCN1CC(N)=O]

wherein the dotted line represents the covalent connection to said connector or to said ankyrin repeat domain with binding specificity for MSLN.

In another aspect, the invention relates to a conjugate or pharmaceutically acceptable salt thereof, the conjugate having a structure of Formula (VI):

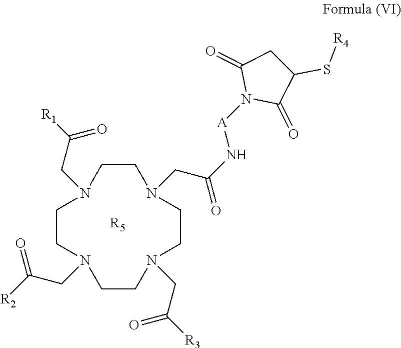

wherein R1, R2, and R3 are independently NH₂ or OH;
wherein A is CₐH_{b}N_{c}O_{d}, wherein a, b, c, and d are integers;
wherein R4 is an ankyrin repeat protein comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 29 to 34, 38, or 51 to 56, wherein said ankyrin repeat protein has binding specificity for MSLN and for human serum albumin, wherein said ankyrin repeat protein comprises a Cysteine, and wherein said Cysteine forms a thioether bond connecting said ankyrin repeat protein with a maleimide ring;
and wherein R5 is a chelated radionuclide, wherein said radionuclide is Ac-225, Lu-177, Pb-203, Pb-212, In-111, Tb-161, Cu-64, Cu-67, Zr-89, Y-86, or Ce-134, preferably Ac-225, Lu-177, Pb-203, Pb-212, or In-111;
or the conjugate having a structure of Formula (VII):

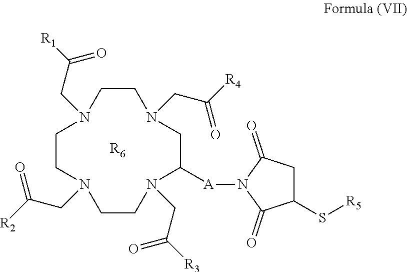

wherein R1, R2, R3 and R4 are independently NH₂ or OH;
wherein A is CₐH_{b}N_{c}O_{d}, wherein a, b, c, and d are integers;
wherein R5 is an ankyrin repeat protein comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 29 to 34, 38, or 51 to 56, wherein said ankyrin repeat protein has binding specificity for MSLN and for human serum albumin, wherein said ankyrin repeat protein comprises a Cysteine, and wherein said Cysteine forms a thioether bond connecting said ankyrin repeat protein with a maleimide ring;
and wherein R6 is a chelated radionuclide, wherein said radionuclide is Ac-225, Lu-177, Pb-203, Pb-212, In-111, Tb-161, Cu-64, Cu-67, Zr-89, Y-86, or Ce-134, preferably Ac-225, Lu-177, Pb-203, Pb-212, or In-111.

Thus, in one aspect, the invention relates to a conjugate or pharmaceutically acceptable salt thereof, the conjugate having a structure of Formula (VI):

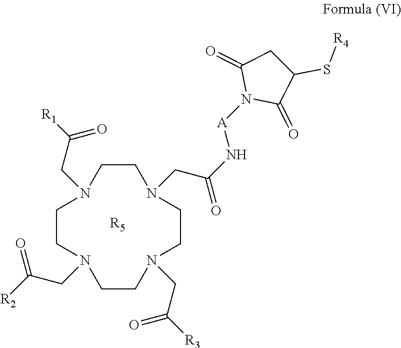

wherein R1, R2, and R3 are independently NH₂ or OH;
wherein A is CₐH_{b}N_{c}O_{d}, wherein a, b, c, and d are integers;
wherein R4 is an ankyrin repeat protein comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 29 to 34, 38, or 51 to 56, wherein said ankyrin repeat protein has binding specificity for MSLN and for human serum albumin, wherein said ankyrin repeat protein comprises a Cysteine, and wherein said Cysteine forms a thioether bond connecting said ankyrin repeat protein with a maleimide ring;
and wherein R5 is a chelated radionuclide, wherein said radionuclide is Ac-225, Lu-177, Pb-203, Pb-212, In-111, Tb-161, Cu-64, Cu-67, Zr-89, Y-86, or Ce-134, preferably Ac-225, Lu-177, Pb-203, Pb-212, or In-111. In one embodiment, said R5 in Formula (VI) is a chelated radionuclide, wherein said radionuclide is Ac-225. In one embodiment, said R5 in Formula (VI) is a chelated radionuclide, wherein said radionuclide is Lu-177. In one embodiment, said R5 in Formula (VI) is a chelated radionuclide, wherein said radionuclide is In-111.

Thus, in one aspect, the invention relates to a conjugate or pharmaceutically acceptable salt thereof, the conjugate having a structure of Formula (VII):

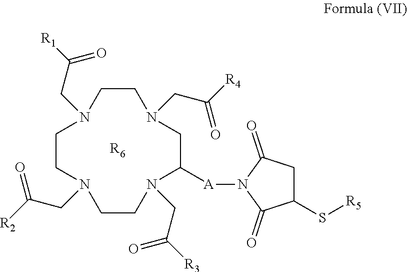

wherein R1, R2, R3 and R4 are independently NH₂ or OH;
wherein A is CₐH_{b}N_{c}O_{d}, wherein a, b, c, and d are integers;
wherein R5 is an ankyrin repeat protein comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 29 to 34, 38, and 51 to 56, wherein said ankyrin repeat protein has binding specificity for MSLN and for human serum albumin, wherein said ankyrin repeat protein comprises a Cysteine, and wherein said Cysteine forms a thioether bond connecting said ankyrin repeat protein with a maleimide ring;
and wherein R6 is a chelated radionuclide, wherein said radionuclide is Ac-225, Lu-177, Pb-203, Pb-212, In-111, Tb-161, Cu-64, Cu-67, Zr-89, Y-86, or Ce-134, preferably Ac-225, Lu-177, Pb-203, Pb-212, or In-111. In one embodiment, said R6 in Formula (VII) is a chelated radionuclide, wherein said radionuclide is Ac-225. In one embodiment, said R6 in Formula (VII) is a chelated radionuclide, wherein said radionuclide is Lu-177. In one embodiment, said R6 in Formula (VII) is a chelated radionuclide, wherein said radionuclide is In-111.

In another main aspect, the invention relates to a conjugate or pharmaceutically acceptable salt thereof, the conjugate having a structure of Formula (VI):

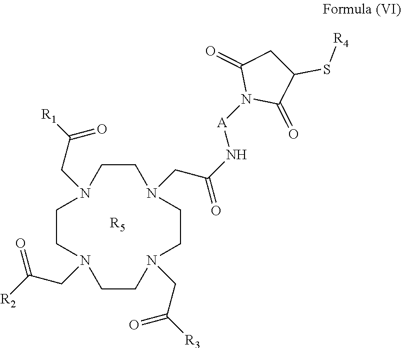

wherein R1, R2, and R3 are independently NH₂ or OH;
wherein A is CₐH_{b}N_{c}O_{d}, wherein a, b, c, and d are integers;
wherein R4 is an ankyrin repeat protein comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 29 to 34, 38, and 51 to 56, wherein said ankyrin repeat protein has binding specificity for MSLN and for human serum albumin, wherein said ankyrin repeat protein comprises a Cysteine, and wherein said Cysteine forms a thioether bond connecting said ankyrin repeat protein with a maleimide ring;
and wherein R5 is a chelated radionuclide, wherein said radionuclide is Pb-212 or Pb-203;
or the conjugate having a structure of Formula (VII):

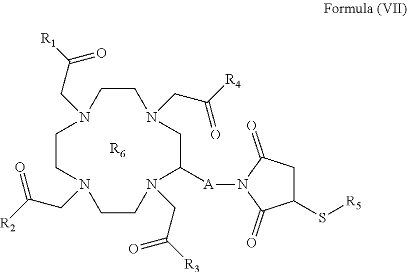

wherein R1, R2, R3 and R4 are independently NH₂ or OH;
wherein A is CₐH_{b}N_{c}O_{d}, wherein a, b, c, and d are integers;
wherein R5 is an ankyrin repeat protein comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 29 to 34, 38, and 51 to 56, wherein said ankyrin repeat protein has binding specificity for MSLN and for human serum albumin, wherein said ankyrin repeat protein comprises a Cysteine, and wherein said Cysteine forms a thioether bond connecting said ankyrin repeat protein with a maleimide ring;
and wherein R6 is a chelated radionuclide, wherein said radionuclide is Pb-212 or Pb-203.

In one embodiment, said R5 in Formula (VI) or said R6 in Formula (VII) is a chelated radionuclide, wherein said radionuclide is Pb-212. In one embodiment, said R5 in Formula (VI) or said R6 in Formula (VII) is a chelated radionuclide, wherein said radionuclide is Pb-203.

Thus, in one aspect, the invention relates to a conjugate or pharmaceutically acceptable salt thereof, the conjugate having a structure of Formula (VI):

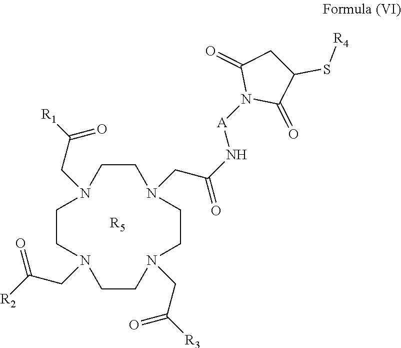

wherein R1, R2, and R3 are independently NH₂ or OH;
wherein A is CₐH_{b}N_{c}O_{d}, wherein a, b, c, and d are integers;
wherein R4 is an ankyrin repeat protein comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 29 to 34, 38, and 51 to 56, wherein said ankyrin repeat protein has binding specificity for MSLN and for human serum albumin, wherein said ankyrin repeat protein comprises a Cysteine, and wherein said Cysteine forms a thioether bond connecting said ankyrin repeat protein with a maleimide ring;
and wherein R5 is a chelated radionuclide, wherein said radionuclide is Pb-212 or Pb-203. In one embodiment, said R5 in Formula (VI) is a chelated radionuclide, wherein said radionuclide is Pb-212. In one embodiment, said R5 in Formula (VI) is a chelated radionuclide, wherein said radionuclide is Pb-203.

Thus, in one aspect, the invention relates to a conjugate or pharmaceutically acceptable salt thereof, the conjugate having a structure of Formula (VII):

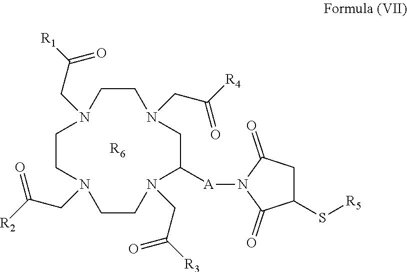

wherein R1, R2, R3 and R4 are independently NH₂ or OH;
wherein A is CₐH_{b}N_{c}O_{d}, wherein a, b, c, and d are integers;
wherein R5 is an ankyrin repeat protein comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 29 to 34, 51 to 56, wherein said ankyrin repeat protein has binding specificity for MSLN and for human serum albumin, wherein said ankyrin repeat protein comprises a Cysteine, and wherein said Cysteine forms a thioether bond connecting said ankyrin repeat protein with a maleimide ring;
and wherein R6 is a chelated radionuclide, wherein said radionuclide is Pb-212 or Pb-203. In one embodiment, said R6 in Formula (VII) is a chelated radionuclide, wherein said radionuclide is Pb-212. In one embodiment, said R6 in Formula (VII) is a chelated radionuclide, wherein said radionuclide is Pb-203.

In another aspect, the invention relates to such a conjugate or pharmaceutically acceptable salt thereof, wherein said R4 in Formula (VI) or said R5 in Formula (VII) is an ankyrin repeat protein comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 29 to 34, 38, and 51 to 56. Thus, in one embodiment, said R4 in Formula (VI) or said R5 in Formula (VII) is an ankyrin repeat protein comprising an amino acid sequence that is at least 80% identical to any one of SEQ ID NOs: 29 to 34, 38, and 51 to 56. In one embodiment, said R4 in Formula (VI) or said R5 in Formula (VII) is an ankyrin repeat protein comprising an amino acid sequence that is at least 85% identical to any one of SEQ ID NOs: 29 to 34, 38, and 51 to 56. In one embodiment, said R4 in Formula (VI) or said R5 in Formula (VII) is an ankyrin repeat protein comprising an amino acid sequence that is at least 88% identical to any one of SEQ ID NOs: 29 to 34, 38, and 51 to 56. In one embodiment, said R4 in Formula (VI) or said R5 in Formula (VII) is an ankyrin repeat protein comprising an amino acid sequence that is at least 90% identical to any one of SEQ ID NOs: 29 to 34, 38, and 51 to 56. In one embodiment, said R4 in Formula (VI) or said R5 in Formula (VII) is an ankyrin repeat protein comprising an amino acid sequence that is at least 91% identical to any one of SEQ ID NOs: 29 to 34, 38, and 51 to 56. In one embodiment, said R4 in Formula (VI) or said R5 in Formula (VII) is an ankyrin repeat protein comprising an amino acid sequence that is at least 92% identical to any one of SEQ ID NOs: 29 to 34, 38, and 51 to 56. In one embodiment, said R4 in Formula (VI) or said R5 in Formula (VII) is an ankyrin repeat protein comprising an amino acid sequence that is at least 93% identical SEQ ID NO: 29 to 34, 38, and 51 to 56. In one embodiment, said R4 in Formula (VI) or said R5 in Formula (VII) is an ankyrin repeat protein comprising an amino acid sequence that is at least 94% identical to any one of SEQ ID NOs: 29 to 34, 38, and 51 to 56. In one embodiment, said R4 in Formula (VI) or said R5 in Formula (VII) is an ankyrin repeat protein comprising an amino acid sequence that is at least 95% identical to any one of SEQ ID NOs: 29 to 34, 38, and 51 to 56. In one embodiment, said R4 in Formula (VI) or said R5 in Formula (VII) is an ankyrin repeat protein comprising an amino acid sequence that is at least 96% identical to any one of SEQ ID NOs: 29 to 34, 38, and 51 to 56. In one embodiment, said R4 in Formula (VI) or said R5 in Formula (VII) is an ankyrin repeat protein comprising an amino acid sequence that is at least 97% identical to any one of SEQ ID NOs: 29 to 34, 38, and 51 to 56. In one embodiment, said R4 in Formula (VI) or said R5 in Formula (VII) is an ankyrin repeat protein comprising an amino acid sequence that is at least 98% identical to any one of SEQ ID NOs: 29 to 34, 38, and 51 to 56. In one embodiment, said R4 in Formula (VI) or said R5 in Formula (VII) is an ankyrin repeat protein comprising an amino acid sequence that is at least 99% identical to any one of SEQ ID NOs: 29 to 34, 38, and 51 to 56. In one embodiment, any amino acid sequence differences between said amino acid sequence comprised in said ankyrin repeat protein (R4) in Formula (VI) or in said ankyrin repeat protein (R5) in Formula (VII) and said any one of SEQ ID NOs: 29 to 34, 38, and 51 to 56 represent amino acid substitutions in positions other than positions of potential target interaction residues of ankyrin repeat domains. In one embodiment, any amino acid sequence differences between said amino acid sequence comprised in said ankyrin repeat protein (R4) in Formula (VI) or in said ankyrin repeat protein (R5) in Formula (VII) and said any one of SEQ ID NOs: 29 to 34, 38, and 51 to 56 represent amino acid substitutions in (i) framework positions of ankyrin repeat domain(s), (ii) a peptide linker, (iii) a Glycine-Serine (GS), and/or (iv) a Cysteine-containing tag. In one embodiment, the C-terminal Cysteine of said any one of SEQ ID NOs: 29 to 34, 38, and 51 to 56 is not substituted with another amino acid. In one embodiment, said amino acid sequence comprised in said ankyrin repeat protein (R4) in Formula (VI) or in said ankyrin repeat protein (R5) in Formula (VII) has a Cysteine at the C-terminal end. In one embodiment, said R4 in Formula (VI) or said R5 in Formula (VII) is an ankyrin repeat protein comprising an amino acid sequence that is 100% identical to any one of SEQ ID NOs: 29 to 34, 38, and 51 to 56.

In another aspect, the invention relates to such a conjugate or pharmaceutically acceptable salt thereof, wherein said R4 in Formula (VI) or said R5 in Formula (VII) is an ankyrin repeat protein comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 30. Thus, in one embodiment, said R4 in Formula (VI) or said R5 in Formula (VII) is an ankyrin repeat protein comprising an amino acid sequence that is at least 80% identical to SEQ ID NO: 30. In one embodiment, said R4 in Formula (VI) or said R5 in Formula (VII) is an ankyrin repeat protein comprising an amino acid sequence that is at least 81% identical to SEQ ID NO: 30. In one embodiment, said R4 in Formula (VI) or said R5 in Formula (VII) is an ankyrin repeat protein comprising an amino acid sequence that is at least 82% identical to SEQ ID NO: 30. In one embodiment, said R4 in Formula (VI) or said R5 in Formula (VII) is an ankyrin repeat protein comprising an amino acid sequence that is at least 83% identical to SEQ ID NO: 30. In one embodiment, said R4 in Formula (VI) or said R5 in Formula (VII) is an ankyrin repeat protein comprising an amino acid sequence that is at least 84% identical to SEQ ID NO: 30. In one embodiment, said R4 in Formula (VI) or said R5 in Formula (VII) is an ankyrin repeat protein comprising an amino acid sequence that is at least 85% identical to SEQ ID NO: 30. In one embodiment, said R4 in Formula (VI) or said R5 in Formula (VII) is an ankyrin repeat protein comprising an amino acid sequence that is at least 86% identical to SEQ ID NO: 30. In one embodiment, said R4 in Formula (VI) or said R5 in Formula (VII) is an ankyrin repeat protein comprising an amino acid sequence that is at least 87% identical to SEQ ID NO: 30. In one embodiment, said R4 in Formula (VI) or said R5 in Formula (VII) is an ankyrin repeat protein comprising an amino acid sequence that is at least 88% identical to SEQ ID NO: 30. In one embodiment, said R4 in Formula (VI) or said R5 in Formula (VII) is an ankyrin repeat protein comprising an amino acid sequence that is at least 89% identical to SEQ ID NO: 30. In one embodiment, said R4 in Formula (VI) or said R5 in Formula (VII) is an ankyrin repeat protein comprising an amino acid sequence that is at least 90% identical to SEQ ID NO: 30. In one embodiment, said R4 in Formula (VI) or said R5 in Formula (VII) is an ankyrin repeat protein comprising an amino acid sequence that is at least 91% identical to SEQ ID NO: 30. In one embodiment, said R4 in Formula (VI) or said R5 in Formula (VII) is an ankyrin repeat protein comprising an amino acid sequence that is at least 92% identical to SEQ ID NO: 30. In one embodiment, said R4 in Formula (VI) or said R5 in Formula (VII) is an ankyrin repeat protein comprising an amino acid sequence that is at least 93% identical SEQ ID NO: 30. In one embodiment, said R4 in Formula (VI) or said R5 in Formula (VII) is an ankyrin repeat protein comprising an amino acid sequence that is at least 94% identical to SEQ ID NO: 30. In one embodiment, said R4 in Formula (VI) or said R5 in Formula (VII) is an ankyrin repeat protein comprising an amino acid sequence that is at least 95% identical to SEQ ID NO: 30. In one embodiment, said R4 in Formula (VI) or said R5 in Formula (VII) is an ankyrin repeat protein comprising an amino acid sequence that is at least 96% identical to SEQ ID NO: 30. In one embodiment, said R4 in Formula (VI) or said R5 in Formula (VII) is an ankyrin repeat protein comprising an amino acid sequence that is at least 97% identical to SEQ ID NO: 30. In one embodiment, said R4 in Formula (VI) or said R5 in Formula (VII) is an ankyrin repeat protein comprising an amino acid sequence that is at least 98% identical to SEQ ID NO: 30. In one embodiment, said R4 in Formula (VI) or said R5 in Formula (VII) is an ankyrin repeat protein comprising an amino acid sequence that is at least 99% identical to SEQ ID NO: 30. In one embodiment, any amino acid sequence differences between said amino acid sequence comprised in said ankyrin repeat protein (R4) in structure (I) or in said ankyrin repeat protein (R5) in structure (II) and SEQ ID NO: 30 represent amino acid substitutions in positions other than positions of potential target interaction residues of ankyrin repeat domains. In one embodiment, any amino acid sequence differences between said amino acid sequence comprised in said ankyrin repeat protein (R4) in structure (I) or in said ankyrin repeat protein (R5) in structure (II) and SEQ ID NO: 30 represent amino acid substitutions in (i) framework positions of ankyrin repeat domain(s), (ii) a peptide linker, (iii) a Glycine-Serine (GS), and/or (iv) a Cysteine-containing tag. In one embodiment, the C-terminal Cysteine of SEQ ID NO: 30 is not substituted with another amino acid. In one embodiment, said amino acid sequence comprised in said ankyrin repeat protein (R4) in structure (I) or in said ankyrin repeat protein (R5) in structure (II) has a Cysteine at the C-terminal end. In one embodiment, said R4 in Formula (VI) or said R5 in Formula (VII) is an ankyrin repeat protein comprising an amino acid sequence that is 100% identical to SEQ ID NO: 30.

In another aspect, the invention relates to such a conjugate or pharmaceutically acceptable salt thereof, wherein said R4 in Formula (VI) or said R5 in Formula (VII) is an ankyrin repeat protein comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 33. Thus, in one embodiment, said R4 in Formula (VI) or said R5 in Formula (VII) is an ankyrin repeat protein comprising an amino acid sequence that is at least 80% identical to SEQ ID NO: 33. In one embodiment, said R4 in Formula (VI) or said R5 in Formula (VII) is an ankyrin repeat protein comprising an amino acid sequence that is at least 85% identical to SEQ ID NO: 33. In one embodiment, said R4 in Formula (VI) or said R5 in Formula (VII) is an ankyrin repeat protein comprising an amino acid sequence that is at least 88% identical to SEQ ID NO: 33. In one embodiment, said R4 in Formula (VI) or said R5 in Formula (VII) is an ankyrin repeat protein comprising an amino acid sequence that is at least 90% identical to SEQ ID NO: 33. In one embodiment, said R4 in Formula (VI) or said R5 in Formula (VII) is an ankyrin repeat protein comprising an amino acid sequence that is at least 91% identical to SEQ ID NO: 33. In one embodiment, said R4 in Formula (VI) or said R5 in Formula (VII) is an ankyrin repeat protein comprising an amino acid sequence that is at least 92% identical to SEQ ID NO: 33. In one embodiment, said R4 in Formula (VI) or said R5 in Formula (VII) is an ankyrin repeat protein comprising an amino acid sequence that is at least 93% identical SEQ ID NO: 33. In one embodiment, said R4 in Formula (VI) or said R5 in Formula (VII) is an ankyrin repeat protein comprising an amino acid sequence that is at least 94% identical to SEQ ID NO: 33. In one embodiment, said R4 in Formula (VI) or said R5 in Formula (VII) is an ankyrin repeat protein comprising an amino acid sequence that is at least 95% identical to SEQ ID NO: 33. In one embodiment, said R4 in Formula (VI) or said R5 in Formula (VII) is an ankyrin repeat protein comprising an amino acid sequence that is at least 96% identical to SEQ ID NO: 33. In one embodiment, said R4 in Formula (VI) or said R5 in Formula (VII) is an ankyrin repeat protein comprising an amino acid sequence that is at least 97% identical to SEQ ID NO: 33. In one embodiment, said R4 in Formula (VI) or said R5 in Formula (VII) is an ankyrin repeat protein comprising an amino acid sequence that is at least 98% identical to SEQ ID NO: 33. In one embodiment, said R4 in Formula (VI) or said R5 in Formula (VII) is an ankyrin repeat protein comprising an amino acid sequence that is at least 99% identical to SEQ ID NO: 33. In one embodiment, any amino acid sequence differences between said amino acid sequence comprised in said ankyrin repeat protein (R4) in structure (I) or in said ankyrin repeat protein (R5) in structure (II) and SEQ ID NO: 33 represent amino acid substitutions in positions other than positions of potential target interaction residues of ankyrin repeat domains. In one embodiment, any amino acid sequence differences between said amino acid sequence comprised in said ankyrin repeat protein (R4) in structure (I) or in said ankyrin repeat protein (R5) in structure (II) and SEQ ID NO: 33 represent amino acid substitutions in (i) framework positions of ankyrin repeat domain(s), (ii) a peptide linker, (iii) a Glycine-Serine (GS), and/or (iv) a Cysteine-containing tag. In one embodiment, the C-terminal Cysteine of SEQ ID NO: 33 is not substituted with another amino acid. In one embodiment, said R4 in Formula (VI) or said R5 in Formula (VII) is an ankyrin repeat protein comprising an amino acid sequence that is 100% identical to SEQ ID NO: 33.

In one embodiment, said R4 in Formula (VI) or said R5 in Formula (VII) is an ankyrin repeat protein comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 51. Thus, in one embodiment, said R4 in Formula (VI) or said R5 in Formula (VII) is an ankyrin repeat protein comprising an amino acid sequence that is at least 80% identical to SEQ ID NO: 51. In one embodiment, said R4 in Formula (VI) or said R5 in Formula (VII) is an ankyrin repeat protein comprising an amino acid sequence that is at least 85% identical to SEQ ID NO: 51. In one embodiment, said R4 in Formula (VI) or said R5 in Formula (VII) is an ankyrin repeat protein comprising an amino acid sequence that is at least 88% identical to SEQ ID NO: 51. In one embodiment, said R4 in Formula (VI) or said R5 in Formula (VII) is an ankyrin repeat protein comprising an amino acid sequence that is at least 90% identical to SEQ ID NO: 51. In one embodiment, said R4 in Formula (VI) or said R5 in Formula (VII) is an ankyrin repeat protein comprising an amino acid sequence that is at least 91% identical to SEQ ID NO: 51. In one embodiment, said R4 in Formula (VI) or said R5 in Formula (VII) is an ankyrin repeat protein comprising an amino acid sequence that is at least 92% identical to SEQ ID NO: 51. In one embodiment, said R4 in Formula (VI) or said R5 in Formula (VII) is an ankyrin repeat protein comprising an amino acid sequence that is at least 93% identical SEQ ID NO: 51. In one embodiment, said R4 in Formula (VI) or said R5 in Formula (VII) is an ankyrin repeat protein comprising an amino acid sequence that is at least 94% identical to SEQ ID NO: 51. In one embodiment, said R4 in Formula (VI) or said R5 in Formula (VII) is an ankyrin repeat protein comprising an amino acid sequence that is at least 95% identical to SEQ ID NO: 51. In one embodiment, said R4 in Formula (VI) or said R5 in Formula (VII) is an ankyrin repeat protein comprising an amino acid sequence that is at least 96% identical to SEQ ID NO: 51. In one embodiment, said R4 in Formula (VI) or said R5 in Formula (VII) is an ankyrin repeat protein comprising an amino acid sequence that is at least 97% identical to SEQ ID NO: 51. In one embodiment, said R4 in Formula (VI) or said R5 in Formula (VII) is an ankyrin repeat protein comprising an amino acid sequence that is at least 98% identical to SEQ ID NO: 51. In one embodiment, said R4 in Formula (VI) or said R5 in Formula (VII) is an ankyrin repeat protein comprising an amino acid sequence that is at least 99% identical to SEQ ID NO: 51. In one embodiment, any amino acid sequence differences between said amino acid sequence comprised in said ankyrin repeat protein (R4) in structure (I) or in said ankyrin repeat protein (R5) in structure (II) and SEQ ID NO: 51 represent amino acid substitutions in positions other than positions of potential target interaction residues of ankyrin repeat domains. In one embodiment, any amino acid sequence differences between said amino acid sequence comprised in said ankyrin repeat protein (R4) in structure (I) or in said ankyrin repeat protein (R5) in structure (II) and SEQ ID NO: 51 represent amino acid substitutions in (i) framework positions of ankyrin repeat domain(s), (ii) a peptide linker, (iii) a Glycine-Serine (GS), and/or (iv) a Cysteine-containing tag. In one embodiment, the C-terminal Cysteine of SEQ ID NO: 51 is not substituted with another amino acid. In one embodiment, said R4 in Formula (VI) or said R5 in Formula (VII) is an ankyrin repeat protein comprising an amino acid sequence that is 100% identical to SEQ ID NO: 51.

In one embodiment, said R4 in Formula (VI) or said R5 in Formula (VII) is an ankyrin repeat protein comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 52. Thus, in one embodiment, said R4 in Formula (VI) or said R5 in Formula (VII) is an ankyrin repeat protein comprising an amino acid sequence that is at least 80% identical to SEQ ID NO: 52. In one embodiment, said R4 in Formula (VI) or said R5 in Formula (VII) is an ankyrin repeat protein comprising an amino acid sequence that is at least 85% identical to SEQ ID NO: 52. In one embodiment, said R4 in Formula (VI) or said R5 in Formula (VII) is an ankyrin repeat protein comprising an amino acid sequence that is at least 88% identical to SEQ ID NO: 52. In one embodiment, said R4 in Formula (VI) or said R5 in Formula (VII) is an ankyrin repeat protein comprising an amino acid sequence that is at least 90% identical to SEQ ID NO: 52. In one embodiment, said R4 in Formula (VI) or said R5 in Formula (VII) is an ankyrin repeat protein comprising an amino acid sequence that is at least 91% identical to SEQ ID NO: 52. In one embodiment, said R4 in Formula (VI) or said R5 in Formula (VII) is an ankyrin repeat protein comprising an amino acid sequence that is at least 92% identical to SEQ ID NO: 52. In one embodiment, said R4 in Formula (VI) or said R5 in Formula (VII) is an ankyrin repeat protein comprising an amino acid sequence that is at least 93% identical SEQ ID NO: 52. In one embodiment, said R4 in Formula (VI) or said R5 in Formula (VII) is an ankyrin repeat protein comprising an amino acid sequence that is at least 94% identical to SEQ ID NO: 52. In one embodiment, said R4 in Formula (VI) or said R5 in Formula (VII) is an ankyrin repeat protein comprising an amino acid sequence that is at least 95% identical to SEQ ID NO: 52. In one embodiment, said R4 in Formula (VI) or said R5 in Formula (VII) is an ankyrin repeat protein comprising an amino acid sequence that is at least 96% identical to SEQ ID NO: 52. In one embodiment, said R4 in Formula (VI) or said R5 in Formula (VII) is an ankyrin repeat protein comprising an amino acid sequence that is at least 97% identical to SEQ ID NO: 52. In one embodiment, said R4 in Formula (VI) or said R5 in Formula (VII) is an ankyrin repeat protein comprising an amino acid sequence that is at least 98% identical to SEQ ID NO: 52. In one embodiment, said R4 in Formula (VI) or said R5 in Formula (VII) is an ankyrin repeat protein comprising an amino acid sequence that is at least 99% identical to SEQ ID NO: 52. In one embodiment, any amino acid sequence differences between said amino acid sequence comprised in said ankyrin repeat protein (R4) in structure (I) or in said ankyrin repeat protein (R5) in structure (II) and SEQ ID NO: 52 represent amino acid substitutions in positions other than positions of potential target interaction residues of ankyrin repeat domains. In one embodiment, any amino acid sequence differences between said amino acid sequence comprised in said ankyrin repeat protein (R4) in structure (I) or in said ankyrin repeat protein (R5) in structure (II) and SEQ ID NO: 52 represent amino acid substitutions in (i) framework positions of ankyrin repeat domain(s), (ii) a peptide linker, (iii) a Glycine-Serine (GS), and/or (iv) a Cysteine-containing tag. In one embodiment, the C-terminal Cysteine of SEQ ID NO: 52 is not substituted with another amino acid. In one embodiment, said R4 in Formula (VI) or said R5 in Formula (VII) is an ankyrin repeat protein comprising an amino acid sequence that is 100% identical to SEQ ID NO: 52.

In one embodiment, said R4 in Formula (VI) or said R5 in Formula (VII) is an ankyrin repeat protein comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 53. Thus, in one embodiment, said R4 in Formula (VI) or said R5 in Formula (VII) is an ankyrin repeat protein comprising an amino acid sequence that is at least 80% identical to SEQ ID NO: 53. In one embodiment, said R4 in Formula (VI) or said R5 in Formula (VII) is an ankyrin repeat protein comprising an amino acid sequence that is at least 85% identical to SEQ ID NO: 53. In one embodiment, said R4 in Formula (VI) or said R5 in Formula (VII) is an ankyrin repeat protein comprising an amino acid sequence that is at least 88% identical to SEQ ID NO: 53. In one embodiment, said R4 in Formula (VI) or said R5 in Formula (VII) is an ankyrin repeat protein comprising an amino acid sequence that is at least 90% identical to SEQ ID NO: 53. In one embodiment, said R4 in Formula (VI) or said R5 in Formula (VII) is an ankyrin repeat protein comprising an amino acid sequence that is at least 91% identical to SEQ ID NO: 53. In one embodiment, said R4 in Formula (VI) or said R5 in Formula (VII) is an ankyrin repeat protein comprising an amino acid sequence that is at least 92% identical to SEQ ID NO: 53. In one embodiment, said R4 in Formula (VI) or said R5 in Formula (VII) is an ankyrin repeat protein comprising an amino acid sequence that is at least 93% identical SEQ ID NO: 53. In one embodiment, said R4 in Formula (VI) or said R5 in Formula (VII) is an ankyrin repeat protein comprising an amino acid sequence that is at least 94% identical to SEQ ID NO: 53. In one embodiment, said R4 in Formula (VI) or said R5 in Formula (VII) is an ankyrin repeat protein comprising an amino acid sequence that is at least 95% identical to SEQ ID NO: 53. In one embodiment, said R4 in Formula (VI) or said R5 in Formula (VII) is an ankyrin repeat protein comprising an amino acid sequence that is at least 96% identical to SEQ ID NO: 53. In one embodiment, said R4 in Formula (VI) or said R5 in Formula (VII) is an ankyrin repeat protein comprising an amino acid sequence that is at least 97% identical to SEQ ID NO: 53. In one embodiment, said R4 in Formula (VI) or said R5 in Formula (VII) is an ankyrin repeat protein comprising an amino acid sequence that is at least 98% identical to SEQ ID NO: 53. In one embodiment, said R4 in Formula (VI) or said R5 in Formula (VII) is an ankyrin repeat protein comprising an amino acid sequence that is at least 99% identical to SEQ ID NO: 53. In one embodiment, any amino acid sequence differences between said amino acid sequence comprised in said ankyrin repeat protein (R4) in structure (I) or in said ankyrin repeat protein (R5) in structure (II) and SEQ ID NO: 53 represent amino acid substitutions in positions other than positions of potential target interaction residues of ankyrin repeat domains. In one embodiment, any amino acid sequence differences between said amino acid sequence comprised in said ankyrin repeat protein (R4) in structure (I) or in said ankyrin repeat protein (R5) in structure (II) and SEQ ID NO: 53 represent amino acid substitutions in (i) framework positions of ankyrin repeat domain(s), (ii) a peptide linker, (iii) a Glycine-Serine (GS), and/or (iv) a Cysteine-containing tag. In one embodiment, the C-terminal Cysteine of SEQ ID NO: 53 is not substituted with another amino acid. In one embodiment, said R4 in Formula (VI) or said R5 in Formula (VII) is an ankyrin repeat protein comprising an amino acid sequence that is 100% identical to SEQ ID NO: 53.

In one embodiment, said R4 in Formula (VI) or said R5 in Formula (VII) is an ankyrin repeat protein comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 54. Thus, in one embodiment, said R4 in Formula (VI) or said R5 in Formula (VII) is an ankyrin repeat protein comprising an amino acid sequence that is at least 80% identical to SEQ ID NO: 54. In one embodiment, said R4 in Formula (VI) or said R5 in Formula (VII) is an ankyrin repeat protein comprising an amino acid sequence that is at least 85% identical to SEQ ID NO: 54. In one embodiment, said R4 in Formula (VI) or said R5 in Formula (VII) is an ankyrin repeat protein comprising an amino acid sequence that is at least 88% identical to SEQ ID NO: 54. In one embodiment, said R4 in Formula (VI) or said R5 in Formula (VII) is an ankyrin repeat protein comprising an amino acid sequence that is at least 90% identical to SEQ ID NO: 54. In one embodiment, said R4 in Formula (VI) or said R5 in Formula (VII) is an ankyrin repeat protein comprising an amino acid sequence that is at least 91% identical to SEQ ID NO: 54. In one embodiment, said R4 in Formula (VI) or said R5 in Formula (VII) is an ankyrin repeat protein comprising an amino acid sequence that is at least 92% identical to SEQ ID NO: 54. In one embodiment, said R4 in Formula (VI) or said R5 in Formula (VII) is an ankyrin repeat protein comprising an amino acid sequence that is at least 93% identical SEQ ID NO: 54. In one embodiment, said R4 in Formula (VI) or said R5 in Formula (VII) is an ankyrin repeat protein comprising an amino acid sequence that is at least 94% identical to SEQ ID NO: 54. In one embodiment, said R4 in Formula (VI) or said R5 in Formula (VII) is an ankyrin repeat protein comprising an amino acid sequence that is at least 95% identical to SEQ ID NO: 54. In one embodiment, said R4 in Formula (VI) or said R5 in Formula (VII) is an ankyrin repeat protein comprising an amino acid sequence that is at least 96% identical to SEQ ID NO: 54. In one embodiment, said R4 in Formula (VI) or said R5 in Formula (VII) is an ankyrin repeat protein comprising an amino acid sequence that is at least 97% identical to SEQ ID NO: 54. In one embodiment, said R4 in Formula (VI) or said R5 in Formula (VII) is an ankyrin repeat protein comprising an amino acid sequence that is at least 98% identical to SEQ ID NO: 54. In one embodiment, said R4 in Formula (VI) or said R5 in Formula (VII) is an ankyrin repeat protein comprising an amino acid sequence that is at least 99% identical to SEQ ID NO: 54. In one embodiment, any amino acid sequence differences between said amino acid sequence comprised in said ankyrin repeat protein (R4) in structure (I) or in said ankyrin repeat protein (R5) in structure (II) and SEQ ID NO: 54 represent amino acid substitutions in positions other than positions of potential target interaction residues of ankyrin repeat domains. In one embodiment, any amino acid sequence differences between said amino acid sequence comprised in said ankyrin repeat protein (R4) in structure (I) or in said ankyrin repeat protein (R5) in structure (II) and SEQ ID NO: 54 represent amino acid substitutions in (i) framework positions of ankyrin repeat domain(s), (ii) a peptide linker, (iii) a Glycine-Serine (GS), and/or (iv) a Cysteine-containing tag. In one embodiment, the C-terminal Cysteine of SEQ ID NO: 54 is not substituted with another amino acid. In one embodiment, said R4 in Formula (VI) or said R5 in Formula (VII) is an ankyrin repeat protein comprising an amino acid sequence that is 100% identical to SEQ ID NO: 54.

In one embodiment, said R4 in Formula (VI) or said R5 in Formula (VII) is an ankyrin repeat protein comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 55. Thus, in one embodiment, said R4 in Formula (VI) or said R5 in Formula (VII) is an ankyrin repeat protein comprising an amino acid sequence that is at least 80% identical to SEQ ID NO: 55. In one embodiment, said R4 in Formula (VI) or said R5 in Formula (VII) is an ankyrin repeat protein comprising an amino acid sequence that is at least 85% identical to SEQ ID NO: 55. In one embodiment, said R4 in Formula (VI) or said R5 in Formula (VII) is an ankyrin repeat protein comprising an amino acid sequence that is at least 88% identical to SEQ ID NO: 55. In one embodiment, said R4 in Formula (VI) or said R5 in Formula (VII) is an ankyrin repeat protein comprising an amino acid sequence that is at least 90% identical to SEQ ID NO: 55. In one embodiment, said R4 in Formula (VI) or said R5 in Formula (VII) is an ankyrin repeat protein comprising an amino acid sequence that is at least 91% identical to SEQ ID NO: 55. In one embodiment, said R4 in Formula (VI) or said R5 in Formula (VII) is an ankyrin repeat protein comprising an amino acid sequence that is at least 92% identical to SEQ ID NO: 55. In one embodiment, said R4 in Formula (VI) or said R5 in Formula (VII) is an ankyrin repeat protein comprising an amino acid sequence that is at least 93% identical SEQ ID NO: 55. In one embodiment, said R4 in Formula (VI) or said R5 in Formula (VII) is an ankyrin repeat protein comprising an amino acid sequence that is at least 94% identical to SEQ ID NO: 55. In one embodiment, said R4 in Formula (VI) or said R5 in Formula (VII) is an ankyrin repeat protein comprising an amino acid sequence that is at least 95% identical to SEQ ID NO: 55. In one embodiment, said R4 in Formula (VI) or said R5 in Formula (VII) is an ankyrin repeat protein comprising an amino acid sequence that is at least 96% identical to SEQ ID NO: 55. In one embodiment, said R4 in Formula (VI) or said R5 in Formula (VII) is an ankyrin repeat protein comprising an amino acid sequence that is at least 97% identical to SEQ ID NO: 55. In one embodiment, said R4 in Formula (VI) or said R5 in Formula (VII) is an ankyrin repeat protein comprising an amino acid sequence that is at least 98% identical to SEQ ID NO: 55. In one embodiment, said R4 in Formula (VI) or said R5 in Formula (VII) is an ankyrin repeat protein comprising an amino acid sequence that is at least 99% identical to SEQ ID NO: 55. In one embodiment, any amino acid sequence differences between said amino acid sequence comprised in said ankyrin repeat protein (R4) in structure (I) or in said ankyrin repeat protein (R5) in structure (II) and SEQ ID NO: 55 represent amino acid substitutions in positions other than positions of potential target interaction residues of ankyrin repeat domains. In one embodiment, any amino acid sequence differences between said amino acid sequence comprised in said ankyrin repeat protein (R4) in structure (I) or in said ankyrin repeat protein (R5) in structure (II) and SEQ ID NO: 55 represent amino acid substitutions in (i) framework positions of ankyrin repeat domain(s), (ii) a peptide linker, (iii) a Glycine-Serine (GS), and/or (iv) a Cysteine-containing tag. In one embodiment, the C-terminal Cysteine of SEQ ID NO: 55 is not substituted with another amino acid. In one embodiment, said R4 in Formula (VI) or said R5 in Formula (VII) is an ankyrin repeat protein comprising an amino acid sequence that is 100% identical to SEQ ID NO: 55.

In one embodiment, said R4 in Formula (VI) or said R5 in Formula (VII) is an ankyrin repeat protein comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 56. Thus, in one embodiment, said R4 in Formula (VI) or said R5 in Formula (VII) is an ankyrin repeat protein comprising an amino acid sequence that is at least 80% identical to SEQ ID NO: 56. In one embodiment, said R4 in Formula (VI) or said R5 in Formula (VII) is an ankyrin repeat protein comprising an amino acid sequence that is at least 85% identical to SEQ ID NO: 56. In one embodiment, said R4 in Formula (VI) or said R5 in Formula (VII) is an ankyrin repeat protein comprising an amino acid sequence that is at least 88% identical to SEQ ID NO: 56. In one embodiment, said R4 in Formula (VI) or said R5 in Formula (VII) is an ankyrin repeat protein comprising an amino acid sequence that is at least 90% identical to SEQ ID NO: 56. In one embodiment, said R4 in Formula (VI) or said R5 in Formula (VII) is an ankyrin repeat protein comprising an amino acid sequence that is at least 91% identical to SEQ ID NO: 56. In one embodiment, said R4 in Formula (VI) or said R5 in Formula (VII) is an ankyrin repeat protein comprising an amino acid sequence that is at least 92% identical to SEQ ID NO: 56. In one embodiment, said R4 in Formula (VI) or said R5 in Formula (VII) is an ankyrin repeat protein comprising an amino acid sequence that is at least 93% identical SEQ ID NO: 56. In one embodiment, said R4 in Formula (VI) or said R5 in Formula (VII) is an ankyrin repeat protein comprising an amino acid sequence that is at least 94% identical to SEQ ID NO: 56. In one embodiment, said R4 in Formula (VI) or said R5 in Formula (VII) is an ankyrin repeat protein comprising an amino acid sequence that is at least 95% identical to SEQ ID NO: 56. In one embodiment, said R4 in Formula (VI) or said R5 in Formula (VII) is an ankyrin repeat protein comprising an amino acid sequence that is at least 96% identical to SEQ ID NO: 56. In one embodiment, said R4 in Formula (VI) or said R5 in Formula (VII) is an ankyrin repeat protein comprising an amino acid sequence that is at least 97% identical to SEQ ID NO: 56. In one embodiment, said R4 in Formula (VI) or said R5 in Formula (VII) is an ankyrin repeat protein comprising an amino acid sequence that is at least 98% identical to SEQ ID NO: 56. In one embodiment, said R4 in Formula (VI) or said R5 in Formula (VII) is an ankyrin repeat protein comprising an amino acid sequence that is at least 99% identical to SEQ ID NO: 56. In one embodiment, any amino acid sequence differences between said amino acid sequence comprised in said ankyrin repeat protein (R4) in structure (I) or in said ankyrin repeat protein (R5) in structure (II) and SEQ ID NO: 56 represent amino acid substitutions in positions other than positions of potential target interaction residues of ankyrin repeat domains. In one embodiment, any amino acid sequence differences between said amino acid sequence comprised in said ankyrin repeat protein (R4) in structure (I) or in said ankyrin repeat protein (R5) in structure (II) and SEQ ID NO: 56 represent amino acid substitutions in (i) framework positions of ankyrin repeat domain(s), (ii) a peptide linker, (iii) a Glycine-Serine (GS), and/or (iv) a Cysteine-containing tag. In one embodiment, the C-terminal Cysteine of SEQ ID NO: 56 is not substituted with another amino acid. In one embodiment, said R4 in Formula (VI) or said R5 in Formula (VII) is an ankyrin repeat protein comprising an amino acid sequence that is 100% identical to SEQ ID NO: 56.

In another aspect, the invention relates to such a conjugate or pharmaceutically acceptable salt thereof, wherein said ankyrin repeat protein binds human MSLN (hMSLN) with a K_{D} value of or below 100 nM, of or below 30 nM, of or below 10 nM, of or below 3 nM, or of or below 1 nM, of or below 300 μM, of or below 100 μM, of or below 30 μM, or of or below 10 μM. Thus, in one embodiment, said ankyrin repeat protein binds hMSLN with a K_{D} value of or below 100 nM. In one embodiment, said ankyrin repeat protein binds hMSLN with a K_{D} value of or below 30 nM. In one embodiment, said ankyrin repeat protein binds hMSLN with a K_{D} value of or below 10 nM. In one embodiment, said ankyrin repeat protein binds hMSLN with a K_{D} value of or below 3 nM. In one embodiment, said ankyrin repeat protein binds hMSLN with a K_{D} value or of or below 1 nM. In one embodiment, said ankyrin repeat protein binds hMSLN with a K_{D} value of or below 400 μM. In one embodiment, said ankyrin repeat protein binds hMSLN with a K_{D} value of or below 350 μM. In one embodiment, said ankyrin repeat protein binds hMSLN with a K_{D} value of or below 300 μM. In one embodiment, said ankyrin repeat protein binds hMSLN with a K_{D} value of or below 200 μM. In one embodiment, said ankyrin repeat protein binds hMSLN with a K_{D} value of or below 100 μM. In one embodiment, said ankyrin repeat protein binds hMSLN with a K_{D} value of or below 35 μM. In one embodiment, said ankyrin repeat protein binds hMSLN with a K_{D} value of or below 30 μM. In one embodiment, said ankyrin repeat protein binds hMSLN with a K_{D} value of or below 10 μM. Furthermore, in one embodiment, said ankyrin repeat protein binds to the extracellular domain of hMSLN. In one embodiment, said conjugate does not specifically bind to human soluble MSLN (sMSLN). In another embodiment, said conjugate binds to cells expressing human MSLN on their surface, wherein said binding to cells expressing human MSLN on their surface is not inhibited by the presence of human soluble MSLN (sMSLN).

In another aspect, the invention relates to such a conjugate or pharmaceutically acceptable salt thereof, wherein said ankyrin repeat protein binds human serum albumin with a K_{D} value of or below 500 nM, of or below 250 nM, or of or below 100 nM. In one embodiment, said ankyrin repeat protein binds human serum albumin with a K_{D} value of or below 500 nM. In one embodiment, said ankyrin repeat protein binds human serum albumin with a K_{D} value of or below 250 nM. In one embodiment, said ankyrin repeat protein binds human serum albumin with a K_{D} value of or below 100 nM.

In another aspect, the invention relates to any one of said conjugates or pharmaceutically acceptable salts thereof described herein in any of the disclosed aspects and embodiments, wherein said conjugate binds to cells expressing human MSLN on their surface. In one embodiment, said cells are hMSLN-MC38 cells, wherein hMSLN-MC38 cells are MC38 cells engineered to express human MSLN on their surface. In one embodiment, said conjugate binds human MSLN-expressing hMSLN-MC38 cells with an EC50 below 10-7M, or about or below 5×10⁻⁸M, or about or below 2×10⁻⁸M, or about or below 10⁻⁸M, or about or below 5 x 10⁻⁹M, or about or below 10⁻⁹M, or about or below 5×10⁻¹⁰M, or about or below 2×10⁻¹⁰M, or about or below 10⁻¹⁰M. Thus, in one embodiment, said conjugate binds human MSLN-expressing hMSLN-MC38 cells with an EC50 below 10⁻⁷M, and in another embodiment, said conjugate binds human MSLN-expressing hMSLN-MC38 cells with an EC50 about or below 5×10⁻⁸M. In one embodiment, said conjugate binds human MSLN-expressing hMSLN-MC38 cells with an EC50 about or below 2×10⁻⁸M. In one embodiment, said conjugate binds human MSLN-expressing hMSLN-MC38 cells with an EC50 about or below 10⁻⁸M. In one embodiment, said conjugate binds human MSLN-expressing hMSLN-MC38 cells with an EC50 about or below 5×10⁻⁹M. In one embodiment, said conjugate binds human MSLN-expressing hMSLN-MC38 cells with an EC50 about or below 10⁻⁹M. In one embodiment, said conjugate binds human MSLN-expressing hMSLN-MC38 cells with an EC50 about or below 5×10⁻¹⁰M. In one embodiment, said conjugate binds human MSLN-expressing hMSLN-MC38 cells with an EC50 about or below 2×10⁻¹⁰M. In another embodiment, said conjugate binds human MSLN-expressing hMSLN-MC38 cells with an EC50 about or below 10⁻¹⁰M.

In another aspect, the invention relates to any one of said conjugates or pharmaceutically acceptable salts thereof described herein in any of the disclosed aspects and embodiments, wherein said conjugate binds to cells expressing human MSLN on their surface, and wherein said cells are OVCAR-3 cells. In another embodiment, said conjugate binds to cells expressing human MSLN on their surface, and wherein said cells are OVCAR-8 cells. OVCAR-3/OVCAR-8 ovarian carcinoma cells naturally (endogenously) express human MSLN on their cell surface. In one embodiment, said conjugate binds OVCAR-3 cells with an EC50 below 10⁻⁷M, or about or below 3×10⁻⁸M, or about or below 10⁻⁸M, or about or below 6×10⁻⁹M, or about or below 3×10⁻⁹M, or about or below 10⁻⁹M, or about or below 6×10⁻¹⁰M, or about or below 3×10⁻¹⁰M, or about or below 10⁻¹⁰M. Thus, in one embodiment, said conjugate binds OVCAR-3 cells with an EC50 below 10⁻⁷M, and in another embodiment, said conjugate binds OVCAR-3 cells with an EC50 about or below 3 x 10⁻⁸M. In one embodiment, said conjugate binds OVCAR-3 cells with an EC50 about or below 5×10⁻⁷M. In one embodiment, said conjugate binds OVCAR-3 cells with an EC50 about or below 1.5×10⁻⁷M. In one embodiment, said conjugate binds OVCAR-3 cells with an EC50 about or below 5×10⁻⁸M. In one embodiment, said conjugate binds OVCAR-3 cells with an EC50 about or below 1.5×10⁻⁸M. In one embodiment, said conjugate binds OVCAR-3 cells with an EC50 about or below 10⁻⁸M. In one embodiment, said conjugate binds OVCAR-3 cells with an EC50 about or below 6×10⁻⁹M. In one embodiment, said conjugate binds OVCAR-3 cells with an EC50 or about or below 5×10⁻⁹M. In one embodiment, said conjugate binds OVCAR-3 cells with an EC50 about or below 3×10⁻⁹M. In one embodiment, said conjugate binds OVCAR-3 cells with an EC50 about or below 2×10⁻⁹M. In one embodiment, said conjugate binds OVCAR-3 cells with an EC50 or about or below 10⁻⁹M. In one embodiment, said conjugate binds OVCAR-3 cells with an EC50 or about or below 6×10⁻¹⁰M. In one embodiment, said conjugate binds OVCAR-3 cells with an EC50 or about or below 5×10⁻¹⁰M. In one embodiment, said conjugate binds OVCAR-3 cells with an EC50 or about or below 3×10⁻¹⁰M. In another embodiment, said conjugate binds OVCAR-3 cells with an EC50 or about or below 10⁻¹⁰M.

In another aspect, the invention relates to any one of said conjugates or pharmaceutically acceptable salts thereof described herein in any of the disclosed aspects and embodiments, wherein said conjugate has a terminal half-life in a mouse model of at least about 5 hours, at least about 7.5 hours, at least about 10 hours, at least about 12.5 hours, at least about 15 hours, at least about 17.5 hours, at least about 20 hours, at least about 22.5 hours, at least about 25 hours, at least about 27.5 hours, at least about 30 hours, at least about 32.5 hours, at least about 35 hours, or at least about 40 hours. In one embodiment, said conjugate has a terminal half-life in a mouse model of about 5 to 45 hours, about 5 to 40 hours, about 5 to 35 hours, about 10 to 35 hours, about 10 to 30 hours, about 15 to 35 hours, about 15 to 30 hours, or about 13 to 27 hours. In one embodiment, said conjugate has a terminal half-life in a mouse model of at least about 12.5 hours. In another embodiment, said conjugate has a terminal half-life in a mouse model of about 13 to 27 hours. In one embodiment, said terminal half-life of said conjugate is measured in a BALB/c mouse model. In one embodiment, said terminal half-life of said conjugate is measured after intravenous injection of 1 mg/kg of conjugate into said mouse model.

In another aspect, the invention relates to any one of said conjugates or pharmaceutically acceptable salts thereof described herein in any of the disclosed aspects and embodiments, wherein said radionuclide is Pb-212 or Ac-225, and wherein said conjugate is capable of inhibiting tumor growth in a human MSLN-expressing mouse tumor model. In one embodiment, said human MSLN-expressing mouse tumor model is an hMSLN-expressing MC38 (hMSLN-MC38) colon carcinoma mouse model, an OVCAR-3 ovarian carcinoma mouse model, a Capan-2 pancreatic carcinoma mouse model, or an OVCAR-8 ovarian carcinoma mouse model. In one embodiment, said human MSLN-expressing mouse tumor model is an hMSLN-expressing MC38 (hMSLN-MC38) colon carcinoma mouse model. To generate the hMSLN-MC38 mouse tumor model, MC38 cells are engineered to express human MSLN on their surface. In one embodiment, said conjugate is capable of inhibiting tumor growth in a hMSLN-expressing MC38 (hMSLN-MC38) colon carcinoma mouse model. In another embodiment, said human MSLN-expressing mouse tumor model is an OVCAR-3 ovarian carcinoma mouse model. OVCAR-3 ovarian carcinoma cells naturally (endogenously) express human MSLN on their cell surface. In one embodiment, said conjugate is capable of inhibiting tumor growth in an OVCAR-3 ovarian carcinoma mouse model.

### Pharmaceutical Compositions and Kits

In another aspect, the invention relates to a pharmaceutical composition comprising any one of said conjugates or pharmaceutically acceptable salts thereof described herein in any of the aforementioned aspects and embodiments, and optionally a pharmaceutically acceptable carrier, excipient, stabilizer and/or diluent. In one exemplary embodiment, the invention relates to a pharmaceutical composition comprising a conjugate or pharmaceutically acceptable salt thereof, and optionally a pharmaceutically acceptable carrier, excipient, stabilizer and/or diluent, wherein said conjugate comprises (i) an ankyrin repeat domain with binding specificity for MSLN, (ii) a chelator, and (iii) a radionuclide, wherein said chelator is covalently connected to said ankyrin repeat domain with binding specificity for MSLN, wherein said radionuclide is bound to said chelator, wherein said radionuclide is Ac-225, Lu-177, Pb-203, Pb-212, In-111, Tb-161, Cu-64, Cu-67, Zr-89, Y-86, or Ce-134, preferably Ac-225, Lu-177, Pb-203, Pb-212, or In-111, and wherein said ankyrin repeat domain with binding specificity for MSLN comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 1 to 4, or 42 to 44. In another exemplary embodiment, the invention relates to a pharmaceutical composition comprising a conjugate or pharmaceutically acceptable salt thereof, and optionally a pharmaceutically acceptable carrier, excipient, stabilizer and/or diluent, wherein said conjugate comprises (i) an ankyrin repeat domain with binding specificity for MSLN, (ii) a connector, (iii) a chelator, and (iv) a radionuclide, wherein said connector is covalently connected to said ankyrin repeat domain, wherein said chelator is covalently connected to said connector, wherein said radionuclide is bound to said chelator, wherein said radionuclide is Ac-225, Lu-177, Pb-203, Pb-212, In-111, Tb-161, Cu-64, Cu-67, Zr-89, Y-86, or Ce-134, preferably Ac-225, Lu-177, Pb-203, Pb-212, or In-111, and wherein said ankyrin repeat domain with binding specificity for MSLN comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 1 to 4, or 42 to 44.

In another aspect, the invention relates to a pharmaceutical composition comprising any one of said conjugates or pharmaceutically acceptable salts thereof described herein in any of the aforementioned aspects and embodiments, and optionally a pharmaceutically acceptable carrier, excipient, stabilizer and/or diluent. In one exemplary embodiment, the invention relates to a pharmaceutical composition comprising a conjugate or pharmaceutically acceptable salt thereof, and optionally a pharmaceutically acceptable carrier, excipient, stabilizer and/or diluent, wherein said conjugate comprises (i) an ankyrin repeat domain with binding specificity for MSLN, (ii) a chelator, and (iii) a radionuclide, wherein said chelator is covalently connected to said ankyrin repeat domain with binding specificity for MSLN, wherein said radionuclide is bound to said chelator, wherein said radionuclide is Pb-212 or Ac-225, and wherein said ankyrin repeat domain with binding specificity for MSLN comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 1 to 4, or 42 to 44.

In another exemplary embodiment, the invention relates to a pharmaceutical composition comprising a conjugate or pharmaceutically acceptable salt thereof, and optionally a pharmaceutically acceptable carrier, excipient, stabilizer and/or diluent, wherein said conjugate comprises (i) an ankyrin repeat domain with binding specificity for MSLN, (ii) a connector, (iii) a chelator, and (iv) a radionuclide, wherein said connector is covalently connected to said ankyrin repeat domain, wherein said chelator is covalently connected to said connector, wherein said radionuclide is bound to said chelator, wherein said radionuclide is Pb-212 or Ac-225, and wherein said ankyrin repeat domain with binding specificity for MSLN comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 1 to 4, or 42 to 44.

Pharmaceutically acceptable carriers, excipients, stabilizers and/or diluents are known to the person skilled in the art and are explained in more detail below.

In one embodiment, a pharmaceutical composition comprises a conjugate or pharmaceutically acceptable salt thereof according to the present invention, and a pharmaceutically acceptable carrier, excipient, stabilizer and/or diluent, for example as described in Remington, The Science and Practice of Pharmacy; 23rd edition; Adeboye A. Ed., 2020.

Pharmaceutically acceptable carriers, excipients, stabilizers and/or diluents known to one of skill in the art include, for example, saline, Ringer's solution, dextrose solution, Hank's solution, fixed oils, ethyl oleate, 5% dextrose in saline, substances that enhance isotonicity and chemical stability, buffers and preservatives. Other potentially suitable carriers include any carrier that does not itself induce the production of antibodies harmful to the individual receiving the composition such as proteins, polysaccharides, polylactic acids, polyglycolic acids, polymeric amino acids and amino acid copolymers. A pharmaceutical composition may also comprise an antioxidant and/or a scavenger. A pharmaceutical composition may further be a combination formulation, comprising an additional active agent, such as an anti-cancer agent or an anti-angiogenic agent, or an additional bioactive compound.

In one embodiment, a pharmaceutical composition comprises a conjugate or pharmaceutically acceptable salt thereof according to the present invention, and a detergent such as nonionic detergent, a buffer such as phosphate buffer, and/or a sugar such as sucrose. In one embodiment, a pharmaceutical composition comprises a conjugate or pharmaceutically acceptable salt thereof according to the present invention, and PBS.

The formulations to be used for in vivo administration must be aseptic or sterile. This can be readily accomplished, e.g., by filtration through sterile filtration membranes.

In one embodiment, provided is the use of a conjugate or pharmaceutically acceptable salt thereof, as described herein, for manufacturing a pharmaceutical composition. In one embodiment, the use of a conjugate or pharmaceutically acceptable salt thereof, as described herein, is for manufacturing a pharmaceutical composition, wherein said conjugate comprises (i) an ankyrin repeat domain with binding specificity for MSLN, (ii) a chelator, and (iii) a radionuclide, wherein said chelator is covalently connected to said ankyrin repeat domain with binding specificity for MSLN, wherein said radionuclide is bound to said chelator, wherein said radionuclide is Ac-225, Lu-177, Pb-203, Pb-212, In-111, Tb-161, Cu-64, Cu-67, Zr-89, Y-86, or Ce-134, preferably Ac-225, Lu-177, Pb-203, Pb-212, or In-111, and wherein said ankyrin repeat domain with binding specificity for MSLN comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 1 to 4, or 42 to 44.

In another embodiment, the use of a conjugate or pharmaceutically acceptable salt thereof, as described herein, is for manufacturing a pharmaceutical composition, wherein said conjugate comprises (i) an ankyrin repeat domain with binding specificity for MSLN, (ii) a connector, (iii) a chelator, and (iv) a radionuclide, wherein said connector is covalently connected to said ankyrin repeat domain with binding specificity for MSLN, wherein said chelator is covalently connected to said connector, wherein said radionuclide is bound to said chelator, wherein said radionuclide is Ac-225, Lu-177, Pb-203, Pb-212, In-111, Tb-161, Cu-64, Cu-67, Zr-89, Y-86, or Ce-134, preferably Ac-225, Lu-177, Pb-203, Pb-212, or In-111, and wherein said ankyrin repeat domain with binding specificity for MSLN comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 1 to 4, or 42 to 44.

In one embodiment, the present invention relates to the use of a conjugate or pharmaceutically acceptable salt thereof, as described herein, for manufacturing a pharmaceutical composition. In one exemplary embodiment, the present invention relates to the use of a conjugate or pharmaceutically acceptable salt thereof, as described herein, for manufacturing a pharmaceutical composition, wherein said conjugate comprises (i) an ankyrin repeat domain with binding specificity for MSLN, (ii) a chelator, and (iii) a radionuclide, wherein said chelator is covalently connected to said ankyrin repeat domain with binding specificity for MSLN, wherein said radionuclide is bound to said chelator, wherein said radionuclide is Pb-212, and wherein said ankyrin repeat domain with binding specificity for MSLN comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 1 to 4, or 42 to 44. In another exemplary embodiment, the present invention relates to the use of a conjugate or pharmaceutically acceptable salt thereof, as described herein, for manufacturing a pharmaceutical composition, wherein said conjugate comprises (i) an ankyrin repeat domain with binding specificity for MSLN, (ii) a connector, (iii) a chelator, and (iv) a radionuclide, wherein said connector is covalently connected to said ankyrin repeat domain with binding specificity for MSLN, wherein said chelator is covalently connected to said connector, wherein said radionuclide is bound to said chelator, wherein said radionuclide is Pb-212, and wherein said ankyrin repeat domain with binding specificity for MSLN comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 1 to 4, or 42 to 44.

In another aspect, the invention relates to a kit comprising (i) a first container containing any one of said conjugates or pharmaceutically acceptable salts thereof or said pharmaceutical compositions described herein in any of the aforementioned aspects and embodiments; and (ii) a second container containing a buffered solution.

Methods of Imaging, Diagnosing and/or Treating Medical Conditions

In another aspect, the invention relates to any one of said conjugates or pharmaceutically acceptable salts thereof or said pharmaceutical composition, described herein in any of the aforementioned aspects and embodiments, for use in a method of imaging, diagnosing and/or treating a medical condition, the method comprising the step of administering to a subject in need thereof an amount of said conjugate or salt or of said pharmaceutical composition effective for imaging, diagnosing and/or treating said medical condition.

In a more particular aspect, the invention relates to any one of said conjugates or pharmaceutically acceptable salts thereof or said pharmaceutical composition, described herein in any of the aforementioned aspects and embodiments, for use in a method of treating a medical condition, the method comprising the step of administering to a subject in need thereof an amount of said conjugate or salt or of said pharmaceutical composition effective for treating said medical condition. In one embodiment, said radionuclide comprised in said conjugates or pharmaceutically acceptable salts thereof or in said pharmaceutical compositions for use in a method of treating a medical condition is Pb-212. In one embodiment, said radionuclide comprised in said conjugates or pharmaceutically acceptable salts thereof or in said pharmaceutical compositions for use in a method of treating a medical condition is Ac-225. In one embodiment, said radionuclide comprised in said conjugates or pharmaceutically acceptable salts thereof or in said pharmaceutical compositions for use in a method of treating a medical condition is Lu-177. In one embodiment, said radionuclide comprised in said conjugates or pharmaceutically acceptable salts thereof or in said pharmaceutical compositions for use in a method of treating a medical condition is Th-227. In one embodiment, said radionuclide comprised in said conjugates or pharmaceutically acceptable salts thereof or in said pharmaceutical compositions for use in a method of treating a medical condition is Tb-149. In one embodiment, said radionuclide comprised in said conjugates or pharmaceutically acceptable salts thereof or in said pharmaceutical compositions for use in a method of treating a medical condition is Tb-161. In one embodiment, said radionuclide comprised in said conjugates or pharmaceutically acceptable salts thereof or in said pharmaceutical compositions for use in a method of treating a medical condition is Y-90.

In another more particular aspect, the invention relates to any one of said conjugates or pharmaceutically acceptable salts thereof or said pharmaceutical compositions, described herein in any of the aforementioned aspects and embodiments, for use in a method of imaging and/or diagnosing a medical condition, the method comprising the step of administering to a subject an amount of said conjugate or salt or of said pharmaceutical composition effective for imaging and/or diagnosing said medical condition. In one embodiment, said radionuclide comprised in said conjugates or pharmaceutically acceptable salts thereof or in said pharmaceutical compositions for use in a method of imaging and/or diagnosing a medical condition is Pb-203. In one embodiment, said radionuclide comprised in said conjugates or pharmaceutically acceptable salts thereof or in said pharmaceutical compositions for use in a method of imaging and/or diagnosing a medical condition is Lu-177. In one embodiment, said radionuclide comprised in said conjugates or pharmaceutically acceptable salts thereof or in said pharmaceutical compositions for use in a method of imaging and/or diagnosing a medical condition is Tc-99m. In one embodiment, said radionuclide comprised in said conjugates or pharmaceutically acceptable salts thereof or in said pharmaceutical compositions for use in a method of imaging and/or diagnosing a medical condition is In-111. In one embodiment, said radionuclide comprised in said conjugates or pharmaceutically acceptable salts thereof or in said pharmaceutical compositions for use in a method of imaging and/or diagnosing a medical condition is Ga-68. In one embodiment, said radionuclide comprised in said conjugates or pharmaceutically acceptable salts thereof or in said pharmaceutical compositions for use in a method of imaging and/or diagnosing a medical condition is Cu-64. In one embodiment, said radionuclide comprised in said conjugates or pharmaceutically acceptable salts thereof or in said pharmaceutical compositions for use in a method of imaging and/or diagnosing a medical condition is Zr-89. In one embodiment, said radionuclide comprised in said conjugates or pharmaceutically acceptable salts thereof or in said pharmaceutical compositions for use in a method of imaging and/or diagnosing a medical condition is Y-86. In one embodiment, said radionuclide comprised in said conjugates or pharmaceutically acceptable salts thereof or in said pharmaceutical compositions for use in a method of imaging and/or diagnosing a medical condition is Ce-134.

In another aspect, the invention relates to a method of treating a medical condition, the method comprising the step of administering to a subject in need thereof a therapeutically effective amount of any one of said conjugates or pharmaceutically acceptable salts thereof or said pharmaceutical compositions described herein in any of the aforementioned aspects and embodiments. In one embodiment, said radionuclide comprised in said conjugate or pharmaceutically acceptable salt thereof or in said pharmaceutical composition administered to said subject is Pb-212. In one embodiment, said radionuclide comprised in said conjugate or pharmaceutically acceptable salt thereof or in said pharmaceutical composition administered to said subject is Ac-225. In one embodiment, said radionuclide comprised in said conjugate or pharmaceutically acceptable salt thereof or in said pharmaceutical composition administered to said subject is Lu-177. In one embodiment, said radionuclide comprised in said conjugate or pharmaceutically acceptable salt thereof or in said pharmaceutical composition administered to said subject is Th-227. In one embodiment, said radionuclide comprised in said conjugate or pharmaceutically acceptable salt thereof or in said pharmaceutical composition administered to said subject is Tb-149. In one embodiment, said radionuclide comprised in said conjugate or pharmaceutically acceptable salt thereof or in said pharmaceutical composition administered to said subject is Tb-161. In one embodiment, said radionuclide comprised in said conjugate or pharmaceutically acceptable salt thereof or in said pharmaceutical composition administered to said subject is Y-90. In one embodiment, said radionuclide comprised in said conjugate or pharmaceutically acceptable salt thereof or in said pharmaceutical composition administered to said subject is Cu-67.

In another aspect, the invention relates to a method of imaging and/or diagnosing a medical condition, the method comprising the step of administering to a subject an amount of any one of said conjugates or pharmaceutically acceptable salts thereof or said pharmaceutical compositions described herein in any of the aforementioned aspects and embodiments, effective for imaging and/or diagnosing said medical condition. In one embodiment, said method of imaging and/or diagnosing a medical condition comprises the steps of (i) administering to a subject an amount of any one of said conjugates or pharmaceutically acceptable salts thereof or said pharmaceutical compositions, effective for binding of conjugate or salt thereof to cells expressing MSLN on their surface, and (ii) detecting cells bound by conjugate or salt thereof and/or tissues comprising cells bound by conjugate or salt thereof. In one embodiment, said radionuclide comprised in said conjugate or pharmaceutically acceptable salt thereof or in said pharmaceutical composition administered to said subject is In-111, Cu-64, Zr-89, Y-86, Lu-177, Tb-161, Pb-203 or Ce-134.

In one embodiment, said detecting in step (ii) is performed by in vivo imaging. In one embodiment, said detecting in step (ii) is performed by in vivo imaging, wherein said in vivo imaging uses single photon emission computed tomography (SPECT). In one embodiment, said radionuclide comprised in said conjugate or pharmaceutically acceptable salt thereof or in said pharmaceutical composition administered to said subject is Pb-203. In one embodiment, said radionuclide comprised in said conjugate or pharmaceutically acceptable salt thereof or in said pharmaceutical composition administered to said subject is Lu-177. In one embodiment, said radionuclide comprised in said conjugate or pharmaceutically acceptable salt thereof or in said pharmaceutical composition administered to said subject is Tc-99m. In one embodiment, said radionuclide comprised in said conjugate or pharmaceutically acceptable salt thereof or in said pharmaceutical composition administered to said subject is In-111.

In one embodiment, in any of said conjugates or pharmaceutically acceptable salts thereof or said pharmaceutical compositions for use in a method of imaging, diagnosing and/or treating a medical condition, or in any of said methods of treating a medical condition or of imaging and/or diagnosing a medical condition, said subject is a mammal, preferably a human. In one embodiment, said medical condition is cancer. In one embodiment, said cancer comprises cells that express MSLN on their surface.

In one embodiment, said cancer is ovarian cancer, mesothelioma, pancreatic cancer, gastric cancer, or breast cancer. In one embodiment, said cancer is epithelial ovarian cancer, epithelioid mesothelioma, pancreatic adenocarcinoma, gastric adenocarcinoma, or triple negative breast cancer. In one embodiment, said cancer is ovarian cancer. In one embodiment, said cancer is mesothelioma. In one embodiment, said cancer is pancreatic cancer. In one embodiment, said cancer is gastric cancer. In one embodiment, said cancer is breast cancer.

For use of a conjugate or pharmaceutically acceptable salt thereof or of a pharmaceutical composition in a method of imaging, diagnosing and/or treating a medical condition, said conjugates or pharmaceutically acceptable salts thereof or said pharmaceutical compositions according to the present invention are typically administered to a subject by parenteral administration. For parental administration, said conjugates or pharmaceutically acceptable salts thereof or said pharmaceutical compositions according to the present invention will typically be formulated in a unit dosage injectable form such as a solution, suspension or emulsion, in association with pharmaceutically acceptable carriers, excipients, stabilizers and/or diluents as defined above. The dosage and mode of administration will depend on the individual to be imaged, diagnosed and/or treated, the particular medical condition, and the purpose of the administration.

Parenteral administration may occur, for example, by injection. Parenteral injections may be done via different routes, such as, e.g., intradermal (IM), subcutaneous (SQ), intramuscular (IM), and intravenous (IV) injections. Furthermore, parenteral injections may be done as bolus injection or by slow infusion.

Any of the above-mentioned conjugates or pharmaceutically acceptable salts thereof or pharmaceutical compositions according to the present invention are considered for use in the imaging, diagnosing and/or treatment of a disorder, disease or medical condition. The terms disorder, disease and medical condition are used interchangeably herein.

In one embodiment, any of said conjugates or pharmaceutically acceptable salts thereof or pharmaceutical compositions according to the present invention is administered intravenously or subcutaneously. In one embodiment, any of said conjugates or pharmaceutically acceptable salts thereof or pharmaceutical compositions according to the present invention is administered intravenously. In one embodiment, any of said conjugates or pharmaceutically acceptable salts thereof or pharmaceutical compositions according to the present invention is administered subcutaneously.

The use of a conjugate or pharmaceutically acceptable salt thereof or of a pharmaceutical composition according to the present invention for the treatment of a medical condition, such as cancer, can also be in combination with one or more other therapies known in the art. The term “use in combination with”, as used herein, shall refer to a co-administration, which is carried out under a given regimen. This includes synchronous administration of different compounds as well as time-shifted administration of different compounds (e.g. compound A is given once and compound B is given several times thereafter, or vice versa, or both compounds are given synchronously and one of the two is also given at later stages). In another aspect, the invention relates to the use of the conjugate or pharmaceutically acceptable salt thereof or the pharmaceutical composition as described herein in the manufacture of a medicament.

Suitably, said medicament is for treatment of cancer, optionally for treatment of ovarian cancer, mesothelioma, pancreatic cancer, gastric cancer, or breast cancer.

In another aspect, the invention relates to any one of said conjugates or pharmaceutically acceptable salts thereof or said pharmaceutical compositions according to the present invention, for use in a process of manufacturing a medicament. In one embodiment, said medicament is for the treatment of a medical condition, e.g. cancer. In one embodiment, said medicament is for the treatment of ovarian cancer, mesothelioma, pancreatic cancer, gastric cancer, or breast cancer. In one embodiment, said medicament is for the treatment of epithelial ovarian cancer, epithelioid mesothelioma, pancreatic adenocarcinoma, gastric adenocarcinoma, or triple negative breast cancer.

In another aspect, the invention relates to a process of manufacturing a medicament for the treatment of a medical condition, wherein a conjugate or pharmaceutically acceptable salt thereof or a pharmaceutical composition according to the present invention is an active ingredient of said medicament. In one embodiment, said medical condition is cancer. In one embodiment, said medical condition is ovarian cancer, mesothelioma, pancreatic cancer, gastric cancer, or breast cancer. In one embodiment, said medical condition is epithelial ovarian cancer, epithelioid mesothelioma, pancreatic adenocarcinoma, gastric adenocarcinoma, or triple negative breast cancer.

The above Detailed Description of the Invention provides numerous aspects and embodiments, describing different features of the disclosed subject matter. It should be understood that certain features described in these aspects and embodiments may be combined, as indicated for example in the embodiments E1 to E180 provided in the Summary of the Invention section.

### Definitions

Unless otherwise defined herein, scientific and technical terms used in connection with the present invention shall have the meanings that are commonly understood by those of ordinary skill in the art.

Further, unless otherwise required by context, singular terms shall include pluralities and plural terms shall include the singular. Generally, nomenclatures used in connection with, and techniques of, cell and tissue culture, molecular biology, chemistry, immunology, microbiology, genetics and protein and nucleic acid chemistry described herein are those well-known and commonly used in the art.

The terms “comprising”, “having”, “including” and “containing” are to be construed as open-ended terms unless otherwise noted. If aspects of the invention are described as “comprising” or “having” a feature, embodiments are also contemplated “consisting of” or “consisting essentially of” the feature. The use of any and all examples, or exemplary language (e.g., “such as”) provided herein, is intended merely to better illustrate the disclosure and does not pose a limitation on the scope of the disclosure unless otherwise stated. No language in the specification should be construed as indicating any non-claimed element as essential to the practice of the disclosure. The term “about” as used herein is equivalent to ±10% of a given numerical value, unless otherwise stated.

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein.

The term “nucleic acid” refers to a polynucleotide molecule, which may be a ribonucleic acid (RNA) or deoxyribonucleic acid (DNA) molecule, either single stranded or double stranded, and includes modified and artificial forms of DNA or RNA. A nucleic acid may either be present in isolated form or be comprised in recombinant nucleic acid molecules or vectors.

In the context of the present invention the term “protein” refers to a molecule comprising a polypeptide, wherein at least part of the polypeptide has, or is able to acquire, a defined three-dimensional arrangement by forming secondary, tertiary, and/or quaternary structures within a single polypeptide chain and/or between multiple polypeptide chains. If a protein comprises two or more polypeptide chains, the individual polypeptide chains may be linked non-covalently or covalently, e.g. by a disulfide bond between two polypeptides. A part of a protein, which individually has, or is able to acquire, a defined three-dimensional arrangement by forming secondary and/or tertiary structure, is termed “protein domain”. Such protein domains are well known to the practitioner skilled in the art.

The term “recombinant” as used in recombinant protein, recombinant polypeptide and the like, means that said protein or polypeptide is produced by the use of recombinant DNA technologies well known to the practitioner skilled in the art. For example, a recombinant DNA molecule (e.g. produced by gene synthesis) encoding a polypeptide can be cloned into a bacterial expression plasmid (e.g. pQE30, QIAgen), yeast expression plasmid, mammalian expression plasmid, or plant expression plasmid, or a DNA enabling in vitro expression. If, for example, such a recombinant bacterial expression plasmid is inserted into appropriate bacteria (e.g. Escherichia col), these bacteria can produce the polypeptide(s) encoded by this recombinant DNA. The correspondingly produced polypeptide or protein is called a recombinant polypeptide or recombinant protein.

In the context of the present invention, the term “polypeptide” relates to a molecule consisting of a chain of multiple, i.e. two or more, amino acids linked via peptide bonds. Preferably, a polypeptide consists of more than eight amino acids linked via peptide bonds. The term “polypeptide” also includes multiple chains of amino acids, linked together by S—S bridges of cysteines. Polypeptides are well-known to the person skilled in the art.

The term “target” refers to an individual molecule such as a nucleic acid, a polypeptide or protein, a carbohydrate, or any other naturally or non-naturally occurring molecule or moiety, including any part of such individual molecule, or complexes of two or more of such molecules. The target may be a whole cell or a tissue sample. Preferably, the target is a naturally occurring or non-natural polypeptide or a polypeptide containing chemical modifications, for example modified by natural or non-natural phosphorylation, acetylation, or methylation.

Patent application WO2002020565 and Forrer et al., 2003 (Forrer, P., Stumpp, M. T., Binz, H. K., Pluckthun, A., 2003. FEBS Letters 539, 2-6), contain a general description of repeat protein features and repeat domain features, techniques and applications. The term “repeat protein” refers to a protein comprising one or more repeat domains. Preferably, a repeat protein comprises one, two, three, four, five or six repeat domains. Furthermore, said repeat protein may comprise additional non-repeat protein domains, polypeptide tags and/or peptide linkers.

The term “repeat domain” refers to a protein domain comprising two or more consecutive repeat modules as structural units, wherein said repeat modules have structural and sequence homology. Preferably, a repeat domain also comprises an N-terminal and/or a C-terminal capping module. For clarity, a capping module can be a repeat module. Such repeat domains, repeat modules, and capping modules, sequence motifs, as well as structural homology and sequence homology are well known to the practitioner in the art from examples of ankyrin repeat domains (Binz et al., J. Mol. Biol. 332, 489-503, 2003; Binz et al., 2004, loc. cit.; WO2002020565; WO2012069655), leucine-rich repeat domains (WO2002020565), tetratricopeptide repeat domains (Main, E. R., Xiong, Y., Cocco, M. J., D'Andrea, L., Regan, L., Structure 11(5), 497-508, 2003), and armadillo repeat domains (WO2009040338). It is further well known to the practitioner in the art that such repeat domains are different from proteins comprising repeated amino acid sequences, where every repeated amino acid sequence is able to form an individual domain (for example FN3 domains of Fibronectin). The repeat domains can be binding domains.

The term “ankyrin repeat domain” refers to a repeat domain comprising two or more consecutive ankyrin repeat modules as structural units, wherein said ankyrin repeat modules have structural and sequence homology.

The term “designed” as used in designed repeat protein, designed repeat domain and the like refers to the property that such repeat proteins and repeat domains, respectively, are man-made and do not occur in nature. The binding domains of the instant invention are designed repeat domains. Preferably, a designed repeat domain of the invention is a designed ankyrin repeat domain.

A residue or amino acid residue refers to an amino acid comprised in a peptide chain. The term “target interaction residues” refers to amino acid residues of a repeat module, which contribute to the direct interaction with a target. Such contribution of a residue can be tested, e.g., in a binding assay, for example in a mutagenesis study performed to identify residues required, sufficient, and/or necessary for a repeat domain to bind a target with its original binding affinity or quantity (i.e. its binding affinity or quantity in the absence of any mutations). Target interaction residues can also be determined by structural analyses of a repeat domain bound to a target.

The term “framework residues” refers to amino acid residues of a repeat module, which contribute to the folding topology, i.e. which contribute to the fold of said repeat module or which contribute to the interaction with a neighboring module. Such contribution may be the interaction with other residues in the repeat module, or the influence on the polypeptide backbone conformation as found in a-helices or p-sheets, or the participation in amino acid stretches forming linear polypeptides or loops.

Such framework and target interaction residues may be identified by analysis of the structural data obtained by physicochemical methods, such as X-ray crystallography, NMR and/or CD spectroscopy, or by comparison with known and related structural information well known to practitioners in structural biology and/or bioinformatics.

Preferably, framework residues shall correspond to residues occupying specific positions within repeat modules as described in Table 1:

TABLE 1

| | Reference | Position of |
|---|---|---|
| Repeat modules | sequence | framework residues |
| N-terminal capping | SEQ ID NO: 35 | 1 to 3, 5 to 7, 9, |
| module | | 10, 13 to 30 |
| Internal repeat | SEQ ID NO: 36 | 1, 2, 5, 7 to 10, 12, |
| module | | 13, 16 to 33 |
| C-terminal capping | SEQ ID NO: 37 | 1, 2, 5, 7 to 13, |
| module | | 16 to 28 |

Table 2 shows preferred positions of potential target interaction residues in designed ankyrin repeat domains with binding specificity for a target.

TABLE 2

| | | Position of potential |
|---|---|---|
| | Reference | target interaction |
| Repeat modules | sequence | residues |
| N-terminal capping module | SEQ ID NO: 35 | 4, 8, 11 and 12 |
| Internal repeat module | SEQ ID NO: 36 | 3, 4, 6, 11, 14 and 15 |
| C-terminal capping module | SEQ ID NO: 37 | 3, 4, 6, 14 and 15 |

Specifically for the purpose of defining positions in amino acid sequences of ankyrin repeat domains and proteins provided herein (including SEQ ID NOs: 1 to 8, 17 to 26, 29 to 34, and 38 to 40, 42 to 45), at which amino acid substitutions are permitted or are not permitted in some embodiments of the invention described herein, the term “framework residues” includes the amino acid residues located at the positions within a designed ankyrin repeat domain that correspond to the positions listed in Table 1 for the representative N-terminal capping module (i.e. positions 1 to 3, 5 to 7, 9, 10, and 13 to 30 of SEQ ID NO: 35), the representative internal repeat module (i.e. positions 1, 2, 5, 7 to 10, 12, 13, and 16 to 33 of SEQ ID NO: 36) and the representative C-terminal capping module (i.e. positions 1, 2, 5, 7 to 13, and 16 to 28 of SEQ ID NO: 37). The term “framework residues” does not include the amino acid residues located at the positions within a designed ankyrin repeat domain that correspond to the positions listed in Table 2 for the representative N-terminal capping module (i.e. positions 4, 8, 11 and 12 of SEQ ID NO: 35), the representative internal repeat module (i.e. positions 3, 4, 6, 11, 14 and 15 of SEQ ID NO: 36) and the representative C-terminal capping module (i.e. positions 3, 4, 6, 14 and 15 of SEQ ID NO: 37).

Specifically for the purpose of defining positions in amino acid sequences of ankyrin repeat domains and proteins provided herein (including SEQ ID NOs: 1 to 8, 17 to 26, 29 to 34, and 38 to 40, 42 to 44), at which amino acid substitutions are permitted or are not permitted in some embodiments of the invention described herein, the term “potential target interaction residues” includes the amino acid residues located at the positions within a designed ankyrin repeat domain that correspond to the positions listed in Table 2 for the representative N-terminal capping module (i.e. positions 4, 8, 11 and 12 of SEQ ID NO: 35), the representative internal repeat module (i.e. positions 3, 4, 6, 11, 14 and 15 of SEQ ID NO: 36) and the representative C-terminal capping module (i.e. positions 3, 4, 6, 14 and 15 of SEQ ID NO: 37). The term “potential target interaction residues” does not include the amino acid residues located at the positions within a designed ankyrin repeat domain that correspond to the positions listed in Table 1 for the representative N-terminal capping module (i.e. positions 1 to 3, 5 to 7, 9, 10, and 13 to 30 of SEQ ID NO: 35), the representative internal repeat module (i.e. positions 1, 2, 5, 7 to 10, 12, 13, and 16 to 33 of SEQ ID NO: 36) and the representative C-terminal capping module (i.e. positions 1, 2, 5, 7 to 13, and 16 to 28 of SEQ ID NO: 37).

In some embodiments, an amino acid substitution in a sequence provided herein (such as, e.g., SEQ ID NOs: 1 to 8, 17 to 26, 29 to 34, and 38 to 40, 42 to 44) is an exemplary substitution according to Table 3.

In some embodiments, an amino acid substitution in a sequence provided herein (such as, e.g., SEQ ID NOs: i to 8, 17 to 26, 29 to 34, and 38 to 40, 42 to 44) is a conservative substitution according to Table 3.

In some embodiments, the substitution is made outside the structural core residues of an ankyrin repeat domain, e.g., in the beta loops that connect the alpha-helices.

TABLE 3
Amino acid substitutions

| Original | Conservative | Exemplary |
|---|---|---|
| Residue | Substitutions | Substitutions |
| Ala (A) | Val | Val; Leu; Ile |
| Arg (R) | Lys | Lys; Gln; Asn |
| Asn (N) | Gln | Gln; His; Asp, Lys; Arg |
| Asp (D) | Glu | Glu; Asn |
| Cys (C) | Ser | Ser; Ala |
| Gln (Q) | Asn | Asn; Glu |
| Glu (E) | Asp | Asp; Gln |
| Gly (G) | Ala | Ala |
| His (H) | Arg | Asn; Gln; Lys; Arg |
| Ile (I) | Leu | Leu; Val; Met; Ala; Phe; Norleucine |
| Leu (L) | Ile | Norleucine; Ile; Val; Met; Ala; Phe |
| Lys (K) | Arg | Arg; Gln; Asn |
| Met (M) | Leu | Leu; Phe; Ile |
| Phe (F) | Tyr | Leu; Val; Ile; Ala; Tyr |
| Pro (P) | Ala | Ala |
| Ser (S) | Thr | Thr |
| Thr (T) | Ser | Ser |
| Trp (W) | Tyr | Tyr; Phe |
| Tyr(Y) | Phe | Trp; Phe; Thr; Ser |
| Val (V) | Leu | Ile; Leu; Met; Phe; Ala; Norleucine |

The term “binding specificity”, “has binding specificity for a target”, “specifically binding to a target”, “binding to a target with high specificity”, “specific for a target” or “target specificity” and the like means that a binding protein or binding domain binds to a target with a lower dissociation constant (i.e. it binds with higher affinity) than it binds to an unrelated protein such as the E. coli maltose binding protein (MBP). Preferably, the dissociation constant (“K_{D}”) for the target is at least 10²; more preferably, at least 10³; more preferably, at least 10⁴; or more preferably, at least 105 times lower than the corresponding dissociation constant for MBP. Methods to determine dissociation constants of protein-protein interactions, such as surface plasmon resonance (SPR) based technologies (e.g. SPR equilibrium analysis) or isothermal titration calorimetry (ITC) are well known to the person skilled in the art. The measured K_{D} values of a particular protein-protein interaction can vary if measured under different conditions (e.g., salt concentration, pH). Thus, measurements of K_{D} values are preferably made with standardized solutions of protein and a standardized buffer, such as PBS.

Binding of any molecule to another is governed by two forces, namely the association rate (kₒₙ) and the dissociation rate (k_{off}). The affinity of any binder [B] to a target [T] can then be expressed by the equilibrium dissociation constant K_{D}, which is the quotient of k_{off}/kₒₙ.

$$[ B ] + [ T ] {⇌}_{{k}_{off}}^{{k}_{on}} [ BT ]$$

kₒₙ is a second-order rate constant of the binding reaction, with the unit M⁻¹s⁻¹, whereas the dissociation reaction k_{off} is a first-order rate constant with the unit s⁻¹. From this it becomes clear that the association reaction depends on the concentration of the reactants, whereas the dissociation is independent of the concentration, following a simple exponential decay function.

A variety of methods of measuring binding affinity are known in the art, any of which can be used for purposes of the present invention. For example, as exemplified herein, the binding affinity of a particular binding moiety to a drug molecule target can be expressed as K_{D} value, which refers to the dissociation constant of the binding moiety and the drug molecule target. K_{D} is the ratio of the rate of dissociation, also called the “off-rate (k_{off})”, to the association rate, or “on-rate (kₒₙ)”. Thus, K_{D} equals k_{off}/kₒₙ and is expressed as a molar concentration (M), and the smaller the K_{D}, the stronger the affinity of binding.

K_{D} values can be determined using any suitable method. One exemplary method for measuring K_{D} is surface plasmon resonance (SPR) (see, e.g., Nguyen et al. Sensors (Basel). 2015 May 5; 15(5):10481-510). K_{D} value may be measured by SPR using a biosensor system such as a BIACORE®system. BIAcore kinetic analysis comprises, e.g., analysing the binding and dissociation of an antigen from chips with immobilized molecules (e.g., molecules comprising epitope binding domains), on their surface. Another method for determining the K_{D} of a protein is by using Bio-Layer Interferometry (see, e.g., Shah et al. J Vis Exp. 2014; (84): 51383). A K_{D} value may be measured using OCTET® technology (Octet Qke system, ForteBio). Alternatively, or in addition, a KinExA® (Kinetic Exclusion Assay) assay, available from Sapidyne Instruments (Boise, Id.) can also be used. Any method suitable for assessing the binding affinity between two binding partners is encompassed herein. Surface plasmon resonance (SPR) is particularly preferred.

Most preferably, the K_{D} values are determined in PBS and by SPR.

The term “PBS” means a phosphate buffered water solution containing 137 mM NaCl, 10 mM phosphate and 2.7 mM KCl and having a pH of 7.4.

The term “mouse serum albumin” refers to UniProt accession number P07724, the term “cynomolgus monkey serum albumin” (i.e. Macaca fascioularis) refers to UniProt accession number A2V9Z4, and the term “human serum albumin” refers to UniProt accession number PG2768.

Mesothelin may be derived from different isoforms of the mesothelin precursor proteins. The conjugates comprising an ankyrin repeat domain with binding specificity for MSLN described herein may bind to mesothelin derived from any precursor isoform. The MSLN gene encodes a 622-residue (69 kDa) precursor protein recognized as pre-pro mesothelin (e.g. the major isoform referenced in UniProt ID Q13421-3). Upon insertion into the cell membrane, the N-terminal signal peptide, comprising residues 1-33 of the major isoform, undergoes excision. Truncation of C-terminal residues 599-622 prompts the addition of a GPI anchor at S598. Cleavage of the precursor protein generates a GPI-anchored 303 amino acid residue (40 kDa) fragment termed “membrane-bound mesothelin”, alongside a 31 kDa secreted fragment, also referred to as megakaryocyte-potentiating factor (MPF). Mesothelin can undergo membrane shedding through proteolytic cleavage at one of several cleavage sites at the membrane-proximal region of mesothelin, resulting in truncated mesothelin protein that remains on the cell surface (tMSLN) as well as soluble mesothelin. As used herein, the term “membrane-proximal region” refers to the C-terminal portion of mature mesothelin that is located adjacent to the GPI anchor attachment site at S598, typically encompassing residues within approximately 30 amino acids of the GPI anchor, including but not limited to residues 570-598, and frequently characterized as residues 580-595. Epitopes within this membrane-proximal region are distinguished by their proximity to the cell membrane and may exhibit preferential binding to membrane-bound mesothelin over shed or soluble forms of mesothelin. As used herein, the term “soluble mesothelin” (or “sMSLN”) refers to amino acid sequences 296-580 of human mesothelin, while “membrane-bound mesothelin” refers to amino acid sequences 296-597, both with reference to UniProt ID Q13421-3.

The term pharmacokinetic properties refers to various pharmacokinetic parameters, including area under the curve, clearance, and terminal half-life (or serum half-life). These parameters of pharmacokinetic properties and ways to determine them are well known in the art (see, e.g., Mahmood, I., Methods to determine pharmacokinetic profiles of therapeutic proteins, Drug Discov Today: Technol (2009), doi:10.1016/j.ddtec.2008.12.001).

The term “linked” or “linkage” refers to any covalent or non-covalent linkage between two chemical and/or biochemical moieties, e.g. between a chemical moiety and a protein such as a designed repeat domain or a designed repeat protein. The term “connected”, “covalently connected” or “covalent connection” refers to any covalent linkage between two chemical and/or biochemical moieties. Such moieties include, for example, a connector, a chelator, and a polypeptide such as a designed repeat domain or a designed repeat protein.

The term “radionuclide” or “radioisotope” refers to isotopes of natural or artificial origin with an unstable neutron to proton ratio that disintegrates with the emission of corpuscular (i.e. protons (alpha-radiation) or electrons (beta-radiation)) or electromagnetic radiation (gamma-radiation). In other words, radionuclides undergo radioactive decay. Such radionuclides include, without limitation, ⁹⁴Tc, ^{99m}Tc, ⁹⁰In, ¹¹¹In, ⁶⁷Ga, ⁶⁸Ga, ⁸⁶Y, ⁹⁰Y, ¹⁷⁷Lu, ¹⁵¹Tb, ²²³Ra, ¹⁸⁶Re, ¹⁸⁸Re, ⁶⁴Cu, ⁶⁷Cu, ⁵⁵Co, ⁵⁷Co, ⁴³Sc, ⁴⁴Sc, ⁴⁷Sc, ²³⁵Ac, ²¹³Bi, ²¹²Bi, ²⁰³Pb, ²¹²Pb, ²²⁷Th, ¹⁵³Sm, ¹⁶⁶Ho, ¹⁵²Gd, ¹⁵³Gd, ¹⁵⁷Gd, ²²⁵Ac or ¹⁶⁶Dy. The choice of suitable radionuclides may depend on the chemical structure and chelating capability of the chelating agent (or chelator), and the intended application of the resulting compound (e.g. diagnostic, therapeutic or imaging). The term “radionuclide” or “radioisotope” as used herein includes ions thereof. Thus, for example, the terms lead, Pb, ²¹²Pb or ²⁰³Pb are intended to encompass the ionic form of the radioisotope element.

The terms “chelator” or “chelating agent” refer to polydentate (multiple bonded) ligands capable of forming two or more separate coordinate bonds with (“coordinating”) a central (metal) ion. Specifically, such molecules donating one or more electron pairs may also be referred to as “Lewis bases”. The central (metal) ion is usually coordinated by two or more electron pairs to the chelating agent. Usually, the electron pairs of a chelating agent forms coordinate bonds with a single central (metal) ion; however, in certain examples, a chelating agent may form coordinate bonds with more than one metal ion, with a variety of binding modes being possible. The terms “coordinating” and “coordination” refer to an interaction in which one or more electron pair donor coordinatively bonds (“is coordinated”) to, i.e. donates two or more pairs of electrons with, one central (metal) ion. The chelating agent is preferably chosen based on its ability to coordinate (or bind) the desired central (metal) ion, usually a radionuclide as specified herein. Examples of chelators include DOTA (1,4,7,10-tetraazacyclododecane-1,4,7,10-tetraacetic acid) and DOTAM (1,4,7,10-tetraazacyclododecane-1,4,7,10-tetraacetamide) (also called TCMC), and analogues or derivatives thereof. Such analogues or derivatives include, for example, monoacid forms of TCMC. Other examples of suitable chelators are diethylenetriaminepentaacetic acid (DTPA), 1,4,7-triazacyclononane-1,4,7-triacetic acid (NOTA), 1,4,7,10-tetraazacyclododecane-1-(glutaric acid)-4,7,10-triacetic acid (DOTAGA), pyridine-containing macrocyclic chelators (PSC class), deferoxamine (DFO), 1,4,7-triazacyclononane-1-glutaric acid-4,7-diacetic acid (NODAGA), macrocyclic pyridinophane tetraacetic acid (MACROPA), sarcophagine derivative, or derivatives thereof. A chelator is appropriately matched to a specific radioisotope by the person skilled in the art. Such matching is based on coordination chemistry requirements, ionic radius, and oxidation state of the specific radioisotope. A chelator should also provide thermodynamic stability for the metal-chelator complex under physiological conditions, exhibit kinetic inertness with a dissociation half-life of at least 24 hours in human serum, and maintain radiochemical purity of at least 90% for a period equivalent to at least two half-lives of the radioisotope. Examples of chelators are also provided by Price and Orvig (Chem. Soc. Rev., 2014, 43, 260).

The term “connector” refers to any chemical moiety that covalently connects a peptide or polypeptide, such as, e.g., an ankyrin repeat protein, such as, e.g., a protein comprising an ankyrin repeat domain with binding specificity for MSLN, with a chelator. Examples of connectors include chemical moieties comprising maleimide or a derivative thereof and chemical moieties comprising phenyl isothiocyanate or a derivative thereof.

Examples of a chelator connected to a connector include p-SCN-Bn-TCMC (2-[4,7,10-tris(2-amino-2-oxoethyl)-6-[(4-isothiocyanatophenyl)methyl]-1,4,7,10-tetrazacyclododec-1-yl]acetamide) (PubChem CID 10076170) and monoacid forms of p-SCN-Bn-TCMC, wherein the chelator TCMC or a monoacid form thereof is connected to a chemical moiety that allows covalent connection to a peptide or polypeptide.

The term “physiological conditions” refers to conditions normally present in a mammalian body. Thus, for example for humans, physiological conditions mean a pH between 7.35 and 7.45, with the average at 7.40, and a temperature between 36.1° C. and 37.2° C., with the average at 37° C.

Embodiments of the present disclosure are further defined in the following Examples. It should be understood that these Examples are given by way of illustration only and that the invention is not restricted to the particular embodiments described in the Examples. From the above discussion and these Examples, one skilled in the art can ascertain the essential characteristics of the present disclosure, and without departing from the spirit and scope thereof, can make various changes and modifications of the embodiments of the disclosure to adapt it to various usages and conditions. Thus, various modifications of the embodiments of the disclosure, in addition to those shown and described herein, will be apparent to those skilled in the art from the foregoing description. Such modifications are also intended to fall within the scope of the appended claims. The disclosure of each reference set forth herein is incorporated herein by reference in its entirety, and for the disclosure referenced herein. This specification refers to a number of amino acid sequences and SEQ ID NOs that are disclosed in the Sequence Table and the associated Sequence Listing, which is herewith incorporated by reference in its entirety.

### EXAMPLES

### Materials

Chemicals were purchased from Sigma-Aldrich (USA). Oligonucleotides were from Microsynth (Switzerland). Unless stated otherwise, DNA polymerases, restriction enzymes and buffers were from New England Biolabs (USA) or Fermentas/Thermo Fisher Scientific (USA). Inducible E. coli expression strains were used for cloning and protein production, e.g. E. coli XL1-blue (Stratagene, USA) or BL21 (Novagen, USA). TEV protease was from Sigma-Aldrich (USA). Double-stranded gene fragments (eBlocks) were obtained from IDT (US). Maleimide DTPA was purchased from Chematech, metal-free PBS was purchased from VWR and Chelex 100 chelating resins were purchased from BioRad.

### Molecular Biology

Unless stated otherwise, methods are performed according to known protocols (see, e.g., Sambrook J., Fritsch E. F. and Maniatis T., Molecular Cloning: A Laboratory Manual, Cold Spring Harbor Laboratory 1989, New York or subsequent editions).

### Designed Ankyrin Repeat Protein Libraries

Methods to generate designed ankyrin repeat protein libraries have been described, e.g. in U.S. Pat. No. 7,417,130; Binz et al. 2003, loc. cit.; Binz et al. 2004, loc. cit. By such methods designed ankyrin repeat protein libraries having randomized ankyrin repeat modules and/or randomized capping modules can be constructed. For example, such libraries could accordingly be assembled based on a fixed N-terminal capping module or a randomized N-terminal capping module, one or more randomized repeat modules, and a fixed C-terminal capping module or a randomized C-terminal capping module (see, e.g., the N-terminal capping modules and C-terminal capping modules provided in WO2021116462 and WO2021116469). Preferably, such libraries are assembled to not have any of the amino acids C, G, M, N (in front of a G residue) and P at randomized positions of repeat or capping modules.

Furthermore, such randomized modules in such libraries may comprise additional polypeptide loop insertions with randomized amino acid positions. Examples of such polypeptide loop insertions are complement determining region (CDR) loop libraries of antibodies or de novo generated peptide libraries.

For example, such a loop insertion could be designed using the structure of the N-terminal ankyrin repeat domain of human ribonuclease L (Tanaka, N., Nakanishi, M, Kusakabe, Y, Goto, Y., Kitade, Y, Nakamura, K. T., EMBO J. 23(30), 3929-3938, 2004) as guidance. In analogy to this ankyrin repeat domain where ten amino acids are inserted in the beta-turn present close to the border of two ankyrin repeats, ankyrin repeat protein libraries may contain randomized loops (with fixed and randomized positions) of variable length (e.g. 1 to 20 amino acids) inserted in one or more beta-turns of an ankyrin repeat domain.

The design of such an ankyrin repeat protein library may be guided by known structures of an ankyrin repeat domain interacting with a target. Examples of such structures, identified by their Protein Data Bank (PDB) unique accession or identification codes (PDB-IDs), are 1WDY, 3V31, 3V30, 3V2X, 3V20, 3UXG, 3TWQ-3TWX, 1N11, 1S70 and 2ZGD.

Examples of designed ankyrin repeat protein libraries, such as N2C and N3C designed ankyrin repeat protein libraries, have been described (U.S. Pat. No. 7,417,130; Binz et al. 2003, loc. cit.; Binz et al. 2004, loc. cit.). The digit in N2C and N3C describes the number of randomized repeat modules present between the N-terminal and C-terminal capping modules.

g specificity for MSLN and their kinetic binding parameters and binding affinities
Ankyrin Repeat Domains with Binding Specificity for MSLN and Generation of Half-Life Extended MSLN Specific Binding Proteins

Ankyrin repeat domains with binding specificity for MSLN have been described in patent application PCT/EP2025/052531 (incorporated by reference in its entirety). Examples of such MSLN-specific ankyrin repeat domains include SEQ ID NOs: 1 to 4 and 8. The MSLN-specific ankyrin repeat domains of SEQ ID NOs: 1 to 4 bind to membrane-proximal epitopes of MSLN, which are part of truncated mesothelin protein (tMSLN) that remains on the cell surface after shedding and/or which are not part of soluble mesothelin (sMSLN), while the MSLN-specific ankyrin repeat domain of SEQ ID NO: 8 binds to a more distal epitope of MSLN, which is part of the shed portion of mesothelin (sMSLN) (see PCT/EP2025/052531). Ankyrin repeat domains with binding specificity for human serum albumin (HSA) have been described previously, e.g. in WO 2020/245171. Examples of such serum albumin-specific ankyrin repeat domains include SEQ ID NOs: 5 to 7. Linking a HSA-specific ankyrin repeat domain to an ankyrin repeat protein without binding specificity for HSA may extend the serum half-life of such ankyrin repeat protein. Applicant investigated the effects of linking a HSA-specific ankyrin repeat domain to an ankyrin repeat protein with binding specificity for MSLN on the biological properties of ankyrin repeat proteins with binding specificity for MSLN.

In order to generate half-life extended MSLN-specific ankyrin repeat proteins, nucleic acid sequences encoding the MSLN-specific ankyrin repeat domains of SEQ ID NOs: 1 to 4 and 8 were first subcloned into an expression cassette, resulting in constructs comprising a MSLN-specific ankyrin repeat domain either N- or C-terminally of a serum albumin-specific ankyrin repeat domain (such as one of SEQ ID NOs: 5 to 7), and a peptide linker (such as one of SEQ ID NOs: 9 to 15) connecting the two ankyrin repeat domains. Examples of sequences comprising two ankyrin repeat domains (2D), one with binding specificity for MSLN and one with binding specificity for serum albumin, include SEQ ID NOs: 17 to 26. Constructs optionally further comprised an N-terminal His tag (SEQ ID NO: 27) or His-TEV tag (with a TEV cleavage site) (SEQ ID NO: 28), and/or a GS at the N-terminal end of the 2D ankyrin repeat domain cassette. Constructs were also generated with a cysteine-containing tag (such as SEQ ID NO: 16) at the C-terminal end, intended for conjugation to a chelator. Examples of sequences comprising two ankyrin repeat domains (2D) and a cysteine-containing tag include SEQ ID NOs: 29 to 34, 38 to 40, and 51 to 56. Resulting constructs comprising two ankyrin repeat domains (2D), one with binding specificity for MSLN and one with binding specificity for serum albumin, were expressed in E. coli and purified using a N-terminal His-tag according to standard protocols. Similarly, constructs comprising only one ankyrin repeat domain (1D), with binding specificity for MSLN (such as SEQ ID NOs: 1 to 4 and 8), were also expressed in E. coli and purified using a N-terminal His-tag.

### Kinetic Binding Parameters and Binding Affinities of Recombinant Ankyrin Repeat Proteins

Kinetic binding parameters and binding affinities of two domain (2D) ankyrin repeat proteins comprising an ankyrin repeat domain with binding specificity for MSLN and an ankyrin repeat domain with binding specificity for serum albumin were determined by surface plasmon resonance (SPR) multi-trace analysis. For this purpose, the 2D ankyrin repeat proteins were generated with a cysteine-containing tag at their C-terminal end and conjugated to a chelator (DOTA) using maleimide chemistry, as described in more detail in Example 3. The conjugated 2D ankyrin repeat proteins were compared with proteins comprising only a MSLN-specific ankyrin repeat domain (1D), but not a peptide linker, a serum albumin-specific ankyrin repeat domain, a cysteine-containing tag, or a conjugated chelator. Binding parameters were determined with human MSLN and cynomolgus MSLN as target proteins.

In brief, a SPR assay was used to determine the binding kinetics to the target MSLN. The data were generated using BioRad ProteOn instrument in SPR running buffer (PBS, pH 7.4 containing 0.005% Tween 20®). A Streptavidin chip SAHC200M (Xantec) was conditioned according to the manufacturer's protocol. The chip was coated with biotinylated target (bio-MLSN). All analytes (3-fold dilution starting from 60, 30 or 10 down to 0.74, 0.37 or 0.12 nM) were injected in succession for 240 s (100 μl/min) and dissociation was recorded for 1800 s (100 μl/min). Each injection was followed by a regeneration step with 25 mM NaOH for 18 or 60 s. The data was double referenced (control spot and buffer injection) and fitted to a 1:1 Langmuir model. The bio-MSLN target used was as described in PCT/EP2025/05253, i.e. the extracellular domain (ECD) of human MSLN protein (Q13421-3, residues 296 to 597 of isoform2 with a C-terminal hIgG1 knob in hole tag; purchased from Evitria expressed in CHO-S cells, named MPEXT137) or cynoMSLN_296-597_Fcₖᵢₕ (XM_005590816.3, residues 296 to 597 with a C-terminal hIgG1 knob in hole tag, named JWA064, produced in-house with Expi293F suspension cells) and both comprising an Avi-tag (for site-directed biotinylation). The obtained SPR traces were used to determine the ankyrin repeat protein-MSLN interaction parameters, summarized in Table 4.

TABLE 4

| | Human Mesothelin | | | Cyno Mesothelin | | |
|---|---|---|---|---|---|---|
| DARPin | kₒₙ [1/Ms] | k_{off} [1/s] | K_{D} [M] | kₒₙ [1/Ms] | k_{off} [1/s] | K_{D} [M] |
| DARPin #1 | 3.96E+06 up | 2.73E−04 up | 6.90E−11 up | 4.11E+06 | 1.82E−04 | 4.43E−11 |
| (SEQ ID NO: 1) | to 4.26E+06 | to 3.74E−04 | to 8.78E−11 | | | |
| (1D DARPin) | | | | | | |
| DARPin #4 | 1.45E+06 up | 1.49E−04 up | 3.88E−11 up | 2.57E+06 up | 6.04E−05 up | 1.68E−11 up |
| (SEQ ID NO: 4) | to 3.83E+06 | to 3.51E−04 | to 1.55E−10 | to 3.59E+06 | to 1.86E−04 | to 7.26E−11 |
| (1D DARPin) | | | | | | |
| DARPin #21 | 8.40E+06 | 2.81E−04 | 3.35E−11 | 8.82E+06 | 1.77E−04 | 2.00E−11 |
| (SEQ ID NO: 31), | | | | | | |
| conjugated to | | | | | | |
| chelator (DOTA) | | | | | | |
| (2D DARPin | | | | | | |
| conjugate) | | | | | | |
| DARPin #24 | 4.26E+05 | 1.21E−04 | 2.85E−10 | 14.80E+05 | 5.43E−05 | 1.13E−10 |
| (SEQ ID NO: 34), | | | | | | |
| conjugated to | | | | | | |
| chelator (DOTA) | | | | | | |
| (2D DARPin | | | | | | |
| conjugate) | | | | | | |

The results showed that 1D ankyrin repeat proteins comprising only a MSLN-specific ankyrin repeat domain (SEQ ID NO: 1 or 4) bind with high affinity (in the pM range) to human MSLN or cyno MSLN. Furthermore, the results demonstrated that a MSLN-specific ankyrin repeat domain can be connected to a half-life extending moiety and conjugated to a chelator, such as DOTA, —without significantly affecting the binding affinity of the protein to human or cyno MSLN.

### Example 2: Binding of Ankyrin Repeat Proteins to Cells Expressing MSLN on their Surface

Cell binding titration assays were performed for selected His-tagged single domain (1D) and two domain (2D) designed ankyrin repeat proteins to determine binding to human MSLN expressed on MC38 or OVACR3 cells in absence and presence of human serum albumin. hMSLN-MC38 cells are genetically engineered to express human MSLN by inserting the human MSLN coding sequence into exon2 of the murine gene, so that only the human MSLN is expressed on their surface. OVCAR3 cells are a human ovarian adenocarcinoma cell line which endogenously expresses MSLN on the surface. Controls included Anetumab, an anti-MSLN antibody produced in-house based on the sequence published in the WHO INN registry List #71 (2014) as a positive control and a non-binding DARPin comprising SEQ ID NO: 41 as a negative control. The designed ankyrin repeat proteins had a His tag (SEQ ID NO: 27) at their N-terminal end for ease of purification.

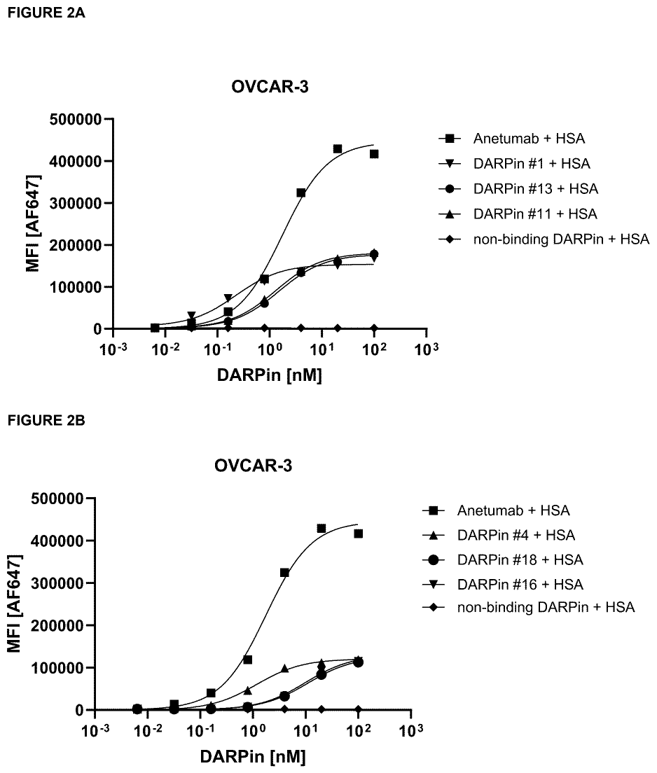

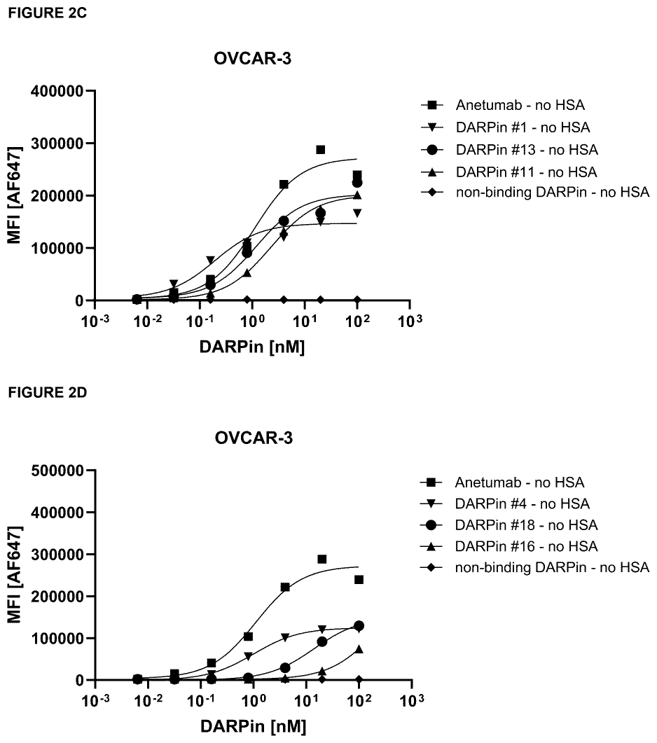

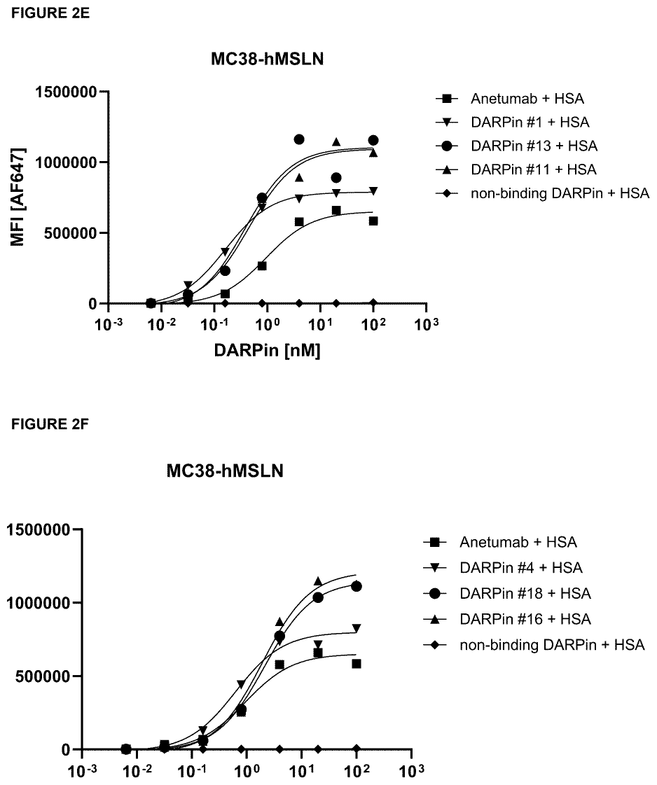

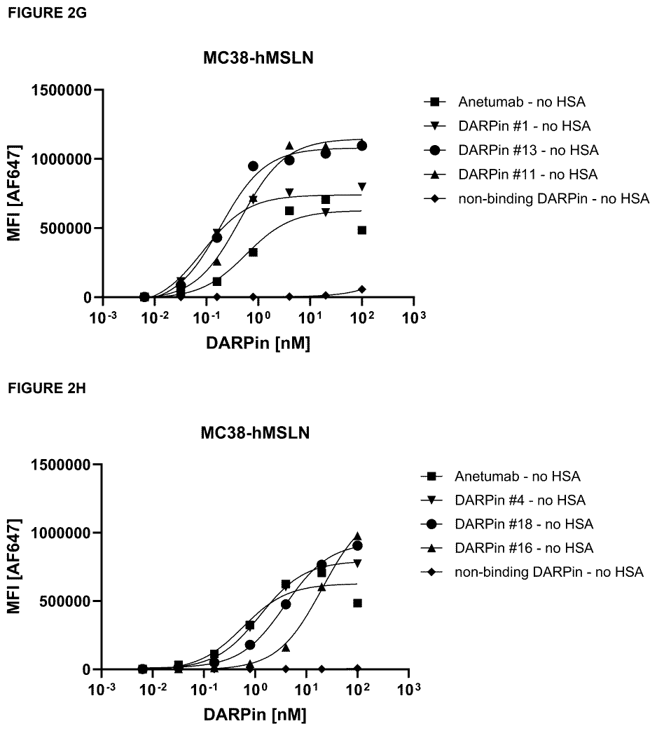

In brief, 5×10⁴ cells/well were resuspended in 100 μL DARPin dilution (500 nM, 1:5 serial diluted) and incubated for 45 min on ice. Cells were washed 2.5× with PBS (fill up to 200 μL after the incubation) and resuspended in 50 μL anti-DARPin antibody (rabbit anti-DARPin 1.4.8, 2 μg/mL) including LIVE/DEAD fixable green cell stain (1:3000, Thermo Fisher #L34970) and incubated for 30 min on ice. This step was performed in absence and presence of 10 μM Human Serum Albumin (CSL Behring #150570). After incubation and washing, cells were incubated with detection antibody for 30 min (goat anti-human IgG (H+L) AF647, Thermo Fisher #21445). Cells were fixed in 1% Paraformaldehyde solution diluted in PBS (Himedia, TCL119-100ML) and acquired at Quanteon (Novocyte). Raw fcs files were exported and analyzed using FlowJo software. MFI values of live AF647-positive cells were exported from FlowJo (version 10.10.0) and plotted using GraphPad Prism software (version 10.2.3). Results are shown in FIG. 2 and summarized in Table 5.

TABLE 5

| | hMSLN-MC38 | | OVCAR3 | |
|---|---|---|---|---|
| | Cell binding | Cell binding | Cell binding | Cell binding |
| | EC50; | EC50; | EC50; | EC50; |
| DARPin | w/o HSA | with HSA | w/o HSA | with HSA |
| (or control) | [nM] | [nM] | [nM] | [nM] |
| Anetumab | 0.65 | 1.04 | 1.26 | 1.87 |
| DARPin #1 | 0.17 | 0.28 | 0.39 | 0.32 |
| DARPin #4 | 1.37 | 0.64 | 1.49 | 1.50 |
| DARPin #13 | 0.22 | 0.41 | 2.01 | 1.66 |
| DARPin #18 | 4.13 | 2.05 | 17.08 | 13.17 |
| DARPin #11 | 0.52 | 0.47 | 3.00 | 1.70 |
| DARPin #16 | 15.8 | 1.90 | 128.1 | 9.73 |
| Non-binding | n.a | n.a | n.a. | n.a. |
| DARPin | | | | |
| control | | | | |

The cell binding titration assays confirmed that the selected ankyrin repeat proteins bind to human MSLN expressed on cells, both in absence and presence of human serum albumin. The results further showed that different peptide linkers, e.g. PT-rich or PAS linkers, can be used to connect the MSLN-specific ankyrin repeat domain and the serum albumin-specific ankyrin repeat domain, retaining the ability of the constructs to bind to MSLN expressed on cells.

### Investigation of the Effect of DARPin Formatting on Binding to Cells Expressing MSLN

Studies were performed to determine whether the positioning of the MSLN-specific ankyrin repeat domain in relation to the serum albumin-specific ankyrin repeat domain (SEQ ID NO: 7) is important for optimal binding to cells expressing MSLN. For this purpose, different 2D ankyrin repeat protein constructs (with an N-terminal His-TEV tag (SEQ ID NO: 28) and a C-terminal cysteine-containing tag (SEQ ID NO: 16)) were generated as described in Example 1. One construct (DARPin #25, SEQ ID NO: 38) comprised a MSLN-specific ankyrin repeat domain connected at its C-terminal end by a peptide linker to a serum albumin-specific ankyrin repeat domain. Another construct (DARPin #26, SEQ ID NO: 39) had the opposite orientation of the same two ankyrin repeat domains, i.e. it comprised a serum albumin-specific ankyrin repeat domain connected at its C-terminal end by a peptide linker to a MSLN-specific ankyrin repeat domain. The constructs were expressed in E. coli and purified over a Ni-NTA column before desalting over a HiLoad 26/600 Superdex 200 column. Main fractions were pooled, digested by TEV protease (Sigma Aldrich) on a roller shaker at room temperature (RT) overnight (~18-20 h). Non-cleaved DARPins still containing the His-tag as well as the His-tagged TEV protease were removed by incubating for 30 min with Ni-NTA resin on a roller shaker at RT, before centrifugation and removal of the Ni-NTA resins by decanting and filtration on empty columns. Supernatant/flow-through was incubated with 5 mM TCEP for 20 min and purified by size-exclusion chromatography, before up-concentration. Final purified samples were stored in 50 mM NaPO4, 150 mM NaCl, pH 6.5. DARPins having a C-terminal Cysteine were subsequently conjugated to a chelator (DTPA, DOTA or DOTAM, or an analogue or derivative thereof) using a connector comprising maleimide. Detailed methods for the production and purification of proteins and for the conjugation of chelators to proteins are well known to the practitioner in the art (see also Example 3).

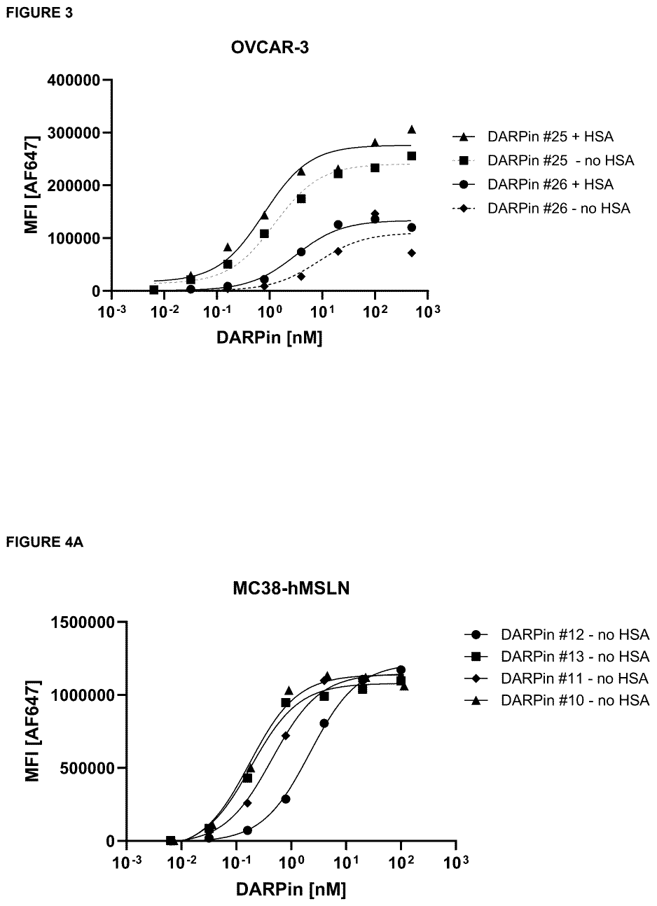

The binding of 2D ankyrin repeat proteins DARPin #25 and DARPin #26, conjugated to the DOTA chelator, to OVCAR3 cells expressing endogenous level of mesothelin was analyzed by flow cytometry. In brief, 5×10⁴ cells/well were resuspended in 100 μL DARPin dilution (500 nM, 1:5 serial diluted) and incubated for 45 min on ice. Cells were washed 2.5× with PBS (fill up to 200 μL after the incubation) and resuspended in 50 μL anti-DARPin antibody (rabbit anti-DARPin 1.4.8) including LIVE/DEAD fixable cell stain (1:3000, Thermo Fisher #L34957) and incubated 30 min on ice. This step was performed in absence and presence of 10 μM Human Serum Albumin (CSL Behring #150570). After incubation and washing, cells were incubated with detection antibody for 30 min on ice (goat anti-human IgG (H+L) AF647, Thermo Fisher #21244). Cells were fixed in 1% Paraformaldehyde diluted in PBS (Himedia, TCL119-100ML) and acquired at Attune NxT (Thermo Life Technologies). Raw fcs files were exported and analyzed using FlowJo software. MFI values of live AF647-positive cells were exported from FlowJo (version 10.10.0) and plotted using GraphPad Prism software (version 10.2.3). Results are shown in FIG. 3 and summarized in Table 6.

TABLE 6

| | OVCAR3 | |
|---|---|---|
| | Cell binding | Cell binding |
| DARPin | EC50; | EC50; |
| (domain orientation) | w/o HSA [nM] | with HSA [nM] |
| DARPin #25 | 8.4 | 3.1 |
| (HSA-specific domain- | | |
| MSLN-specific domain) | | |
| DARPin #26 | 1.2 | 0.8 |
| (MSLN-specific domain- | | |
| HSA-specific domain) | | |

The results summarized in Table 6 demonstrated that the orientation of the two ankyrin repeat domains respective to each other can impact the ability of the constructs to bind MSLN expressing cells. Having the MSLN-specific ankyrin repeat domain N-terminally of the serum albumin-specific ankyrin repeat domain resulted in a construct with a lower EC50 for binding to MSLN-expressing cells, as compared to a construct having the opposite orientation of the two ankyrin repeat domains. This effect of the orientation of the two ankyrin repeat domains was observed in the absence and presence of serum albumin.

### Investigation of the Effect of Linker Length on Binding to Cells Expressing MSLN

Cell binding titration assays were performed with various two domain (2D) DARPins with different linkers connecting the two ankyrin repeat domains, to determine binding to human MSLN expressed on hMSLN-MC38 or OVACR3 cells in absence or presence of human serum albumin. For this purpose, different 2D ankyrin repeat protein constructs (with an N-terminal His tag (SEQ ID NO: 27)) were generated as described in Example 1. The peptide linkers (such as SEQ ID NOs: 9 to 12 and 14) used to connect the two ankyrin repeat domains (MSLN-specific (such as SEQ ID NOs: 1 and 4) and HSA-specific (such as SEQ ID NO: 7)) in the various constructs varied in length between one amino acid and 24 amino acids, and also had different amino acid compositions, such as PT-rich and PAS-rich linkers.

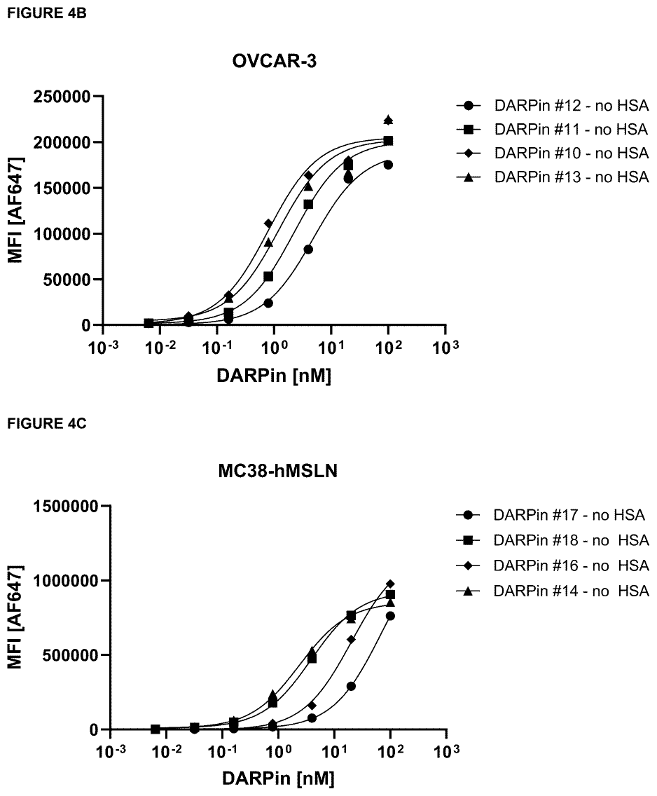

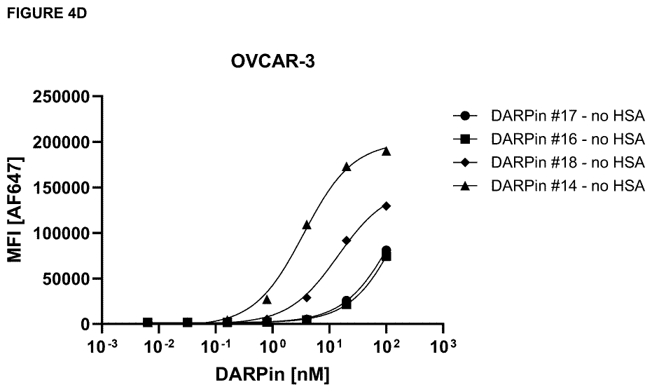

In brief, 5×10⁴ cells/well were resuspended in 100 μL DARPin dilution (500 nM, 1:5 serial diluted) and incubated for 45 min on ice. Cells were washed 2.5× with PBS (fill up to 200 μL after the incubation) and resuspended in 50 μL anti-DARPin antibody (rabbit anti-DARPin 1.4.8, 2 μg/mL) including LIVE/DEAD fixable green cell stain (1:3000, Thermo Fisher #L34970) and incubated for 30 min on ice. This step was performed in absence and presence of 10 μM Human Serum Albumin (CSL Behring #150570). After incubation and washing, cells were incubated with detection antibody for 30 min (goat anti-human IgG (H+L), AF647, Thermo Fisher #21445). Cells were fixed in 1% Paraformaldehyde solution diluted in PBS (Himedia, TCL119-100ML) and acquired at Quanteon (Novocyte). Raw fcs files were exported and analyzed using FlowJo software (version 10.10.0). MFI values of live AF647-positive cells were exported from FlowJo and plotted using GraphPad Prism software (version 10.2.3). Results are shown in FIG. 4 and summarized in Table 7.

TABLE 7

| | OVCAR3 | hMSLN-MC38 |
|---|---|---|
| | Cell binding | Cell binding |
| DARPin | EC50; w/o | EC50; w/o |
| (or control) | HSA [nM] | HSA [nM] |
| Anetumab control | 1.26 | 0.64 |
| DARPin #12 | 6.16 | 2.08 |
| MSLN-specific domain #1-24AA | | |
| Linker-HSA-specific domain | | |
| DARPin #11 | 3.00 | 0.52 |
| MSLN-specific domain #1-11AA | | |
| Linker-HSA-specific domain | | |
| DARPin #10 | 1.24 | 0.21 |
| MSLN-specific domain #1-8AA | | |
| Linker1-HSA-specific domain | | |
| DARPin #13 | 2.00 | 0.21 |
| MSLN-specific domain #1-8AA | | |
| Linker2-HSA-specific domain | | |
| DARPin #17 | 92.95 | 42.58 |
| MSLN-specific domain #2-24AA | | |
| Linker-HSA-specific domain | | |
| DARPin #16 | 128.05 | 15.76 |
| MSLN-specific domain #2-11AA | | |
| Linker-HSA-specific domain | | |
| DARPin #18 | 17.07 | 4.13 |
| MSLN-specific domain #2-8AA | | |
| Linker2-HSA-specific domain | | |
| DARPin #14 | 5.96 | 2.74 |
| MSLN-specific domain #2-1AA | | |
| Linker-HSA-specific domain | | |
| Non-binding DARPin control | n.a. | |

The results shown in FIG. 4 and summarized in Table 7 demonstrated that the length of the peptide linker between the MSLN-specific ankyrin repeat domain and the serum albumin-specific ankyrin repeat domain has an effect on the ability of the constructs to bind MSLN expressing cells. Linkers shorter than 24 amino acids resulted in lower EC50 for binding to MSLN expressing cells as compared to a 24 amino acid-long linker. Furthermore, linkers shorter than 11 amino acids resulted in lower EC50 for binding to MSLN expressing cells as compared to a 11 amino acid-long linker.

### Example 3: Biodistribution of Radio-Labelled DARPin Conjugates in hMSLN-MC38 Tumor Model

Generation of DARPins with a C-Terminal Cysteine for Site-Specific Conjugation and Radio-Labeling

Various ankyrin repeat proteins comprising a MSLN-specific ankyrin repeat domain and a HSA-specific ankyrin repeat domain were designed, such as, e.g., SEQ ID NOs: 17 to 26 and 29 to 34. Selected proteins were produced at high quality for biodistribution (BioD) in vivo experiments. DARPins (such as, e.g., DARPin #13 and DARPin #18) were subcloned into a derivative of the pQE30 (Qiagen) expression vector (pMPDVO25), generating constructs containing a His-TEV tag (SEQ ID NO: 28) fused to the N-terminus of the subcloned ankyrin repeat protein (having a N-terminal GS), and a C-terminal cysteine-containing tag (such as SEQ ID NO: 16) fused to the C-terminus of the subcloned ankyrin repeat protein. These ankyrin repeat protein constructs were expressed in E. coli and purified over a Ni-NTA column before desalting over a HiLoad 26/600 Superdex 200 column. Main fractions were pooled, digested by TEV protease (Sigma Aldrich) on a roller shaker at room temperature (RT) overnight (~18-20 h). Samples were taken after overnight digestion and analyzed by SDS-PAGE to assess the degree of cleavage. Non-cleaved DARPins still containing the His-tag as well as the His-tagged TEV protease were removed by incubating for 30 min with Ni-NTA resin on a roller shaker at RT, before centrifugation and removal of the Ni-NTA resins by decanting and filtration on empty columns. Supernatant/flow-through was incubated with 5 mM TCEP for 20 min and purified by size-exclusion chromatography, before up-concentration. Final purified samples were stored in 50 mM NaPO4, 150 mM NaCl, pH 6.5. Detailed methods for the production and purification of proteins are well known to the practitioner in the art. Examples of resulting DARPins with a C-terminal cysteine include DARPin #21 (SEQ ID NO: 31) and DARPin #24 (SEQ ID NO: 34).

### Site-Specific Conjugation and Radio-Labeling of DARPins Having a C-Terminal Cysteine

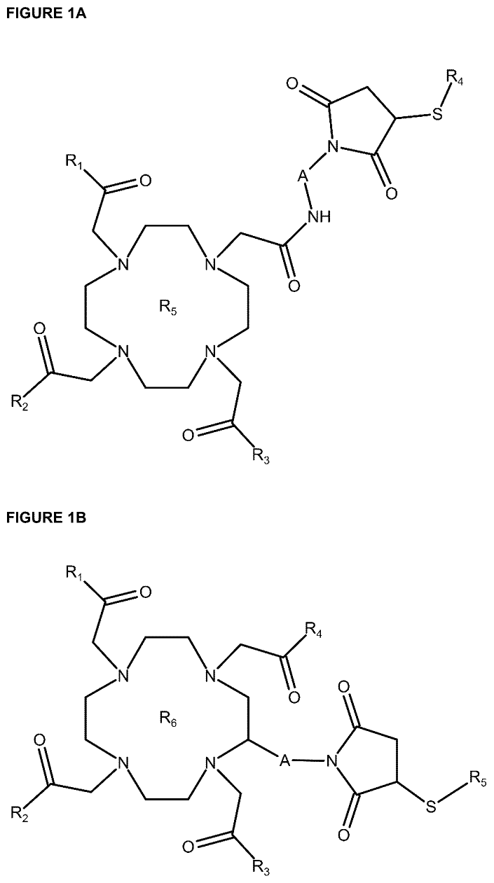

DARPins having a C-terminal cysteine were conjugated to a chelator (DTPA, DOTA or DOTAM, or an analogue or derivative thereof) using a connector comprising maleimide. Resulting DARPin-chelator conjugates were then labeled with lead, such as Pb-212. Methods for the generation and purification of Pb-203 or Pb-212, for the conjugation of chelators, such as DTPA, DOTA, DOTAM, or analogues or derivatives thereof, to (poly)peptides, and for the subsequent radio-labeling of chelator-(poly)peptide conjugates with lead, such as Pb-203 or Pb-212, have been described (see, e.g., US 2022/0037046; US 2023/0372552; U.S. Pat. No. 11,541,133; Baidoo et al., Nucl Med Biol. 2013 July, 40(5): 592-599; McNeil et al., Nature Scientific Reports (2023) 13:10623; WO 2017/220767; Morais and Ma, Drug Discovery Today: Technologies|Antibody—drug Conjugates (ADC), Vol. 30, pp. 91-104, 2018; Tsuchikama and An, Protein Cell 2018, 9(1):33-46); Kang et al., Chem. Sci., 2021, 12, 13613). Furthermore, methods for using a maleimide-comprising connector to connect a cysteine-comprising polypeptide (e.g. an ankyrin repeat protein) to a chemical moiety have been described (see, e.g., WO 2011/135067). The structures of resulting radio-labeled DARPin conjugates are illustrated by the examples shown in FIGS. 1A and 1B (whereby the protein moieties R4 in FIG. 1A and R5 in FIG. 1B vary depending on the DARPin used for the conjugation). Control protein conjugates (e.g. antibody) used in some experiments were generated similarly as the radio-labeled DARPin conjugates. As demonstrated in Example 1, DARPin conjugates (e.g. conjugated to the chelator DOTA) described herein bind to human mesothelin (see Table 4).

### Biodistribution of Radio-Labeled DARPin Conjugates

hMSLN-MC38 tumor models. ATH mice (Rj:ATHYM-Foxn1^{nu/nu}) were purchased from Janvier-Labs (France). All studies were conducted under the approval of the Swiss Vet committee (license ZH159/2022). Animals were maintained under specific-pathogen-free (SPF) conditions with daily cycles of light and darkness (12 h/12 h), in line with ethical guidelines. No manipulations were performed during the first 7 days after arrival, to allow the animals to acclimatize to the new environment. All mice were monitored daily for assessment of physical condition and general well-being. hMSLN-MC38 cells were purchased from Biocytogen (Catalog No. 332181). Solid xenografts were established by subcutaneous (SQ) injection of hMSLN-MC38 cells in PBS. Cells were quantified using Countess cell counter. Each mouse was injected SQ with 1×10⁶ hMSLN-MC38 cells in 200 μL PBS into the right flank of each mouse. Tumor volumes were estimated three times per week through calipering, according to the formula: volume=0.5×length×width² and grown until they reached 200-300 mm³. hMSLN level expressed in tumors, tumor vascularization profile and hMSLN soluble form in mouse serum were tested.

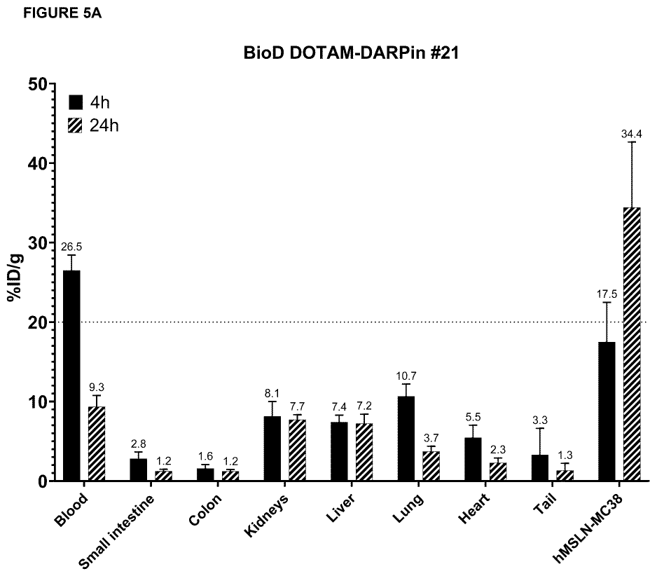

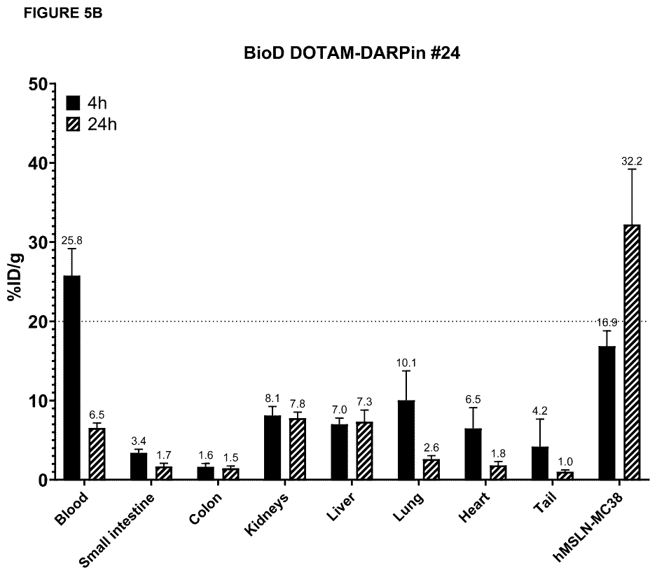

Biodistribution study. Radio-conjugated DARPins were injected at 0.01 mg/kg (10 μCi DARPins) into the tail vein of ATH mice (7 weeks females) xenografted subcutaneously with hMSLN-MC38 cells. Biodistribution was monitored at 4 h and 24 h post-injection. Mice were euthanized by CO2 inhalation and cervical dislocation. Blood, small intestine, colon, kidneys, liver, lung, heart, tail and tumor were extracted, weighed and the radioactivity was determined with a y-counter. The data were expressed as the % injected dose/gram (% ID/g). Results are shown in FIGS. 5A and 5B and in Table 8.

TABLE 8

| % ID/g | DARPin #21 | | DARPin #24 | |
|---|---|---|---|---|
| Time after injection | 4 h | 24 h | 4 h | 24 h |
| Blood | 26.5 | 9.3 | 25.8 | 6.5 |
| Kidneys | 8.1 | 7.7 | 8.1 | 7.8 |
| Tumors | 17.5 | 34.4 | 16.9 | 32.2 |
| T:K ratio | 2.16 | 4.46 | 2.08 | 4.12 |

Consistent with PK data (see Example 4), DARPin #21 and DARPin #24 displayed similar blood exposure profiles. DARPin #21 and DARPin #24 showed a low kidney uptake (below 10% ID/g) and a good tumor uptake (above 15% ID/g at 4 h and above 30% ID/g at 24 h). DARPin #21 and DARPin #24 showed a tumor to kidney (T:K) ratio above 2 at 4 h and above 4 at 24 h in hMSLN-MC38 tumors (Table 8). No unexpected uptake in other organs was observed in the organs tested.

### Example 4: Pharmacokinetic (PK) Analysis of Lead-Labelled DARPin Conjugates in Mice

PK studies were performed in wild-type (WT) mice and in tumor bearing mice.

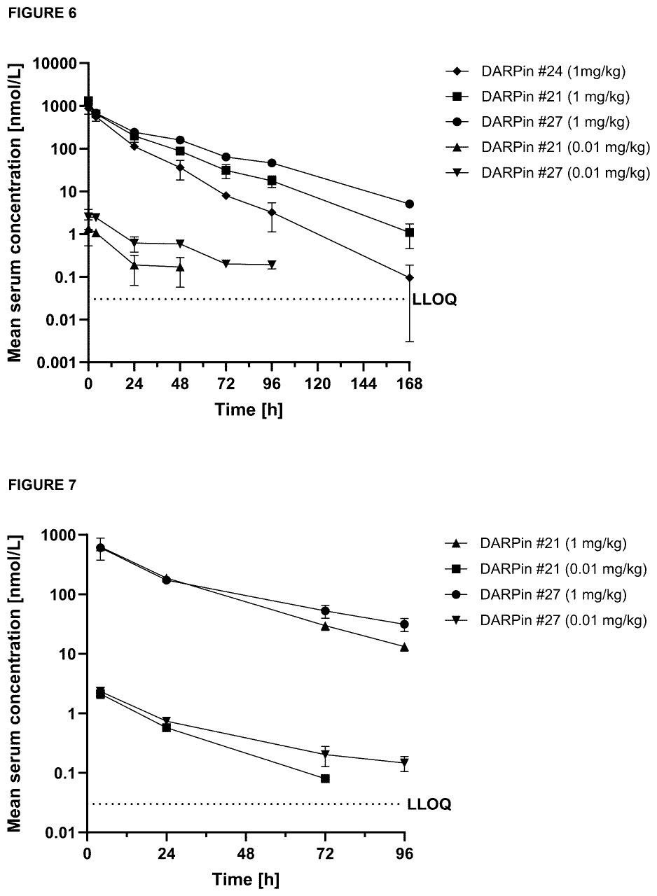

PK study in WT mice. DARPin #21, conjugated with DOTAM and labeled with natural lead (NatPb), was injected i.v. at 0.01 mg/kg or 1 mg/kg into the tail vein of WT Athymic nude mice. DARPin #27, conjugated with DOTAM and natural lead, was injected iv. at 0.01 mg/kg or 1 mg/kg into the tail vein of DT Athymic nude mice. DARPin #24, conjugated with DOTAM and natural lead, was injected i. at 1 mg/kg into the tail vein of WT Athymic nude mice. Serum was collected 5 min, 4 h, 24 h, 48 h, 72 h, 96 h and 168 h post injection. DARPin detection was measured by ELISA. DARPin PK characteristics were calculated with time points between 4 to 168 h. The data were expressed as the molecule concentration in serum (nmol/L). Results are shown in FIG. 6 and Table 9.

TABLE 9
PK characteristics in WT mice

| | | DARPin | DARPin | DARPin | DARPin | DARPin |
|---|---|---|---|---|---|---|
| | | #24 | #21 | #27 | #21 | #27 |
| Parameter | Unit | (1 mg/kg) | (1 mg/kg) | (1 mg/kg) | (0.01 mg/kg) | (0.01 mg/kg) |
| AUCINF_pred | (hr*nmol/L) | 12205.6 | 18645.1 | 23587.2 | 24.9 | 74.7 |
| AUClast | (hr*hr*nmol/L) | 12204.4 | 18617.7 | 23411.0 | 21.7 | 69.1 |
| Cmax | (nmol/L) | 880.6 | 1326.0 | 1101.1 | 1.3 | 2.5 |
| Tmax | (hr) | 0.083 | 0.083 | 0.083 | 0.083 | 0.083 |
| CI_pred | (L/hr/kg) | 0.0027 | 0.0020 | 0.0015 | 0.0147 | 0.0046 |
| Vss_pred | (L/kg) | 0.034 | 0.040 | 0.046 | 0.284 | 0.151 |
| HL_Lambda_z | (hr) | 13.4 | 18.4 | 24.3 | 17.1 | 26.3 |
| AUC_%Extrap_pred | (%) | 0 | 0 | 1 | 13 | 7 |
| AUC_%Back_Ext_pred | (%) | 1 | 1 | 0 | 0 | 0 |
| Time point | (h) | 4-168 | 4-168 | 4-168 | 4-48 | 4-96 |
| considered for HL | | | | | | |
| calculations | | | | | | |

PK study in tumor bearing mice. Athymic nude mice were subcutaneously engrafted with hMSLN-MC38PP-4T cells. Once tumor volumes reached the predetermined volume (~300-500 mm³), animals were randomized and injected with DARPins. DARPin #21, conjugated with DOTAM and natural lead, was injected iv. at 0.01 mg/kg or 1 mg/kg into the tail vein of Athymic nude mice. DARPin #27, conjugated with DOTAM and natural lead, was injected i.v. at 0.01 mg/kg or 1 mg/kg into the tail vein of Athymic nude mice. Serum was collected 4 h, 24 h, 72 h and 96 h post injection. DARPin detection was measured by ELISA. DARPin PK characteristics were calculated with time points between 4 to 168 h. The data were expressed as the molecule concentration in serum (nmol/L). Results are shown in FIG. 7 and Table 10.

TABLE 10
PK characteristics in tumor bearing mice

| | | DARPin | DARPin | DARPin | DARPin |
|---|---|---|---|---|---|
| | | #21 | #21 | #27 | #27 |
| Parameter | Unit | (1 mg/kg) | (0.01 mg/kg) | (1 mg/kg) | (0.01 mg/kg) |
| AUCINF_pred | (hr*nmol/L) | 17045.7 | 53.9 | 17890.8 | 72.4 |
| AUClast | (hr*hr*nmol/L) | 16754.1 | 52.3 | 16986.7 | 68.3 |
| Cmax | (nmol/L) | 628.3 | 2.1 | 606 | 2.3 |
| Tmax | (hr) | 4.0 | 4.0 | 4.0 | 4.0 |
| Cl_pred | (L/hr/kg) | 0.0022 | 0.0068 | 0.0019 | 0.0048 |
| Vss_pred | (L/kg) | 0.039 | 0.107 | 0.05 | 0.129 |
| HL_Lambda_z | (hr) | 16.9 | 14.6 | 22.8 | 23.7 |
| AUC_%Extrap_pred | (%) | 2 | 3 | 5 | 6 |
| AUC_%Back_Ext_pred | (%) | 17 | 18 | 15 | 15 |
| Time point considered for | (h) | 4-96 | 4-72 | 4-96 | 4-96 |
| HL calculations | | | | | |

### Conclusion

Pharmacokinetic studies in female WT and tumor bearing athymic nude mice were performed. After iv. bolus administration of 0.01 mg/kg or 1 mg/kg NatPb-DOTAM-DARPin #21, NatPb-DOTAM-DARPin #24 or NatPb-DOTAM-DARPin #27, the concentration-time profile showed a steady decline in each DARPin serum concentration. The calculated half-life of 1 mg/kg NatPb-DOTAM-DARPin #24 was 13.4 h. For NatPb-DOTAM-DARPin #21, the calculated half-life was between 14.6 and 18.4 h, and for NatPb-DOTAM-DARPin #27, it was between 22.8 and 26.3 h.

### Example 5: Biodistribution of Radio-Labelled DARPin Conjugates in OVCAR-3 Tumor Model

DARPins #21 (SEQ ID NO: 31), #42 (SEQ ID NO: 56), and #37 (SEQ ID NO: 51) comprising a MSLN-specific ankyrin repeat domain and a HSA-specific ankyrin repeat domain were designed and expressed as described in Example 3. DARPins were conjugated with chelator (DOTAM) and labelled with Pb-212 as described in Example 3.

ATH mice (Rj:ATHYM-Foxn1^{nu/nu}) were maintained under specific-pathogen-free (SPF) conditions with daily cycles of light and darkness (12 h/12 h), in line with ethical guidelines. No manipulations were performed during the first 7 days after arrival, to allow the animals to acclimatize to the new environment.

All mice were monitored daily for assessment of physical condition and general well-being. Solid xenografts were established by subcutaneous (SQ) injection of OVCAR-3 (ATCC, cat number HTB-161) cells in PBS. Cells were quantified using Countess cell counter. Each mouse was injected SQ with 5×10⁶ OVCAR-3 cells in 100 μL RPMI/HC-Matrigel into the right flank of each mouse. Tumor volumes were estimated three times per week through calipering, according to the formula: volume=0.5×length×width² and grown until they reached 200-300 mm³.

Radio-conjugated DARPins were injected at 0.01 mg/kg (10 μCi DARPins) into the tail vein of ATH mice (7 weeks females) xenografted subcutaneously with OVCAR-3 cells. Biodistribution was monitored at 1 h, 4 h and 24 h post-injection. Mice were euthanized by CO2 inhalation and cervical dislocation. Blood, heart, lung, liver, kidneys, spleen, small intestine, colon, tail, and tumor were extracted, weighed and the radioactivity was determined with a y-counter. The data were expressed as the % injected dose/gram (% ID/g). Results are shown in Table 11.

TABLE 11

| | % ID/g | | | | | | | | |
|---|---|---|---|---|---|---|---|---|---|
| | DARPin #21 | | | DARPin #42 | | | DARPin #37 | | |
| | Time after injection | | | | | | | | |
| | 1 h | 4 h | 24 h | 1 h | 4 h | 24 h | 1 h | 4 h | 24 h |
| Blood | 23.0 | 15.3 | 6.3 | 27.5 | 24.2 | 12.2 | 20.3 | 20.4 | 6.5 |
| Kidneys | 8.3 | 7.1 | 13.7 | 9.1 | 13.0 | 22.6 | 11.2 | 16.3 | 26.0 |
| Tumors | 5.2 | 3.3 | 5.0 | 3.1 | 4.0 | 8.6 | 5.0 | 3.7 | 7.3 |

### Example 6: De-Immunisation of DARPins

Using the in silico prediction model of immunogenicity risk of modified peptides, disclosed in EP25171959.7 and incorporated herein by reference, specific amino acids in the N-capping and/or the internal repeat modules of the MSLN-specific ankyrin repeat domains with SEQ ID NOs: 1 and 42 were substituted. More specifically, in the N-capping module Q14N, D15P and K23Q mutations were made, while both internal repeats had L18P and K26E mutations introduced, resulting in DARPin proteins #29 (comprising SEQ ID NO: 43) and #30 (SEQ ID NO: 44), respectively.

In the HSA-specific designed ankyrin repeat domain with SEQ ID NO: 7, amino acid substitutions Q14N, D15P and K23Q were made in the N-capping module, while both internal repeats had L18P and K26E mutations introduced. In addition, K19E substitution was introduced in the first internal repeat, resulting in DARPin protein #31 (SEQ ID NO: 45).

In order to generate half-life extended MSLN-specific ankyrin repeat proteins, nucleic acid sequences encoding the MSLN-specific ankyrin repeat domains of SEQ ID NOs: 43 and 44, respectively, were first subcloned C-terminally of an expression cassette consisting of a His-tag and a TEV cleavage site (SEQ ID NO: 28), a serum albumin-specific designed ankyrin repeat domain with SEQ ID NO: 7 or SEQ ID NO: 45 and a peptide linker (SEQ ID NO: 11). Examples of resulting constructs comprising two ankyrin repeat domains (2D), one with binding specificity for MSLN and one with binding specificity for serum albumin include SEQ ID NOs: 51 to 55. The resulting constructs comprising two ankyrin repeat domains were expressed in E. coli and purified using the N-terminal His-tag according to standard protocols.

### Kinetic Binding Parameters and Binding Affinities of Recombinant Ankyrin Repeat Proteins

Kinetic binding parameters and binding affinities for MSLN of two domain (2D) ankyrin repeat proteins comprising an ankyrin repeat domain with binding specificity for MSLN and an ankyrin repeat domain with binding specificity for serum albumin were determined by surface plasmon resonance (SPR) multi-trace analysis.

In brief, a SPR assay was used to determine the binding kinetics to the target MSLN. The data were generated using BioRad ProteOn instrument in SPR running buffer (PBS, pH 7.4 containing 0.005% Tween 20*). A Streptavidin chip SAHC200M (Xantec) was conditioned according to the manufacturer's protocol. The chip was coated with biotinylated target (bio-MLSN). All analytes (3-fold dilution starting from 10 down to 0.12 nM) were injected in succession for 240 s (100 μl/min) and dissociation was recorded for 1800 s (100 μl/min). Each injection was followed by a regeneration step with 25 mM NaOH for 18 or 60 s. The data was double referenced (control spot and buffer injection) and fitted to a 1:1 Langmuir model. The bio-MSLN target used was as described in PCT/EP2025/052531, i.e. the extracellular domain (ECD) of human MSLN protein (Q13421-3, residues 296 to 597 of isoform2 with a C-terminal hIgG1 knob in hole tag; purchased from Evitria expressed in CHO-S cells, named MPEXT137) comprising an Avi-tag (for site-directed biotinylation). The obtained SPR traces were used to determine the ankyrin repeat protein-MSLN interaction parameters, summarized in Table 12. The results demonstrate that the introduction of certain amino acid mutations do not affect the binding affinities of the ankyrin repeat domains to the target.

TABLE 12

| DARPin | kₒₙ [1/Ms] | k_{off} [1/s] | K_{D} [M] |
|---|---|---|---|
| #42 | 4.11E+06 | 1.00E−04 | 2.43E−11 |
| #37 | 4.52E+06 | 2.36E−04 | 5.23E−11 |
| #38 | 3.99E+06 | 2.54E−04 | 6.38E−11 |
| #39 | 4.19E+06 | 2.56E−04 | 6.11E−11 |
| #40 | 1.94E+06 | 3.10E−04 | 1.60E−10 |
| #41 | 3.60E+06 | 8.16E−04 | 2.27E−10 |

Cell Binding of DARPins to hMSLN-MC38 and OVCAR-3 Cells

Cell binding titration assays were performed essentially as described in Example 2 to determine binding to OVCAR-3 cells, as well as human MSLN expressed on MC38 cells (hMSLN-MC38) in absence and presence of human serum albumin. hMSLN-MC38 cells are genetically engineered to express human MSLN by inserting the human MSLN coding sequence into exon2 of the murine gene, so that only the human MSLN is expressed on their surface.

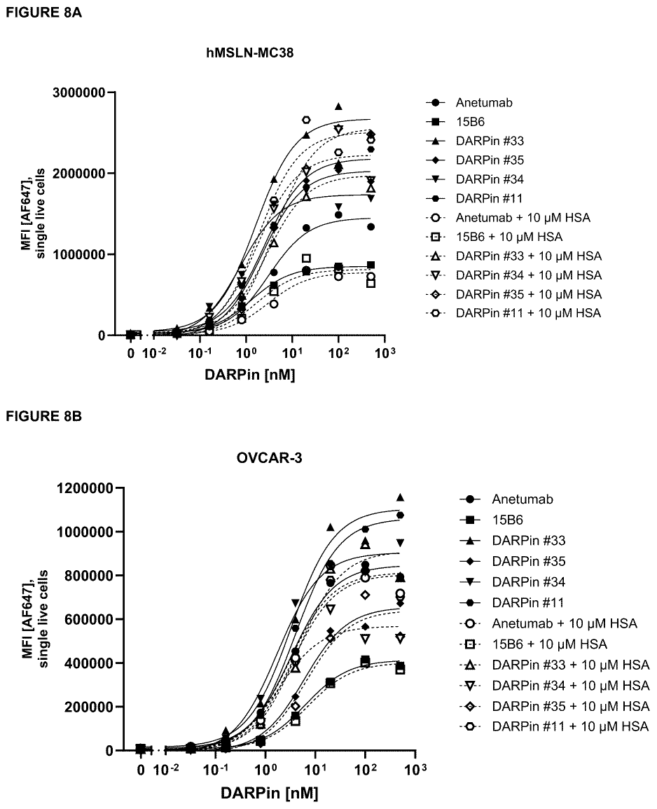

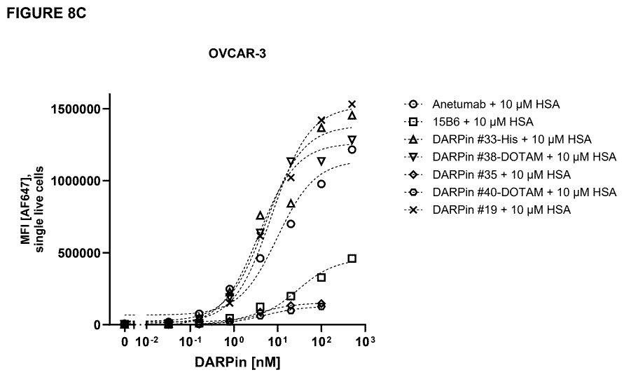

In brief, DARPin titrations were performed in FACS buffer. U-bottom 96-well plates were seeded with 50 μL of cell suspension at 1×10⁶ cells/mL, yielding 50,000 cells perwell. Antibodies anetumab and 15B6 were used as controls. An equal volume (50 μL) of 2x DARPins prepared in FACS buffer was added to each well and incubated for 45 minutes on ice, with or without 10 μM HSA. Following incubation, cells were washed 2.5 times with cold FACS buffer by centrifugation at 420×g for 2 minutes. The washing protocol consisted of filling wells to 200 μL after the initial incubation, followed by two additional washes with 200 μL FACS buffer each. After washing, 50 μL of antibody solution was added according to the experimental plate layout, including Live/Dead Green viability dye diluted 1:3000. Cells were incubated for 30 minutes at 4° C., then washed 2.5 times with cold FACS buffer using the same centrifugation conditions. Cell pellets were resuspended in 50 μL of 1% paraformaldehyde in PBS (Himedia, TCL119-100ML) and fixed for 20 minutes at room temperature. Following fixation, cells were washed once with PBS and resuspended in 200 μL of PBS containing 2 mM EDTA. Prior to acquisition, 150 μL of PBS was added to each sample. Flow cytometry data acquisition was performed using a Quanteon flow cytometer, and analysis was conducted using Novocyte Express software. Raw fcs files were exported and analyzed using FlowJo software. MFI values of live AF647-positive cells were exported from FlowJo and plotted using GraphPad Prism software (version 10.2.3). The cell binding titration assays (see Table 13 and FIGS. 8A and 8B) confirmed that the de-immunized ankyrin repeat proteins maintain binding to hMSLN expressed by the MC38 cells as well as by OVCAR-3 cells with a similar profile as their parental versions, both in absence and presence of human serum albumin. No binding was detected on MC38 only cells.

TABLE 13

| | hMSLN-MC38 | | OVCAR-3 | |
|---|---|---|---|---|
| Binding | EC50 without | EC50 with | EC50 without | EC50 with |
| protein | HSA [nM] | HSA [nM] | HSA [nM] | HSA [nM] |
| Anetumab | 3.06 | 2.96 | 3.24 | 2.99 |
| 15B6 | 1.28 | 1.74 | 6.37 | 7.15 |
| DARPin #33 | 1.61 | 2.80 | 3.26 | 4.46 |
| DARPin #34 | 0.75 | 1.73 | 1.73 | 1.77 |
| DARPin #35 | 2.22 | 4.04 | 5.99 | 6.49 |
| DARPin #11 | 2.51 | 1.88 | 4.01 | 3.36 |

### Cell Binding of DOTAM-Conjugated DARPins to OVCAR-3 Cells

Cell binding titration assays of DARPins conjugated to DOTAM chelator were performed essentially as described previously to determine binding to OVCAR-3 cells in presence of human serum albumin.

Briefly, OVCAR-3 cells were seeded at 50,000 cells per well by adding 50 μL of 1×10⁶ cells/mL suspension to U-bottom plates. Two-fold serial dilutions of His-tagged or DOTAM-conjugated DARPins were prepared in FACS buffer supplemented with 10 μM HSA, starting at 500 nM concentration across 7 dilution points plus a negative control. Fifty microliters of 2× concentrated DARPin solution was added to each well containing the cells and incubated for 45 minutes on ice. Antibodies anetumab and 15B6 were used as controls. Following incubation, cells were washed 2.5 times with cold FACS buffer by filling wells to 200 μL, then performing two additional 200 μL washes, with centrifugation at 420×g for 2 minutes between each wash step. Cells were then incubated with 50 μL of Live/Dead Green viability dye (1:3000 dilution) for 30 minutes at 4° C., followed by another 2.5x wash cycle with cold FACS buffer and centrifugation. After washing, cells were fixed by resuspending in 50 μL of 1% paraformaldehyde and incubating for 20 minutes at room temperature. A final wash with PBS was performed before resuspending the cell pellet in 200 μL of PBS containing 2 mM EDTA, with 150 μL of PBS added prior to sample acquisition. Flow cytometry data were acquired using a Quanteon cytometer and analyzed using Novocyte Express software, with final data visualization performed in GraphPad Prism version 10.2.3.

The cell binding titration assays (see Table 14 and FIG. 8C) confirmed that the de-immunized and DOTAM-conjugated ankyrin repeat proteins maintain binding to hMSLN expressed by OVCAR-3 in the presence of 10 μM human serum albumin (HSA). DOTAM-conjugation did not affect cell binding.

TABLE 14

| Binding protein | EC50 with HSA [nM] |
|---|---|
| Anetumab | 9.76 |
| 15B6 | 28.29 |
| DARPin #33 | 5.27 |
| DARPin #38 + DOTAM | 3.71 |
| DARPin #35 | 3.17 |
| DARPin #40 + DOTAM | 4.93 |
| DARPin #19 | 7.40 |

n.d. not determined

### Example 7: Biodistribution of Radio-Labelled DARPin Conjugates in hMSLN-MC38 Tumor Model

### Site-Specific Conjugation and Radio-Labeling of DARPins Having a C-Terminal Cysteine

DARPins DARPin #38, DARPin #39, DARPin #40, and DARPin #41, comprising a MSLN-specific ankyrin repeat domain and a HSA-specific ankyrin repeat domain, were produced as described in Example 3. DARPins having a C-terminal cysteine were conjugated to DOTAM using a connector comprising maleimide and labelled with lead, as described in Example 3.

### Biodistribution of Radio-Labeled DARPin Conjugates

ATH mice (Rj:ATHYM-Foxn1^{nu/nu}) were maintained under specific-pathogen-free (SPF) conditions with daily cycles of light and darkness (12 h/12 h), in line with ethical guidelines. No manipulations were performed during the first 7 days after arrival, to allow the animals to acclimatize to the new environment. All mice were monitored daily for assessment of physical condition and general well-being. hMSLN-MC38 cells were purchased from Biocytogen (Catalog No. 332181). Solid xenografts were established by subcutaneous (SQ) injection of hMLSN-MC38 cells in GFR Matrigel. Cells were quantified using Countess cell counter. Each mouse was injected SQ with 5×10⁶ hMSLN-MC38 cells in 100 μL GFR Matrigel into the right flank of each mouse. Tumor volumes were estimated three times per week through calipering, according to the formula: volume=0.5×length×width² and grown until they reached 200-300 mm³.

Radio-conjugated DARPins were injected at 0.01 mg/kg (1 μCi DARPins) into the tail vein of ATH mice (7 weeks females) xenografted subcutaneously with hMSLN-MC38 cells. Biodistribution was monitored at 4 h and 24 h post-injection. Mice were euthanized by CO2 inhalation and cervical dislocation. Blood, kidneys, and tumor were extracted, weighed and the radioactivity was determined with a γ-counter. The data were expressed as the % injected dose/gram (% ID/g). Results are shown in Table 15.

TABLE 15

| | % ID/g | | | | | | | |
|---|---|---|---|---|---|---|---|---|
| | DARPin #38 | | DARPin #39 | | DARPin #40 | | DARPin #41 | |
| | Time after injection | | | | | | | |
| | 4 h | 24 h | 4 h | 24 h | 4 h | 24 h | 4 h | 24 h |
| Blood | 31.3 | 10.0 | 35.1 | 6.0 | 23.0 | 6.1 | 17.4 | 1.0 |
| Kidneys | 8.8 | 11.2 | 19.6 | 17.3 | 12.2 | 11.1 | 18.3 | 13.1 |
| Tumors | 24.1 | 38.8 | 21.3 | 25.9 | 18.2 | 26.5 | 17.5 | 15.7 |
| T:K ratio | 2.73 | 3.47 | 1.09 | 1.5 | 1.49 | 2.38 | 0.95 | 1.2 |

### Example 8: Biodistribution of Radio-Labelled DARPin Conjugates in OVCAR-3 Tumor Model

### Site-Specific Conjugation and Radio-Labeling of DARPins Having a C-Terminal Cysteine

DARPins DARPin #38 and DARPin #42 comprising a MSLN-specific ankyrin repeat domain and an HSA-specific ankyrin repeat domain were produced as described in Example 3. DARPins having a C-terminal cysteine were conjugated to DOTAM using a connector comprising maleimide and labelled with lead, as described in Example 3.

### Biodistribution of Radio-Labeled DARPin Conjugates

ATH mice (Rj:ATHYM-Foxn1^{nu/nu}) were maintained under specific-pathogen-free (SPF) conditions with daily cycles of light and darkness (12 h/12 h), in line with ethical guidelines. No manipulations were performed during the first 7 days after arrival, to allow the animals to acclimatize to the new environment.

All mice were monitored daily for assessment of physical condition and general well-being. OVCAR-3 cells were purchased from ATCC (cat number HTB-161). Solid xenografts were established by subcutaneous (SQ) injection of OVCAR-3 cells in HC Matrigel. Cells were quantified using Countess cell counter. Each mouse was injected SQ with 5×10⁶ OVCAR-3 cells in 100 μL HC Matrigel into the right flank of each mouse. Tumor volumes were estimated three times per week through calipering, according to the formula: volume=0.5×length×width² and grown until they reached 200-300 mm³.

Radio-conjugated DARPins were injected at 0.01 mg/kg (10 μCi DARPins) into the tail vein of ATH mice (7 weeks females) xenografted subcutaneously with OVCAR-3 cells. Biodistribution was monitored at 1 h, 4 h and 24 h post-injection. Mice were euthanized by CO2 inhalation and cervical dislocation. Selected organs were extracted, weighed and the radioactivity was determined with a γ-counter. The data were expressed as the % injected dose/gram (% ID/g). Results are shown in Table 16. As demonstrated by these results, de-immunizing the DARPins did not have a negative effect on biodistribution, and in particular no negative effect was observed on tumor uptake.

TABLE 16

| % ID/g | DARPin #42 | | DARPin #38 | |
|---|---|---|---|---|
| Time after injection | 4 h | 24 h | 4 h | 24 h |
| Blood | 16.4 | 5.4 | 24.8 | 10.2 |
| Kidneys | 19.8 | 34.8 | 13.5 | 23.3 |
| Tumors | 3.8 | 7.8 | 4.5 | 8.9 |

### Example 9: Biodistribution of Radio-Labelled DARPin Conjugates in hMSLN-MC38 Tumor Model

### Site-Specific Conjugation and Radio-Labeling of DARPins Having a C-Terminal Cysteine

DARPin #38 comprising a MSLN-specific ankyrin repeat domain and an HSA-specific ankyrin repeat domain were produced as described in Example 3. Once purified, EDTA (pH 7.4) was added to the DARPin solution to achieve a final concentration of 10 mM, providing at least a 10-fold molar excess of EDTA over the DARPin protein to prevent the chelator from collecting trace amounts of metal ions present in the buffer. Following EDTA addition, maleimide-functionalized chelator (DOTA or DOTAM) was added to the protein solution. The chelator (DOTA or DOTAM) was provided from a 50 mM stock solution in DMSO. Maleimide-DOTA or Maleimide-DOTAM was added in a 2-fold molar excess over DARPin protein and mixed thoroughly by pipetting. The conjugation reaction was incubated for approximately 60-120 minutes at room temperature (20-25° C.). To facilitate removal of unreacted chelator, ammonium acetate was added to the conjugation reaction mixture to achieve a final concentration of 500 mM. The sample was then applied to a size exclusion chromatography column (PD-10 column) pre-equilibrated with 500 mM ammonium acetate buffer, pH 6.5 (treated with Chelex to remove trace metals). Up to 2.5 mL of sample was applied to the column, and the DARPin-chelator conjugate was eluted with 500 mM ammonium acetate buffer, pH 6.5. In a second purification step, the eluate from the size exclusion column was subjected to diafiltration using ultrafiltration units (Amicon Ultra-15 Centrifugal Filter Units with a 3 kDa molecular weight cut-off) to further remove unreacted chelator and to exchange the buffer into the final storage buffer. The centrifugal filter units were pre-equilibrated with the final buffer (50 mM ammonium acetate, pH 5.6, treated with Chelex to remove trace metals). The sample was added to the pre-equilibrated filter unit along with approximately 10 mL of the final buffer (50 mM ammonium acetate, pH 5.6, metal-free) and centrifuged at approximately 3000 x g for about 30 minutes at 20° C. This process was repeated five times with fresh buffer additions to achieve thorough removal of unreacted chelator and complete buffer exchange. After diafiltration, the purified DARPin-chelator conjugate was filtered through a sterile 0.22 μm membrane filter and the protein concentration was determined by UV absorbance at 280 nm. The DARPin-chelator solution was stored at ≤−60° C. until loading with a radioactive isotope. The conjugation efficiency was assessed by reversed-phase high-performance liquid chromatography (RP-HPLC). Protein samples were diluted to 0.5 mg/mL and filtered through a 0.22 μm filter prior to analysis. RP-HPLC analysis was performed using a C4 column with a gradient elution using Buffer A (0.1% trifluoroacetic acid in water) and Buffer B (0.1% trifluoroacetic acid in acetonitrile). Successful conjugation was confirmed by a shift in retention time between conjugated and unconjugated DARPin samples, and by the absence or minimal presence of free chelator peaks. For loading of the conjugate with radionuclide, the DARPin-chelator conjugate was mixed with the appropriate radioisotope: For labeling DARPin-DOTA conjugates with lutetium-177 (Lu177), 9 μL of radionuclide solution containing 43 MBq of Lu177 was mixed with 40 μL of DARPin-DOTA conjugate at a concentration of 5.18 mg/mL in buffer (pH 5.0-5.6). The mixture was incubated at 40° C. for 60 minutes to allow complexation of the radiometal with the DOTA chelator. Following incubation, 5 μL of 50 mM diethylenetriaminepentaacetic acid (DTPA) was added as a scavenger to complex any unreacted radiometal. Quality control analysis by radio-HPLC confirmed radiochemical purity of >97%. The resulting radiolabeled conjugate exhibited a corrected specific activity of 0.193 MBq/μg. For labeling DARPin-DOTAM conjugates with lead-203 (Pb203), 50 μL of radionuclide solution containing 34 MBq of Pb203 was mixed with 40 μL of DARPin-DOTAM conjugate at a concentration of 5.5 mg/mL in buffer (pH 5.0-5.6). The mixture was incubated at 40° C. for 60 minutes to allow complexation of the radiometal with the DOTAM chelator. Following incubation, 5 μL of 50 mM DTPA was added as a scavenger to complex any unreacted radiometal. Quality control analysis confirmed radiochemical purity of >97%. The resulting radiolabeled conjugate exhibited a corrected specific activity of 0.138 MBq/μg.

### Biodistribution of Radio-Labeled DARPin Conjugates

ATH mice (Rj:ATHYM-Foxn1^{nu/nu}) were maintained under specific-pathogen-free (SPF) conditions with daily cycles of light and darkness (12 h/12 h), in line with ethical guidelines. No manipulations were performed during the first 7 days after arrival, to allow the animals to acclimatize to the new environment. All mice were monitored daily for assessment of physical condition and general well-being. Solid xenografts were established by subcutaneous (SQ) injection of hMSLN-MC38 or Capan-2 cells. Each mouse was injected subcutaneously with 1×10⁶ hMSLN-MC38 or 5×10⁶ Capan-2 cells in PBS into the right flank of each mouse. Tumor volumes were estimated twice per week through calipering, according to the formula: volume=0.5×length×width² and grown until they reached 200-300 mm³.

Radioisotope-conjugated DARPins were injected at 0.1 mg/kg (150 kBq) into the tail vein of xenografted mice. Biodistribution was monitored at 4 h, 24 h, 72 h, 120 h and 168 h post-injection for mice xenografted with hMSLN-MC38 cells and at 4 h, 24 h, 72 h, 168 h, 240 h, and 336 h post-injection for mice xenografted with Capan-2 cells. Mice were euthanized by CO2 inhalation and cervical dislocation. Selected organs were extracted, weighed and the radioactivity was determined with a γ-counter. The data were expressed as the % injected dose/gram (% ID/g). Results are shown in Table 17 (hMSLN-MC38) and Table 18 (Capan-2).

TABLE 17

| | % ID/g | | | | | | | | | |
|---|---|---|---|---|---|---|---|---|---|---|
| | DARPin #38-DOTAM-Pb203 | | | | | DARPin #38-DOTA-Lu177 | | | | |
| | Time after injection | | | | | | | | | |
| | 4 h | 24 h | 72 h | 120 h | 168 h | 4 h | 24 h | 72 h | 120 h | 168 h |
| Blood | 27.2 | 9.4 | 3.0 | 0.8 | 0.3 | 22.8 | 11.4 | 2.5 | 1.0 | 0.2 |
| Kidneys | 12.2 | 18.4 | 19.8 | 8.7 | 8.9 | 12.6 | 27.4 | 18.4 | 9.0 | 6.8 |
| Liver | 8.9 | 9.4 | 8.7 | 4.5 | 3.5 | 6.9 | 11.7 | 8.7 | 5.7 | 3.7 |
| Tumor | 15.6 | 28.4 | 25.4 | 8.0 | 4.3 | 11.4 | 26.1 | 19.8 | 12.5 | 4.4 |

Both conjugates demonstrated comparable biodistribution profiles across all time points in the hMSLN-MC38 tumor model: Tumor uptake peaked at 24 hours post-injection for both conjugates (28.4% ID/g for Pb203 and 26.1% ID/g for Lu177), followed by gradual clearance. Blood clearance was rapid for both radiopharmaceuticals, declining from 27.2 and 22.8% ID/g at 4 hours to 0.3 and 0.2% ID/g at 168 hours for the Pb203 and Lu177 conjugates, respectively. Kidney accumulation showed similar patterns, peaking at 24 hours (18.4% ID/g for Pb203 and 27.4% ID/g for Lu177) before declining. These results demonstrate that the MSLN-binding DARPin targeting vector functions effectively with both DOTA-Lu177 and DOTAM-Pb203 chelator-radionuclide combinations, yielding comparable tumor targeting and pharmacokinetic properties.

TABLE 18

% ID/g
DARPin #38-DOTA-Lu177
Time after injection

| | 4 h | 24 h | 72 h | 168 h | 240 h | 336 h |
|---|---|---|---|---|---|---|
| Blood | 26.0 | 11.7 | 3.8 | 0.2 | 0.1 | 0 |
| Kidneys | 16.3 | 32.1 | 27.4 | 14.5 | 9.1 | 3.2 |
| Liver | 8.1 | 8.4 | 5.0 | 2.9 | 2.0 | 1.2 |
| Tumor | 5.0 | 7.6 | 8.1 | 3.2 | 2.2 | 1.0 |

In the Capan-2 tumor model, DARPin #38-DOTA-Lu177 was evaluated over an extended time course up to 336 hours post-injection. Blood clearance was rapid, declining from 26.0% ID/g at 4 hours to undetectable levels by 336 hours. Kidney uptake peaked at 24 hours and declined progressively by 336 hours. Tumor accumulation reached maximum levels at 72 hours before gradually clearing. Liver uptake remained low throughout.

Comparison with the hMSLN-MC38 tumor model revealed similar biodistribution trends, including rapid blood clearance, peak kidney accumulation at 24 hours followed by clearance, and low liver uptake. While absolute tumor uptake values differed between the two models, this reflects expected variation in tumor targeting across different xenograft models and likely reflects the expression of MSLN in the different models. The comparable pharmacokinetic profiles in blood, kidney, and liver confirm the consistency of DARPin-conjugate's biodistribution characteristics.

The specification is most thoroughly understood in light of the teachings of the references cited within the specification. The embodiments within the specification provide an illustration of embodiments of the invention and should not be construed to limit the scope of the invention. The skilled artisan readily recognizes that many other embodiments are encompassed by the invention. All publications, patents, and GenBank sequences cited in this disclosure are incorporated by reference in their entirety. To the extent the material incorporated by reference contradicts or is inconsistent with this specification, the specification will supersede any such material. The citation of any references herein is not an admission that such references are prior art to the present invention.

Those skilled in the art will recognize, or be able to ascertain using no more than routine experimentation, many equivalents to the specific embodiments of the invention described herein. Such equivalents are intended to be encompassed by the following claims.

SEQUENCE TABLE

| SEQ | Description | Alternative | |
|---|---|---|---|
| ID NO | of sequence | names | Sequence |
| 1 | Ankyrin repeat | DARPin #1 | DLGLKLLQAAARGQDDEVRELLKAGADVNAKDEWGHTP |
| | domain specific | | LHLAAYTGHLEIVEVLLKAGADVNAKDQFGITPLHVAAYR |
| | for MSLN | | GHLEIVEVLLKAGADVNAQDEAGLTPADLAARLGHEDIAE |
| | | | VLQKAA |
| 2 | Ankyrin repeat | DARPin #2 | DLGLQLLQAAARGQDDTVRELLQAGADVNASDEWGHTP |
| | domain specific | | LHLAAYTGHLEIVEVLLEAGADVNASDQFGITPLHVAAYR |
| | for MSLN | | GHLEIVEVLLEAGADVNAQDEAGLTPADLAARLGNEDIAE |
| | | | VLQQAA |
| 3 | Ankyrin repeat | DARPin #3 | DLGKKLLQAARAGQLDEVRELLKAGADVNAKDEHGHTPL |
| | domain specific | | HLAAYIGHLEIVEVLLKAGADVNAKDEYGITPLHVAAYRGH |
| | for MSLN | | LEIVEVLLKAGADVNAKDVYGWTPLHIAAEHGHLEIVEVLL |
| | | | KAGADVNAQDKSGKTPADLAARAGHQDIAEVLQKAA |
| 4 | Ankyrin repeat | DARPin #4 | DLGEKLLLAAARGQDDEVRELLRAGADVNAKDEHGHTPL |
| | domain specific | | HLAAYIGHLEIVEVLLKAGADVNAKDEYGITPLHVAAYRGH |
| | for MSLN | | LEIVEVLLKAGADVNAKDVYGWTPLHIAAEHGHLEIVEVLL |
| | | | KAGADVNAQDKSGKTPADLAARAGHQDIAEVLQKAA |
| 5 | Ankyrin repeat | DARPin #5 | DLGKKLLEAARAGQDDEVRELLKAGADVNAKDYFSHTPL |
| | domain specific | | HLAARNGHLKIVEVLLKAGADVNAKDFAGKTPLHLAANEG |
| | for human | | HLEIVEVLLKAGADVNAQDIFGKTPADIAADAGHEDIAEVL |
| | serum albumin | | QKAA |
| 6 | Ankyrin repeat | DARPin #6 | DLGKKLLEAARAGQDDEVRELLKAGADVNAKDYFSHTPL |
| | domain specific | | HLAARNGHLKIVEVLLKAGADVNAKDFAGKTPLHLAAADG |
| | for human | | HLEIVEVLLKAGADVNAQDIFGKTPADIAADAGHEDIAEVL |
| | serum albumin | | QKAA |
| 7 | Ankyrin repeat | DARPin #7 | DLGKKLLEAARAGQDDEVRELLKAGADVNAKDYFSHTPL |
| | domain specific | | HLAARNGHLKIVEVLLKAGADVNAKDFAGKTPLHLAADAG |
| | for human | | HLEIVEVLLKAGADVNAQDIFGKTPADIAADAGHEDIAEVL |
| | serum albumin | | QKAA |
| 8 | Ankyrin repeat | DARPin #8 | DLGEKLLEAAKQGQGDEVRELLKAGADVSAKDQLGATPL |
| | domain specific | | HLAAAYGHLEIVEVLLKAGADVNAKDYLGWTPLHVAALAG |
| | for MSLN | | HLEIVEVLLKFGADVNAQDRKGRTPADLAAKKGHEDIAEV |
| | | | LQKAA |
| 9 | G linker | G_1 aa | G |
| 10 | PT linker | PT1n_8 aa | GSPTPTGS |
| 11 | PT linker | PT1n | GSPTPTPTTGS |
| 12 | PT linker | PT3n | GSPTPTPTTPTPTPTTPTPTPTGS |
| 13 | Consensus GS | | [GGGGS]n, wherein n is 1, 2, 3, 4, 5, or 6 |
| | linker | | |
| 14 | PAS linker | PAS08 | PAASASAP |
| 15 | PAS linker | PAS04 | APSPPSASPAAASPAAAPAA |
| 16 | Tag | | GSGSC |
| 17 | Ankyrin repeat | DARPin #9 | DLGLKLLQAAARGQDDEVRELLKAGADVNAKDEWGHTP |
| | protein | | LHLAAYTGHLEIVEVLLKAGADVNAKDQFGITPLHVAAYR |
| | | | GHLEIVEVLLKAGADVNAQDEAGLTPADLAARLGHEDIAE |
| | | | VLQKAAGDLGKKLLEAARAGQDDEVRELLKAGADVNAK |
| | | | DYFSHTPLHLAARNGHLKIVEVLLKAGADVNAKDFAGKTP |
| | | | LHLAADAGHLEIVEVLLKAGADVNAQDIFGKTPADIAADA |
| | | | GHEDIAEVLQKAA |
| 18 | Ankyrin repeat | DARPin #10 | DLGLKLLQAAARGQDDEVRELLKAGADVNAKDEWGHTP |
| | protein | | LHLAAYTGHLEIVEVLLKAGADVNAKDQFGITPLHVAAYR |
| | | | GHLEIVEVLLKAGADVNAQDEAGLTPADLAARLGHEDIAE |
| | | | VLQKAAGSPTPTGSDLGKKLLEAARAGQDDEVRELLKAG |
| | | | ADVNAKDYFSHTPLHLAARNGHLKIVEVLLKAGADVNAK |
| | | | DFAGKTPLHLAADAGHLEIVEVLLKAGADVNAQDIFGKTP |
| | | | ADIAADAGHEDIAEVLQKAA |
| 19 | Ankyrin repeat | DARPin #11 | DLGLKLLQAAARGQDDEVRELLKAGADVNAKDEWGHTP |
| | protein | | LHLAAYTGHLEIVEVLLKAGADVNAKDQFGITPLHVAAYR |
| | | | GHLEIVEVLLKAGADVNAQDEAGLTPADLAARLGHEDIAE |
| | | | VLQKAAGSPTPTPTTGSDLGKKLLEAARAGQDDEVRELL |
| | | | KAGADVNAKDYFSHTPLHLAARNGHLKIVEVLLKAGADV |
| | | | NAKDFAGKTPLHLAADAGHLEIVEVLLKAGADVNAQDIFG |
| | | | KTPADIAADAGHEDIAEVLQKAA |
| 20 | Ankyrin repeat | DARPin #12 | DLGLKLLQAAARGQDDEVRELLKAGADVNAKDEWGHTP |
| | protein | | LHLAAYTGHLEIVEVLLKAGADVNAKDQFGITPLHVAAYR |
| | | | GHLEIVEVLLKAGADVNAQDEAGLTPADLAARLGHEDIAE |
| | | | VLQKAAGSPTPTPTTPTPTPTTPTPTPTGSDLGKKLLEAA |
| | | | RAGQDDEVRELLKAGADVNAKDYFSHTPLHLAARNGHL |
| | | | KIVEVLLKAGADVNAKDFAGKTPLHLAADAGHLEIVEVLLK |
| | | | AGADVNAQDIFGKTPADIAADAGHEDIAEVLQKAA |
| 21 | Ankyrin repeat | DARPin #13 | DLGLKLLQAAARGQDDEVRELLKAGADVNAKDEWGHTP |
| | protein | | LHLAAYTGHLEIVEVLLKAGADVNAKDQFGITPLHVAAYR |
| | | | GHLEIVEVLLKAGADVNAQDEAGLTPADLAARLGHEDIAE |
| | | | VLQKAAPAASASAPDLGKKLLEAARAGQDDEVRELLKAG |
| | | | ADVNAKDYFSHTPLHLAARNGHLKIVEVLLKAGADVNAK |
| | | | DFAGKTPLHLAADAGHLEIVEVLLKAGADVNAQDIFGKTP |
| | | | ADIAADAGHEDIAEVLQKAA |
| 22 | Ankyrin repeat | DARPin #14 | DLGEKLLLAAARGQDDEVRELLRAGADVNAKDEHGHTPL |
| | protein | | HLAAYIGHLEIVEVLLKAGADVNAKDEYGITPLHVAAYRG |
| | | | HLEIVEVLLKAGADVNAKDVYGWTPLHIAAEHGHLEIVEV |
| | | | LLKAGADVNAQDKSGKTPADLAARAGHQDIAEVLQKAAG |
| | | | DLGKKLLEAARAGQDDEVRELLKAGADVNAKDYFSHTPL |
| | | | HLAARNGHLKIVEVLLKAGADVNAKDFAGKTPLHLAADA |
| | | | GHLEIVEVLLKAGADVNAQDIFGKTPADIAADAGHEDIAEV |
| | | | LQKAA |
| 23 | Ankyrin repeat | DARPin #15 | DLGEKLLLAAARGQDDEVRELLRAGADVNAKDEHGHTPL |
| | protein | | HLAAYIGHLEIVEVLLKAGADVNAKDEYGITPLHVAAYRG |
| | | | HLEIVEVLLKAGADVNAKDVYGWTPLHIAAEHGHLEIVEV |
| | | | LLKAGADVNAQDKSGKTPADLAARAGHQDIAEVLQKAAG |
| | | | SPTPTGSDLGKKLLEAARAGQDDEVRELLKAGADVNAKD |
| | | | YFSHTPLHLAARNGHLKIVEVLLKAGADVNAKDFAGKTPL |
| | | | HLAADAGHLEIVEVLLKAGADVNAQDIFGKTPADIAADAG |
| | | | HEDIAEVLQKAA |
| 24 | Ankyrin repeat | DARPin #16 | DLGEKLLLAAARGQDDEVRELLRAGADVNAKDEHGHTPL |
| | protein | | HLAAYIGHLEIVEVLLKAGADVNAKDEYGITPLHVAAYRG |
| | | | HLEIVEVLLKAGADVNAKDVYGWTPLHIAAEHGHLEIVEV |
| | | | LLKAGADVNAQDKSGKTPADLAARAGHQDIAEVLQKAAG |
| | | | SPTPTPTTGSDLGKKLLEAARAGQDDEVRELLKAGADVN |
| | | | AKDYFSHTPLHLAARNGHLKIVEVLLKAGADVNAKDFAG |
| | | | KTPLHLAADAGHLEIVEVLLKAGADVNAQDIFGKTPADIAA |
| | | | DAGHEDIAEVLQKAA |
| 25 | Ankyrin repeat | DARPin #17 | DLGEKLLLAAARGQDDEVRELLRAGADVNAKDEHGHTPL |
| | protein | | HLAAYIGHLEIVEVLLKAGADVNAKDEYGITPLHVAAYRG |
| | | | HLEIVEVLLKAGADVNAKDVYGWTPLHIAAEHGHLEIVEV |
| | | | LLKAGADVNAQDKSGKTPADLAARAGHQDIAEVLQKAAG |
| | | | SPTPTPTTPTPTPTTPTPTPTGSDLGKKLLEAARAGQDDE |
| | | | VRELLKAGADVNAKDYFSHTPLHLAARNGHLKIVEVLLKA |
| | | | GADVNAKDFAGKTPLHLAADAGHLEIVEVLLKAGADVNA |
| | | | QDIFGKTPADIAADAGHEDIAEVLQKAA |
| 26 | Ankyrin repeat | DARPin #18 | DLGEKLLLAAARGQDDEVRELLRAGADVNAKDEHGHTPL |
| | protein | | HLAAYIGHLEIVEVLLKAGADVNAKDEYGITPLHVAAYRG |
| | | | HLEIVEVLLKAGADVNAKDVYGWTPLHIAAEHGHLEIVEV |
| | | | LLKAGADVNAQDKSGKTPADLAARAGHQDIAEVLQKAAP |
| | | | AASASAPDLGKKLLEAARAGQDDEVRELLKAGADVNAKD |
| | | | YFSHTPLHLAARNGHLKIVEVLLKAGADVNAKDFAGKTPL |
| | | | HLAADAGHLEIVEVLLKAGADVNAQDIFGKTPADIAADAG |
| | | | HEDIAEVLQKAA |
| 27 | His tag | | MRGSHHHHHH |
| 28 | His-TEV tag | | MRGSHHHHHHENLYFQ |
| 29 | Ankyrin repeat | DARPin #19 | GSDLGLKLLQAAARGQDDEVRELLKAGADVNAKDEWGH |
| | protein | | TPLHLAAYTGHLEIVEVLLKAGADVNAKDQFGITPLHVAA |
| | | | YRGHLEIVEVLLKAGADVNAQDEAGLTPADLAARLGHEDI |
| | | | AEVLQKAAGDLGKKLLEAARAGQDDEVRELLKAGADVN |
| | | | AKDYFSHTPLHLAARNGHLKIVEVLLKAGADVNAKDFAG |
| | | | KTPLHLAADAGHLEIVEVLLKAGADVNAQDIFGKTPADIAA |
| | | | DAGHEDIAEVLQKAAGSGSC |
| 30 | Ankyrin repeat | DARPin #20 | GSDLGLKLLQAAARGQDDEVRELLKAGADVNAKDEWGH |
| | protein | | TPLHLAAYTGHLEIVEVLLKAGADVNAKDQFGITPLHVAA |
| | | | YRGHLEIVEVLLKAGADVNAQDEAGLTPADLAARLGHEDI |
| | | | AEVLQKAAGSPTPTGSDLGKKLLEAARAGQDDEVRELLK |
| | | | AGADVNAKDYFSHTPLHLAARNGHLKIVEVLLKAGADVN |
| | | | AKDFAGKTPLHLAADAGHLEIVEVLLKAGADVNAQDIFGK |
| | | | TPADIAADAGHEDIAEVLQKAAGSGSC |
| 31 | Ankyrin repeat | DARPin #21 | GSDLGLKLLQAAARGQDDEVRELLKAGADVNAKDEWGH |
| | protein | | TPLHLAAYTGHLEIVEVLLKAGADVNAKDQFGITPLHVAA |
| | | | YRGHLEIVEVLLKAGADVNAQDEAGLTPADLAARLGHEDI |
| | | | AEVLQKAAPAASASAPDLGKKLLEAARAGQDDEVRELLK |
| | | | AGADVNAKDYFSHTPLHLAARNGHLKIVEVLLKAGADVN |
| | | | AKDFAGKTPLHLAADAGHLEIVEVLLKAGADVNAQDIFGK |
| | | | TPADIAADAGHEDIAEVLQKAAGSGSC |
| 32 | Ankyrin repeat | DARPin #22 | GSDLGEKLLLAAARGQDDEVRELLRAGADVNAKDEHGH |
| | protein | | TPLHLAAYIGHLEIVEVLLKAGADVNAKDEYGITPLHVAAY |
| | | | RGHLEIVEVLLKAGADVNAKDVYGWTPLHIAAEHGHLEIV |
| | | | EVLLKAGADVNAQDKSGKTPADLAARAGHQDIAEVLQKA |
| | | | AGDLGKKLLEAARAGQDDEVRELLKAGADVNAKDYFSH |
| | | | TPLHLAARNGHLKIVEVLLKAGADVNAKDFAGKTPLHLAA |
| | | | DAGHLEIVEVLLKAGADVNAQDIFGKTPADIAADAGHEDI |
| | | | AEVLQKAAGSGSC |
| 33 | Ankyrin repeat | DARPin #23 | GSDLGEKLLLAAARGQDDEVRELLRAGADVNAKDEHGH |
| | protein | | TPLHLAAYIGHLEIVEVLLKAGADVNAKDEYGITPLHVAAY |
| | | | RGHLEIVEVLLKAGADVNAKDVYGWTPLHIAAEHGHLEIV |
| | | | EVLLKAGADVNAQDKSGKTPADLAARAGHQDIAEVLQKA |
| | | | AGSPTPTGSDLGKKLLEAARAGQDDEVRELLKAGADVN |
| | | | AKDYFSHTPLHLAARNGHLKIVEVLLKAGADVNAKDFAG |
| | | | KTPLHLAADAGHLEIVEVLLKAGADVNAQDIFGKTPADIAA |
| | | | DAGHEDIAEVLQKAAGSGSC |
| 34 | Ankyrin repeat | DARPin #24 | GSDLGEKLLLAAARGQDDEVRELLRAGADVNAKDEHGH |
| | protein | | TPLHLAAYIGHLEIVEVLLKAGADVNAKDEYGITPLHVAAY |
| | | | RGHLEIVEVLLKAGADVNAKDVYGWTPLHIAAEHGHLEIV |
| | | | EVLLKAGADVNAQDKSGKTPADLAARAGHQDIAEVLQKA |
| | | | APAASASAPDLGKKLLEAARAGQDDEVRELLKAGADVNA |
| | | | KDYFSHTPLHLAARNGHLKIVEVLLKAGADVNAKDFAGKT |
| | | | PLHLAADAGHLEIVEVLLKAGADVNAQDIFGKTPADIAAD |
| | | | AGHEDIAEVLQKAAGSGSC |
| 35 | N-terminal | | DLGKKLLEAARAGQDDEVRELLKAGADVNA |
| | capping module | | |
| 36 | Internal repeat | | KDAAGETPLHLAAWFGHLEIVEVLLKAGADVNA |
| | module | | |
| 37 | C-terminal | | QDLQGKTPADLAAKEGHEDIAEVLQKAA |
| | capping module | | |
| 38 | Ankyrin repeat | DARPin #25 | GSDLGLQLLQAAARGQDDTVRELLQAGADVNASDEWG |
| | protein | | HTPLHLAAYTGHLEIVEVLLEAGADVNASDQFGITPLHVA |
| | | | AYRGHLEIVEVLLEAGADVNAQDEAGLTPADLAARLGNE |
| | | | DIAEVLQQAAGSPTPTPTTPTPTPTTPTPTPTGSDLGKKL |
| | | | LEAARAGQDDEVRELLKAGADVNAKDYFSHTPLHLAARN |
| | | | GHLKIVEVLLKAGADVNAKDFAGKTPLHLAADAGHLEIVE |
| | | | VLLKAGADVNAQDIFGKTPADIAADAGHEDIAEVLQKAAG |
| | | | SGSC |
| 39 | Ankyrin repeat | DARPin #26 | GSDLGKKLLEAARAGQDDEVRELLKAGADVNAKDYFSHT |
| | protein | | PLHLAARNGHLKIVEVLLKAGADVNAKDFAGKTPLHLAAD |
| | | | AGHLEIVEVLLKAGADVNAQDIFGKTPADIAADAGHEDIAE |
| | | | VLQKAAGSPTPTPTTPTPTPTTPTPTPTGSDLGLQLLQAA |
| | | | ARGQDDTVRELLQAGADVNASDEWGHTPLHLAAYTGHL |
| | | | EIVEVLLEAGADVNASDQFGITPLHVAAYRGHLEIVEVLLE |
| | | | AGADVNAQDEAGLTPADLAARLGNEDIAEVLQQAAGSGS |
| | | | C |
| 40 | Ankyrin repeat | DARPin #27 | GSDLGKKLLEAARAGQDDEVRELLKAGADVNAKDYFSHT |
| | protein | | PLHLAARNGHLKIVEVLLKAGADVNAKDFAGKTPLHLAAD |
| | | | AGHLEIVEVLLKAGADVNAQDIFGKTPADIAADAGHEDIAE |
| | | | VLQKAAGSPTPTPTTPTPTPTTPTPTPTGSDLGEKLLEAA |
| | | | KQGQGDEVRELLKAGADVSAKDQLGATPLHLAAAYGHLE |
| | | | IVEVLLKAGADVNAKDYLGWTPLHVAALAGHLEIVEVLLKF |
| | | | GADVNAQDRKGRTPADLAAKKGHEDIAEVLQKAAGSGS |
| | | | C |
| 41 | Ankyrin repeat | Non-binding | DLGKKLLEAARAGQDDEVRELLKAGADVNAKDKDGYTPL |
| | domain | DARPin | HLAAREGHLEIVEVLLKAGADVNAKDKDGYTPLHLAAREG |
| | | | HLEIVEVLLKAGADVNAQDKSGKTPADLAADAGHEDIAEV |
| | | | LQKAA |
| 42 | Ankyrin repeat | DARPin #28 | DLGEKLLRAAAQGQDDEVRELLKAGADVNAKDHYGHTPL |
| | domain specific | | HVAAYWGHLEIVEVLLKAGADVNAKDEYGITPLHVAAYRG |
| | for MSLN | | HLEIVEVLLKAGADVNAQDAAGLTPADLAARLGHDDIAEV |
| | | | LQKAA |
| 43 | Ankyrin repeat | DARPin #29 | DLGLKLLQAAARGNPDEVRELLQAGADVNAKDEWGHTP |
| | domain specific | | LHLAAYTGHPEIVEVLLEAGADVNAKDQFGITPLHVAAYR |
| | for MSLN | | GHPEIVEVLLEAGADVNAQDEAGLTPADLAARLGHEDIAE |
| | | | VLQKAA |
| 44 | Ankyrin repeat | DARPin #30 | DLGEKLLRAAAQGNPDEVRELLQAGADVNAKDHYGHTPL |
| | domain specific | | HVAAYWGHPEIVEVLLEAGADVNAKDEYGITPLHVAAYR |
| | for MSLN | | GHPEIVEVLLEAGADVNAQDAAGLTPADLAARLGHDDIAE |
| | | | VLQKAA |
| 45 | Ankyrin repeat | DARPin #31 | DLGKKLLEAARAGNPDEVRELLQAGADVNAKDYFSHTPL |
| | domain specific | | HLAARNGHPEIVEVLLEAGADVNAKDFAGKTPLHLAADA |
| | for human | | GHPEIVEVLLEAGADVNAQDIFGKTPADIAADAGHEDIAEV |
| | serum albumin | | LQKAA |
| 46 | Ankyrin repeat | DARPin #32 | DLGEKLLRAAAQGQDDEVRELLKAGADVNAKDHYGHTPL |
| | protein | | HVAAYWGHLEIVEVLLKAGADVNAKDEYGITPLHVAAYRG |
| | | | HLEIVEVLLKAGADVNAQDAAGLTPADLAARLGHDDIAEV |
| | | | LQKAAGSPTPTPTTGSDLGKKLLEAARAGQDDEVRELLK |
| | | | AGADVNAKDYFSHTPLHLAARNGHLKIVEVLLKAGADVNA |
| | | | KDFAGKTPLHLAADAGHLEIVEVLLKAGADVNAQDIFGKT |
| | | | PADIAADAGHEDIAEVLQKAA |
| 47 | Ankyrin repeat | DARPin #33 | DLGEKLLRAAAQGNPDEVRELLQAGADVNAKDHYGHTPL |
| | protein | | HVAAYWGHPEIVEVLLEAGADVNAKDEYGITPLHVAAYR |
| | | | GHPEIVEVLLEAGADVNAQDAAGLTPADLAARLGHDDIAE |
| | | | VLQKAAGSPTPTPTTGSDLGKKLLEAARAGQDDEVRELL |
| | | | KAGADVNAKDYFSHTPLHLAARNGHLKIVEVLLKAGADVN |
| | | | AKDFAGKTPLHLAADAGHLEIVEVLLKAGADVNAQDIFGK |
| | | | TPADIAADAGHEDIAEVLQKAA |
| 48 | Ankyrin repeat | DARPin #34 | DLGEKLLRAAAQGNPDEVRELLQAGADVNAKDHYGHTPL |
| | protein | | HVAAYWGHPEIVEVLLEAGADVNAKDEYGITPLHVAAYR |
| | | | GHPEIVEVLLEAGADVNAQDAAGLTPADLAARLGHDDIAE |
| | | | VLQKAAGSPTPTPTTGSDLGKKLLEAARAGNPDEVRELL |
| | | | QAGADVNAKDYFSHTPLHLAARNGHPEIVEVLLEAGADV |
| | | | NAKDFAGKTPLHLAADAGHPEIVEVLLEAGADVNAQDIFG |
| | | | KTPADIAADAGHEDIAEVLQKAA |
| 49 | Ankyrin repeat | DARPin #35 | DLGLKLLQAAARGNPDEVRELLQAGADVNAKDEWGHTP |
| | protein | | LHLAAYTGHPEIVEVLLEAGADVNAKDQFGITPLHVAAYR |
| | | | GHPEIVEVLLEAGADVNAQDEAGLTPADLAARLGHEDIAE |
| | | | VLQKAAGSPTPTPTTGSDLGKKLLEAARAGQDDEVRELL |
| | | | KAGADVNAKDYFSHTPLHLAARNGHLKIVEVLLKAGADVN |
| | | | AKDFAGKTPLHLAADAGHLEIVEVLLKAGADVNAQDIFGK |
| | | | TPADIAADAGHEDIAEVLQKAA |
| 50 | Ankyrin repeat | DARPin #36 | DLGLKLLQAAARGNPDEVRELLQAGADVNAKDEWGHTP |
| | protein | | LHLAAYTGHPEIVEVLLEAGADVNAKDQFGITPLHVAAYR |
| | | | GHPEIVEVLLEAGADVNAQDEAGLTPADLAARLGHEDIAE |
| | | | VLQKAAGSPTPTPTTGSDLGKKLLEAARAGNPDEVRELL |
| | | | QAGADVNAKDYFSHTPLHLAARNGHPEIVEVLLEAGADV |
| | | | NAKDFAGKTPLHLAADAGHPEIVEVLLEAGADVNAQDIFG |
| | | | KTPADIAADAGHEDIAEVLQKAA |
| 51 | Ankyrin repeat | DARPin #37 | GSDLGEKLLRAAAQGQDDEVRELLKAGADVNAKDHYGH |
| | protein | | TPLHVAAYWGHLEIVEVLLKAGADVNAKDEYGITPLHVAA |
| | | | YRGHLEIVEVLLKAGADVNAQDAAGLTPADLAARLGHDDI |
| | | | AEVLQKAAGSPTPTPTTGSDLGKKLLEAARAGQDDEVRE |
| | | | LLKAGADVNAKDYFSHTPLHLAARNGHLKIVEVLLKAGAD |
| | | | VNAKDFAGKTPLHLAADAGHLEIVEVLLKAGADVNAQDIF |
| | | | GKTPADIAADAGHEDIAEVLQKAAGSGSC |
| 52 | Ankyrin repeat | DARPin #38 | GSDLGEKLLRAAAQGNPDEVRELLQAGADVNAKDHYGH |
| | protein | | TPLHVAAYWGHPEIVEVLLEAGADVNAKDEYGITPLHVAA |
| | | | YRGHPEIVEVLLEAGADVNAQDAAGLTPADLAARLGHDDI |
| | | | AEVLQKAAGSPTPTPTTGSDLGKKLLEAARAGQDDEVRE |
| | | | LLKAGADVNAKDYFSHTPLHLAARNGHLKIVEVLLKAGAD |
| | | | VNAKDFAGKTPLHLAADAGHLEIVEVLLKAGADVNAQDIF |
| | | | GKTPADIAADAGHEDIAEVLQKAAGSGSC |
| 53 | Ankyrin repeat | DARPin #39 | GSDLGEKLLRAAAQGNPDEVRELLQAGADVNAKDHYGH |
| | protein | | TPLHVAAYWGHPEIVEVLLEAGADVNAKDEYGITPLHVAA |
| | | | YRGHPEIVEVLLEAGADVNAQDAAGLTPADLAARLGHDDI |
| | | | AEVLQKAAGSPTPTPTTGSDLGKKLLEAARAGNPDEVRE |
| | | | LLQAGADVNAKDYFSHTPLHLAARNGHPEIVEVLLEAGAD |
| | | | VNAKDFAGKTPLHLAADAGHPEIVEVLLEAGADVNAQDIF |
| | | | GKTPADIAADAGHEDIAEVLQKAAGSGSC |
| 54 | Ankyrin repeat | DARPin #40 | GSDLGLKLLQAAARGNPDEVRELLQAGADVNAKDEWGH |
| | protein | | TPLHLAAYTGHPEIVEVLLEAGADVNAKDQFGITPLHVAA |
| | | | YRGHPEIVEVLLEAGADVNAQDEAGLTPADLAARLGHEDI |
| | | | AEVLQKAAGSPTPTPTTGSDLGKKLLEAARAGQDDEVRE |
| | | | LLKAGADVNAKDYFSHTPLHLAARNGHLKIVEVLLKAGAD |
| | | | VNAKDFAGKTPLHLAADAGHLEIVEVLLKAGADVNAQDIF |
| | | | GKTPADIAADAGHEDIAEVLQKAAGSGSC |
| 55 | Ankyrin repeat | DARPin #41 | GSDLGLKLLQAAARGNPDEVRELLQAGADVNAKDEWGH |
| | protein | | TPLHLAAYTGHPEIVEVLLEAGADVNAKDQFGITPLHVAA |
| | | | YRGHPEIVEVLLEAGADVNAQDEAGLTPADLAARLGHEDI |
| | | | AEVLQKAAGSPTPTPTTGSDLGKKLLEAARAGNPDEVRE |
| | | | LLQAGADVNAKDYFSHTPLHLAARNGHPEIVEVLLEAGAD |
| | | | VNAKDFAGKTPLHLAADAGHPEIVEVLLEAGADVNAQDIF |
| | | | GKTPADIAADAGHEDIAEVLQKAAGSGSC |
| 56 | Ankyrin repeat | DARPin #42 | GSDLGLKLLQAAARGQDDEVRELLKAGADVNAKDEWGH |
| | protein | | TPLHLAAYTGHLEIVEVLLKAGADVNAKDQFGITPLHVAAY |
| | | | RGHLEIVEVLLKAGADVNAQDEAGLTPADLAARLGHEDIA |
| | | | EVLQKAAGSPTPTPTTGSDLGKKLLEAARAGQDDEVREL |
| | | | LKAGADVNAKDYFSHTPLHLAARNGHLKIVEVLLKAGADV |
| | | | NAKDFAGKTPLHLAADAGHLEIVEVLLKAGADVNAQDIFG |
| | | | KTPADIAADAGHEDIAEVLQKAAGSGSC |

1. A conjugate, or a pharmaceutically acceptable salt thereof, comprising (i) an ankyrin repeat domain with binding specificity for MSLN, (ii) a chelator, and (iii) a radionuclide.

2. The conjugate, or pharmaceutically acceptable salt thereof, of claim 1, wherein said chelator is covalently connected to said ankyrin repeat domain, wherein said radionuclide is bound to said chelator.

3. The conjugate, or pharmaceutically acceptable salt thereof, of claim 1, wherein said radionuclide is Ac-225, Lu-177, Pb-203, Pb-212, In-111, Tb-161, Cu-64, Cu-67, Zr-89, Y-86, or Ce-134.

4. The conjugate, or pharmaceutically acceptable salt thereof, of wherein said radionuclide is bound to said chelator, and wherein said radionuclide is Pb-212 or Pb-203.

5. The conjugate, or pharmaceutically acceptable salt thereof, of claim 1, wherein said ankyrin repeat domain with binding specificity for MSLN comprises an amino acid sequence that is at least 80% identical to any one of SEQ ID NOs: 1 to 4, or 42 to 44.

6. The conjugate or pharmaceutically acceptable salt thereof, of claim 1, wherein said chelator has a structure of Formula (I): 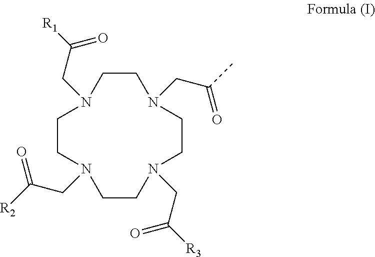
wherein R1, R2 and R3 are independently NH₂ or OH, and wherein the dotted line represents the covalent connection to said ankyrin repeat domain with binding specificity for MSLN or to a connector; or wherein said chelator has a structure of Formula (III): 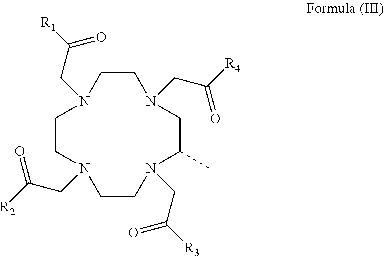
wherein R1, R2, R3 and R4 are independently NH₂ or OH, and wherein the dotted line represents the covalent connection to said ankyrin repeat domain with binding specificity for MSLN or to a connector.

7. The conjugate, or pharmaceutically acceptable salt thereof, of claim 6, wherein said connector comprises a maleimide or a derivative thereof.

8. The conjugate, or pharmaceutically acceptable salt thereof, of claim 1, further comprising a tag, wherein said tag comprises a Cysteine and/or further comprising a half-life extending moiety.

9. The conjugate, or pharmaceutically acceptable salt thereof, of claim 8, wherein said half-life extending moiety is an ankyrin repeat domain with binding specificity for human serum albumin.

10. The conjugate, or pharmaceutically acceptable salt thereof, of claim 1, wherein said conjugate comprises an amino acid sequence that is at least 80% identical to any one of SEQ ID NOs: 17 to 26, or 46 to 50.

11. A conjugate, or pharmaceutically acceptable salt thereof, wherein said conjugate has a structure of Formula (VI): 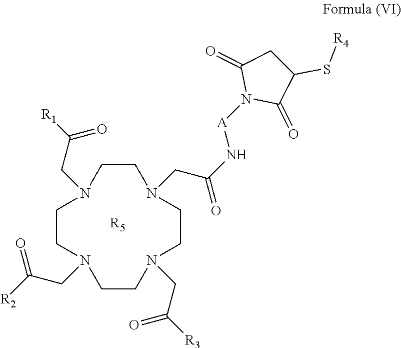
wherein R1, R2, and R3 are independently NH₂ or OH;
wherein A is CₐH_{b}N_{c}O_{d}, wherein a, b, c, and d are integers;
wherein R4 is an ankyrin repeat protein comprising an amino acid sequence that is at least 80% identical to any one of SEQ ID NOs: 29 to 34, 38, or 51 to 56, wherein said ankyrin repeat protein has binding specificity for MSLN and for human serum albumin, and wherein said ankyrin repeat protein comprises a Cysteine;
and wherein R5 is a chelated radionuclide, wherein said radionuclide is Ac-225, Lu-177, Pb-203, Pb-212, In-111, Tb-161, Cu-64, Cu-67, Zr-89, Y-86, or Ce-134.

12. A conjugate; or pharmaceutically acceptable salt thereof, wherein said conjugate has a structure of Formula (VII): 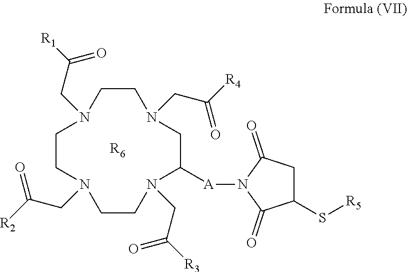
wherein R1, R2, R3 and R4 are independently NH₂ or OH;
wherein A is CₐH_{b}N_{c}O_{d}, wherein a, b, c, and d are integers;
wherein R5 is an ankyrin repeat protein comprising an amino acid sequence that is at least 80% identical to any one of SEQ ID NOs: 29 to 34, 38, or 51 to 56, wherein said ankyrin repeat protein has binding specificity for MSLN and for human serum albumin, and wherein said ankyrin repeat protein comprises a Cysteine;
and wherein R6 is a chelated radionuclide, wherein said radionuclide is Ac-225, Lu-177, Pb-203, Pb-212, In-111, Tb-161, Cu-64, Cu-67, Zr-89, Y-86, or Ce-134.

13. A pharmaceutical composition, comprising the conjugate, or pharmaceutically acceptable salt thereof, of claim 1, and optionally a pharmaceutically acceptable carrier or excipient.

14. A method of treating cancer, the method comprising the step of administering to a subject in need thereof a therapeutically effective amount of the conjugate, or pharmaceutically acceptable salt thereof, of claim 1.


SEQUENCE LISTING (56 sequences)

SEQ ID NO: 1 - protein, 124 aa, synthetic construct
  source 1..124: note = Ankyrin repeat domain specific for MSLN (DARPin #1)
DLGLKLLQAAARGQDDEVRELLKAGADVNAKDEWGHTPLHLAAYTGHLEIVEVLLKAGADVNAKDQFGITPLHVAAYRGHLEIVEVLLKAGADVNAQDEAGLTPADLAARLGHEDIAEVLQKAA

SEQ ID NO: 2 - protein, 124 aa, synthetic construct
  source 1..124: note = Ankyrin repeat domain specific for MSLN (DARPin #2)
DLGLQLLQAAARGQDDTVRELLQAGADVNASDEWGHTPLHLAAYTGHLEIVEVLLEAGADVNASDQFGITPLHVAAYRGHLEIVEVLLEAGADVNAQDEAGLTPADLAARLGNEDIAEVLQQAA

SEQ ID NO: 3 - protein, 157 aa, synthetic construct
  source 1..157: note = Ankyrin repeat domain specific for MSLN (DARPin #3)
DLGKKLLQAARAGQLDEVRELLKAGADVNAKDEHGHTPLHLAAYIGHLEIVEVLLKAGADVNAKDEYGITPLHVAAYRGHLEIVEVLLKAGADVNAKDVYGWTPLHIAAEHGHLEIVEVLLKAGADVNAQDKSGKTPADLAARAGHQDIAEVLQKAA

SEQ ID NO: 4 - protein, 157 aa, synthetic construct
  source 1..157: note = Ankyrin repeat domain specific for MSLN (DARPin #4)
DLGEKLLLAAARGQDDEVRELLRAGADVNAKDEHGHTPLHLAAYIGHLEIVEVLLKAGADVNAKDEYGITPLHVAAYRGHLEIVEVLLKAGADVNAKDVYGWTPLHIAAEHGHLEIVEVLLKAGADVNAQDKSGKTPADLAARAGHQDIAEVLQKAA

SEQ ID NO: 5 - protein, 124 aa, synthetic construct
  source 1..124: note = Ankyrin repeat domain specific for human serum albumin (DARPin #5)
DLGKKLLEAARAGQDDEVRELLKAGADVNAKDYFSHTPLHLAARNGHLKIVEVLLKAGADVNAKDFAGKTPLHLAANEGHLEIVEVLLKAGADVNAQDIFGKTPADIAADAGHEDIAEVLQKAA

SEQ ID NO: 6 - protein, 124 aa, synthetic construct
  source 1..124: note = Ankyrin repeat domain specific for human serum albumin (DAPRin #6)
DLGKKLLEAARAGQDDEVRELLKAGADVNAKDYFSHTPLHLAARNGHLKIVEVLLKAGADVNAKDFAGKTPLHLAAADGHLEIVEVLLKAGADVNAQDIFGKTPADIAADAGHEDIAEVLQKAA

SEQ ID NO: 7 - protein, 124 aa, synthetic construct
  source 1..124: note = Ankyrin repeat domain specific for human serum albumin (DAPRin #7)
DLGKKLLEAARAGQDDEVRELLKAGADVNAKDYFSHTPLHLAARNGHLKIVEVLLKAGADVNAKDFAGKTPLHLAADAGHLEIVEVLLKAGADVNAQDIFGKTPADIAADAGHEDIAEVLQKAA

SEQ ID NO: 8 - protein, 124 aa, synthetic construct
  source 1..124: note = Ankyrin repeat domain specific for MSLN (DAPRin #8)
DLGEKLLEAAKQGQGDEVRELLKAGADVSAKDQLGATPLHLAAAYGHLEIVEVLLKAGADVNAKDYLGWTPLHVAALAGHLEIVEVLLKFGADVNAQDRKGRTPADLAAKKGHEDIAEVLQKAA

SEQ ID NO: 9 - skipped

SEQ ID NO: 10 - protein, 8 aa, synthetic construct
  source 1..8: note = PT linker (PT1n_8aa)
GSPTPTGS

SEQ ID NO: 11 - protein, 11 aa, synthetic construct
  source 1..11: note = PT linker (PT1n)
GSPTPTPTTGS

SEQ ID NO: 12 - protein, 24 aa, synthetic construct
  source 1..24: note = PT linker (PT3n)
GSPTPTPTTPTPTPTTPTPTPTGS

SEQ ID NO: 13 - protein, 5 aa, synthetic construct
  source 1..5: note = Consensus GS linker
  REGION 1..>5: note = The entire sequence of amino acids 1-5 can be repeated 1, 2, 3, 4, 5 or 6 times.
GGGGS

SEQ ID NO: 14 - protein, 8 aa, synthetic construct
  source 1..8: note = PAS linker (PAS08)
PAASASAP

SEQ ID NO: 15 - protein, 20 aa, synthetic construct
  source 1..20: note = PAS linker (PAS04)
APSPPSASPAAASPAAAPAA

SEQ ID NO: 16 - protein, 5 aa, synthetic construct
  source 1..5: note = Tag
GSGSC

SEQ ID NO: 17 - protein, 249 aa, synthetic construct
  source 1..249: note = Ankyrin repeat protein (DAPRin #9)
DLGLKLLQAAARGQDDEVRELLKAGADVNAKDEWGHTPLHLAAYTGHLEIVEVLLKAGADVNAKDQFGITPLHVAAYRGHLEIVEVLLKAGADVNAQDEAGLTPADLAARLGHEDIAEVLQKAAGDLGKKLLEAARAGQDDEVRELLKAGADVNAKDYFSHTPLHLAARNGHLKIVEVLLKAGADVNAKDFAGKTPLHLAADAGHLEIVEVLLKAGADVNAQDIFGKTPADIAADAGHEDIAEVLQKAA

SEQ ID NO: 18 - protein, 256 aa, synthetic construct
  source 1..256: note = Ankyrin repeat protein (DAPRin #10)
DLGLKLLQAAARGQDDEVRELLKAGADVNAKDEWGHTPLHLAAYTGHLEIVEVLLKAGADVNAKDQFGITPLHVAAYRGHLEIVEVLLKAGADVNAQDEAGLTPADLAARLGHEDIAEVLQKAAGSPTPTGSDLGKKLLEAARAGQDDEVRELLKAGADVNAKDYFSHTPLHLAARNGHLKIVEVLLKAGADVNAKDFAGKTPLHLAADAGHLEIVEVLLKAGADVNAQDIFGKTPADIAADAGHEDIAEVLQKAA

SEQ ID NO: 19 - protein, 259 aa, synthetic construct
  source 1..259: note = Ankyrin repeat protein (DARPin #11)
DLGLKLLQAAARGQDDEVRELLKAGADVNAKDEWGHTPLHLAAYTGHLEIVEVLLKAGADVNAKDQFGITPLHVAAYRGHLEIVEVLLKAGADVNAQDEAGLTPADLAARLGHEDIAEVLQKAAGSPTPTPTTGSDLGKKLLEAARAGQDDEVRELLKAGADVNAKDYFSHTPLHLAARNGHLKIVEVLLKAGADVNAKDFAGKTPLHLAADAGHLEIVEVLLKAGADVNAQDIFGKTPADIAADAGHEDIAEVLQKAA

SEQ ID NO: 20 - protein, 272 aa, synthetic construct
  source 1..272: note = Ankyrin repeat protein (DARPin #12)
DLGLKLLQAAARGQDDEVRELLKAGADVNAKDEWGHTPLHLAAYTGHLEIVEVLLKAGADVNAKDQFGITPLHVAAYRGHLEIVEVLLKAGADVNAQDEAGLTPADLAARLGHEDIAEVLQKAAGSPTPTPTTPTPTPTTPTPTPTGSDLGKKLLEAARAGQDDEVRELLKAGADVNAKDYFSHTPLHLAARNGHLKIVEVLLKAGADVNAKDFAGKTPLHLAADAGHLEIVEVLLKAGADVNAQDIFGKTPADIAADAGHEDIAEVLQKAA

SEQ ID NO: 21 - protein, 256 aa, synthetic construct
  source 1..256: note = Ankyrin repeat protein (DARPin #13)
DLGLKLLQAAARGQDDEVRELLKAGADVNAKDEWGHTPLHLAAYTGHLEIVEVLLKAGADVNAKDQFGITPLHVAAYRGHLEIVEVLLKAGADVNAQDEAGLTPADLAARLGHEDIAEVLQKAAPAASASAPDLGKKLLEAARAGQDDEVRELLKAGADVNAKDYFSHTPLHLAARNGHLKIVEVLLKAGADVNAKDFAGKTPLHLAADAGHLEIVEVLLKAGADVNAQDIFGKTPADIAADAGHEDIAEVLQKAA

SEQ ID NO: 22 - protein, 282 aa, synthetic construct
  source 1..282: note = Ankyrin repeat protein (DARPin #14)
DLGEKLLLAAARGQDDEVRELLRAGADVNAKDEHGHTPLHLAAYIGHLEIVEVLLKAGADVNAKDEYGITPLHVAAYRGHLEIVEVLLKAGADVNAKDVYGWTPLHIAAEHGHLEIVEVLLKAGADVNAQDKSGKTPADLAARAGHQDIAEVLQKAAGDLGKKLLEAARAGQDDEVRELLKAGADVNAKDYFSHTPLHLAARNGHLKIVEVLLKAGADVNAKDFAGKTPLHLAADAGHLEIVEVLLKAGADVNAQDIFGKTPADIAADAGHEDIAEVLQKAA

SEQ ID NO: 23 - protein, 289 aa, synthetic construct
  source 1..289: note = Ankyrin repeat protein (DARPin #15)
DLGEKLLLAAARGQDDEVRELLRAGADVNAKDEHGHTPLHLAAYIGHLEIVEVLLKAGADVNAKDEYGITPLHVAAYRGHLEIVEVLLKAGADVNAKDVYGWTPLHIAAEHGHLEIVEVLLKAGADVNAQDKSGKTPADLAARAGHQDIAEVLQKAAGSPTPTGSDLGKKLLEAARAGQDDEVRELLKAGADVNAKDYFSHTPLHLAARNGHLKIVEVLLKAGADVNAKDFAGKTPLHLAADAGHLEIVEVLLKAGADVNAQDIFGKTPADIAADAGHEDIAEVLQKAA

SEQ ID NO: 24 - protein, 292 aa, synthetic construct
  source 1..292: note = Ankyrin repeat protein (DARPin #16)
DLGEKLLLAAARGQDDEVRELLRAGADVNAKDEHGHTPLHLAAYIGHLEIVEVLLKAGADVNAKDEYGITPLHVAAYRGHLEIVEVLLKAGADVNAKDVYGWTPLHIAAEHGHLEIVEVLLKAGADVNAQDKSGKTPADLAARAGHQDIAEVLQKAAGSPTPTPTTGSDLGKKLLEAARAGQDDEVRELLKAGADVNAKDYFSHTPLHLAARNGHLKIVEVLLKAGADVNAKDFAGKTPLHLAADAGHLEIVEVLLKAGADVNAQDIFGKTPADIAADAGHEDIAEVLQKAA

SEQ ID NO: 25 - protein, 305 aa, synthetic construct
  source 1..305: note = Ankyrin repeat protein (DARPin #17)
DLGEKLLLAAARGQDDEVRELLRAGADVNAKDEHGHTPLHLAAYIGHLEIVEVLLKAGADVNAKDEYGITPLHVAAYRGHLEIVEVLLKAGADVNAKDVYGWTPLHIAAEHGHLEIVEVLLKAGADVNAQDKSGKTPADLAARAGHQDIAEVLQKAAGSPTPTPTTPTPTPTTPTPTPTGSDLGKKLLEAARAGQDDEVRELLKAGADVNAKDYFSHTPLHLAARNGHLKIVEVLLKAGADVNAKDFAGKTPLHLAADAGHLEIVEVLLKAGADVNAQDIFGKTPADIAADAGHEDIAEVLQKAA

SEQ ID NO: 26 - protein, 289 aa, synthetic construct
  source 1..289: note = Ankyrin repeat protein (DARPin #18)
DLGEKLLLAAARGQDDEVRELLRAGADVNAKDEHGHTPLHLAAYIGHLEIVEVLLKAGADVNAKDEYGITPLHVAAYRGHLEIVEVLLKAGADVNAKDVYGWTPLHIAAEHGHLEIVEVLLKAGADVNAQDKSGKTPADLAARAGHQDIAEVLQKAAPAASASAPDLGKKLLEAARAGQDDEVRELLKAGADVNAKDYFSHTPLHLAARNGHLKIVEVLLKAGADVNAKDFAGKTPLHLAADAGHLEIVEVLLKAGADVNAQDIFGKTPADIAADAGHEDIAEVLQKAA

SEQ ID NO: 27 - protein, 10 aa, synthetic construct
  source 1..10: note = His tag
MRGSHHHHHH

SEQ ID NO: 28 - protein, 16 aa, synthetic construct
  source 1..16: note = His-TEV tag
MRGSHHHHHHENLYFQ

SEQ ID NO: 29 - protein, 256 aa, synthetic construct
  source 1..256: note = Ankyrin repeat protein (DARPin #19)
GSDLGLKLLQAAARGQDDEVRELLKAGADVNAKDEWGHTPLHLAAYTGHLEIVEVLLKAGADVNAKDQFGITPLHVAAYRGHLEIVEVLLKAGADVNAQDEAGLTPADLAARLGHEDIAEVLQKAAGDLGKKLLEAARAGQDDEVRELLKAGADVNAKDYFSHTPLHLAARNGHLKIVEVLLKAGADVNAKDFAGKTPLHLAADAGHLEIVEVLLKAGADVNAQDIFGKTPADIAADAGHEDIAEVLQKAAGSGSC

SEQ ID NO: 30 - protein, 263 aa, synthetic construct
  source 1..263: note = Ankyrin repeat protein (DARPin #20)
GSDLGLKLLQAAARGQDDEVRELLKAGADVNAKDEWGHTPLHLAAYTGHLEIVEVLLKAGADVNAKDQFGITPLHVAAYRGHLEIVEVLLKAGADVNAQDEAGLTPADLAARLGHEDIAEVLQKAAGSPTPTGSDLGKKLLEAARAGQDDEVRELLKAGADVNAKDYFSHTPLHLAARNGHLKIVEVLLKAGADVNAKDFAGKTPLHLAADAGHLEIVEVLLKAGADVNAQDIFGKTPADIAADAGHEDIAEVLQKAAGSGSC

SEQ ID NO: 31 - protein, 263 aa, synthetic construct
  source 1..263: note = Ankyrin repeat protein (DARPin #21)
GSDLGLKLLQAAARGQDDEVRELLKAGADVNAKDEWGHTPLHLAAYTGHLEIVEVLLKAGADVNAKDQFGITPLHVAAYRGHLEIVEVLLKAGADVNAQDEAGLTPADLAARLGHEDIAEVLQKAAPAASASAPDLGKKLLEAARAGQDDEVRELLKAGADVNAKDYFSHTPLHLAARNGHLKIVEVLLKAGADVNAKDFAGKTPLHLAADAGHLEIVEVLLKAGADVNAQDIFGKTPADIAADAGHEDIAEVLQKAAGSGSC

SEQ ID NO: 32 - protein, 289 aa, synthetic construct
  source 1..289: note = Ankyrin repeat protein (DARPin #22)
GSDLGEKLLLAAARGQDDEVRELLRAGADVNAKDEHGHTPLHLAAYIGHLEIVEVLLKAGADVNAKDEYGITPLHVAAYRGHLEIVEVLLKAGADVNAKDVYGWTPLHIAAEHGHLEIVEVLLKAGADVNAQDKSGKTPADLAARAGHQDIAEVLQKAAGDLGKKLLEAARAGQDDEVRELLKAGADVNAKDYFSHTPLHLAARNGHLKIVEVLLKAGADVNAKDFAGKTPLHLAADAGHLEIVEVLLKAGADVNAQDIFGKTPADIAADAGHEDIAEVLQKAAGSGSC

SEQ ID NO: 33 - protein, 296 aa, synthetic construct
  source 1..296: note = Ankyrin repeat protein (DARPin #23)
GSDLGEKLLLAAARGQDDEVRELLRAGADVNAKDEHGHTPLHLAAYIGHLEIVEVLLKAGADVNAKDEYGITPLHVAAYRGHLEIVEVLLKAGADVNAKDVYGWTPLHIAAEHGHLEIVEVLLKAGADVNAQDKSGKTPADLAARAGHQDIAEVLQKAAGSPTPTGSDLGKKLLEAARAGQDDEVRELLKAGADVNAKDYFSHTPLHLAARNGHLKIVEVLLKAGADVNAKDFAGKTPLHLAADAGHLEIVEVLLKAGADVNAQDIFGKTPADIAADAGHEDIAEVLQKAAGSGSC

SEQ ID NO: 34 - protein, 296 aa, synthetic construct
  source 1..296: note = Ankyrin repeat protein (DARPin #24)
GSDLGEKLLLAAARGQDDEVRELLRAGADVNAKDEHGHTPLHLAAYIGHLEIVEVLLKAGADVNAKDEYGITPLHVAAYRGHLEIVEVLLKAGADVNAKDVYGWTPLHIAAEHGHLEIVEVLLKAGADVNAQDKSGKTPADLAARAGHQDIAEVLQKAAPAASASAPDLGKKLLEAARAGQDDEVRELLKAGADVNAKDYFSHTPLHLAARNGHLKIVEVLLKAGADVNAKDFAGKTPLHLAADAGHLEIVEVLLKAGADVNAQDIFGKTPADIAADAGHEDIAEVLQKAAGSGSC

SEQ ID NO: 35 - protein, 30 aa, synthetic construct
  source 1..30: note = N-terminal capping module
DLGKKLLEAARAGQDDEVRELLKAGADVNA

SEQ ID NO: 36 - protein, 33 aa, synthetic construct
  source 1..33: note = Internal repeat module
KDAAGETPLHLAAWFGHLEIVEVLLKAGADVNA

SEQ ID NO: 37 - protein, 28 aa, synthetic construct
  source 1..28: note = C-terminal capping module
QDLQGKTPADLAAKEGHEDIAEVLQKAA

SEQ ID NO: 38 - protein, 279 aa, synthetic construct
  source 1..279: note = Ankyrin repeat protein (DARPin #25)
GSDLGLQLLQAAARGQDDTVRELLQAGADVNASDEWGHTPLHLAAYTGHLEIVEVLLEAGADVNASDQFGITPLHVAAYRGHLEIVEVLLEAGADVNAQDEAGLTPADLAARLGNEDIAEVLQQAAGSPTPTPTTPTPTPTTPTPTPTGSDLGKKLLEAARAGQDDEVRELLKAGADVNAKDYFSHTPLHLAARNGHLKIVEVLLKAGADVNAKDFAGKTPLHLAADAGHLEIVEVLLKAGADVNAQDIFGKTPADIAADAGHEDIAEVLQKAAGSGSC

SEQ ID NO: 39 - protein, 279 aa, synthetic construct
  source 1..279: note = Ankyrin repeat protein (DARPin #26)
GSDLGKKLLEAARAGQDDEVRELLKAGADVNAKDYFSHTPLHLAARNGHLKIVEVLLKAGADVNAKDFAGKTPLHLAADAGHLEIVEVLLKAGADVNAQDIFGKTPADIAADAGHEDIAEVLQKAAGSPTPTPTTPTPTPTTPTPTPTGSDLGLQLLQAAARGQDDTVRELLQAGADVNASDEWGHTPLHLAAYTGHLEIVEVLLEAGADVNASDQFGITPLHVAAYRGHLEIVEVLLEAGADVNAQDEAGLTPADLAARLGNEDIAEVLQQAAGSGSC

SEQ ID NO: 40 - protein, 279 aa, synthetic construct
  source 1..279: note = Ankyrin repeat protein (DARPin #27)
GSDLGKKLLEAARAGQDDEVRELLKAGADVNAKDYFSHTPLHLAARNGHLKIVEVLLKAGADVNAKDFAGKTPLHLAADAGHLEIVEVLLKAGADVNAQDIFGKTPADIAADAGHEDIAEVLQKAAGSPTPTPTTPTPTPTTPTPTPTGSDLGEKLLEAAKQGQGDEVRELLKAGADVSAKDQLGATPLHLAAAYGHLEIVEVLLKAGADVNAKDYLGWTPLHVAALAGHLEIVEVLLKFGADVNAQDRKGRTPADLAAKKGHEDIAEVLQKAAGSGSC

SEQ ID NO: 41 - protein, 124 aa, synthetic construct
  source 1..124: note = Ankyrin repeat domain (non-binding DARPin)
DLGKKLLEAARAGQDDEVRELLKAGADVNAKDKDGYTPLHLAAREGHLEIVEVLLKAGADVNAKDKDGYTPLHLAAREGHLEIVEVLLKAGADVNAQDKSGKTPADLAADAGHEDIAEVLQKAA

SEQ ID NO: 42 - protein, 124 aa, synthetic construct
DLGEKLLRAAAQGQDDEVRELLKAGADVNAKDHYGHTPLHVAAYWGHLEIVEVLLKAGADVNAKDEYGITPLHVAAYRGHLEIVEVLLKAGADVNAQDAAGLTPADLAARLGHDDIAEVLQKAA

SEQ ID NO: 43 - protein, 124 aa, synthetic construct
DLGLKLLQAAARGNPDEVRELLQAGADVNAKDEWGHTPLHLAAYTGHPEIVEVLLEAGADVNAKDQFGITPLHVAAYRGHPEIVEVLLEAGADVNAQDEAGLTPADLAARLGHEDIAEVLQKAA

SEQ ID NO: 44 - protein, 124 aa, unidentified
DLGEKLLRAAAQGNPDEVRELLQAGADVNAKDHYGHTPLHVAAYWGHPEIVEVLLEAGADVNAKDEYGITPLHVAAYRGHPEIVEVLLEAGADVNAQDAAGLTPADLAARLGHDDIAEVLQKAA

SEQ ID NO: 45 - protein, 124 aa, synthetic construct
DLGKKLLEAARAGNPDEVRELLQAGADVNAKDYFSHTPLHLAARNGHPEIVEVLLEAGADVNAKDFAGKTPLHLAADAGHPEIVEVLLEAGADVNAQDIFGKTPADIAADAGHEDIAEVLQKAA

SEQ ID NO: 46 - protein, 259 aa, synthetic construct
DLGEKLLRAAAQGQDDEVRELLKAGADVNAKDHYGHTPLHVAAYWGHLEIVEVLLKAGADVNAKDEYGITPLHVAAYRGHLEIVEVLLKAGADVNAQDAAGLTPADLAARLGHDDIAEVLQKAAGSPTPTPTTGSDLGKKLLEAARAGQDDEVRELLKAGADVNAKDYFSHTPLHLAARNGHLKIVEVLLKAGADVNAKDFAGKTPLHLAADAGHLEIVEVLLKAGADVNAQDIFGKTPADIAADAGHEDIAEVLQKAA

SEQ ID NO: 47 - protein, 259 aa, synthetic construct
DLGEKLLRAAAQGNPDEVRELLQAGADVNAKDHYGHTPLHVAAYWGHPEIVEVLLEAGADVNAKDEYGITPLHVAAYRGHPEIVEVLLEAGADVNAQDAAGLTPADLAARLGHDDIAEVLQKAAGSPTPTPTTGSDLGKKLLEAARAGQDDEVRELLKAGADVNAKDYFSHTPLHLAARNGHLKIVEVLLKAGADVNAKDFAGKTPLHLAADAGHLEIVEVLLKAGADVNAQDIFGKTPADIAADAGHEDIAEVLQKAA

SEQ ID NO: 48 - protein, 259 aa, synthetic construct
DLGEKLLRAAAQGNPDEVRELLQAGADVNAKDHYGHTPLHVAAYWGHPEIVEVLLEAGADVNAKDEYGITPLHVAAYRGHPEIVEVLLEAGADVNAQDAAGLTPADLAARLGHDDIAEVLQKAAGSPTPTPTTGSDLGKKLLEAARAGNPDEVRELLQAGADVNAKDYFSHTPLHLAARNGHPEIVEVLLEAGADVNAKDFAGKTPLHLAADAGHPEIVEVLLEAGADVNAQDIFGKTPADIAADAGHEDIAEVLQKAA

SEQ ID NO: 49 - protein, 259 aa, synthetic construct
DLGLKLLQAAARGNPDEVRELLQAGADVNAKDEWGHTPLHLAAYTGHPEIVEVLLEAGADVNAKDQFGITPLHVAAYRGHPEIVEVLLEAGADVNAQDEAGLTPADLAARLGHEDIAEVLQKAAGSPTPTPTTGSDLGKKLLEAARAGQDDEVRELLKAGADVNAKDYFSHTPLHLAARNGHLKIVEVLLKAGADVNAKDFAGKTPLHLAADAGHLEIVEVLLKAGADVNAQDIFGKTPADIAADAGHEDIAEVLQKAA

SEQ ID NO: 50 - protein, 259 aa, synthetic construct
DLGLKLLQAAARGNPDEVRELLQAGADVNAKDEWGHTPLHLAAYTGHPEIVEVLLEAGADVNAKDQFGITPLHVAAYRGHPEIVEVLLEAGADVNAQDEAGLTPADLAARLGHEDIAEVLQKAAGSPTPTPTTGSDLGKKLLEAARAGNPDEVRELLQAGADVNAKDYFSHTPLHLAARNGHPEIVEVLLEAGADVNAKDFAGKTPLHLAADAGHPEIVEVLLEAGADVNAQDIFGKTPADIAADAGHEDIAEVLQKAA

SEQ ID NO: 51 - protein, 266 aa, unidentified
GSDLGEKLLRAAAQGQDDEVRELLKAGADVNAKDHYGHTPLHVAAYWGHLEIVEVLLKAGADVNAKDEYGITPLHVAAYRGHLEIVEVLLKAGADVNAQDAAGLTPADLAARLGHDDIAEVLQKAAGSPTPTPTTGSDLGKKLLEAARAGQDDEVRELLKAGADVNAKDYFSHTPLHLAARNGHLKIVEVLLKAGADVNAKDFAGKTPLHLAADAGHLEIVEVLLKAGADVNAQDIFGKTPADIAADAGHEDIAEVLQKAAGSGSC

SEQ ID NO: 52 - protein, 266 aa, synthetic construct
GSDLGEKLLRAAAQGNPDEVRELLQAGADVNAKDHYGHTPLHVAAYWGHPEIVEVLLEAGADVNAKDEYGITPLHVAAYRGHPEIVEVLLEAGADVNAQDAAGLTPADLAARLGHDDIAEVLQKAAGSPTPTPTTGSDLGKKLLEAARAGQDDEVRELLKAGADVNAKDYFSHTPLHLAARNGHLKIVEVLLKAGADVNAKDFAGKTPLHLAADAGHLEIVEVLLKAGADVNAQDIFGKTPADIAADAGHEDIAEVLQKAAGSGSC

SEQ ID NO: 53 - protein, 266 aa, synthetic construct
GSDLGEKLLRAAAQGNPDEVRELLQAGADVNAKDHYGHTPLHVAAYWGHPEIVEVLLEAGADVNAKDEYGITPLHVAAYRGHPEIVEVLLEAGADVNAQDAAGLTPADLAARLGHDDIAEVLQKAAGSPTPTPTTGSDLGKKLLEAARAGNPDEVRELLQAGADVNAKDYFSHTPLHLAARNGHPEIVEVLLEAGADVNAKDFAGKTPLHLAADAGHPEIVEVLLEAGADVNAQDIFGKTPADIAADAGHEDIAEVLQKAAGSGSC

SEQ ID NO: 54 - protein, 266 aa, synthetic construct
GSDLGLKLLQAAARGNPDEVRELLQAGADVNAKDEWGHTPLHLAAYTGHPEIVEVLLEAGADVNAKDQFGITPLHVAAYRGHPEIVEVLLEAGADVNAQDEAGLTPADLAARLGHEDIAEVLQKAAGSPTPTPTTGSDLGKKLLEAARAGQDDEVRELLKAGADVNAKDYFSHTPLHLAARNGHLKIVEVLLKAGADVNAKDFAGKTPLHLAADAGHLEIVEVLLKAGADVNAQDIFGKTPADIAADAGHEDIAEVLQKAAGSGSC

SEQ ID NO: 55 - protein, 266 aa, synthetic construct
GSDLGLKLLQAAARGNPDEVRELLQAGADVNAKDEWGHTPLHLAAYTGHPEIVEVLLEAGADVNAKDQFGITPLHVAAYRGHPEIVEVLLEAGADVNAQDEAGLTPADLAARLGHEDIAEVLQKAAGSPTPTPTTGSDLGKKLLEAARAGNPDEVRELLQAGADVNAKDYFSHTPLHLAARNGHPEIVEVLLEAGADVNAKDFAGKTPLHLAADAGHPEIVEVLLEAGADVNAQDIFGKTPADIAADAGHEDIAEVLQKAAGSGSC

SEQ ID NO: 56 - protein, 266 aa, synthetic construct
GSDLGLKLLQAAARGQDDEVRELLKAGADVNAKDEWGHTPLHLAAYTGHLEIVEVLLKAGADVNAKDQFGITPLHVAAYRGHLEIVEVLLKAGADVNAQDEAGLTPADLAARLGHEDIAEVLQKAAGSPTPTPTTGSDLGKKLLEAARAGQDDEVRELLKAGADVNAKDYFSHTPLHLAARNGHLKIVEVLLKAGADVNAKDFAGKTPLHLAADAGHLEIVEVLLKAGADVNAQDIFGKTPADIAADAGHEDIAEVLQKAAGSGSC